(19) United States
(12) Patent Application Publication
(10) Pub. No.: US 2025/0041393 A1
(43) Pub. Date: Feb. 6, 2025

(54) POLYNUCLEOTIDES ENCODING INTEGRIN BETA-6 AND METHODS OF USE THEREOF

(71) Applicant: MODERNATX, INC., Cambridge, MA (US)

(72) Inventors: Eric Yi-Chun Huang, Boston, MA (US); Paul Stein, Watertown, MA (US); Laurie Kenney, Boston, MA (US); Michelle Dutra, Framingham, MA (US); Myoungjoo Kim, Cambridge, MA (US); Pranitha Vangala, Natick, MA (US)

(21) Appl. No.: 18/706,652

(22) PCT Filed: Nov. 1, 2022
(86) PCT No.: PCT/US2022/079095

§ 371 Date: May 1, 2024

Related U.S. Application Data
(60) Provisional application No. 63/274,211, filed on Nov. 1, 2021.

(51) Int. Cl. A61K 39/00; A61K 9/127; A61K 9/51; A61P 37/06; G01N 33/50

(52) U.S. Cl. CPC A61K 39/001; A61K 9/1271; A61K 9/5123; A61K 39/0008; A61P 37/06; G01N 33/5023; A61K 2039/53; A61K 2039/55555; G01N 2333/46; G01N 2333/70546; G01N 2333/82; G01N 2800/52

(57) ABSTRACT

The disclosure features lipid nanoparticle (LNP) compositions ITB6 molecules and uses thereof. The LNP compositions of the present disclosure comprise mRNA therapeutics encoding ITB6 polypeptides. The LNP compositions of the present disclosure can reprogram dendritic cells, suppress T cells and/or induce immune tolerance in vivo.

### CROSS REFERENCE TO RELATED APPLICATIONS

This application claims the benefit of U.S. Provisional Application No. 63/274,211, filed Nov. 1, 2021. The contents of the aforementioned application is hereby incorporated by reference in its entirety.

### BACKGROUND OF THE DISCLOSURE

Integrin β (ITGB) superfamily members play important roles in multiple biological functions, including immunosuppression.

Immune cells are considered to contribute to the development and/or progression of a wide variety of diseases, e.g., autoimmune diseases and/or inflammatory diseases. Much effort has been given to the development of therapies to suppress immune cells. However, there is an unmet need to develop therapies that can suppress immune cells for the treatment of autoimmune and/or inflammatory diseases.

### SUMMARY OF THE DISCLOSURE

The present disclosure provides, inter alia, lipid nanoparticle (LNP) compositions comprising a polynucleotide (e.g., an mRNA) encoding an ITB6 molecule and uses thereof. The LNP compositions of the present disclosure comprise nucleic acid (e.g., mRNA) therapeutics encoding an ITB6 polypeptide. In an aspect, the LNP compositions of the present disclosure can reprogram myeloid and/or dendritic cells, suppress T cells, and/or induce immune tolerance in vivo. Also disclosed herein are methods of using an LNP composition comprising a polynucleotide (e.g., an mRNA) encoding an ITB6 molecule, for treating a disease associated with an aberrant T cell function, or for inhibiting an immune response in a subject. While both ITG6 and ITB8 have similar function and can be used in the methods described herein, ITB6 was found to yield superior results when employed in the subject compositions. Additional aspects of the disclosure are described in further detail below.

In an aspect, disclosed herein is a lipid nanoparticle (LNP) composition comprising a polynucleotide comprising an mRNA which encodes an ITB6 molecule.

In another aspect, the disclosure provides a lipid nanoparticle (LNP) composition for immunomodulation, e.g., for inducing immune tolerance or reprogramming immune cells, the composition comprising a polynucleotide comprising an mRNA which encodes an ITB6 molecule.

In some embodiments, the ITB6 molecule comprises an amino acid sequence having at least 85%, 90%, 95%, 96%, 97%, 98%, 99%, or 100% identity to, or differing by no more than 1, 2, 5, 10, 15, 20, 25, 30, 40, 50, 60, 70, 80, 90, or 100 amino acids from, an amino acid sequence of ITB6 provided in Table 1A or Table 2A, e.g., any one of SEQ ID NOs: 17, 1, 7, 9, 11, 13, or 15, or a functional fragment thereof.

In some embodiments, the LNP composition comprises an amino acid sequence of any one of SEQ ID NOs: 17, 1, 7, 9, 11, 13, or 15, or a functional fragment thereof.

In some embodiments, the LNP composition comprises a nucleic acid sequence having at least 85%, 90%, 95%, 96%, 97%, 98%, 99% or 100% identity to, or differing by no more than 10, 25, 50, 100, 150, 200, 250, or 300 nucleotides from, a nucleic acid sequence of any one of SEQ ID NOs: 18, 2-6, 8, 10, 12, 14, 16, or 160-175, or a functional fragment thereof.

In some embodiments, the LNP composition results in suppression of a T cell activity and/or a T cell function (e.g., T cell anergy and/or T cell apoptosis) in a population of immune cells, e.g., as compared to a T cell activity and/or a T cell function in an otherwise similar or identical population of immune cells which has not been contacted with the LNP composition comprising a polynucleotide comprising an mRNA which encodes an ITB6 molecule.

In some embodiments, suppression of a T cell activity and/or a T cell function comprises any one, two, three, four, or all of the following:
(i) reduced level and/or activity of an effector cytokine, e.g., IFNg (e.g., secreted IFNg);
(ii) reduced T cell proliferation, survival, and/or expansion;
(iii) increased T cell apoptosis;
(iv) reduced expression and/or activity of a T cell transcription factor, e.g., T-bet; and/or
(v) increased Treg differentiation.

In some embodiments, the LNP composition results in:
(i) reduced engraftment of donor cells, e.g., donor immune cells, e.g., T cells, in a subject or host, e.g., a human, rat or mouse;
(ii) reduction in the level, activity and/or secretion of IFNg from engrafted donor immune cells, e.g., T cells, in a subject or host, e.g., a human, rat or mouse; and/or
(iii) an absence of, prevention of, or delay in the onset of, graft vs host disease (GvHD) in a subject or a host, e.g., a human, rat or mouse.

In some embodiments, the LNP composition results in amelioration or reduction of a symptom of GvHD, e.g., reduction of weight loss, host B cell depletion, and/or donor T cell engraftment, in a subject, e.g., as measured by an assay described in Example 6. In some embodiments, the LNP composition further results in Treg expansion in the subject, e.g., as measured by an assay described in Example 6.

In an aspect, disclosed herein is a pharmaceutical composition comprising an LNP comprising a polynucleotide comprising an mRNA which encodes an ITB6 molecule, e.g., as described herein.

In another aspect, the disclosure provides a method of modulating, e.g., suppressing, an immune response, comprising administering to a subject in need thereof an effective amount of a polynucleotide comprising an mRNA which encodes an ITB6 molecule, or an LNP composition comprising the same, thereby modulating the immune response.

More specifically, the subject compositions can increase Treg cell function (e.g., by increasing the number of Treg cells) and reduce T cell proliferation, thereby leading to reduction in symptoms and/or severity of autoimmune disease. In one embodiment, the increase in Treg cells occurs in an antigen-dependent manner.

In yet another aspect, provided herein is a method of treating or preventing a disease or a symptom thereof, comprises administering to a subject in need thereof an effective amount of a polynucleotide comprising an mRNA which encodes an ITB6 molecule, or an LNP composition comprising the same, thereby treating or preventing the disease.

In some embodiments, the disease is an ITB6-associated disease, e.g., a disease associated with the expression and/or an activity of ITB6. In some embodiments, the disease is a disease associated with an aberrant immune cell (e.g., T cell) function, e.g., an autoimmune disease or an inflammatory disease.

In another aspect, the disclosure provides a polynucleotide comprising an mRNA which encodes an ITB6 molecule, or an LNP composition comprising the same, for use in a method of modulating, e.g., suppressing, an immune response in a subject.

In yet another aspect, provided herein is a polynucleotide comprising an mRNA which encodes an ITB6 molecule, or an LNP composition comprising the same, for use in a method of treating or preventing a disease or a symptom thereof.

In some embodiments, the disease is an ITB6-associated disease, e.g., a disease associated with the expression and/or an activity of ITB6. In some embodiments, the disease is a disease associated with an aberrant immune cell (e.g., T cell) function, e.g., an autoimmune disease or an inflammatory disease.

In another aspect, the disclosure provides use of a polynucleotide comprising an mRNA which encodes an ITB6 molecule, or an LNP composition comprising the same, in the manufacture of a medicament for modulating, e.g., suppressing, an immune response in a subject.

In yet another aspect, provided herein is use of a polynucleotide comprising an mRNA which encodes an ITB6 molecule, or an LNP composition comprising the same, in the manufacture of a medicament for treating or preventing a disease or a symptom thereof.

In some embodiments, the disease is an ITB6-associated disease, e.g., a disease associated with the expression and/or an activity of ITB6. In some embodiments, the disease is a disease associated with an aberrant immune cell (e.g., T cell) function, e.g., an autoimmune disease or an inflammatory disease.

In another aspect, the disclosure provides use of a polynucleotide comprising an mRNA which encodes an ITB6 molecule, or an LNP composition comprising the same, for modulating, e.g., suppressing, an immune response in a subject.

In yet another aspect, provided herein is use a polynucleotide comprising an mRNA which encodes an ITB6 molecule, or an LNP composition comprising the same, for treating or preventing a disease or a symptom thereof.

In another aspect, the disclosure provides a method of assessing the responsiveness of a subject to a therapy comprising an LNP composition comprising an mRNA which encodes an ITB6 molecule, the method comprising:
(a) measuring the expression level of one or more (e.g., 2, 3, 4, or 5) biomarkers (e.g., selected from the group consisting of: PMEPA1, ITGAE/CD103, SMAD7, SKIL, and SKI) in a sample from the subject collected following administration of the therapy; and
(b) comparing the expression level in the sample with a reference expression level, wherein an increase in the expression level of the one or more biomarkers is indicative of a response to the therapy.

In some embodiments, the one or more biomarkers are one or more (e.g., 2, 3, 4, or 5) of PMEPA1, ITGAE/CD103, SMAD7, SKIL, and SKI.

In some embodiments, the level of one or more of the one or more biomarkers in the sample from the subject following treatment is at least 2-fold (e.g., at least 3-fold, 4-fold, 5-fold, 6-fold, 7-fold, 8-fold, 9-fold, or 10-fold) greater than a reference expression level of the one or more biomarkers, wherein the reference expression level comprises:
(a) the expression level of the one or more biomarkers in a sample from the subject prior to treatment with an ITB6 mRNA; or
(b) the expression level of o the one or more biomarkers in a sample from a subject who has not received the treatment with an ITB6 mRNA.

In some embodiments, where the expression level of the one or more (e.g., 2, 3, 4, or 5) biomarkers compared to a reference expression level indicates a responsiveness to the therapy (e.g., a therapy comprising an ITB6 mRNA), the method further includes administering one or more additional doses of the therapy to the subject.

In some embodiments, the disease is an ITB6-associated disease, e.g., a disease associated with the expression and/or an activity of ITB6. In some embodiments, the disease is a disease associated with an aberrant immune cell (e.g., T cell) function, e.g., an autoimmune disease or an inflammatory disease.

In another aspect, the disclosure provides use of a polynucleotide comprising an mRNA which encodes an ITB6 molecule, or an LNP composition comprising the same, as a medicament.

Additional features of any of the LNP compositions, pharmaceutical compositions comprising said LNPs, methods or compositions for use, as disclosed herein, can include the following embodiments.

In some embodiments of any of the methods disclosed herein, the LNP composition comprises a polynucleotide (e.g., mRNA) described herein. In some embodiments, the ITB6 molecule comprises a naturally occurring ITB6 molecule, a fragment of a naturally occurring ITB6 molecule, or a variant thereof. In some embodiments, the ITB6 molecule comprises an amino acid sequence having at least 85%, 90%, 95%, 96%, 97%, 98%, 99%, or 100% identity to, or differing by no more than 1, 2, 5, 10, 15, 20, 25, 30, 40, 50, 60, 70, 80, 90, or 100 amino acids from, an ITB6 amino acid sequence provided in Table 1A or Table 2A, e.g., any one of SEQ ID NOs: 17, 1, 7, 9, 11, 13, or 15, or a functional fragment thereof. In some embodiments, the ITB6 molecule comprises the amino acid sequence of an ITB6 amino acid sequence provided in Table 1A or Table 2A, e.g., any one of SEQ ID NOs: 17, 1, 7, 9, 11, 13, or 15, or a functional fragment thereof. In some embodiments, the ITB6 molecule comprises the amino acid sequence of SEQ ID NO: 17. In some embodiments, the ITB6 molecule comprises the amino acid sequence of SEQ ID NO: 1. In some embodiments, the ITB6 molecule comprises the amino acid sequence of SEQ ID NO: 7. In some embodiments, the ITB6 molecule comprises the amino acid sequence of SEQ ID NO:9. In some embodiments, the ITB6 molecule comprises the amino acid sequence of SEQ ID NO: 11. In some embodiments, the ITB6 molecule comprises the amino acid sequence of SEQ ID NO: 13. In some embodiments, the ITB6 molecule comprises the amino acid sequence of SEQ ID NO: 15.

In some embodiments, the ITB6 molecule comprises an amino acid sequence for a leader sequence and/or an affinity tag. In some embodiments, the ITB6 molecule does not comprise an amino acid sequence for a leader sequence and/or an affinity tag.

In some embodiments, the ITB6 molecule comprises an amino acid sequence having at least 85%, 90%, 95%, 96%, 97%, 98%, 99%, or 100% identity to, or differing by no more than 1, 2, 5, 10, 15, 20, 25, 30, 40, 50, 60, 70, 80, 90, or 100 amino acids from, an amino acid sequence lacking a leader sequence and/or an affinity tag (e.g., a leader sequence and/or an affinity tag described in Table 1A or Table 2A) but otherwise identical to an amino acid sequence of ITB6 provided in Table 1A or Table 2A, e.g., any one of SEQ ID NOs: 17, 1, 7, 9, 11, 13, or 15, or a functional fragment thereof.

In some embodiments, the polynucleotide (e.g., mRNA) encoding an ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of any one of SEQ ID NOs: 18, 2-6, 8, 10, 12, 14, 16, or 160-175, or a functional fragment thereof. In some embodiments, the polynucleotide (e.g., mRNA) encoding an ITB6 molecule comprises a nucleotide sequence of any one of SEQ ID NOs: 18, 2-6, 8, 10, 12, 14, 16, or 160-175, or a functional fragment thereof.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 18. In some embodiments, the polynucleotide (e.g., mRNA) encoding the ITB6 molecule comprises the nucleotide sequence of SEQ ID NO: 18. In some embodiments, the polynucleotide (e.g., mRNA) encoding the ITB6 molecule comprises the nucleotide sequence of SEQ ID NO: 175, which comprises from 5′ to 3′ end: the 5′ UTR sequence of SEQ ID NO: 50, the ORF sequence of SEQ ID NO:18, and the 3′ UTR sequence of SEQ ID NO: 142.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 2. In some embodiments, the polynucleotide (e.g., mRNA) encoding the ITB6 molecule comprises the nucleotide sequence of SEQ ID NO: 2. In some embodiments, the polynucleotide (e.g., mRNA) encoding the ITB6 molecule comprises the nucleotide sequence of SEQ ID NO: 160, which comprises from 5′ to 3′ end: the 5′ UTR sequence of SEQ ID NO: 56, the ORF sequence of SEQ ID NO: 2, and the 3′ UTR sequence of SEQ ID NO: 110.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 3. In some embodiments, the polynucleotide (e.g., mRNA) encoding the ITB6 molecule comprises the nucleotide sequence of SEQ ID NO: 3. In some embodiments, the polynucleotide (e.g., mRNA) encoding the ITB6 molecule comprises the nucleotide sequence of SEQ ID NO: 161, which comprises from 5′ to 3′ end: the 5′ UTR sequence of SEQ ID NO: 50, the ORF sequence of SEQ ID NO: 3, and the 3′ UTR sequence of SEQ ID NO: 110.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 4. In some embodiments, the polynucleotide (e.g., mRNA) encoding the ITB6 molecule comprises the nucleotide sequence of SEQ ID NO: 4. In some embodiments, the polynucleotide (e.g., mRNA) encoding the ITB6 molecule comprises the nucleotide sequence of SEQ ID NO: 162, which comprises from 5′ to 3′ end: the 5′ UTR sequence of SEQ ID NO: 50, ORF sequence of SEQ ID NO: 4, and the 3′ UTR sequence of SEQ ID NO: 143.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 5. In some embodiments, the polynucleotide (e.g., mRNA) encoding the ITB6 molecule comprises the nucleotide sequence of SEQ ID NO: 5. In some embodiments, the polynucleotide (e.g., mRNA) encoding the ITB6 molecule comprises the nucleotide sequence of SEQ ID NO: 163, which comprises from 5′ to 3′ end: the 5′ UTR sequence of SEQ ID NO: 50, the ORF sequence of SEQ ID NO: 5, and the 3′ UTR sequence of SEQ ID NO: 110. In some embodiments, the polynucleotide (e.g., mRNA) encoding the ITB6 molecule comprises the nucleotide sequence of SEQ ID NO: 164, which comprises from 5′ to 3′ end: the 5′ UTR sequence of SEQ ID NO: 50, the ORF sequence of SEQ ID NO: 5, and the 3′ UTR sequence of SEQ ID NO: 144.

In some embodiments, the polynucleotide (e.g., mRNA) encoding the ITB6 molecule comprises the nucleotide sequence of SEQ ID NO: 169, which comprises from 5′ to 3′ end: the 5′ UTR sequence of SEQ ID NO: 50, the ORF sequence of SEQ ID NO: 5, and the 3′ UTR sequence of SEQ ID NO: 145.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 6. In some embodiments, the polynucleotide (e.g., mRNA) encoding the ITB6 molecule comprises the nucleotide sequence of SEQ ID NO: 6. In some embodiments, the polynucleotide (e.g., mRNA) encoding the ITB6 molecule comprises the nucleotide sequence of SEQ ID NO: 165, which comprises from 5′ to 3′ end: the 5′ UTR sequence of SEQ ID NO: 50, the ORF sequence of SEQ ID NO: 6, and the 3′ UTR sequence of SEQ ID NO: 110. In some embodiments, the polynucleotide (e.g., mRNA) encoding the ITB6 molecule comprises the nucleotide sequence of SEQ ID NO: 166, which comprises from 5′ to 3′ end: the 5′ UTR sequence of SEQ ID NO: 50, the ORF sequence of SEQ ID NO: 6, and the 3′ UTR sequence of SEQ ID NO: 110. In some embodiments, the polynucleotide (e.g., mRNA) encoding the ITB6 molecule comprises the nucleotide sequence of SEQ ID NO: 167, which comprises from 5′ to 3′ end: the 5′ UTR sequence of SEQ ID NO: 50, the ORF sequence of SEQ ID NO: 6, and the 3′ UTR sequence of SEQ ID NO: 145. In some embodiments, the polynucleotide (e.g., mRNA) encoding the ITB6 molecule comprises the nucleotide sequence of SEQ ID NO: 168, which comprises from 5′ to 3′ end: the 5′ UTR sequence of SEQ ID NO: 50, the ORF sequence of SEQ ID NO: 6, and the 3′ UTR sequence of SEQ ID NO: 143.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 8. In some embodiments, the polynucleotide (e.g., mRNA) encoding the ITB6 molecule comprises the nucleotide sequence of SEQ ID NO: 8. In some embodiments, the polynucleotide (e.g., mRNA) encoding the ITB6 molecule comprises the nucleotide sequence of SEQ ID NO: 170, which comprises from 5′ to 3′ end: the 5′ UTR sequence of SEQ ID NO: 50, the ORF sequence of SEQ ID NO: 8, and the 3′ UTR sequence of SEQ ID NO: 110.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 10. In some embodiments, the polynucleotide (e.g., mRNA) encoding the ITB6 molecule comprises the nucleotide sequence of SEQ ID NO: 10. In some embodiments, the polynucleotide (e.g., mRNA) encoding the ITB6 molecule comprises the nucleotide sequence of SEQ ID NO: 171, which comprises from 5′ to 3′ end: the 5′ UTR sequence of SEQ ID NO: 50, the ORF sequence of SEQ ID NO: 10, and the 3′ UTR sequence of SEQ ID NO: 110.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 12. In some embodiments, the polynucleotide (e.g., mRNA) encoding the ITB6 molecule comprises the nucleotide sequence of SEQ ID NO: 12. In some embodiments, the polynucleotide (e.g., mRNA) encoding the ITB6 molecule comprises the nucleotide sequence of SEQ ID NO: 172, which comprises from 5′ to 3′ end: the 5′ UTR sequence of SEQ ID NO: 50, the ORF sequence of SEQ ID NO: 12, and the 3′ UTR sequence of SEQ ID NO: 110.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the sequence of SEQ ID NO: 14. In some embodiments, the polynucleotide (e.g., mRNA) encoding the ITB6 molecule comprises the nucleotide sequence of SEQ ID NO: 14. In some embodiments, the polynucleotide (e.g., mRNA) encoding the ITB6 molecule comprises the nucleotide sequence of SEQ ID NO: 173, which comprises from 5′ to 3′ end: the 5′ UTR sequence of SEQ ID NO: 50, the ORF sequence of SEQ ID NO: 14, and the 3′ UTR sequence of SEQ ID NO: 110.

In some embodiments, the polynucleotide (e.g., mRNA) encoding the ITB6 molecule comprises a nucleotide sequence having at least 85%, 90%, 95%, 96%, 97%, 98%, 99%, or 100% identity to, or differing by no more than 10, 25, 50, 100, 150, 200, 250, or 300 nucleotides from, the nucleotide sequence of SEQ ID NO: 16. In some embodiments, the polynucleotide (e.g., mRNA) encoding the ITB6 molecule comprises the nucleotide sequence of SEQ ID NO: 16. In some embodiments, the polynucleotide (e.g., mRNA) encoding the ITB6 molecule comprises the nucleotide sequence of SEQ ID NO: 174, which comprises from 5′ to 3′ end: the 5′ UTR sequence of SEQ ID NO: 56, the ORF sequence of SEQ ID NO: 16, and the 3′ UTR sequence of SEQ ID NO: 110.

In some embodiments, the polynucleotide (e.g., mRNA) encoding the ITB6 molecule comprises the nucleotide sequence of any of ITB6 human variant 5.1, human variant 1.1, human variant 1.2, human variant 1.3, human variant 1.4, human variant 1.5, human variant 1.6, human variant 1.7, human variant 1.8, human variant 1.9, human variant 1.10, human variant 2.1, human variant 3.1, or human variant 4.1, rat variant 1.1, or mouse variant 1.1, as described in Table 2A.

In some embodiments, the polynucleotide (e.g., mRNA) encoding the ITB6 molecule comprises a nucleotide sequence that encodes for a leader sequence and/or an affinity tag. In some embodiments, the polynucleotide (e.g., mRNA) encoding the ITB6 molecule does not comprise a nucleotide sequence that encodes for a leader sequence and/or an affinity tag.

In some embodiments, the polynucleotide (e.g., mRNA) encoding the ITB6 molecule comprises a nucleic acid sequence having at least 85%, 90%, 95%, 96%, 97%, 98%, 99% or 100% identity to, or differing by no more than 10, 25, 50, 100, 150, 200, 250, or 300 nucleotides from, a nucleic acid sequence lacking a nucleotide sequence that encodes for a leader sequence and/or an affinity tag (e.g., a leader sequence and/or an affinity tag described in Table 1A or Table 2A) but otherwise identical to the nucleotide sequence of any one of SEQ ID NOs: 18, 2-6, 8, 10, 12, 14, 16, or 160-175, or a functional fragment thereof.

In some embodiments, the polynucleotide (e.g., mRNA) comprises at least one chemical modification. In some embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, the chemical modification is selected from the group consisting of pseudouridine, N1-methylpseudouridine, 5-methylcytosine, 5-methoxyuridine, and a combination thereof. In some embodiments, the chemical modification is N1-methylpseudouridine. In some embodiments, each mRNA in the lipid nanoparticle comprises fully modified N1-methylpseudouridine.

In some embodiments of any of the methods disclosed herein, the LNP composition is an LNP composition described herein. In some embodiments, the LNP composition comprises: (i) an ionizable lipid, e.g., an amino lipid; (ii) a sterol or other structural lipid; (iii) a non-cationic helper lipid or phospholipid; and (iv) a PEG-lipid.

In some embodiments, the LNP composition comprises an ionizable lipid comprising an amino lipid. In some embodiments, the ionizable lipid comprises a compound of any of Formulae (I), (I-a), (I-b), (I-c), (II), (II-a), (II-b), (II-c), (II-d), (II-e), (II-f), (II-g), (II-h), or (III). In an embodiment, the ionizable lipid comprises a compound of Formula (I). In an embodiment, the ionizable lipid comprises Compound 18. In an embodiment, the ionizable lipid comprises Compound 25.

In some embodiments, the lipid nanoparticle comprises a compound of Formula (I):

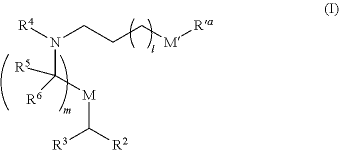

or a salt or isomer thereof,
wherein R′^{a} is R′^{branched}, wherein
   R′^{branched} is:

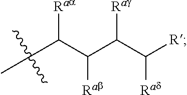

wherein

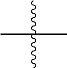

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

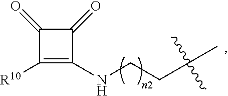

wherein

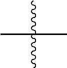

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the compound of Formula (I) is selected from:

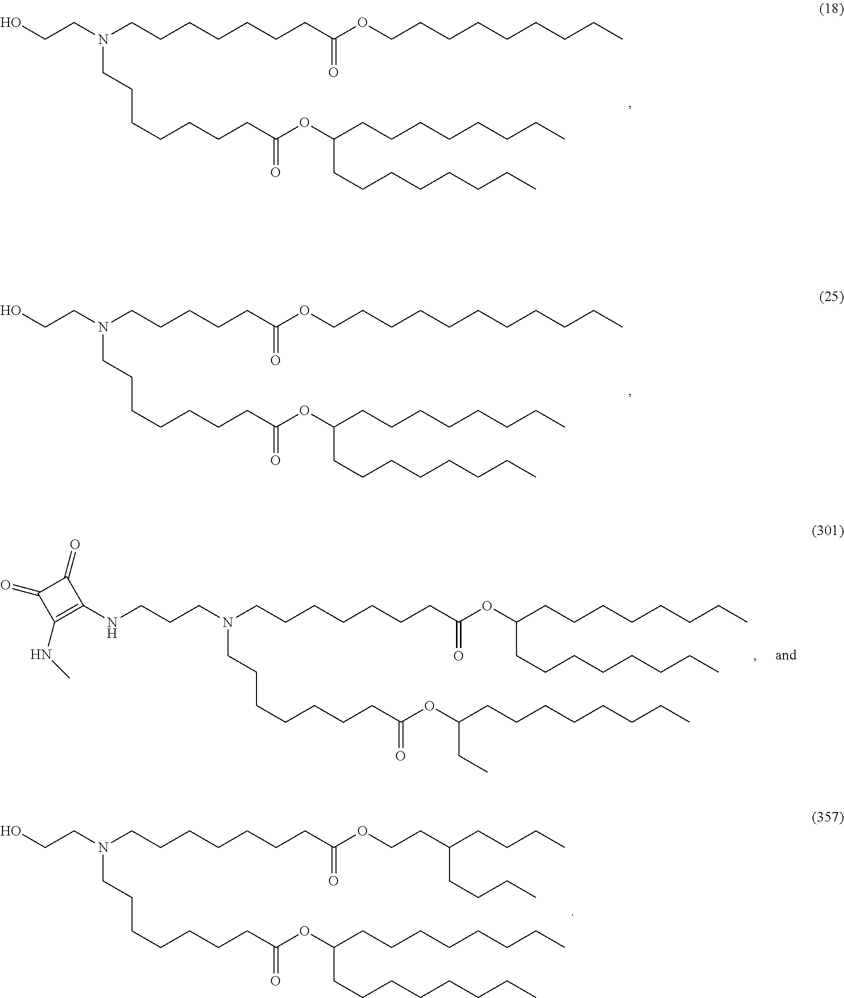 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the lipid nanoparticle further comprises a phospholipid, a structural lipid, and a PEG-lipid.

In some embodiments, the PEG-lipid is Compound I.

In some embodiments, the lipid nanoparticle comprises:
(i) 40-50 mol % of the compound of Formula (I), 30-45 mol % of the structural lipid, 5-15 mol % of the phospholipid, and 1-5 mol % of the PEG-lipid; or
(ii) 45-50 mol % of the compound of Formula (I), 35-45 mol % of the structural lipid, 8-12 mol % of the phospholipid, and 1.5 to 3.5 mol % of the PEG-lipid.

In some embodiments, the lipid nanoparticle comprises:
(i) Compound 18, (ii) Cholesterol, and (iii) PEG-DMG or Compound VI;
(i) Compound 25, (ii) Cholesterol, and (iii) PEG-DMG or Compound VI;
(i) Compound 301, (ii) Cholesterol, and (iii) PEG-DMG or Compound VI;
(i) Compound 357, (ii) Cholesterol, and (iii) PEG-DMG or Compound VI;
(i) Compound 18, (ii) DSPC or DOPE, (iii) Cholesterol, and (iv) PEG-DMG or Compound VI;
(i) Compound 25, (ii) DSPC or DOPE, (iii) Cholesterol, and (iv) PEG-DMG or Compound VI;
(i) Compound 301, (ii) DSPC or DOPE, (iii) Cholesterol, and (iv) PEG-DMG or Compound VI;
(i) Compound 357, (ii) DSPC or DOPE, (iii) Cholesterol, and (iv) PEG-DMG or Compound VI.

In some embodiments, the LNP comprises about 20 mol % to about 60 mol % ionizable lipid, about 5 mol % to about 25 mol % non-cationic helper lipid or phospholipid, about 25 mol % to about 55 mol % sterol or other structural lipid, and about 0.5 mol % to about 15 mol % PEG lipid. In some embodiments, the LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In some embodiments, the LNP comprises about 50 mol % ionizable lipid, about 10 mol % non-cationic helper lipid or phospholipid, about 38.5 mol % sterol or other structural lipid, and about 1.5 mol % PEG lipid. In some embodiments, the LNP comprises about 49.83 mol % ionizable lipid, about 9.83 mol % non-cationic helper lipid or phospholipid, about 30.33 mol % sterol or other structural lipid, and about 2.0 mol % PEG lipid.

In some embodiments, the polynucleotide (e.g., a RNA, e.g., a mRNA) disclosed herein is formulated with a delivery agent comprising, e.g., a compound having the Formula (II). In some embodiments, the delivery agent comprises an ionizable amino lipid, a helper lipid (e.g., DSPC), a sterol (e.g., Cholesterol), and a PEG lipid (e.g., Compound VI or PEG-DMG), e.g., with a mole ratio in the range of about (i) 40-50 mol % ionizable amino lipid, optionally 45-50 mol % ionizable amino lipid, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %; (ii) 30-45 mol % sterol (e.g., cholesterol), optionally 35-42 mol % sterol, for example, 30-31 mol %, 31-32 mol %, 32-33 mol %, 33-34 mol %, 35-35 mol %, 35-36 mol %, 36-37 mol %, 37-38 mol %, 38-39 mol %, or 39-40 mol %, or 40-42 mol % sterol; (iii) 5-15 mol % helper lipid (e.g., DSPC), optionally 10-15 mol % helper lipid, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % helper lipid; and (iv) 1-5% PEG lipid (e.g., Compound I or PEG-DMG), optionally 1-5 mol % PEG lipid, for example 1.5 to 2.5 mol %, 1-2 mol %, 2-3 mol %, 3-4 mol %, or 4-5 mol % PEG lipid. In some embodiments, the delivery agent comprises an ionizable amino lipid, Cholesterol, DSPC, and Compound I with a mole ratio of 47:39:11:3.

In some embodiments of any of the LNP compositions, methods or compositions for use disclosed herein, the LNP comprises about 45 mol % to about 50 mol % ionizable lipid. In some embodiments, the LNP comprises about 45.5 mol % to about 49.5 mol % ionizable lipid. In some embodiments, the LNP comprises about 46 mol % to about 49 mol % ionizable lipid. In some embodiments, the LNP comprises about 46.5 mol % to about 48.5 mol % ionizable lipid. In some embodiments, the LNP comprises about 47 mol % to about 48 mol % ionizable lipid.

In some embodiments, the LNP comprises about 45 mol % to about 49.5 mol % ionizable lipid. In some embodiments, the LNP comprises about 45 mol % to about 49 mol % ionizable lipid. In some embodiments, the LNP comprises about 45 mol % to about 48.5 mol % ionizable lipid. In some embodiments, the LNP comprises about 45 mol % to about 48 mol % ionizable lipid. In some embodiments, the LNP comprises about 45 mol % to about 47.5 mol % ionizable lipid. In some embodiments, the LNP comprises about 45 mol % to about 47 mol % ionizable lipid. In some embodiments, the LNP comprises about 45 mol % to about 46.5 mol % ionizable lipid. In some embodiments, the LNP comprises about 45 mol % to about 46 mol % ionizable lipid. In some embodiments, the LNP comprises about 45 mol % to about 45.5 mol % ionizable lipid.

In some embodiments, the LNP comprises about 45.5 mol % to about 50 mol % ionizable lipid. In some embodiments, the LNP comprises about 46 mol % to about 50 mol % ionizable lipid. In some embodiments, the LNP comprises about 46.5 mol % to about 50 mol % ionizable lipid. In some embodiments, the LNP comprises about 47 mol % to about 50 mol % ionizable lipid. In some embodiments, the LNP comprises about 47.5 mol % to about 50 mol % ionizable lipid. In some embodiments, the LNP comprises about 48 mol % to about 50 mol % ionizable lipid. In some embodiments, the LNP comprises about 48.5 mol % to about 50 mol % ionizable lipid. In some embodiments, the LNP comprises about 49 mol % to about 50 mol % ionizable lipid. In some embodiments, the LNP comprises about 49.5 mol % to about 50 mol % ionizable lipid.

In some embodiments, the LNP comprises about 45 mol % to about 46 mol % ionizable lipid. In some embodiments, the LNP comprises about 45.5 mol % to about 46.5 mol % ionizable lipid. In some embodiments, the LNP comprises about 46 mol % to about 47 mol % ionizable lipid. In some embodiments, the LNP comprises about 46.5 mol % to about 47.5 mol % ionizable lipid. In some embodiments, the LNP comprises about 47 mol % to about 48 mol % ionizable lipid. In some embodiments, the LNP comprises about 47.5 mol % to about 48.5 mol % ionizable lipid. In some embodiments, the LNP comprises about 48 mol % to about 49 mol % ionizable lipid. In some embodiments, the LNP comprises about 48.5 mol % to about 49.5 mol % ionizable lipid. In some embodiments, the LNP comprises about 49 mol % to about 50 mol % ionizable lipid.

In some embodiments, the LNP comprises about 45 mol % ionizable lipid. In some embodiments, the LNP comprises about 45.5 mol % ionizable lipid. In some embodiments, the LNP comprises about 46 mol % ionizable lipid. In some embodiments, the LNP comprises about 46.5 mol % ionizable lipid. In some embodiments, the LNP comprises about 47 mol % ionizable lipid. In some embodiments, the LNP comprises about 47.5 mol % ionizable lipid. In some embodiments, the LNP comprises about 48 mol % ionizable lipid. In some embodiments, the LNP comprises about 48.5 mol % ionizable lipid. In some embodiments, the LNP comprises about 49 mol % ionizable lipid. In some embodiments, the LNP comprises about 49.5 mol % ionizable lipid. In some embodiments, the LNP comprises about 50 mol % ionizable lipid.

In some embodiments, the LNP comprises about 1 mol % to about 5 mol % PEG lipid. In some embodiments, the LNP comprises about 1.5 mol % to about 4.5 mol % PEG lipid. In some embodiments, the LNP comprises about 2 mol % to about 4 mol % PEG lipid. In some embodiments, the LNP comprises about 2.5 mol % to about 3.5 mol % PEG lipid.

In some embodiments, the LNP comprises about 1 mol % to about 4.5 mol % PEG lipid. In some embodiments, the LNP comprises about 1 mol % to about 4 mol % PEG lipid. In some embodiments, the LNP comprises about 1 mol % to about 3.5 mol % PEG lipid. In some embodiments, the LNP comprises about 1 mol % to about 3 mol % PEG lipid. In some embodiments, the LNP comprises about 1 mol % to about 2.5 mol % PEG lipid. In some embodiments, the LNP comprises about 1 mol % to about 2 mol % PEG lipid. In some embodiments, the LNP comprises about 1 mol % to about 1.5 mol % PEG lipid.

In some embodiments, the LNP comprises about 1.5 mol % to about 5 mol % PEG lipid. In some embodiments, the LNP comprises about 2 mol % to about 5 mol % PEG lipid. In some embodiments, the LNP comprises about 2.5 mol % to about 5 mol % PEG lipid. In some embodiments, the LNP comprises about 3 mol % to about 5 mol % PEG lipid. In some embodiments, the LNP comprises about 3.5 mol % to about 5 mol % PEG lipid. In some embodiments, the LNP comprises about 4 mol % to about 5 mol % PEG lipid. In some embodiments, the LNP comprises about 4.5 mol % to about 5 mol % PEG lipid.

In some embodiments, the LNP comprises about 1 mol % to about 2 mol % PEG lipid. In some embodiments, the LNP comprises about 1.5 mol % to about 2.5 mol % PEG lipid. In some embodiments, the LNP comprises about 2 mol % to about 3 mol % PEG lipid. In some embodiments, the LNP comprises about 3.5 mol % to about 4.5 mol % PEG lipid. In some embodiments, the LNP comprises about 4 mol % to about 5 mol % PEG lipid.

In some embodiments, the LNP comprises about 1 mol % PEG lipid. In some embodiments of the LNPs or methods of the disclosure, the LNP comprises about 1.5 mol % PEG lipid. In some embodiments of the LNPs or methods of the disclosure, the LNP comprises about 2 mol % PEG lipid. In some embodiments of the LNPs or methods of the disclosure, the LNP comprises about 2.5 mol % PEG lipid. In some embodiments of the LNPs or methods of the disclosure, the LNP comprises about 3 mol % PEG lipid. In some embodiments of the LNPs or methods of the disclosure, the LNP comprises about 3.5 mol % PEG lipid. In some embodiments of the LNPs or methods of the disclosure, the LNP comprises about 4 mol % PEG lipid. In some embodiments of the LNPs or methods of the disclosure, the LNP comprises about 4.5 mol % PEG lipid. In some embodiments of the LNPs or methods of the disclosure, the LNP comprises about 5 mol % PEG lipid.

In some embodiments of the LNPs or methods of the disclosure, the LNP comprises about 50 mol % Compound 18 and about 10 mol % non-cationic helper lipid or phospholipid. In some embodiments of the LNPs or methods of the disclosure, the LNP comprises 50 mol % Compound 18 and about 10 mol % non-cationic helper lipid or phospholipid. In some embodiments of the LNPs or methods of the disclosure, the LNP comprises about 50 mol % Compound 18 and 10 mol % non-cationic helper lipid or phospholipid. In some embodiments of the LNPs or methods of the disclosure, the LNP comprises 50 mol % Compound 18 and 10 mol % non-cationic helper lipid or phospholipid. In some embodiments of the LNPs or methods of the disclosure, the LNP comprises about 49.83 mol % Compound 18, about 9.83 mol % non-cationic helper lipid or phospholipid, about 30.33 mol % sterol or other structural lipid, and about 2.0 mol % PEG lipid.

In some embodiments of the LNPs or methods of the disclosure, the LNP comprises about 50 mol % Compound 25 and about 10 mol % non-cationic helper lipid or phospholipid. In some embodiments of the LNPs or methods of the disclosure, the LNP comprises 50 mol % Compound 25 and about 10 mol % non-cationic helper lipid or phospholipid. In some embodiments of the LNPs or methods of the disclosure, the LNP comprises about 50 mol % Compound 25 and 10 mol % non-cationic helper lipid or phospholipid. In some embodiments of the LNPs or methods of the disclosure, the LNP comprises 50 mol % Compound 25 and 10 mol % non-cationic helper lipid or phospholipid. In some embodiments of the LNPs or methods of the disclosure, the LNP comprises about 49.83 mol % Compound 25, about 9.83 mol % non-cationic helper lipid or phospholipid, about 30.33 mol % sterol or other structural lipid, and about 2.0 mol % PEG lipid.

In some embodiments, the LNP is formulated for intravenous, subcutaneous, intramuscular, intraocular, intranasal, rectal, or oral delivery. In some embodiments, the LNP is formulated for intravenous delivery. In some embodiments, the LNP is formulated for subcutaneous delivery. In some embodiments, the LNP is formulated for intramuscular delivery. In some embodiments, the LNP is formulated for intraocular delivery. In some embodiments, the LNP is formulated for intranasal delivery. In some embodiments, the LNP is formulated for rectal delivery. In some embodiments, the LNP is formulated for oral delivery.

In some embodiments, the disease associated with an aberrant immune cell (e.g., T cell) function is an autoimmune disease, or a disease with hyper-activated immune function. In some embodiments, the disease is an autoimmune disease. In some embodiments, the autoimmune disease e.g., rheumatoid arthritis (RA); graft versus host disease (GVHD) (e.g., acute GVHD or chronic GVHD); diabetes, e.g., Type 1 diabetes; inflammatory bowel disease (IBD); lupus (e.g., systemic lupus erythematosus (SLE)), multiple sclerosis; autoimmune hepatitis (e.g., Type 1 or Type 2); primary biliary cholangitis; organ transplant associated rejection; psoriasis; or polymyositis (also known as dermatomyositis).

In some embodiments, the autoimmune disease is rheumatoid arthritis (RA). In some embodiments, the autoimmune disease is graft versus host disease (GVHD) (e.g., acute GVHD or chronic GVHD). In some embodiments, the autoimmune disease is diabetes, e.g., Type 1 diabetes. In some embodiments, the autoimmune disease is inflammatory bowel disease (IBD). In some embodiments, IBD comprises colitis, ulcerative colitis or Crohn's disease. In some embodiments, the autoimmune disease is lupus, e.g., systemic lupus erythematosus (SLE). In some embodiments, the autoimmune disease is multiple sclerosis. In some embodiments, the autoimmune disease is autoimmune hepatitis, e.g., Type 1 or Type 2. In some embodiments, the autoimmune disease is primary biliary cholangitis.

In some embodiments, the autoimmune disease is organ transplant associated rejection. In some embodiments, an organ transplant associated rejection comprises allograft rejection; e.g., renal transplant rejection; liver transplant rejection; bone marrow transplant rejection; or stem cell transplant rejection. In some embodiments, a stem cell transplant comprises a transplant of any one or all of the following types of cells: stem cells, cord blood stem cells, hematopoietic stem cells, embryonic stem cells, cells derived from or comprising mesenchymal stem cells, and/or induced stem cells (e.g., induced pluripotent stem cells). In some embodiments, the stem cell comprises a pluripotent stem cell.

In some embodiments of any of the methods or compositions for use disclosed herein, the subject is a mammal, e.g., a human.

Additional features of any of the aforesaid polynucleotides, LNP compositions, or methods of using said polynucleotides or LNP compositions, include one or more of the following enumerated embodiments. Those skilled in the art will recognize or be able to ascertain using no more than routine experimentation, many equivalents to the specific embodiments of the invention described herein. Such equivalents are intended to be encompassed by the following enumerated embodiments.

### Other Embodiments of the Disclosure

E1. A lipid nanoparticle (LNP) composition comprising a polynucleotide comprising an mRNA which encodes an ITB6 molecule.

E2. An LNP composition for immunomodulation, e.g., for inhibiting an immune response, the composition comprising a polynucleotide comprising an mRNA which encodes an ITB6 molecule.

E3. The LNP composition of embodiment any one of E1-E2, wherein the ITB6 molecule comprises a naturally occurring ITB6 molecule, a fragment of a naturally occurring ITB6 molecule, or a variant thereof.

E4. The LNP composition of any one of embodiments E1-E3, wherein the ITB6 molecule comprises an amino acid sequence having at least 85%, 90%, 95%, 96%, 97%, 98%, 99% or 100% identity, or differing by no more than 1, 2, 5, 10, 15, 20, 25, 30, 40, 50, 60, 70, 80, 90, or 100 amino acids from, an ITB6 amino acid sequence provided in Table 1A or Table 2A, e.g., any one of SEQ ID NOs: 17, 1, 7, 9, 11, 13, or 15, or a functional fragment thereof.

E5. The LNP composition of any one of embodiments E1-E4, wherein the ITB6 molecule comprises the amino acid sequence of any one of SEQ ID NOs: 17, 1, 7, 9, 11, 13, or 15, or a functional fragment thereof.

E6. The LNP composition of any one of embodiments E1-E4, wherein the polynucleotide encoding the ITB6 molecule comprises a nucleotide sequence having at least 85%, 90%, 95%, 96%, 97%, 98%, 99% or 100% identity to, or differing by no more than 10, 25, 50, 100, 150, 200, 250, or 300 nucleotides from, an ITB6 nucleotide sequence provided in Table 1A or Table 2A, e.g., any one of SEQ ID NOs: 18, 2-6, 8, 10, 12, 14, 16, or 60-160-175, or a functional fragment thereof.

E7. The LNP composition of any one of embodiments E1-E6, wherein the polynucleotide encoding the ITB6 molecule comprises:
(I) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 2 or a functional fragment thereof; (b) the nucleotide sequence of SEQ ID NO: 2 or a functional fragment thereof; or (c) the nucleotide sequence of SEQ ID NO: 160, which comprises from 5′ to 3′ end: the 5′ UTR sequence of SEQ ID NO: 56, the ORF sequence of SEQ ID NO: 2, and the 3′ UTR sequence of SEQ ID NO: 110;
(II) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 3 or a functional fragment thereof; (b) the nucleotide sequence of SEQ ID NO: 3 or a functional fragment thereof; or (c) the nucleotide sequence of SEQ ID NO: 161, which comprises from 5′ to 3′ end: the 5′ UTR sequence of SEQ ID NO: 50, the ORF sequence of SEQ ID NO: 3, and the 3′ UTR sequence of SEQ ID NO: 110;
(III) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 4 or a functional fragment thereof; (b) the nucleotide sequence of SEQ ID NO: 4 or a functional fragment thereof; or (c) the nucleotide sequence of SEQ ID NO: 162, which comprises from 5′ to 3′ end: the 5′ UTR sequence of SEQ ID NO: 50, ORF sequence of SEQ ID NO: 4, and the 3′ UTR sequence of SEQ ID NO: 143;
(IV) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 5 or a functional fragment thereof; (b) the nucleotide sequence of SEQ ID NO: 5 or a functional fragment thereof; (c) the nucleotide sequence of SEQ ID NO: 163, which comprises from 5′ to 3′ end: the 5′ UTR sequence of SEQ ID NO: 50, the ORF sequence of SEQ ID NO: 5, and the 3′ UTR sequence of SEQ ID NO: 110; (d) the nucleotide sequence of SEQ ID NO: 164, which comprises from 5′ to 3′ end: the 5′ UTR sequence of SEQ ID NO: 50, the ORF sequence of SEQ ID NO: 5, and the 3′ UTR sequence of SEQ ID NO: 144; or (e) the nucleotide sequence of SEQ ID NO: 169, which comprises from 5′ to 3′ end: the 5′ UTR sequence of SEQ ID NO: 50, the ORF sequence of SEQ ID NO: 5, and the 3′ UTR sequence of SEQ ID NO: 145;
(V) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 6 or a functional fragment thereof; (b) the nucleotide sequence of SEQ ID NO: 6 or a functional fragment thereof; (c) the nucleotide sequence of SEQ ID NO: 165, which comprises from 5′ to 3′ end: the 5′ UTR sequence of SEQ ID NO: 50, the ORF sequence of SEQ ID NO: 6, and the 3′ UTR sequence of SEQ ID NO: 110; (d) the nucleotide sequence of SEQ ID NO: 166, which comprises from 5′ to 3′ end: the 5′ UTR sequence of SEQ ID NO: 50, the ORF sequence of SEQ ID NO: 6, and the 3′ UTR sequence of SEQ ID NO: 110; (e) the nucleotide sequence of SEQ ID NO: 167, which comprises from 5′ to 3′ end: the 5′ UTR sequence of SEQ ID NO: 50, the ORF sequence of SEQ ID NO: 6, and the 3′ UTR sequence of SEQ ID NO: 145; or (g) the nucleotide sequence of SEQ ID NO: 168, which comprises from 5′ to 3′ end: the 5′ UTR sequence of SEQ ID NO: 50, the ORF sequence of SEQ ID NO: 6, and the 3′ UTR sequence of SEQ ID NO: 143;
(VI) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 8 or a functional fragment thereof; (b) the nucleotide sequence of SEQ ID NO: 8 or a functional fragment thereof; or (c) the nucleotide sequence of SEQ ID NO: 170, which comprises from 5′ to 3′ end: the 5′ UTR sequence of SEQ ID NO: 50, the ORF sequence of SEQ ID NO: 8, and the 3′ UTR sequence of SEQ ID NO: 110;
(VII) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 10 or a functional fragment thereof; (b) the nucleotide sequence of SEQ ID NO: 10 or a functional fragment thereof; or (c) the nucleotide sequence of SEQ ID NO: 171, which comprises from 5′ to 3′ end: the 5′ UTR sequence of SEQ ID NO: 50, the ORF sequence of SEQ ID NO: 10, and the 3′ UTR sequence of SEQ ID NO: 110;
(VIII) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 12 or a functional fragment thereof; (b) the nucleotide sequence of SEQ ID NO: 12 or a functional fragment thereof; or (c) the nucleotide sequence of SEQ ID NO: 172, which comprises from 5′ to 3′ end: the 5′ UTR sequence of SEQ ID NO: 50, the ORF sequence of SEQ ID NO: 12, and the 3′ UTR sequence of SEQ ID NO: 110;
(IX) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 14 or a functional fragment thereof; (b) the nucleotide sequence of SEQ ID NO: 14 or a functional fragment thereof; or (c) the nucleotide sequence of SEQ ID NO: 173, which comprises from 5′ to 3′ end: the 5′ UTR sequence of SEQ ID NO: 50, the ORF sequence of SEQ ID NO: 14, and the 3′ UTR sequence of SEQ ID NO: 110;
(X) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 16 or a functional fragment thereof; (b) the nucleotide sequence of SEQ ID NO: 16 or a functional fragment thereof; or (c) the nucleotide sequence of SEQ ID NO: 174, which comprises from 5′ to 3′ end: the 5′ UTR sequence of SEQ ID NO: 56, the ORF sequence of SEQ ID NO: 16, and the 3′ UTR sequence of SEQ ID NO: 110; or
(XI) (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 18 or a functional fragment thereof; (b) the nucleotide sequence of SEQ ID NO: 18 or a functional fragment thereof; or (c) the nucleotide sequence of SEQ ID NO: 175, which comprises from 5′ to 3′ end: the 5′ UTR sequence of SEQ ID NO: 50, the ORF sequence of SEQ ID NO: 18, and the 3′ UTR sequence of SEQ ID NO: 142.

E8. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 17 or a functional fragment thereof; (b) the nucleotide sequence of SEQ ID NO: 18 or a functional fragment thereof; or (c) the nucleotide sequence of SEQ ID NO: 175, which comprises from 5′ to 3′ end: the 5′ UTR sequence of SEQ ID NO: 50, the ORF sequence of SEQ ID NO: 17, and the 3′ UTR sequence of SEQ ID NO: 142.

E9. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 2 or a functional fragment thereof; (b) the nucleotide sequence of SEQ ID NO: 2 or a functional fragment thereof, or (c) the nucleotide sequence of SEQ ID NO: 160, which comprises from 5′ to 3′ end: the 5′ UTR sequence of SEQ ID NO: 56, the ORF sequence of SEQ ID NO: 2, and the 3′ UTR sequence of SEQ ID NO: 110.

E10. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 3 or a functional fragment thereof; (b) the nucleotide sequence of SEQ ID NO: 3 or a functional fragment thereof, or (c) the nucleotide sequence of SEQ ID NO: 161, which comprises from 5′ to 3′ end: the 5′ UTR sequence of SEQ ID NO: 50, the ORF sequence of SEQ ID NO: 3, and the 3′ UTR sequence of SEQ ID NO: 110.

E11. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 4 or a functional fragment thereof; (b) the nucleotide sequence of SEQ ID NO: 4 or a functional fragment thereof, or (c) the nucleotide sequence of SEQ ID NO: 162, which comprises from 5′ to 3′ end: the 5′ UTR sequence of SEQ ID NO: 50, ORF sequence of SEQ ID NO: 4, and the 3′ UTR sequence of SEQ ID NO: 143.

E12. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 5 or a functional fragment thereof; (b) the nucleotide sequence of SEQ ID NO: 5 or a functional fragment thereof, or (c) the nucleotide sequence of SEQ ID NO: 163, which comprises from 5′ to 3′ end: the 5′ UTR sequence of SEQ ID NO: 50, the ORF sequence of SEQ ID NO: 5, and the 3′ UTR sequence of SEQ ID NO: 110.

E13. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 5 or a functional fragment thereof; (b) the nucleotide sequence of SEQ ID NO: 5 or a functional fragment thereof, or (c) the nucleotide sequence of SEQ ID NO: 164, which comprises from 5′ to 3′ end: the 5′ UTR sequence of SEQ ID NO: 50, the ORF sequence of SEQ ID NO: 5, and the 3′ UTR sequence of SEQ ID NO: 144.

E14. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 5 or a functional fragment thereof; (b) the nucleotide sequence of SEQ ID NO: 5 or a functional fragment thereof, or (c) the nucleotide sequence of SEQ ID NO: 169, which comprises from 5′ to 3′ end: the 5′ UTR sequence of SEQ ID NO: 50, the ORF sequence of SEQ ID NO: 5, and the 3′ UTR sequence of SEQ ID NO: 145.

E15. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 6 or a functional fragment thereof; (b) the nucleotide sequence of SEQ ID NO: 6 or a functional fragment thereof, or (c) the nucleotide sequence of SEQ ID NO: 165, which comprises from 5′ to 3′ end: the 5′ UTR sequence of SEQ ID NO: 50, the ORF sequence of SEQ ID NO: 6, and the 3′ UTR sequence of SEQ ID NO: 110.

E16. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 6 or a functional fragment thereof; (b) the nucleotide sequence of SEQ ID NO: 6 or a functional fragment thereof, or (c) the nucleotide sequence of SEQ ID NO: 166, which comprises from 5′ to 3′ end: the 5′ UTR sequence of SEQ ID NO: 50, the ORF sequence of SEQ ID NO: 6, and the 3′ UTR sequence of SEQ ID NO: 110.

E17. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 6 or a functional fragment thereof, (b) the nucleotide sequence of SEQ ID NO: 167, which comprises from 5′ to 3′ end: the 5′ UTR sequence of SEQ ID NO: 50, the ORF sequence of SEQ ID NO: 6, and the 3′ UTR sequence of SEQ ID NO: 145.

E18. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 6 or a functional fragment thereof; (b) the nucleotide sequence of SEQ ID NO: 168, which comprises from 5′ to 3′ end: the 5′ UTR sequence of SEQ ID NO: 50, the ORF sequence of SEQ ID NO: 6, and the 3′ UTR sequence of SEQ ID NO: 143.

E19. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 8 or a functional fragment thereof; (b) the nucleotide sequence of SEQ ID NO: 8 or a functional fragment thereof, or (c) the nucleotide sequence of SEQ ID NO: 170, which comprises from 5′ to 3′ end: the 5′ UTR sequence of SEQ ID NO: 50, the ORF sequence of SEQ ID NO: 8, and the 3′ UTR sequence of SEQ ID NO: 110.

E20. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 10 or a functional fragment thereof; (b) the nucleotide sequence of SEQ ID NO: 10 or a functional fragment thereof; or (c) the nucleotide sequence of SEQ ID NO: 171, which comprises from 5′ to 3′ end: the 5′ UTR sequence of SEQ ID NO: 50, the ORF sequence of SEQ ID NO: 10, and the 3′ UTR sequence of SEQ ID NO: 110.

E21. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 12 or a functional fragment thereof; (b) the nucleotide sequence of SEQ ID NO: 12 or a functional fragment thereof; or (c) the nucleotide sequence of SEQ ID NO: 172, which comprises from 5′ to 3′ end: the 5′ UTR sequence of SEQ ID NO: 50, the ORF sequence of SEQ ID NO: 12, and the 3′ UTR sequence of SEQ ID NO: 110.

E22. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 14 or a functional fragment thereof; (b) the nucleotide sequence of SEQ ID NO: 14 or a functional fragment thereof; or (c) the nucleotide sequence of SEQ ID NO: 173, which comprises from 5′ to 3′ end: the 5′ UTR sequence of SEQ ID NO: 50, the ORF sequence of SEQ ID NO: 14, and the 3′ UTR sequence of SEQ ID NO: 110.

E23. The LNP composition of any one of embodiments E1-E7, wherein the polynucleotide encoding the ITB6 molecule comprises: (a) a nucleotide sequence having at least 85%, 90%, 95%, 96%, 97%, 98%, 99% or 100% identity to, or differing by no more than 10, 25, 50, 100, 150, 200, 250, or 300 nucleotides from, the nucleotide sequence of SEQ ID NO: 16 or a functional fragment thereof; (b) the nucleotide sequence of SEQ ID NO: 16 or a functional fragment thereof; or (c) the nucleotide sequence of SEQ ID NO: 174, which comprises from 5′ to 3′ end: the 5′ UTR sequence of SEQ ID NO: 56, the ORF sequence of SEQ ID NO: 16, and the 3′ UTR sequence of SEQ ID NO: 110.

E24. The LNP composition of any one of embodiments E1-E23, wherein the ITB6 molecule comprises an amino acid sequence that does not comprise a leader sequence and/or an affinity tag.

E25. The LNP composition of any one of embodiments E1-E24, wherein the ITB6 molecule comprises a half-life extender, e.g., a protein (or fragment thereof) that binds to a serum protein such as albumin; an immunoglobulin domain, e.g., an IgG; FcRn or transferrin.

E26. The LNP composition of embodiment E25, wherein the half-life extender is an immunoglobulin Fc region or a variant thereof.

E27. The LNP composition of any one of embodiments E1-E26, which results in a modulation (e.g., suppression) of a T cell activity and/or a T cell function in a population of immune cells, e.g., as compared to a T cell activity and/or a T cell function in an otherwise similar or identical population of immune cells which have not been contacted with the LNP composition comprising a polynucleotide comprising an mRNA which encodes an ITB6 molecule.

E28. The LNP composition of embodiment E27, wherein the population of immune cells comprise T cells (e.g., CD4+ T cells, CD8+ T cells, or regulatory T cells (Tregs)), B cells, dendritic cells, granulocytes, monocytes and/or macrophages.

E29. The LNP composition of embodiment E27 or E28, wherein the T cell activity and/or T cell function is an activity and/or a function of a CD8+ T cell (e.g., antigen-specific CD8+ cell) and/or a CD4+ T cell (an antigen-specific CD4+ T cell).

E30. The LNP composition of any one of embodiments E27-E29, wherein the modulation (e.g., suppression) of a T cell activity and/or a T cell function comprises any one, two, three or all of the following:
(i) increased level of Treg differentiation;
(ii) reduced T cell proliferation, survival and/or expansion (e.g., reduced CD4+ T cell proliferation, survival and/or expansion);
(iii) reduced expression, activity and/or secretion of an effector cytokine (e.g., IFNg);
and/or
(iv) reduced expression and/or activity of a T cell transcription factor (e.g., T-bet).

E31. The LNP composition of any one of embodiments E27-E30, wherein the suppression of a T cell activity and/or a T cell function occurs and/or is determined in vitro, e.g., in a sample.

E32. The LNP composition of any one of embodiments E27-E31, wherein the suppression of a T cell activity and/or a T cell function occurs and/or is determined in vivo, e.g., in a subject.

E33. The LNP composition of any one of embodiments E27-E32, wherein the suppression of a T cell activity and/or a T cell function comprises (i) increased level of Treg differentiation.

E34. The LNP composition of embodiment E33, wherein the level of Treg differentiation (e.g., from antigen-specific CD4+ T cells) is increased by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold), compared to a reference level of Treg differentiation, e.g., as determined by a method described in Examples 2 and 4.

E35. The LNP composition of embodiment E33 or E34, wherein the reference level of Treg differentiation (e.g., from antigen specific CD4+ T cells) is the level of Treg differentiation in an otherwise similar sample or subject which has not been contacted with the LNP composition comprising a polynucleotide comprising an mRNA which encodes an ITB6 molecule.

E36. The LNP composition of any one of embodiments E27-E35, wherein the suppression of a T cell activity and/or a T cell function comprises (ii) reduced T cell proliferation, survival and/or expansion, e.g., reduced CD4+ T cell proliferation, survival and/or expansion.

E37. The LNP composition of embodiment E36, wherein the T cell proliferation, survival and/or expansion is reduced by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold), compared to a reference level of T cell proliferation, survival and/or expansion, e.g., as determined by a method described in Examples 3 and 7.

E38. The LNP composition of embodiment E36 or E37, wherein the reference level of T cell proliferation, survival and/or expansion is the level of T cell proliferation, survival and/or expansion in an otherwise similar sample or subject which has not been contacted with the LNP composition comprising a polynucleotide comprising an mRNA which encodes an ITB6 molecule.

E39. The LNP composition of any one of embodiments E36-E38, wherein the reduction in T cell proliferation, survival and/or expansion occurs upon, or is determined after: (a) co-culture of T cells (e.g., CD4+ T cells) with dendritic cells that have been contacted with an LNP composition comprising a polynucleotide comprising an mRNA which encodes an ITB6 molecule; and/or (b) contact with a cytokine (e.g., TGFbeta).

E40. The LNP composition of any one of embodiments E27-E39, wherein the suppression of a T cell activity and/or a T cell function comprises (iii) reduced expression, activity and/or secretion of an effector cytokine (e.g., IFNg).

E41. The LNP composition of embodiment E40, wherein the expression, activity and/or secretion of an effector cytokine is reduced by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold), compared to a reference level of expression, activity and/or secretion of the effector cytokine.

E42. The LNP composition of embodiment E40 or E41, wherein the reference level of expression, activity and/or secretion of the effector cytokine is the level of expression, activity and/or secretion of the effector cytokine in an otherwise similar or identical sample which has not been contacted with the LNP composition comprising a polynucleotide comprising an mRNA which encodes an ITB6 molecule, or which has not been contacted with an immune cell (e.g., T cell) activating or stimulating agent.

E43. The LNP composition of any one of embodiments E40-E42, wherein the effector cytokine is IFNg, and wherein the expression of IFNg (e.g., produced by antigen-specific CD8+ T cells) is reduced by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold) in a sample, e.g., as determined by a method described in Examples 5 and 9.

E44. The LNP composition of embodiment E43, wherein the sample has been contacted with an immune cell (e.g., T cell) activating or stimulating agent.

E45. The LNP composition of any one of embodiments E43 or E44, wherein the T cells (e.g., CD8+ T cells) in the sample have been stimulated and/or activated, e.g., with a peptide or costimulatory molecule.

E46. The LNP composition of any one of embodiments E27-E45, wherein the suppression of a T cell activity and/or a T cell function comprises (iv) reduced expression and/or activity of a T cell transcription factor (e.g., T-bet).

E47. The LNP composition of embodiment E46, wherein the expression and/or activity of the T cell transcription factor is reduced by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold), compared to a reference level of expression and/or activity of the T cell transcription factor.

E48. The LNP composition of embodiment E46 or E47, wherein the reference level of expression and/or activity of the T cell transcription factor is the level of expression and/or activity of the T cell transcription factor in an otherwise similar sample or subject which has not been contacted with the LNP composition comprising a polynucleotide comprising an mRNA which encodes an ITB6 molecule.

E49. The LNP composition of any one of embodiments E46-E48, wherein the T cell transcription factor is T-bet, and wherein the expression of T-bet is reduced by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold) in a sample or subject, e.g., as determined by a method described in Example 8.

E50. The LNP composition of embodiment E49, wherein the reduction in the expression and/or an activity of T-bet occurs upon, or is determined after, co-culture of T cells with dendritic cells that have been contacted with an LNP composition comprising a polynucleotide comprising an mRNA which encodes an ITB6 molecule.

E51. The LNP composition of any one of embodiments E1-E50, which results in any one, two, three, four or all of the following, in a subject having, or at risk of having, graft vs host disease (GvHD):
(i) reduced donor immune cell (e.g., T cell) proliferation;
(ii) reduced weight loss, host B cell depletion, and/or donor immune cell (e.g., T cell) engraftment, optionally with concurrent Treg expansion;
(iii) reduced Tbet+ cells in T cell population (e.g., CD8+ T cell population);
(iv) reduced expression, activity and/or secretion of a proinflammatory cytokine (e.g., IFNg); and/or
(v) maintained or increased Treg and/or host lymphocyte population.

E52. The LNP composition of embodiment E51, which results in (i) reduced donor immune cell (e.g., T cell) proliferation.

E53. The LNP composition of embodiment E52, wherein the donor immune cell (e.g., T cell) proliferation is reduced by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold), compared to a reference level of donor immune cell (e.g., T cell) proliferation, e.g., as determined by a method described in Example 7.

E54. The LNP composition of embodiment E51 or E52, wherein the reference level of donor cell (e.g., T cell) proliferation is the level of donor immune cell (e.g., T cell) proliferation in the subject before being contacted with the LNP composition comprising a polynucleotide comprising an mRNA which encodes an ITB6 molecule, or in an otherwise similar subject who has not been contacted with the LNP composition comprising a polynucleotide comprising an mRNA which encodes an ITB6 molecule.

E55. The LNP composition of any of embodiments E51-E54, which results in (ii) reduced weight loss, host B cell depletion, and/or donor immune cell (e.g., T cell) engraftment, optionally with concurrent Treg expansion.

E56. The LNP composition of embodiment E55, wherein the weight loss, host B cell depletion, and/or donor immune cell (e.g., T cell) engraftment is reduced by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold), compared to a reference level of weight loss, host B cell depletion, and/or donor immune cell (e.g., T cell) engraftment, e.g., as determined by a method described in Example 6.

E57. The LNP composition of embodiment E56, wherein the reference level of weight loss, host B cell depletion, and/or donor immune cell (e.g., T cell) engraftment is the level of weight loss, host B cell depletion, and/or donor immune cell (e.g., T cell) engraftment in the subject before being contacted with the LNP composition comprising a polynucleotide comprising an mRNA which encodes an ITB6 molecule, or in an otherwise similar subject who has not been contacted with the LNP composition comprising a polynucleotide comprising an mRNA which encodes an ITB6 molecule.

E58. The LNP composition of any of embodiments E51-E57, wherein the donor immune cell specified in (i) or (ii) comprise T cell, e.g., CD8+ T cell, CD4+ T cell, or T regulatory cell (e.g., CD25+ and/or FoxP3+ T cell).

E59. The LNP composition of any of embodiments E51-E58, which results in (iii) reduced Tbet+ cells in T cell population (e.g., CD8+ T cell population).

E60. The LNP composition of any of embodiments E51-E59, wherein the Tbet+ cells in T cell population (e.g., CD8+ T cell population) is reduced by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold), compared to a reference level of Tbet+ cells in T cell population (e.g., CD8+ T cell population), e.g., as determined by a method described in Example 8.

E61. The LNP composition of embodiment E60, wherein the reference level of Tbet+ cells in T cell population (e.g., CD8+ T cell population) is the level of Tbet+ cells in T cell population (e.g., CD8+ T cell population) in the subject before being contacted with the LNP composition comprising a polynucleotide comprising an mRNA which encodes an ITB6 molecule, or in an otherwise similar subject who has not been contacted with the LNP composition comprising a polynucleotide comprising an mRNA which encodes an ITB6 molecule.

E62. The LNP composition of any of embodiments E51-E61, which results in (iv) reduced expression, activity and/or secretion of a proinflammatory cytokine (e.g., IFNg).

E63. The LNP composition of any of embodiments E51-E62, wherein the expression, activity and/or secretion of a proinflammatory cytokine (e.g., IFNg) is reduced by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold), compared to a reference level of expression, activity and/or secretion of a proinflammatory cytokine (e.g., IFNg), e.g., as measured by an assay described in Example 9.

E64. The LNP composition of embodiment E63, the reference level of expression, activity and/or secretion of a proinflammatory cytokine (e.g., IFNg) is the level of expression, activity and/or secretion of a proinflammatory cytokine (e.g., IFNg) in the subject before being contacted with the LNP composition comprising a polynucleotide comprising an mRNA which encodes an ITB6 molecule, or in an otherwise similar subject who has not been contacted with the LNP composition comprising a polynucleotide comprising an mRNA which encodes an ITB6 molecule.

E65. The LNP composition of any of embodiments E51-E64, which results in (v) maintained or increased Treg and/or host lymphocyte population.

E66. The LNP composition of any of embodiments E51-E65, wherein the Treg and/or host lymphocyte population is substantially unchanged or increased by about 1-10-fold (e.g., about 2-8-fold, 3-7-fold, 4-6-fold, 1-8-fold, 1-6-fold, 1-4-fold, 1-2-fold, 8-10-fold, 6-10-fold, 4-10-fold, 2-10-fold, 1-3-fold, 2-4-fold, 3-5-fold, 5-7-fold, 6-8-fold, or 7-9-fold), compared to a reference level of Treg and/or host lymphocyte population, e.g., as measured by an assay described in Example 10.

E67. The LNP composition of embodiment E66, the reference level of Treg and/or host lymphocyte population is the level of Treg and/or host lymphocyte population in the subject before being contacted with the LNP composition comprising a polynucleotide comprising an mRNA which encodes an ITB6 molecule, or in an otherwise similar subject who has not been contacted with the LNP composition comprising a polynucleotide comprising an mRNA which encodes an ITB6 molecule.

E68. The LNP composition of any of embodiments E1-E67, which results in delayed onset of GvHD in a subject.

E69. The LNP composition of E68, wherein the onset of GvHD is delayed by at least 1 week, 2 weeks, 3 weeks, 1 month, 2 months, 3 months, 4 months, 5 months, 6 months, 7 months, 8 months, 9 months, 10 months, 11 months, 12 months, 1.5 years or 2 years, compared to a reference onset of GvHD, e.g., the onset of GvHD in an otherwise similar subject who has not been contacted with the LNP composition comprising a polynucleotide comprising an mRNA which encodes an ITB6 molecule.

E70. The LNP composition of any of embodiments E1-E68, which results in delayed onset of GvHD in a subject.

E71. The LNP composition of E70, wherein the onset of GvHD is delayed by at least 1 week, 2 weeks, 3 weeks, 1 month, 2 months, 3 months, 4 months, 5 months, 6 months, 7 months, 8 months, 9 months, 10 months, 11 months, 12 months, 1.5 years or 2 years, compared to a reference onset of GvHD, e.g., the onset of GvHD in an otherwise similar subject who has not been contacted with the LNP composition comprising a polynucleotide comprising an mRNA which encodes an ITB6 molecule.

E72. The LNP composition of any one of embodiments E1-E71, which results in amelioration of GvHD or a symptom thereof in a subject, e.g., compared to the subject before being contacted with the LNP composition comprising a polynucleotide comprising an mRNA which encodes an ITB6 molecule, or a subject who has not been contacted with the LNP composition comprising a polynucleotide comprising an mRNA which encodes an ITB6 molecule.

E73. A method of assessing the responsiveness of a subject to a therapy comprising an LNP composition comprising an mRNA which encodes an ITB6 molecule, the method comprising:
(a) measuring the expression level of one or more biomarkers (e.g., selected from the group consisting of: PMEPA1, ITGAE/CD103, SMAD7, SKIL, and SKI) in a sample from the subject collected following administration of the therapy; and
(b) comparing the expression level in the sample with a reference expression level, wherein an increase in the expression level of the one or more biomarkers is indicative of a response to the therapy.

E74. The method of embodiment E73, wherein the one or more biomarkers are one or more of PMEPA1, ITGAE/CD103, SMAD7, SKIL, and SKI.

E75. The method of embodiment E73 or E74, wherein the level of one or more of the one or more biomarkers in the sample from the subject following treatment is at least 2-fold (e.g., at least 3-fold, 4-fold, 5-fold, 6-fold, 7-fold, 8-fold, 9-fold, or 10-fold) greater than a reference expression level of the one or more biomarkers, wherein the reference expression level comprises:
(a) the expression level of the one or more biomarkers in a sample from the subject prior to treatment with an ITB6 mRNA; or
(b) the expression level of o the one or more biomarkers in a sample from a subject who has not received the treatment with an ITB6 mRNA.

E76. The LNP composition of any one of the preceding embodiments, wherein the polynucleotide comprising an mRNA encoding the ITB6 molecule, comprises at least one chemical modification.

E77. The LNP composition of embodiment E76, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

E78. The LNP composition of embodiment E76 or E77, wherein the chemical modification is selected from the group consisting of pseudouridine, N1-methylpseudouridine, 5-methylcytosine, 5-methoxyuridine, and a combination thereof.

E79. The LNP composition of any one of embodiments E76-E78, wherein the chemical modification is N1-methylpseudouridine.

E80. The LNP composition of any one of the preceding embodiments, wherein the mRNA in the lipid nanoparticle comprises fully modified N1-methylpseudouridine.

E81. The LNP composition of any one of the preceding embodiments, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

E82. The LNP composition of embodiment E81, wherein the ionizable lipid comprises an amino lipid.

E83. The LNP composition of embodiment E81 or E82, wherein the ionizable lipid comprises a compound of any of Formulae (I), (I-a), (I-b), (I-c), (II), (II-a), (II-b), (II-c), (II-d), (II-e), (II-f), (II-g), (II-h), or (III).

E84. The LNP composition of any one of embodiments E81-E83, wherein the ionizable lipid comprises a compound of Formula (I).

E85. The LNP composition of any one of embodiments E81-E84, wherein the ionizable lipid comprises Compound 18, Compound 25, Compound 301, or Compound 357.

E86. The LNP composition of any one of embodiments E81-E85, wherein the LNP comprises a molar ratio of about 20-60% ionizable lipid:5-25% phospholipid:25-55% cholesterol; and 0.5-15% PEG lipid.

E87. The LNP composition of embodiment E86, wherein the LNP comprises a molar ratio of about 50% ionizable lipid:about 10% phospholipid:about 38.5% cholesterol; and about 1.5% PEG lipid.

E88. The LNP composition of embodiment E86 or E87, wherein the LNP comprises a molar ratio of about 49.83% ionizable lipid:about 9.83% phospholipid:about 30.33% cholesterol; and
about 2.0% PEG lipid.

E89. The LNP composition of any one of embodiments E86-E88, wherein the ionizable lipid comprises a compound of any of Formulae (I), (I-a), (I-b), (I-c), (II), (II-a), (II-b), (II-c), (II-d), (II-e), (II-f), (II-g), (II-h), or (III).

E90. The LNP composition of embodiment E89, wherein the ionizable lipid comprises a compound of Formula (I).

E91. The LNP composition of embodiment E89 or E90, wherein the ionizable lipid comprises Compound 18, Compound 25, Compound 301, or Compound 357.

E92. The LNP composition of any one of the preceding embodiments, which is formulated for intravenous, subcutaneous, intramuscular, intranasal, intraocular, rectal, or oral delivery.

E93. The LNP composition of any one of the preceding embodiments, further comprising a pharmaceutically acceptable carrier or excipient.

E94. A pharmaceutical composition comprising the LNP composition of any one of embodiments E1-E93.

E95. A method of modulating, e.g., suppressing, an immune response in a subject, comprising administering to the subject in need thereof an effective amount of an LNP composition comprising a polynucleotide comprising an mRNA which encodes an ITB6 molecule.

E96. A method of treating, preventing or preventing a symptom of, a disease with aberrant T cell function, e.g., an autoimmune disease or an inflammatory disease, comprising administering to the subject in need thereof an effective amount of an LNP composition comprising a polynucleotide comprising an mRNA which encodes an ITB6 molecule.

E97. A composition comprising an LNP composition which comprises a polynucleotide comprising an mRNA encoding an ITB6 molecule, for use in a method of treating, preventing or preventing a symptom of, a disease with aberrant T cell function, e.g., an autoimmune disease or an inflammatory disease.

E98. The method of E96 or the LNP composition for use of embodiment E97, wherein the disease is chosen from rheumatoid arthritis (RA); graft versus host disease (GVHD) (e.g., acute GVHD or chronic GVHD); diabetes, e.g., Type 1 diabetes; inflammatory bowel disease (IBD); lupus (e.g., systemic lupus erythematosus (SLE)), multiple sclerosis; autoimmune hepatitis (e.g., Type 1 or Type 2); primary biliary cholangitis; organ transplant associated rejection; or myasthenia gravis.

E99. The method, or the LNP composition for use of any one of E95-E98, wherein the ITB6 molecule comprises a naturally occurring ITB6 molecule, a fragment of a naturally occurring ITB6 molecule, or a variant thereof.

E100. The method, or the LNP composition for use of E99, wherein the ITB6 molecule comprises an amino acid sequence having at least 85%, 90%, 95%, 96%, 97%, 98%, 99% or 100% identity to an amino acid sequence of ITB6 provided in Table 1A or Table 2A, e.g., any one of SEQ ID NOs: 17, 1, 7, 9, 11, 13, or 15.

E101. The method, or the LNP composition for use of E99, wherein the ITB6 molecule comprises the amino acid sequence of any one of SEQ ID NOs: 17, 1, 7, 9, 11, 13, or 15.

E102. The method, or the LNP composition for use of E99, wherein the polynucleotide encoding the ITB6 molecule comprises a nucleotide sequence having at least 85%, 90%, 95%, 96%, 97%, 98%, 99% or 100% identity to an ITB6 nucleotide sequence provided in Table 1A or Table 2A, e.g., any one of SEQ ID NOs: 18, 2-6, 8, 10, 12, 14, 16, or 160-175.

E103. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 18;
(b) the nucleotide sequence of SEQ ID NO: 18; or
(c) the nucleotide sequence of SEQ ID NO: 175, which comprises from 5′ to 3′ end: the 5′ UTR sequence of SEQ ID NO: 50, the ORF sequence of SEQ ID NO:18, and the 3′ UTR sequence of SEQ ID NO: 142.

E104. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 2;
(b) the nucleotide sequence of SEQ ID NO: 2; or
(c) the nucleotide sequence of SEQ ID NO: 160, which comprises from 5′ to 3′ end: the 5′ UTR sequence of SEQ ID NO: 56, the ORF sequence of SEQ ID NO: 2, and the 3′ UTR sequence of SEQ ID NO: 110.

E105. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 3;
(b) the nucleotide sequence of SEQ ID NO: 3; or
(c) the nucleotide sequence of SEQ ID NO: 161, which comprises from 5′ to 3′ end: the 5′ UTR sequence of SEQ ID NO: 50, the ORF sequence of SEQ ID NO: 3, and the 3′ UTR sequence of SEQ ID NO: 110.

E106. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 4;
(b) the nucleotide sequence of SEQ ID NO: 4; or
(c) the nucleotide sequence of SEQ ID NO: 162, which comprises from 5′ to 3′ end: the 5′ UTR sequence of SEQ ID NO: 50, ORF sequence of SEQ ID NO: 4, and the 3′ UTR sequence of SEQ ID NO: 143.

E107. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 5;
(b) the nucleotide sequence of SEQ ID NO: 5; or
(c) the nucleotide sequence of SEQ ID NO: 163, which comprises from 5′ to 3′ end: the 5′ UTR sequence of SEQ ID NO: 50, the ORF sequence of SEQ ID NO: 5, and the 3′ UTR sequence of SEQ ID NO: 110.

E108. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 5;
(b) the nucleotide sequence of SEQ ID NO: 5; or
(c) the nucleotide sequence of SEQ ID NO: 164, which comprises from 5′ to 3′ end: the 5′ UTR sequence of SEQ ID NO: 50, the ORF sequence of SEQ ID NO: 5, and the 3′ UTR sequence of SEQ ID NO: 144.

E109. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 5;
(b) the nucleotide sequence of SEQ ID NO: 5; or
(c) the nucleotide sequence of SEQ ID NO: 169, which comprises from 5′ to 3′ end: the 5′ UTR sequence of SEQ ID NO: 50, the ORF sequence of SEQ ID NO: 5, and the 3′ UTR sequence of SEQ ID NO: 145.

E110. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 6;
(b) the nucleotide sequence of SEQ ID NO: 6; or
(c) the nucleotide sequence of SEQ ID NO: 165, which comprises from 5′ to 3′ end: the 5′ UTR sequence of SEQ ID NO: 50, the ORF sequence of SEQ ID NO: 6, and the 3′ UTR sequence of SEQ ID NO: 110.

E111. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 6;
(b) the nucleotide sequence of SEQ ID NO: 6; or
(c) the nucleotide sequence of SEQ ID NO: 166, which comprises from 5′ to 3′ end: the 5′ UTR sequence of SEQ ID NO: 50, the ORF sequence of SEQ ID NO: 6, and the 3′ UTR sequence of SEQ ID NO: 110.

E112. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 6;
(b) the nucleotide sequence of SEQ ID NO: 6; or
(c) the nucleotide sequence of SEQ ID NO: 167, which comprises from 5′ to 3′ end: the 5′ UTR sequence of SEQ ID NO: 50, the ORF sequence of SEQ ID NO: 6, and the 3′ UTR sequence of SEQ ID NO: 145.

E113. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 6;
(b) the nucleotide sequence of SEQ ID NO: 6; or
(c) the nucleotide sequence of SEQ ID NO: 168, which comprises from 5′ to 3′ end: the 5′ UTR sequence of SEQ ID NO: 50, the ORF sequence of SEQ ID NO: 6, and the 3′ UTR sequence of SEQ ID NO: 143.

E114. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 8;
(b) the nucleotide sequence of SEQ ID NO: 8; or
(c) the nucleotide sequence of SEQ ID NO: 170, which comprises from 5′ to 3′ end: the 5′ UTR sequence of SEQ ID NO: 50, the ORF sequence of SEQ ID NO: 8, and the 3′ UTR sequence of SEQ ID NO: 110.

E115. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 10;
(b) the nucleotide sequence of SEQ ID NO: 10; or
(c) the nucleotide sequence of SEQ ID NO: 171, which comprises from 5′ to 3′ end: the 5′ UTR sequence of SEQ ID NO: 50, the ORF sequence of SEQ ID NO: 10, and the 3′ UTR sequence of SEQ ID NO: 110.

E116. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 12;
(b) the nucleotide sequence of SEQ ID NO: 12; or
(c) the nucleotide sequence of SEQ ID NO: 172, which comprises from 5′ to 3′ end: the 5′ UTR sequence of SEQ ID NO: 50, the ORF sequence of SEQ ID NO: 12, and the 3′ UTR sequence of SEQ ID NO: 110.

E117. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 14;
(b) the nucleotide sequence of SEQ ID NO: 14; or
(c) the nucleotide sequence of SEQ ID NO: 173, which comprises from 5′ to 3′ end: the 5′ UTR sequence of SEQ ID NO: 50, the ORF sequence of SEQ ID NO: 14, and the 3′ UTR sequence of SEQ ID NO: 110.

E118. The method, or the LNP composition for use of E99, wherein the polynucleotide encoding the ITB6 molecule comprises:
(a) a nucleotide sequence having at least 85%, 90%, 95%, 96%, 97%, 98%, 99% or 100% identity to the sequence of SEQ ID NO: 16;
(b) the nucleotide sequence of SEQ ID NO: 16; or
(c) the nucleotide sequence of SEQ ID NO: 174, which comprises from 5′ to 3′ end: the 5′ UTR sequence of SEQ ID NO: 56, the ORF sequence of SEQ ID NO: 16, and the 3′ UTR sequence of SEQ ID NO: 110.

E119. The method, or the LNP composition for use of E99, wherein the ITB6 molecule comprises an amino acid sequence that does not comprise a leader sequence and/or an affinity tag.

E120. The method, or the LNP composition for use of any one of embodiments E95-E119, wherein the subject is a mammal, e.g., a human.

E121. The LNP composition for use, or the method of any one of embodiments E95-E120, wherein the LNP composition is administered to a subject according to a dosing interval, e.g., as described herein.

E122. The LNP composition for use, or the method of embodiment E121, wherein the dosing interval comprises an initial dose of the LNP composition and one or more subsequent doses (e.g., 1-50 doses, 5-50 doses, 10-50 doses, 15-50 doses, 20-50 doses, 25-50 doses, 30-50 doses, 35-50 doses, 40-50 doses, 45-50 doses, 1-45 doses, 1-40 doses, 1-35 doses, 1-30 doses, 1-25 doses, 1-20 doses, 1-15 doses, 1-10 doses, or 1-5 doses) of the same LNP composition.

E123. The LNP composition for use, or the method of embodiment E121 or E122, wherein the dosing interval comprises one or more doses of the LNP composition and one or more doses of an additional agent.

E124. The LNP composition for use, or the method of any one of embodiments E121-E123, wherein the dosing interval is performed over at least 1 week, 2 weeks, 3 weeks, or 4 weeks.

E125. The LNP composition for use, or the method of any one of embodiments E120-E124, wherein the dosing interval comprises a cycle, e.g., a seven-day cycle.

E126. The LNP composition for use, or the method of any one of embodiments E120-E125, wherein the dosing interval is repeated at least 1 time, at least 2 times, at least 3 times, at least 4 times, at least 5 times, at least 6 times, at least 7 times, at least 8 times, at least 9 times, or at least 10 times.

E127. The LNP composition for use, or the method of any one of embodiments E120-E126, wherein the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, or 5 years.

E128. The LNP composition for use, or the method of any one of embodiments E120-E127, wherein the LNP composition is administered daily for at least 1 week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 1 year.

E129. The LNP composition for use, or the method of any one of embodiments E120-E128, wherein the LNP composition is administered for at least 2, 3, 4, 5, or 6 consecutive days in a seven-day cycle, e.g., wherein the cycle is repeated about 1-20 times (e.g., 2-15, 5-10, 2-20, 5-20, 10-20, 15-20, 10-15, or 5-15 times).

E130. The LNP composition for use, or the method of any one of embodiments E120-E129, wherein the LNP composition is administered by a route of administration chosen from:
subcutaneous, intramuscular, intravenous, oral, intraocular, or rectal.

E131. The LNP composition for use, or the method of any one of embodiments E120-E130, wherein the LNP composition is administered at a dose of about 0.1-10 mg per kg (e.g., about 0.2-5 mg per kg, 0.5-2 mg per kg, 0.1-5 mg per kg, 0.1-2 mg per kg, 0.1-1 mg per kg, 0.1-0.5 mg per kg, 5-10 mg per kg, 2-10 mg per kg, 1-10 mg per kg, 0.5-10 mg per kg, or 0.2-10 mg per kg), e.g., about 0.2-1 mg per kg (e.g., about 0.5 mg per kg).

E132. The method or LNP composition for use of any one of embodiments E95-E131, further comprising administering an additional agent, e.g., a standard of care.

E133. The LNP composition for use, or the method of any one of embodiments E95-E132, wherein the composition or method results in suppression of T cell activity and/or function (e.g., T cell anergy, and/or T cell apoptosis) in a sample from the subject, e.g., as compared to T cell activity and/or function in an otherwise similar sample from a subject who has not been contacted with the LNP composition comprising a polynucleotide comprising an mRNA which encodes an ITB6 molecule.

E134. The LNP composition for use, or the method of embodiment E133, wherein suppression of T cell activity and/or function comprises any, one, two, three, four, five or all of the following:
(i) reduced donor immune cell (e.g., T cell) proliferation;
(ii) reduced weight loss, host B cell depletion, and/or donor immune cell (e.g., T cell) engraftment, optionally with concurrent Treg expansion;
(iii) reduced Tbet+ cells in T cell population (e.g., CD8+ T cell population);
(iv) reduced expression, activity and/or secretion of a proinflammatory cytokine (e.g., IFNg) in a sample; and/or
(v) maintained or increased Treg and/or host lymphocyte population.
(i) reduced level and/or activity of IFNg, e.g., secreted IFNg, in a sample;
(ii) reduction in T cell proliferation, survival and/or expansion;
(iii) increased T cell apoptosis;
(iv) reduction in expression and/or activity of a T cell transcription factor, e.g., T-bet;
(v) increase in and/or activation of T reg differentiation; and/or
(vi) an increase in the expression level of one or more of (e.g., 2, 3, 4, or 5) of PMEPA1, ITGAE/CD103, SMAD7, SKIL, and SKI.

E135. The LNP composition for use, or the method of embodiment E134, wherein the reduction in expression, activity, and/or secretion of a proinflammatory cytokine (e.g., IFNg) is about 1.2-10 fold (e.g., about 2-25-fold, 5-20-fold, 10-15-fold, 5-30-fold, 10-30-fold, 20-30-fold, 2-20-fold, 2-15-fold, or 2-10-fold).

E136. The LNP composition for use, or the method of embodiment E134 or E135, wherein the sample comprises immune cells, e.g., T cells, e.g., CD8 T cells.

E137. The LNP composition for use, or the method of any one of embodiments E134-E136, wherein the sample has been contacted with an immune cell, e.g., T cell, activating or stimulating agent.

E138. The LNP composition for use, or the method of embodiment E137, wherein the T cells, e.g., CD8 T cells, have been stimulated and/or activated, e.g., with a peptide or costimulatory molecule.

E139. The LNP composition for use, or the method of any one of embodiments E136-E138, wherein the CD8 T cells are antigen-specific.

E140. The LNP composition for use, or the method of any one of embodiments E136-E139, wherein the reduction in donor immune cell (e.g., T cell) proliferation is about 1.2-10 fold (e.g., about 2-25-fold, 5-20-fold, 10-15-fold, 5-30-fold, 10-30-fold, 20-30-fold, 2-20-fold, 2-15-fold, or 2-10-fold).

E141. The LNP composition for use, or the method of embodiment E140, wherein the reduction in donor immune cell (e.g., T cell) proliferation occurs upon: (a) co-culture of T cells with dendritic cells, e.g., CD11C+ cells, that have been contacted with an LNP composition comprising an ITB6 molecule; and/or (b) contact with a cytokine, e.g., TGFbeta.

E142. The LNP composition for use, or the method of embodiment E140 or E141, wherein reduction in Tbet+ cells is about 1.2-10 fold (e.g., about 2-25-fold, 5-20-fold, 10-15-fold, 5-30-fold, 10-30-fold, 20-30-fold, 2-20-fold, 2-15-fold, or 2-10-fold).

E143. The LNP composition for use, or the method of any one of embodiments E134-E142, wherein the reduction T-bet+ cells occurs upon co-culture of T cells with dendritic cells, e.g., CD11C+ cells, that have been contacted with an LNP composition comprising an ITB6 molecule.

E144. The LNP composition for use, or the method of any one of embodiments E134-E143, wherein the T cells comprise CD8+ T cells, CD4+ T cells, or T regulatory cells.

E145. The LNP composition for use, or the method of any one of embodiments E134-E144, wherein the method or composition reduces the level (e.g., expression) and/or activity of a costimulatory molecule, e.g., CD80, CD86, and/or MHCII, in a sample upon stimulation.

E146. The LNP composition for use, or the method of embodiment E145, wherein the reduction in level and/or activity of the costimulatory molecule is about 1.2-5-fold (e.g., about 2-4-fold, 2-5-fold, 2-3-fold, 3-5-fold, or 4-5-fold).

E147. The LNP composition for use, or the method of embodiment E145 or E146, wherein the sample has been contacted with a stimulant, e.g., LPS, or Poly IC.

E148. The LNP composition for use, or the method of any one of embodiments E145-E147, wherein the reduction in level and/or activity of the costimulatory molecule occurs in vitro or in vivo.

E149. The LNP composition for use, or the method of any one of embodiments E95-E133, wherein the disease with aberrant T cell function is graft vs host disease (GvHD).

E150. The LNP composition for use, or the method of embodiment E149, wherein the method or composition results in:
(i) reduced engraftment of donor cells, e.g., donor immune cells, e.g., T cells, in a subject or host, e.g., a human, rat or mouse;
(ii) reduction in the level, activity and/or secretion of IFNg from engrafted donor immune cells, e.g., T cells, in a subject or host, e.g., a human, rat or mouse; and/or
(iii) an absence of, prevention of, or delay in the onset of, graft vs host disease (GvHD) in a subject or a host, e.g., a human, rat or mouse.

E151. The LNP composition for use, or the method of embodiment E150, wherein the donor immune cells specified in (i) or (ii) comprise T cells, e.g., CD8+ T cells, CD4+ T cells, or T regulatory cells (e.g., CD25+ and/or FoxP3+ T cells).

E152. The LNP composition for use, or the method of embodiment E150 or E151, wherein the reduction in donor cell engraftment is about 1.5-30-fold (e.g., about 2-25-fold, 5-20-fold, 10-15-fold, 5-30-fold, 10-30-fold, 20-30-fold, 2-20-fold, 2-15-fold, or 2-10-fold).

E153. The LNP composition for use, or the method of any one of embodiments E150-E152, wherein the reduction in IFNg level, activity and/or secretion of IFNg is about 1.5-10-fold (e.g., about 2-25-fold, 5-20-fold, 10-15-fold, 5-30-fold, 10-30-fold, 20-30-fold, 2-20-fold, 2-15-fold, or 2-10-fold).

E154. The LNP composition for use, or the method of any one of embodiments E149-E153, wherein the delay in onset of GvHD is a delay of at least 1 week, 2 weeks, 3 weeks, 1 month, 2 months, 3 months, 4 months, 5 months, 6 months, 7 months, 8 months, 9 months, 10 months, 11 months, 12 months, 1.5 years, or 2 years.

E155. The LNP composition for use, or the method of any one of embodiments E149-E154, wherein any one of (i)-(iii) specified in embodiment E146 is compared to an otherwise similar host, e.g., a host that has not been contacted with the LNP composition comprising a polynucleotide comprising an mRNA which encodes an ITB6 molecule.

E156. The LNP composition for use, or the method of any one of embodiments E95-E133, wherein the disease with aberrant T cell function is arthritis, e.g., collagen-induced arthritis (CIA).

E157. The LNP composition for use, or the method of embodiment E156, wherein the method or composition results in amelioration or reduction of joint swelling, e.g., severity of joint swelling, e.g., as described herein, in a subject.

E158. The LNP composition for use, or the method of embodiment E156 or E157, wherein swelling is determined by an arthritis score, e.g., as described herein.

E159. The LNP composition for use, or the method of any one of embodiments E156-E158, wherein the reduction of joint swelling is compared to joint swelling in an otherwise similar subject, e.g., a subject who has not been contacted with the LNP composition comprising a polynucleotide comprising an mRNA which encodes an ITB6 molecule.

E160. The LNP composition for use, or the method of any one of embodiments E95-E133, wherein the disease with aberrant T cell function is colitis, e.g., dextran sulfate sodium (DSS)-induced colitis.

E161. The LNP composition for use, or the method of embodiment E160, wherein the method or composition results in:
(i) increased colon length in a subject; and/or
(ii) maintenance of body weight in a subject,
wherein the subject has, or is identified as having colitis, e.g., DSS induced colitis.

E162. The LNP composition for use, or the method of embodiment E160 or E161, wherein the colon length is increased by about 1.2-5-fold (e.g., about 2-4-fold, 2-5-fold, 2-3-fold, 3-5-fold, or 4-5-fold).

E163. The LNP composition for use, or the method of embodiment E160 or E161, wherein the change in colon length or body weight is compared to an otherwise similar subject, e.g., a subject who has not been contacted with the LNP composition comprising a polynucleotide comprising an mRNA which encodes an ITB6 molecule.

E164. The LNP composition for use, or the method of any one of embodiments E95-E133, wherein the disease with aberrant T cell function is diabetes, e.g., Type 1 diabetes (T1D).

E165. The LNP composition for use, or the method of embodiment E164, wherein the method or composition results in a reduction of blood glucose levels in a sample, e.g., a sample from a subject.

E166. The LNP composition for use, or the method of embodiment E164 or E165, wherein the reduction in blood glucose is at least 1.2-10-fold (e.g., about 2-25-fold, 5-20-fold, 10-15-fold, 5-30-fold, 10-30-fold, 20-30-fold, 2-20-fold, 2-15-fold, or 2-10-fold).

E167. The LNP composition for use, or the method of embodiment E164 or E165, wherein the reduction in blood glucose is compared to an otherwise similar subject, e.g., a subject who has not been contacted with the LNP composition comprising a polynucleotide comprising an mRNA which encodes an ITB6 molecule.

E168. The LNP composition for use, or the method of any one of embodiments E95-E167, wherein the polynucleotide comprising an mRNA encoding the ITB6 molecule, comprises at least one chemical modification.

E169. The LNP composition for use, or the method of E168, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

E170. The LNP composition for use, or the method of E168, wherein the chemical modification is selected from the group consisting of pseudouridine, N1-methylpseudouridine, 5-methylcytosine, 5-methoxyuridine, and a combination thereof.

E171. The LNP composition for use, or the method of E170, wherein the chemical modification is N1-methylpseudouridine.

E172. The LNP composition for use, or the method of any one of the preceding embodiments, wherein the mRNA in the lipid nanoparticle comprises fully modified N1-methylpseudouridine.

E173. The LNP composition for use, or the method of any one of the preceding embodiments, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

E174. The LNP composition for use, or the method of embodiment E173, wherein the ionizable lipid comprises an amino lipid.

E175. The LNP composition for use, or the method of embodiment E173 or E174, wherein the ionizable lipid comprises a compound of any of Formulae (I), (I-a), (I-b), (I-c), (II), (II-a), (II-b), (II-c), (II-d), (II-e), (II-f), (II-g), (II-h), or (III).

E176. The LNP composition for use, or the method of any one of embodiments E173-E175, wherein the ionizable lipid comprises a compound of Formula (I).

E177. The LNP composition for use, or the method of any one of embodiments E173-E176, wherein the ionizable lipid comprises Compound 18, Compound 25, Compound 301, or Compound 357.

E178. The LNP composition for use or the method of any one of embodiments E173-E177, wherein the LNP comprises a molar ratio of about 20-60% ionizable lipid:5-25% phospholipid:25-55% cholesterol; and 0.5-15% PEG lipid.

E179. The LNP composition for use or the method of embodiment E178, wherein the LNP comprises a molar ratio of about 50% ionizable lipid:about 10% phospholipid:about 38.5% cholesterol; and about 1.5% PEG lipid.

E180. The LNP composition for use or the method of embodiment E178 or E179, wherein the LNP comprises a molar ratio of about 49.83% ionizable lipid:about 9.83% phospholipid:about 30.33% cholesterol; and about 2.0% PEG lipid.

E181. The LNP composition for use or the method of any one of embodiments E173-E180, wherein the ionizable lipid comprises a compound of any of Formulae (I), (I-a), (I-b), (I-c), (II), (II-a), (II-b), (II-c), (II-d), (II-e), (II-f), (II-g), (II-h), or (III).

E182. The LNP composition for use or the method of embodiment E181, wherein the ionizable lipid comprises a compound of Formula (I).

E183. The LNP composition for use or the method of embodiment E181 or E182, wherein the ionizable lipid comprises Compound 18, Compound 25, Compound 301, or Compound 357.

E184. The LNP composition for use, or the method of any one of embodiments E95-E183, which is formulated for intravenous, subcutaneous, intramuscular, intranasal, intraocular, rectal, or oral delivery.

E185. The LNP composition for use, or the method of any one of embodiments E95-E184, further comprising a pharmaceutically acceptable carrier or excipient.

E186. A kit comprising a container comprising the lipid nanoparticle (LNP) composition of any one of embodiments E1-E93, or the pharmaceutical composition of embodiment E91, and a package insert comprising instructions for administration of the lipid nanoparticle or pharmaceutical composition for treating or delaying a disease with aberrant T cell function in an individual.

E187. The kit of embodiment E186, wherein the lipid nanoparticle composition comprises a pharmaceutically acceptable carrier.

### BRIEF DESCRIPTIONS OF THE DRAWINGS

FIGS. 1A-1C depict ITB6 expression in murine bone marrow derived dendritic cell line (JAWSII). Constructs are tagged with the V5 epitope to facilitate detection. FIG. 1A is a graph showing ITB6 expression in mock transfected cells. FIG. 1B is a graph showing ITB6 expression in cells transfected with a mouse ITB6 mRNA construct. FIG. 1C is a graph showing ITB6 expression in cells transfected with a human ITB6 mRNA construct.

FIG. 2A provides representative flow cytometry graphs depicting the frequency of FOXP3-GFP-expressing cells in CD4+ T cells co-cultured with murine bone marrow derived dendritic cell line (JAWSII) transfected with an LNP formulated with a control mRNA (NTFIX, dOXL40), mouse ITB6 mRNA, or human ITB6 mRNA, with or without latent TGFβ.

FIG. 2B provides a graph depicting the frequency of GFP+ cells in live CD4+ cells co-cultured with murine bone marrow derived dendritic cells (JAWSII) transfected with an LNP formulated with a control mRNA (NTFIX, dOXL40), mouse ITB6 mRNA, or human ITB6 mRNA, with or without latent TGFβ.

FIGS. 3A-3B depicts the frequency and absolute cell counts of OTII (CD45.2+CD4+) cells in the spleen of mice (adoptively transferred with CFSE labeled OTH cells) without OVA treatment, with OVA-only treatment, or OVA plus LNP formulated with a control mRNA (dOXL40), mouse ITB6 mRNA, or human ITB6 mRNA. FIG. 3A is a graph showing the frequency of OTII (CD45.2+CD4+) cells in the spleen. FIG. 3B is a graph showing the absolute cell counts of OTH (CD45.2+CD4+) cells in the spleen.

FIGS. 3C-3D depict the frequency and absolute cell counts of OTII regulatory Tregs (CD25+Fox3+CD45.2+CD4+) in the spleen of mice (adoptively transferred with CFSE labeled OTH cells) without OVA treatment, with OVA-only treatment, or OVA plus treatment with an LNP formulated with a control mRNA (dOXL40), mouse ITB6 mRNA, or human ITB6 mRNA.

FIG. 3C is a graph showing the frequency of OTII Tregs (CD25+Fox3+CD45.2+CD4+) in the spleen. FIG. 3D is a graph showing the absolute cell counts of Tregs (CD25+Fox3+CD45.2+CD4+) in the spleen.

FIGS. 3E-3F depict CFSE dilution and the frequency of CFSE^{low} cells in the spleen of mice (adoptively transferred with CFSE labeled OTII cells) without OVA treatment, or treated with OVA only, or with OVA plus an LNP formulated with a control mRNA (dOXL40), mouse ITB6 mRNA, or human ITB6 mRNA. FIG. 3E provides representative histograms showing CFSE dilution as a measure of proliferation of OTII cells in the spleen. FIG. 3F is a graph showing the frequency of CFSE^{low} cells (cells that have undergone proliferation, diluting CFSE) in the spleen.

FIG. 4A-4B depict the frequency of OTH (CD45.2+CD4+) cells and Tregs (CD25+FoxP3+) in the spleen of mice (adoptively transferred with CFSE labeled RagKO-OTII cells) without OVA treatment, with OVA-only treatment, or treated with OVA plus an LNP formulated with a control mRNA (dOXL40), or mouse ITB6 mRNA. FIG. 4A provides a graph showing the frequency of total OTII (CD45.2+CD4+) cells in the spleen. FIG. 4B provides a graph showing the frequency of OTII Tregs (CD25+Fox3+CD45.2+CD4+) in the spleen.

FIGS. 4C-4D depict CFSE dilution and the frequency of CFSE^{low} cells in the spleen of mice (adoptively transferred with CFSE labeled RagKO-OTII cells) without OVA treatment, or treated with OVA only, or OVA with an LNP formulated with a control mRNA (dOXL40), or mouse ITB6 mRNA. FIG. 4C provides representative histograms showing CFSE dilution as a measure of proliferation of OTII cells in the spleen. FIG. 4D provides a graph showing the frequency of CFSE^{low} cells (cells that have undergone proliferation, diluting CFSE) in the spleen.

FIGS. 5A-5B depict the OTI CD8+ T cell response and the frequency of IFNγ+ cells in OTI (CD45.2+) cells upon peptide restimulation. FIG. 5A is a representative graph showing the frequency of IFNγ+ OTI CD8+ T cells after ex vivo restimulation with peptide (SIINFEKL), from mice with OVA-only treatment or treated with OVA plus a LNP formulated with mouse ITB6 mRNA. FIG. 5B is a graph showing the frequency of IFNγ+ cells in OTI (CD45.2+) cells from mice without OVA treatment, with OVA-only treatment, or treated with rapamycin, or with an LNP formulated with a control mRNA (dOXL40), mouse ITB6 mRNA, or human ITB6 mRNA.

FIGS. 6A-6B depict the body weight change in GvHD mice after treatment. FIG. 6A is a graph from one study showing the body weight change in naïve mice, or mice treated with PBS or an LNP formulated with a control mRNA (dOXL40), or mouse ITB6 mRNA. FIG. 6B is a graph from another study showing the body weight change in naïve mice, or mice treated with an LNP formulated with a control mRNA (dOXL40), mouse ITB6 mRNA, or human ITB6 mRNA.

FIGS. 6C-6D depict the frequency of B cells, CD4+ T cells, and CD8+ T cells in GvHD mice at Day 8 of treatment. FIG. 6C is a graph showing the frequency of total B cells, CD4+ T cells, and CD8+ T cells in the blood of naïve mice, or mice that received donor cells and treated with an LNP formulated with a control mRNA (dOXL40), mouse ITB6 mRNA, or human ITB6 mRNA. FIG. 6D is a graph showing the frequency of donor (H2-K^{b+}) B cells, CD4+ T cells, and CD8+ T cells in the blood of naïve mice, or mice treated with an LNP formulated with a control mRNA (dOXL40), mouse ITB6 mRNA, or human ITB6 mRNA.

FIGS. 7A-7B depict the frequency of B cells, CD4+ T cells, and CD8+ T cells in GvHD mice at Day 13 of treatment. FIG. 7A is a graph showing the frequency of total B cells, CD4+ T cells, and CD8+ T cells in the blood of naïve mice, or mice treated with an LNP formulated with a control mRNA (dOXL40), mouse ITB6 mRNA, or human ITB6 mRNA. FIG. 7B is a graph showing the frequency of donor (H2-K^{b+}) B cells, CD4+ cells, and CD8+ cells in the blood of naïve mice, or mice treated with an LNP formulated with a control mRNA (dOXL40), mouse ITB6 mRNA, or human ITB6 mRNA.

FIGS. 7C-7D depict the frequency of Tregs in GvHD mice at Day 8 and Day 13 of treatment. FIG. 7C is a graph showing the frequency of Tregs (CD25+Foxp3+) in the CD4+ T cell population of naïve mice, or mice treated with an LNP formulated with a control mRNA (dOXL40), mouse ITB6 mRNA, or human ITB6 mRNA, at Day 8. FIG. 7D is a graph showing the frequency of Tregs (CD25+Foxp3+) in the CD4+ T cell population in naïve mice, or mice treated with an LNP formulated with a control mRNA (dOXL40), mouse ITB6 mRNA, or human ITB6 mRNA, at Day 13.

FIGS. 8A-8C depict the frequency and absolute cell counts of B cells, CD4+ cells, and CD8+ cells in the spleen of GvHD mice at Day 15 of treatment. FIG. 8A is a graph showing the frequency of B cells, CD4+ cells, and CD8+ cells in the spleen of naïve mice, or mice treated with PBS, or with an LNP formulated with a control mRNA (dOXL40), mouse ITB6 mRNA, or human ITB6 mRNA. FIG. 8B is a graph showing the absolute cell counts of B cells, CD4+ cells, and CD8+ cells in the spleen of naïve mice, or mice treated with PBS, or with an LNP formulated with a control mRNA (dOXL40), mouse ITB6 mRNA, or human ITB6 mRNA. FIG. 8C is a graph showing the frequency of donor (H2-K^{b+}) B cells, CD4+ cells, and CD8+ cells in the spleen of naïve mice, or mice treated with PBS, or with an LNP formulated with a control mRNA (dOXL40), mouse ITB6 mRNA, or human ITB6 mRNA.

FIGS. 8D-8E depict the frequency of Tregs in the spleen of GvHD mice at Day 15 of treatment. FIG. 8D is a graph showing the frequency of Tregs (CD25+Foxp3+) in the spleen of naïve mice, or mice treated with PBS, or with an LNP formulated with a control mRNA (dOXL40), mouse ITB6 mRNA, or human ITB6 mRNA. FIG. 8E is a graph showing the absolute cell counts of Tregs (CD25+Foxp3+) in the spleen of naïve mice, or mice treated with PBS, or an LNP formulated with a control mRNA (dOXL40), mouse ITB6 mRNA, or human ITB6 mRNA.

FIGS. 9A-9E depicts the proliferation of donor and host cells in the spleen of GvHD mice at Day 15 of treatment as measured by Ki67 staining. FIG. 9A is a graph showing the frequency of Ki67 total B cells, CD4+ cells, and CD8+ cells in the spleen of naïve mice, or in mice treated with PBS, or with an LNP formulated with a control mRNA (dOXL40), mouse ITB6 mRNA, or human ITB6 mRNA. FIG. 9B is a representative graph showing the relative proliferation of donor and host cells in mice treated with PBS, or with an LNP formulated with mouse ITB6 mRNA as measured by Ki67 staining. FIG. 9C is a graph showing the frequency of Ki67 donor and host B cells in naïve mice, or in mice treated with PBS, or with an LNP formulated with a control mRNA (dOXL40), mouse ITB6 mRNA, or human ITB6 mRNA. FIG. 9D is a graph showing the frequency of Ki67 donor and host CD4+ T cells in naïve mice, or in mice treated with PBS, or with an LNP formulated with a control mRNA (dOXL40), mouse ITB6 mRNA, or human ITB6 mRNA. FIG. 9E is a graph showing the frequency of Ki67 donor and host CD8+ T cells in naïve mice, or in mice treated with PBS, or with an LNP formulated with a control mRNA (dOXL40), mouse ITB6 mRNA, or human ITB6 mRNA.

FIGS. 10A-10B depict the frequency of Tbet+ cells in the spleen of GvHD mice. FIG. 10A is a graph showing the frequency of Tbet+ cells in the CD8+ T cell population in naïve mice, or in mice treated with PBS, or with an LNP formulated with a control mRNA (dOXL40), mouse ITB6 mRNA, or human ITB6 mRNA. FIG. 10B is a graph showing the frequency of Tbet+ cells in the Tconv cell (CD25-Foxp3-CD4+) population in naïve mice, or in mice treated with PBS, or with an LNP formulated with a control mRNA (dOXL40), mouse ITB6 mRNA, or human ITB6 mRNA.

FIG. 11 depicts the day 15 serum levels of CXCL1, IFNγ, TNFα, MCP1, M1P1α, IL10, IL13, IL5, IL9, and M1P1β in naïve mice, or in GvHD mice treated with PBS, or with an LNP formulated with a control mRNA (dOXL40), mouse ITB6 mRNA, or human ITB6 mRNA.

FIGS. 12A-12D depicts the frequency and proliferation of B cells, CD4+ cells, and CD8+ cells, and the frequency of Tregs, in the spleen of GvHD mice at Day 22 of treatment. FIG. 12A is a graph showing the frequency of B cells, CD4+ cells, and CD8+ cells in the spleen of naïve mice, or in mice treated with PBS, or with an LNP formulated with a control mRNA (dOXL40), mouse ITB6 mRNA, or human ITB6 mRNA. FIG. 12B is a graph showing the frequency of donor (H2-K^{b+}) B cells, CD4+ cells, and CD8+ cells in the spleen of naïve mice, or in mice treated with PBS, or with an LNP formulated with a control mRNA (dOXL40), mouse ITB6 mRNA, or human ITB6 mRNA. FIG. 12C is a graph showing the frequency of Tregs (CD25+Foxp3+) in the CD4+ cell population in the spleen of naïve mice, or in mice treated with PBS, or with an LNP formulated with a control mRNA (dOXL40), mouse ITB6 mRNA, or human ITB6 mRNA. FIG. 12D is a graph showing the frequency of Ki67+ B cells, CD4+ cells, and CD8+ cells in the spleen of naïve mice, or in mice treated with PBS, or with an LNP formulated with a control mRNA (dOXL40), mouse ITB6 mRNA, or human ITB6 mRNA.

FIG. 13A is a table depicting the experimental groups for the NOD-scid-gamma (NSG) mice model of GvHD experiments.

FIG. 13B is a graph showing survival curves following treatment. Time of LNP treatment indicated by vertical arrows at top of graph.

FIG. 13C is a graph showing body weight changes over time.

FIG. 14 is a series of graphs showing % OTII (14A), % Tregs (14B), proliferation (14C-14E), and OTH (CD45.2+) proliferation and % of CFSEₗₒ population (14F) in the spleen of mice following treatment with an LNP formulated with human ITB6 mRNA.

FIG. 15 is a pair of graphs showing KLH-specific IgG in mice following treatment with an LNP formulated with human ITB6 mRNA.

FIG. 16 is a pair of graphs showing KLH-specific IgM in mice following treatment with an LNP formulated with human ITB6 mRNA.

FIG. 17 is a pair of graphs showing KLH-specific IgM in mice following treatment with an LNP formulated with human ITB6 mRNA.

FIG. 18 is a pair of graphs showing KLH-specific IgG in mice following treatment with an LNP formulated with human ITB6 mRNA.

FIG. 19 is a pair of graphs showing KLH-specific IgG in mice following treatment with an LNP formulated with human ITB6 mRNA.

FIG. 20 is a schematic and pair of graphs showing experimental design, % B cells in the spleen, and % H2-Kb+ donor cell sin the spleen of mice following treatment with an LNP formulated with human ITB6 mRNA.

FIG. 21 is a schematic and table showing experimental design.

FIG. 22 is a heatmap showing genes that changed after exposure to LNPs formulated with ITB6 mRNA compared to LNPs formulated with dOX40L.

FIG. 23 is a summary table showing the fold change and peak expression of several biomarker genes following treatment with LNPs formulated with ITB6 mRNA in mouse, rat, NHP, and human PBMCs.

FIGS. 24A and 24B are a pair of graphs showing mean clinical scores (FIG. 24A) and mean day disease onset (FIG. 24B) in a mouse model of EAE following treatment with vehicle, LNPs formulated with a dmOX40L control, or LNPs formulated with ITB6 mRNA.

### DETAILED DESCRIPTION

Without wishing to be bound by theory, it is believed that, in some embodiments, administration of an LNP comprising a polynucleotide comprising an mRNA which encodes an ITB6 molecule can result in suppression of T cells, e.g., reduction of T cell expansion, T cell proliferation, T cell anergy, and/or T cell apoptosis, e.g., by induction and/or proliferation of Treg cells. Exemplary inhibitory effects on T cells in vitro and in vivo with LNP compositions disclosed herein are provided at least in Examples 3-5 and effects on induction and/or proliferation of Treg cells are provided at least in Example 12. Exemplary protective in vivo effects of LNPs comprising ITB6 molecules are provided at least in Examples 6-11 (in a GvHD model) and Example 16 (in an EAE model).

Accordingly, the present disclosure provides, inter alia, LNP compositions comprising ITB6-encoding nucleic acid (e.g., mRNA) therapeutics. Also disclosed herein are methods of using such LNP compositions for inhibiting an immune response, or for treating or preventing a disease associated with an aberrant immune cell function, in a subject.

### Definitions

Acquiring: As used herein, “acquiring” refers to obtaining possession of a physical entity (e.g., a sample, polypeptide, or nucleic acid), or a value (e.g., a numerical value), by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance (e.g., a starting material). Exemplary changes include separating or purifying a substance, combining two or more separate entities into a mixture, or performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent, performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intravenous or subcutaneous.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean+/−5% of the recited value. For instance, an LNP including a lipid component having about 40% of a given compound may include 30-50% of the compound.

Biomarker: as used herein, the term “biomarker” refers to an indicator, e.g., a predictive and/or prognostic indicator, which can be detected in a sample (e.g., a gene) or derived from one or more indicators detected from a sample. The biomarker may serve as an indicator of efficacy of a therapy (e.g., therapy comprising an ITB6 mRNA) characterized by certain molecular, pathological, histological, and/or clinical features. In some embodiments, a biomarker is a gene.

In other embodiments, a biomarker is a collection of genes. Biomarkers include, but are not limited to, polynucleotides (e.g., DNA, and/or RNA), polynucleotide copy number alterations (e.g., DNA copy numbers), polypeptides, polypeptide and polynucleotide modifications (e.g. posttranslational modifications), carbohydrates, and/or glycolipid-based molecular markers.

Such biomarkers include, but are not limited to, PMEPA1, ITGAE/CD103, SMAD7, SKIL, and SKI.

PMEPA1: As used herein, “PMEPA1” refers to any native Prostate Transmembrane Protein, Androgen Induced 1 from any vertebrate source, including mammals such as primates (e.g., humans) and rodents (e.g., mice and rats), unless otherwise indicated. The term encompasses “full-length,” unprocessed PMEPA1 as well as any form of PMEPA1 that results from processing in the cell. The term also encompasses naturally occurring variants of PMEPA1, e.g., splice variants or allelic variants. PMEPA1 is also referred to in the art as STAG1, TMEPA1, Solid Tumor-Associated 1 Protein, and Transmembrane Prostate Androgen-Induced Protein. The nucleic acid sequence of an exemplary human PMEPA1 is shown under NCBI Reference Sequence: NG_031951.1. The amino acid sequence of an exemplary protein encoded by human PMEPA1 is shown under UniProt Accession No. Q969W9-1.

ITGAE: As used herein, “ITGAE” and “CD103” refer to any native Integrin Subunit Alpha E from any vertebrate source, including mammals such as primates (e.g., humans) and rodents (e.g., mice and rats), unless otherwise indicated. The term encompasses “full-length,” unprocessed ITGAE as well as any form of ITGAE that results from processing in the cell. The term also encompasses naturally occurring variants of ITGAE, e.g., splice variants or allelic variants. ITGAE is also referred to in the art as HUMINAE, Mucosal Lymphocyte 1 Antigen, Integrin Alpha-IEL, HML-1 Antigen, and CD103 Antigen. The nucleic acid sequence of an exemplary human ITGAE is shown under NCBI Reference Sequence: NC_000017.11. The amino acid sequence of an exemplary protein encoded by human ITGAE is shown under UniProt Accession No. P38570.

SMAD7: As used herein, “SMAD7” refers to any native SMAD Family Member 7 from any vertebrate source, including mammals such as primates (e.g., humans) and rodents (e.g., mice and rats), unless otherwise indicated. The term encompasses “full-length,” unprocessed SMAD7 as well as any form of SMAD7 that results from processing in the cell. The term also encompasses naturally occurring variants of SMAD7, e.g., splice variants or allelic variants. SMAD7 is also referred to in the art as SMAD Family Member 7, MADH7, MADH8, Mothers Against Decapentaplegic Homolog 7, MAD Homolog 7, MAD Homolog 8, HSMAD7, and CRCS3. The nucleic acid sequence of an exemplary human SMAD7 is shown under NCBI Reference Sequence: NM_005904.4. The amino acid sequence of an exemplary protein encoded by human SMAD7 is shown under UniProt Accession No. 015105.

SKIL: As used herein, “SKIL” refers to any native SKI Like Proto-Oncogene from any vertebrate source, including mammals such as primates (e.g., humans) and rodents (e.g., mice and rats), unless otherwise indicated. The term encompasses “full-length,” unprocessed SKIL as well as any form of SKIL that results from processing in the cell. The term also encompasses naturally occurring variants of SKIL, e.g., splice variants or allelic variants. SKIL is also referred to in the art as SKI Like Proto-Oncogene, SNO, SnoN, SnoA, Ski-Like Protein, Ski-Related Oncogene SnoN, and SnoI. The nucleic acid sequence of an exemplary human SKIL is shown under NCBI Reference Sequence: NM_005414.5. The amino acid sequence of an exemplary protein encoded by human SKIL is shown under UniProt Accession No. P12757.

SKI: As used herein, “SKI” refers to any native SKI Proto-Oncogene from any vertebrate source, including mammals such as primates (e.g., humans) and rodents (e.g., mice and rats), unless otherwise indicated. The term encompasses “full-length,” unprocessed SKI as well as any form of SKI that results from processing in the cell. The term also encompasses naturally occurring variants of SKI, e.g., splice variants or allelic variants. SKI is also referred to in the art as V-Ski Avian Sarcoma Viral Oncogene Homolog, Sloan-Kettering Institute Proto-Oncogene, Proto-Oncogene C-Ski, Ski Oncogene, SGS, and SKV. The nucleic acid sequence of an exemplary human SKI is shown under NCBI Reference Sequence: NM_003036.4. The amino acid sequence of an exemplary protein encoded by human SKI is shown under UniProt Accession No. P12755.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering an LNP including the therapeutic and/or prophylactic to the subject (e.g., by an intravenous, intramuscular, intradermal, or subcutaneous route). Administration of an LNP to a mammal or mammalian cell may involve contacting one or more cells with the lipid nanoparticle.

Detecting: The term “detecting” is used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of an LNP, relative to the initial total amount of therapeutic and/or prophylactic used in the preparation of an LNP. For example, if 97 mg of therapeutic and/or prophylactic are encapsulated in an LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Expression level: The terms “level of expression” or “expression level” in general are used interchangeably and generally refer to the amount of a biomarker in a biological sample. An expression level for more than one gene of interest may be determined by aggregation methods known to one skilled in the art and also disclosed herein, including, for example, by calculating the median or mean of all the expression levels of the genes of interest. Before aggregation, the expression level of each gene of interest may be normalized by using statistical methods known to one skilled in the art and also disclosed herein, including, for example, normalized to the expression level of one or more housekeeping genes, or normalized to a total library size, or normalized to the median or mean expression level value across all genes measured. In some instances, before aggregation across multiple genes of interest, the normalized expression level of each gene of interest may be standardized by using statistical methods known to one skilled in the art and also disclosed herein, including, for example, by calculating the Z-score of the normalized expression level of each gene of interest.

Reference Expression Level: As used herein, the terms “reference expression level” and “reference level” are used interchangeably to refer to an expression level against which another expression level, e.g., the expression level of one or more genes described herein (e.g., any gene or combination of genes selected from PMEPA1, ITGAE/CD103, SMAD7, SKIL, and SKI) in a sample from an individual is compared, e.g., to make a diagnostic (e.g., predictive and/or prognostic) and/or therapeutic determination. For example, the reference expression level may be derived from expression levels in a reference population (e.g., the median expression level in a reference population, e.g., a population of patients having an autoimmune or inflammatory disease who have not been treated with an ITB6 mRNA therapy), a reference sample, and/or a pre-assigned value (e.g., a cut-off value which was previously determined to significantly (e.g., statistically significantly)) separate a first subset of individuals who exhibited disease progression and a second subset of individuals who did not exhibit disease progression, wherein the reference expression level significantly separates the first and second subsets of individuals based on a significant difference between the expression level in the first subset of individuals compared to that of the second subset of individuals. In some embodiments, the cut-off value may be the median or mean expression level in the reference population. In other embodiments, the reference level may be the top 40%, the top 30%, the top 20%, the top 10%, the top 5%, or the top 1% of the expression level in the reference population. In particular embodiments, the cut-off value may be the median expression level in the reference population. It will be appreciated by one skilled in the art that the numerical value for the reference expression level may vary depending on the indication or disorder, the methodology used to detect expression levels (e.g., RNA-seq, microarray analysis, or RT-qPCR), and/or the specific combinations of genes examined (e.g., any combination of the genes selected from PMEPA1, ITGAE/CD103, SMAD7, SKIL, and SKI).

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

ITB6 molecule: As used herein, the term “ITB6 molecule” refers to a full length naturally occurring ITB6 (e.g., a mammalian ITB6, e.g., human ITB6, e.g., associated with UniProt: P18564; NCBI Gene ID: 3694) a fragment (e.g., a functional fragment) of ITB6, or a variant of ITB6 having at least 80%, 85%, 90%, 95%, 96%, 97%, 98%, or 99% sequence identity to: a naturally-occurring wild type ITB6 or a fragment (e.g., a functional fragment) thereof. In some embodiments, the ITB6 molecule is a ITGB6 gene product, e.g., an ITB6 polypeptide. In some embodiments, the variant, e.g., active variant, is a derivative, e.g., a mutant, of a wild type polypeptide. In some embodiments, the ITB6 variant, e.g., active variant of ITB6, has at least 50%, 60%, 70%, 80%, 85%, 90%, 95%, 96%, 97%, 98%, 99% or 100% activity of wild type ITB6 polypeptide. In some embodiments, the ITB6 molecule comprises a portion of ITB6 (e.g., an extracellular portion of ITB6) and a heterologous sequence, e.g., a sequence other than that of naturally occurring ITB6. In some embodiments, the ITB6 molecule comprises soluble ITB6.

Heterologous: As used herein, “heterologous” indicates that a sequence (e.g., an amino acid sequence or the polynucleotide that encodes an amino acid sequence or a non-coding region of a nucleic acid molecule) is not normally present in a given polypeptide or polynucleotide in nature. For example, an amino acid sequence that corresponds to a domain or motif of one protein may be heterologous to a second protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Modified: As used herein “modified” or “modification” refers to a changed state or a change in composition or structure of a polynucleotide (e.g., mRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

Nanoparticle: As used herein, “nanoparticle” refers to a particle having any one structural feature on a scale of less than about 1000 nm that exhibits novel properties as compared to a bulk sample of the same material Routinely, nanoparticles have any one structural feature on a scale of less than about 500 nm, less than about 200 nm, or about 100 nm. Also, routinely nanoparticles have any one structural feature on a scale of from about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid. As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Responsiveness: “Responsiveness” or “effective response” can be assessed using any endpoint indicating a benefit to the individual and includes, without limitation, (i) inhibition, to some extent, of disease progression, including slowing down and complete arrest; (ii) reduction in the number of disease episodes and/or symptoms; (iii) reduction in lesional size; (iv) inhibition (i.e., reduction, slowing down or complete stopping) of disease cell infiltration into adjacent peripheral organs and/or tissues; (v) inhibition (i.e. reduction, slowing down or complete stopping) of disease spread; (vi) decrease of auto-immune response, which may, but does not have to, result in the regression or ablation of the disease lesion; (vii) relief, to some extent, of one or more symptoms associated with the disorder; (viii) increase in the length of disease-free presentation following treatment; and/or (ix) decreased mortality at a given point of time following treatment.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally occurring, non-naturally occurring, synthetic, engineered, or any combination thereof.

For example, naturally occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian target cell) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition. In some embodiments, a therapy intended to prevent a disease may be given prophylactically (e.g., administered before onset of one or more symptoms).

Unmodified: As used herein, “unmodified” refers to any substance, compound, or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Variant: As used herein, the term “variant” refers to a molecule having at least 50%, 60%, 70%, 80%, 85%, 90%, 95%, 96%, 97%, 98%, 99% or 100% activity of the wild type molecule, e.g., as measured by an art-recognized assay.

### ITB6 Molecules

ITB6 (also known as integrin beta 6) is a subunit of the heterodimeric integrin αvβ6, a membrane-spanning glycoprotein. In an aspect, the disclosure provides an LNP composition comprising a polynucleotide (e.g., an mRNA), e.g., encoding an ITB6 molecule, e.g., as described herein. In an embodiment, the ITB6 molecule comprises a naturally occurring ITB6 molecule, a fragment of a naturally occurring ITB6 molecule, or a variant thereof. In an embodiment, the ITB6 molecule comprises a variant of a naturally occurring ITB6 molecule (e.g., an ITB6 variant, e.g., as described herein), or a fragment thereof. In an embodiment, the LNP composition comprising a polynucleotide (e.g., an mRNA) encoding an ITB6 molecule can be administered alone or in combination with an additional agent, e.g., an LNP composition comprising a polynucleotide (e.g., an mRNA) encoding a different ITB6 variant or fragment thereof or an LNP composition comprising a polynucleotide (e.g., an mRNA) encoding a different molecule.

In an aspect, an LNP composition disclosed herein comprises a polynucleotide (e.g., an mRNA) encoding an ITB6 molecule. In an embodiment, the ITB6 molecule comprises a naturally occurring ITB6 molecule, a fragment of a naturally occurring ITB6 molecule, or a variant thereof. In an embodiment, the ITB6 molecule comprises an amino acid sequence having at least 85%, 90%, 95%, 96%, 97%, 98%, 99%, or 100% identity to an ITB6 amino acid sequence provided in Table 1A or Table 2A, e.g., any one of SEQ ID NOs: 17, 1, 7, 9, 11, 13, or 15, or a functional fragment thereof. In an embodiment, the ITB6 molecule comprises the amino acid sequence of an ITB6 amino acid sequence provided in Table 1A or Table 2A, e.g., any one of SEQ ID NOs: 17, 1, 7, 9, 11, 13, or 15, or a functional fragment thereof. In an embodiment, the ITB6 molecule comprises the amino acid sequence of any one of SEQ ID NOs: 17, 1, 7, 9, 11, 13, or 15 or a functional fragment thereof.

In some embodiments, the ITB6 molecule comprises an amino acid sequence having at least 85%, 90%, 95%, 96%, 97%, 98%, 99%, or 100% identity to, or differing by no more than 1, 2, 5, 10, 15, 20, 25, 30, 40, 50, 60, 70, 80, 90, or 100 amino acids from, an amino acid sequence lacking a leader sequence and/or an affinity tag (e.g., a leader sequence and/or an affinity tag described in Table 1A or Table 2A) but otherwise identical to an amino acid sequence of ITB6 provided in Table 1A or Table 2A, e.g., any one of SEQ ID NOs: 17, 1, 7, 9, 11, 13, or 15, or a functional fragment thereof.

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, 100, 150, 200, 250, or 300 nucleotides from, a nucleotide sequence provided in Table 1A or Table 2A, e.g., any one of SEQ ID NOs: 18, 2-6, 8, 10, 12, 14, 16, or 160-175, or a functional fragment thereof. In an embodiment, the polynucleotide (e.g., mRNA) encoding the ITB6 molecule comprises the nucleotide sequence of SEQ ID NO: 18. In an embodiment, the polynucleotide (e.g., mRNA) encoding the ITB6 molecule comprises the nucleotide sequence of SEQ ID NO: 2. In an embodiment, the polynucleotide (e.g., mRNA) encoding the ITB6 molecule comprises the nucleotide sequence of SEQ ID NO: 3. In an embodiment, the polynucleotide (e.g., mRNA) encoding the ITB6 molecule comprises the nucleotide sequence of SEQ ID NO: 4. In an embodiment, the polynucleotide (e.g., mRNA) encoding the ITB6 molecule comprises the nucleotide sequence of SEQ ID NO: 5. In an embodiment, the polynucleotide (e.g., mRNA) encoding the ITB6 molecule comprises the nucleotide sequence of SEQ ID NO: 6. In an embodiment, the polynucleotide (e.g., mRNA) encoding the ITB6 molecule comprises the nucleotide sequence of SEQ ID NO: 8. In an embodiment, the polynucleotide (e.g., mRNA) encoding the ITB6 molecule comprises the nucleotide sequence of SEQ ID NO: 10. In an embodiment, the polynucleotide (e.g., mRNA) encoding the ITB6 molecule comprises the nucleotide sequence of SEQ ID NO: 12. In an embodiment, the polynucleotide (e.g., mRNA) encoding the ITB6 molecule comprises the nucleotide sequence of SEQ ID NO: 14. In an embodiment, the polynucleotide (e.g., mRNA) encoding the ITB6 molecule comprises the nucleotide sequence of SEQ ID NO: 16.

In some embodiments, the polynucleotide (e.g., mRNA) encoding the ITB6 molecule comprises a nucleic acid sequence having at least 85%, 90%, 95%, 96%, 97%, 98%, 99% or 100% identity to, or differing by no more than 5, 10, 15, 20, 30, 40, 50, 60, 70, 80, 90, 100, 150, 200, 250, or 300 nucleotides from, a nucleic acid sequence lacking a nucleotide sequence that encodes for a leader sequence and/or an affinity tag (e.g., a leader sequence and/or an affinity tag described in Table 1A or Table 2A) but otherwise identical to a nucleotide sequence provided in Table 1A or Table 2A, e.g., any one of SEQ ID NOs: 18, 2-6, 8, 10, 12, 14, 16, or 160-175, or a functional fragment thereof.

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from, the sequence of SEQ ID NO: 18. In an embodiment, the polynucleotide (e.g., mRNA) encoding the ITB6 molecule comprises the nucleotide sequence of SEQ ID NO: 18. In some embodiments, the polynucleotide (e.g., mRNA) encoding the ITB6 molecule comprises the nucleotide sequence of SEQ ID NO: 175, which comprises from 5′ to 3′ end: the 5′ UTR sequence of SEQ ID NO: 50, the ORF sequence of SEQ ID NO:18, and the 3′ UTR sequence of SEQ ID NO: 142.

In some embodiments, the polynucleotide encoding the ITB6 molecule comprises from 5′ to 3′ end
(i) a 5′ cap such as provided herein, e.g., m⁷GpppG_{2′OMe}pA;
(ii) a 5′ UTR, such as the sequences provided herein, for example, SEQ ID NO:50;
(iii) an open reading frame encoding an ITB6 polypeptide, e.g., a sequence optimized nucleic acid sequence encoding ITB6 set forth as SEQ ID NO:18;
(iv) at least one stop codon (if not present at 5′ terminus of 3′UTR);
(v) a 3′ UTR, such as the sequences provided herein, for example, SEQ ID NO:142; and
(vi) a poly-A tail provided herein (e.g., SEQ ID NO:502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from, the sequence of SEQ ID NO: 2. In an embodiment, the polynucleotide (e.g., mRNA) encoding the ITB6 molecule comprises the nucleotide sequence of SEQ ID NO: 2. In some embodiments, the polynucleotide (e.g., mRNA) encoding the ITB6 molecule comprises the nucleotide sequence of SEQ ID NO: 160, which comprises from 5′ to 3′ end: the 5′ UTR sequence of SEQ ID NO: 56, the ORF sequence of SEQ ID NO: 2, and the 3′ UTR sequence of SEQ ID NO: 110.

In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO:56;
(iii) an open reading frame encoding an ITB6 polypeptide, e.g., a sequence optimized nucleic acid sequence encoding ITB6 set forth as SEQ ID NO: 2;
(iv) at least one stop codon (if not present at 5′ terminus of 3′UTR);
(v) a 3′ UTR, such as the sequences provided herein, for example, SEQ ID NO:110; and
(vi) a poly-A tail provided herein (e.g., SEQ ID NO:502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from the sequence of SEQ ID NO: 3. In an embodiment, the polynucleotide (e.g., mRNA) encoding the ITB6 molecule comprises the nucleotide sequence of SEQ ID NO: 3. In some embodiments, the polynucleotide encoding the ITB6 molecule comprises the nucleotide sequence of SEQ ID NO: 161, which comprises from 5′ to 3′ end: the 5′ UTR sequence of SEQ ID NO: 50, the ORF sequence of SEQ ID NO: 3, and the 3′ UTR sequence of SEQ ID NO: 110.

In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO:50;
(iii) an open reading frame encoding an ITB6 polypeptide, e.g., a sequence optimized nucleic acid sequence encoding ITB6 set forth as SEQ ID NO: 3;
(iv) at least one stop codon (if not present at 5′ terminus of 3′UTR);
(v) a 3′ UTR, such as the sequences provided herein, for example, SEQ ID NO: 110; and
(vi) a poly-A tail provided herein (e.g., SEQ ID NO:502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from the sequence of SEQ ID NO: 4. In an embodiment, the polynucleotide (e.g., mRNA) encoding the ITB6 molecule comprises the nucleotide sequence of SEQ ID NO: 4. In some embodiments, the polynucleotide encoding the ITB6 molecule comprises the nucleotide sequence of SEQ ID NO: 162, which comprises from 5′ to 3′ end: the 5′ UTR sequence of SEQ ID NO: 50, ORF sequence of SEQ ID NO: 4, and the 3′ UTR sequence of SEQ ID NO: 143.

In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO:50;
(iii) an open reading frame encoding an ITB6 polypeptide, e.g., a sequence optimized nucleic acid sequence encoding ITB6 set forth as SEQ ID NO: 4;
(iv) at least one stop codon (if not present at 5′ terminus of 3′UTR);
(v) a 3′ UTR, such as the sequences provided herein, for example, SEQ ID NO: 143; and
(vi) a poly-A tail provided herein (e.g., SEQ ID NO: 502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from the sequence of SEQ ID NO: 5. In an embodiment, the polynucleotide (e.g., mRNA) encoding the ITB6 molecule comprises the nucleotide sequence of SEQ ID NO: 5. In some embodiments, the polynucleotide encoding the ITB6 molecule comprises the nucleotide sequence of SEQ ID NO: 163, which comprises from 5′ to 3′ end: the 5′ UTR sequence of SEQ ID NO: 50, the ORF sequence of SEQ ID NO: 5, and the 3′ UTR sequence of SEQ ID NO: 110.

In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO: 50;
(iii) an open reading frame encoding an ITB6 polypeptide, e.g., a sequence optimized nucleic acid sequence encoding ITB6 set forth as SEQ ID NO: 5;
(iv) at least one stop codon (if not present at 5′ terminus of 3′UTR);
(v) a 3′ UTR, such as the sequences provided herein, for example, SEQ ID NO: 110; and
(vi) a poly-A tail provided herein (e.g., SEQ ID NO: 502).

In some embodiments, the polynucleotide (e.g., mRNA) encoding the ITB6 molecule comprises the nucleotide sequence of SEQ ID NO: 164, which comprises from 5′ to 3′ end: the 5′ UTR sequence of SEQ ID NO: 50, the ORF sequence of SEQ ID NO: 5, and the 3′ UTR sequence of SEQ ID NO: 144.

In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO: 50;
(iii) an open reading frame encoding an ITB6 polypeptide, e.g., a sequence optimized nucleic acid sequence encoding ITB6 set forth as SEQ ID NO: 5;
(iv) at least one stop codon (if not present at 5′ terminus of 3′UTR);
(v) a 3′ UTR, such as the sequences provided herein, for example, SEQ ID NO: 144; and
(vi) a poly-A tail provided herein (e.g., SEQ ID NO: 502).

In some embodiments, the polynucleotide (e.g., mRNA) encoding the ITB6 molecule comprises the nucleotide sequence of SEQ ID NO: 169, which comprises from 5′ to 3′ end: the 5′ UTR sequence of SEQ ID NO: 50, the ORF sequence of SEQ ID NO: 5, and the 3′ UTR sequence of SEQ ID NO: 145.

In some embodiments, the polynucleotide encoding the ITB6 molecule comprises from 5′ to 3′ end
(i) a 5′ cap such as provided herein, e.g., Cap II-b;
(ii) a 5′ UTR, such as the sequences provided herein, for example, SEQ ID NO: 50;
(iii) an open reading frame encoding an ITB6 polypeptide, e.g., a sequence optimized nucleic acid sequence encoding ITB6 set forth as SEQ ID NO: 5;
(iv) at least one stop codon (if not present at 5′ terminus of 3′UTR);
(v) a 3′ UTR, such as the sequences provided herein, for example, SEQ ID NO: 145; and
(vi) a poly-A tail provided herein (e.g., SEQ ID NO: 502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from the sequence of SEQ ID NO: 6. In an embodiment, the polynucleotide (e.g., mRNA) encoding the ITB6 molecule comprises the nucleotide sequence of SEQ ID NO: 6. In some embodiments, the polynucleotide encoding the ITB6 molecule comprises the nucleotide sequence of SEQ ID NO: 165, which comprises from 5′ to 3′ end: the 5′ UTR sequence of SEQ ID NO: 50, the ORF sequence of SEQ ID NO: 6, and the 3′ UTR sequence of SEQ ID NO: 110 In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO: 50;
(iii) an open reading frame encoding an ITB6 polypeptide, e.g., a sequence optimized nucleic acid sequence encoding ITB6 set forth as SEQ ID NO: 6;
(iv) at least one stop codon (if not present at 5′ terminus of 3′UTR);
(v) a 3′ UTR, such as the sequences provided herein, for example, SEQ ID NO: 110; and
(vi) a poly-A tail provided herein (e.g., SEQ ID NO:502).

In some embodiments, the polynucleotide (e.g., mRNA) encoding the ITB6 molecule comprises the nucleotide sequence of SEQ ID NO: 166, which comprises from 5′ to 3′ end: the 5′ UTR sequence of SEQ ID NO: 50, the ORF sequence of SEQ ID NO: 6, and the 3′ UTR sequence of SEQ ID NO: 110.

In some embodiments, the polynucleotide encoding the ITB6 molecule comprises from 5′ to 3′ end
(i) a 5′ cap such as provided herein, e.g., Cap II-b;
(ii) a 5′ UTR, such as the sequences provided herein, for example, SEQ ID NO:50;
(iii) an open reading frame encoding an ITB6 polypeptide, e.g., a sequence optimized nucleic acid sequence encoding ITB6 set forth as SEQ ID NO: 6;
(iv) at least one stop codon (if not present at 5′ terminus of 3′UTR);
(v) a 3′ UTR, such as the sequences provided herein, for example, SEQ ID NO: 110; and
(vi) a poly-A tail provided herein (e.g., SEQ ID NO:502).

In some embodiments, the polynucleotide (e.g., mRNA) encoding the ITB6 molecule comprises the nucleotide sequence of SEQ ID NO: 167, which comprises from 5′ to 3′ end: the 5′ UTR sequence of SEQ ID NO: 50, the ORF sequence of SEQ ID NO: 6, and the 3′ UTR sequence of SEQ ID NO: 145.

In some embodiments, the polynucleotide encoding the ITB6 molecule comprises from 5′ to 3′ end
(i) a 5′ cap such as provided herein, e.g., Cap II-b;
(ii) a 5′ UTR, such as the sequences provided herein, for example, SEQ ID NO:50;
(iii) an open reading frame encoding an ITB6 polypeptide, e.g., a sequence optimized nucleic acid sequence encoding ITB6 set forth as SEQ ID NO: 6;
(iv) at least one stop codon (if not present at 5′ terminus of 3′UTR);
(v) a 3′ UTR, such as the sequences provided herein, for example, SEQ ID NO: 145; and
(vi) a poly-A tail provided herein (e.g., SEQ ID NO:502).

In some embodiments, the polynucleotide (e.g., mRNA) encoding the ITB6 molecule comprises the nucleotide sequence of SEQ ID NO: 168, which comprises from 5′ to 3′ end: the 5′ UTR sequence of SEQ ID NO: 50, the ORF sequence of SEQ ID NO: 6, and the 3′ UTR sequence of SEQ ID NO: 143.

In some embodiments, the polynucleotide encoding the ITB6 molecule comprises from 5′ to 3′ end
(i) a 5′ cap such as provided herein, e.g., Cap II-b;
(ii) a 5′ UTR, such as the sequences provided herein, for example, SEQ ID NO:50;
(iii) an open reading frame encoding an ITB6 polypeptide, e.g., a sequence optimized nucleic acid sequence encoding ITB6 set forth as SEQ ID NO: 6;
(iv) at least one stop codon (if not present at 5′ terminus of 3′UTR);
(v) a 3′ UTR, such as the sequences provided herein, for example, SEQ ID NO: 143; and
(vi) a poly-A tail provided herein (e.g., SEQ ID NO:502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from the sequence of SEQ ID NO: 8. In an embodiment, the polynucleotide (e.g., mRNA) encoding the ITB6 molecule comprises the nucleotide sequence of SEQ ID NO: 8. In some embodiments, the polynucleotide encoding the ITB6 molecule comprises the nucleotide sequence of SEQ ID NO: 170, which comprises from 5′ to 3′ end: the 5′ UTR sequence of SEQ ID NO: 50, the ORF sequence of SEQ ID NO: 8, and the 3′ UTR sequence of SEQ ID NO: 110.

In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO: 50;
(iii) an open reading frame encoding an ITB6 polypeptide, e.g., a sequence optimized nucleic acid sequence encoding ITB6 set forth as SEQ ID NO: 8;
(iv) at least one stop codon (if not present at 5′ terminus of 3′UTR);
(v) a 3′ UTR, such as the sequences provided herein, for example, SEQ ID NO: 110; and
(vi) a poly-A tail provided herein (e.g., SEQ ID NO:502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from the sequence of SEQ ID NO: 10. In an embodiment, the polynucleotide (e.g., mRNA) encoding the ITB6 molecule comprises the nucleotide sequence of SEQ ID NO: 10. In some embodiments, the polynucleotide encoding the ITB6 molecule comprises the nucleotide sequence of SEQ ID NO: 171, which comprises from 5′ to 3′ end: the 5′ UTR sequence of SEQ ID NO: 50, the ORF sequence of SEQ ID NO: 10, and the 3′ UTR sequence of SEQ ID NO:
110.

In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO:50;
(iii) an open reading frame encoding an ITB6 polypeptide, e.g., a sequence optimized nucleic acid sequence encoding ITB6 set forth as SEQ ID NO: 10;
(iv) at least one stop codon (if not present at 5′ terminus of 3′UTR);
(v) a 3′ UTR, such as the sequences provided herein, for example, SEQ ID NO: 110; and
(vi) a poly-A tail provided herein (e.g., SEQ ID NO:502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from the sequence of SEQ ID NO: 12. In an embodiment, the polynucleotide (e.g., mRNA) encoding the ITB6 molecule comprises the nucleotide sequence of SEQ ID NO: 12. In some embodiments, the polynucleotide encoding the ITB6 molecule comprises the nucleotide sequence of SEQ ID NO: 172, which comprises from 5′ to 3′ end: the 5′ UTR sequence of SEQ ID NO: 50, the ORF sequence of SEQ ID NO: 12, and the 3′ UTR sequence of SEQ ID NO: 110.

In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO: 50;
(iii) an open reading frame encoding an ITB6 polypeptide, e.g., a sequence optimized nucleic acid sequence encoding ITB6 set forth as SEQ ID NO: 12;
(iv) at least one stop codon (if not present at 5′ terminus of 3′UTR);
(v) a 3′ UTR, such as the sequences provided herein, for example, SEQ ID NO: 110; and
(vi) a poly-A tail provided herein (e.g., SEQ ID NO:502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from the sequence of SEQ ID NO: 14. In an embodiment, the polynucleotide (e.g., mRNA) encoding the ITB6 molecule comprises the nucleotide sequence of SEQ ID NO: 14. In some embodiments, the polynucleotide encoding the ITB6 molecule comprises the nucleotide sequence of SEQ ID NO: 173, which comprises from 5′ to 3′ end: the 5′ UTR sequence of SEQ ID NO: 50, the ORF sequence of SEQ ID NO: 14, and the 3′ UTR sequence of SEQ ID NO: 110.

In some embodiments, the polynucleotide encoding the ITB6 molecule comprises from 5′ to 3′ end
(i) a 5′ cap such as provided herein, e.g., Cap II-b;
(ii) a 5′ UTR, such as the sequences provided herein, for example, SEQ ID NO: 50;
(iii) an open reading frame encoding an ITB6 polypeptide, e.g., a sequence optimized nucleic acid sequence encoding ITB6 set forth as SEQ ID NO: 14;
(iv) at least one stop codon (if not present at 5′ terminus of 3′ UTR);
(v) a 3′ UTR, such as the sequences provided herein, for example, SEQ ID NO: 110; and
(vi) a poly-A tail provided herein (e.g., SEQ ID NO:502).

In an embodiment, the polynucleotide (e.g., mRNA) encoding the ITB6 molecule comprises a nucleotide sequence having at least 85%, 90%, 95%, 96%, 97%, 98%, 99%, or 100% identity to, or differing by no more than 5, 10, 15, 20, 30, 40, 50, 60, 70, 80, 90, or 100 nucleotides from the sequence of SEQ ID NO: 16. In an embodiment, the polynucleotide (e.g., mRNA) encoding the ITB6 molecule comprises the nucleotide sequence of SEQ ID NO: 16. In some embodiments, the polynucleotide encoding the ITB6 molecule comprises the nucleotide sequence of SEQ ID NO: 174, which comprises from 5′ to 3′ end: the 5′ UTR sequence of SEQ ID NO: 56, the ORF sequence of SEQ ID NO: 16, and the 3′ UTR sequence of SEQ ID NO: 110.

In some embodiments, the polynucleotide encoding the ITB6 molecule comprises from 5′ to 3′ end
(i) a 5′ cap such as provided herein, e.g., Cap C1;
(ii) a 5′ UTR, such as the sequences provided herein, for example, SEQ ID NO: 56;
(iii) an open reading frame encoding an ITB6 polypeptide, e.g., a sequence optimized nucleic acid sequence encoding ITB6 set forth as SEQ ID NO: 16;
(iv) at least one stop codon (if not present at 5′ terminus of 3′UTR);
(v) a 3′ UTR, such as the sequences provided herein, for example, SEQ ID NO: 110; and
(vi) a poly-A tail provided herein (e.g., SEQ ID NO:502).

In an embodiment, the polynucleotide (e.g., mRNA) encoding the ITB6 molecule further comprises one or more elements, e.g., a 5′ UTR and/or a 3′ UTR. In an embodiment, the 5′ UTR and/or 3′UTR comprise one or more micro-RNA (mIR) binding sites, e.g., as disclosed herein. Exemplary 5′ UTRs and 3′ UTRs are disclosed in the section entitled “5′ UTR and 3′UTR” herein.

In some embodiments, the polynucleotide encoding the ITB6 molecule comprises the nucleotide sequence of any of human variant 5.1, human variant 1.1, human variant 1.2, human variant 1.3, human variant 1.4, human variant 1.5, human variant 1.6, human variant 1.7, human variant 1.8, human variant 1.9, human variant 1.10, human variant 2.1, human variant 3.1, human variant 4.1, rat variant 1.1, or mouse variant 1.1, as described in Table 2A. In some embodiments, the polynucleotide encoding the ITB6 molecule comprises the chemical modification(s) shown in Table 2A for any of human variant 5.1, human variant 1.1, human variant 1.2, human variant 1.3, human variant 1.4, human variant 1.5, human variant 1.6, human variant 1.7, human variant 1.8, human variant 1.9, human variant 1.10, human variant 2.1, human variant 3.1, human variant 4.1, rat variant 1.1, or mouse variant 1.1. In some embodiments, the polynucleotide encoding the ITB6 molecule does not comprise the chemical modification(s) shown in Table 2A for any of human variant 5.1, human variant 1.1, human variant 1.2, human variant 1.3, human variant 1.4, human variant 1.5, human variant 1.6, human variant 1.7, human variant 1.8, human variant 1.9, human variant 1.10, human variant 2.1, human variant 3.1, human variant 4.1, rat variant 1.1, or mouse variant 1.1.

In an aspect, an LNP composition disclosed herein comprises a polynucleotide (e.g., mRNA) encoding an ITB6 molecule, e.g., as described herein. In an embodiment, the ITB6 molecule comprises a half-life extender, e.g., a protein (or fragment thereof) that binds to a serum protein such as albumin, IgG, FcRn or transferrin. In an embodiment, the half-life extender is an immunoglobulin Fc region or a variant thereof, e.g., an IgG1 Fc.

In an embodiment, an LNP composition described herein comprises a polynucleotide (e.g., mRNA) encoding an ITB6 molecule. In an embodiment, the ITB6 molecule further comprises a targeting moiety. In an embodiment, the targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.

TABLE 1A
Exemplary ITB6 sequences

| SEQ ID | Sequence | |
|---|---|---|
| NO | information | Sequence |
| 1 | Human ITB6 | MGIELLCLFFLFLGRNDHVQGGCALGGAETCEDCLLIGPQCAWCAQENFTHPS |
| | amino acid, | GVGERCDTPANLLAKGCQLNFIENPVSQVEILKNKPLSVGRQKNSSDIVQIAPQSL |
| | variant 1 | ILKLRPGGAQTLQVHVRQTEDYPVDLYYLMDLSASMDDDLNTIKELGSRLSKEMSK |
| | | LTSNFRLGFGSFVEKPVSPFVKTTPEEIANPCSSIPYFCLPTFGFKHILPLINDAE |
| | | RFNEIVKNQKISANIDTPEGGFDAIMQAAVCKEKIGWRNDSLHLLVFVSDADSHFG |
| | | MDSKLAGIVIPNDGLCHLDSKNEYSMSTVLEYPTIGQLIDKLVQNNVLLIFAVTQE |
| | | QVHLYENYAKLIPGATVGLLQKDSGNILQLIISAYEELRSEVELEVLGDTEGLNLS |
| | | FTAICNNGTLFQHQKKCSHMKVGDTASFSVTVNIPHCERRSRHIIIKPVGLGDALE |
| | | LLVSPECNCDCQKEVEVNSSKCHHGNGSFQCGVCACHPGHMGPRCECGEDMLSTDS |
| | | CKEAPDHPSCSGRGDCYCGQCICHLSPYGNIYGPYCQCDNFSCVRHKGLLCGGNGD |
| | | CDCGECVCRSGWTGEYCNCTTSTDSCVSEDGVLCSGRGDCVCGKCVCTNPGASGPT |
| | | CERCPTCGDPCNSKRSCIECHLSAAGQAREECVDKCKLAGATISEEEDFSKDGSVS |
| | | CSLQGENECLITFLITTDNEGKTIIHSINEKDCPKPPNIPMIMLGVSLAILLIGVV |
| | | LLCIWKLLVSFHDRKEVAKFEAERSKAKWQTGTNPLYRGSTSTFKNVTYKHREKQK |
| | | VDLSTDCGKPIPNPLLGLDST |
| 2 | Human ITB6 | AUGGGCAUCGAGCUGCUGUGCCUGUUCUUCCUGUUCCUGGGCAGAAACGACCA |
| | nucleotide, | CGUGCAGGGAGGUUGCGCCCUCGGAGGUGCCGAGACAUGCGAGGACUGCCUGCUGA |
| | variant 1 | UCGGCCCUCAGUGCGCCUGGUGCGCCCAGGAGAACUUCACCCACCCUAGCGGCGUG |
| | | GGCGAGAGGUGCGACACCCCUGCCAACCUGCUGGCCAAGGGCUGCCAGCUGAACUU |
| | | CAUCGAGAACCCUGUGAGCCAGGUGGAGAUCCUGAAGAACAAGCCUCUGAGCGUGG |
| | | GCAGACAGAAGAACAGCAGCGACAUCGUGCAGAUCGCCCCUCAGAGCCUGAUUUUG |
| | | AAGCUCCGGCCUGGAGGAGCACAGACCCUGCAGGUGCACGUGAGACAGACCGAGGA |
| | | CUACCCUGUGGACCUGUACUACCUGAUGGACCUGAGCGCCAGCAUGGACGACGACC |
| | | UGAACACCAUCAAGGAGCUGGGCAGCAGACUGAGCAAGGAGAUGAGCAAGCUGACC |
| | | AGCAACUUCAGACUGGGCUUCGGCAGCUUCGUGGAGAAGCCUGUAUCGCCUUUCGU |
| | | GAAGACCACCCCUGAGGAGAUCGCCAACCCUUGCAGCAGCAUCCCUUACUUCUGCC |
| | | UGCCUACCUUCGGCUUCAAGCACAUCCUGCCUCUGACCAACGACGCCGAGAGAUUC |
| | | AACGAGAUAGUUAAGAACCAGAAGAUCAGCGCCAACAUAGAUACUCCAGAAGGAGG |
| | | UUUCGACGCCAUCAUGCAGGCCGCCGUGUGUAAGGAGAAGAUCGGCUGGCGGAACG |
| | | ACAGCCUGCAUCUUUUGGUGUUCGUGAGUGACGCAGACAGCCACUUCGGCAUGGAC |
| | | UCUAAGUUAGCCGGCAUCGUGAUCCCUAACGACGGCCUGUGCCACCUGGACAGCAA |
| | | GAACGAGUACAGCAUGAGCACCGUGCUGGAGUACCCUACCAUCGGUCAGCUGAUCG |
| | | ACAAGCUGGUGCAGAACAACGUGCUGCUAAUCUUCGCCGUGACCCAGGAACAAGUC |
| | | CACCUGUACGAGAACUACGCCAAGCUGAUCCCUGGCGCCACCGUGGGCCUGCUGCA |
| | | GAAGGACAGCGGCAAUAUCUUGCAGUUGAUUAUUUCCGCAUACGAGGAGCUGAGAA |
| | | GCGAGGUGGAGCUGGAGGUGCUGGGCGACACCGAGGGCCUGAACCUGAGCUUCACC |
| | | GCCAUCUGCAACAACGGCACCCUGUUCCAGCAUCAGAAGAAGUGCAGCCACAUGAA |
| | | GGUGGGCGAUACAGCCAGCUUCAGCGUGACCGUGAACAUCCCUCACUGCGAGAGAA |
| | | GAAGCAGACACAUCAUCAUCAAGCCUGUGGGCCUUGGUGACGCCCUGGAACUGCUG |
| | | GUAUCACCAGAGUGCAACUGCGACUGUCAGAAGGAAGUGGAGGUUAACUCUUCUAA |
| | | GUGCCACCACGGCAACGGAAGUUUCCAGUGCGGCGUCUGUGCGUGCCACCCUGGCC |
| | | ACAUGGGCCCUCGAUGCGAGUGCGGAGAGGACAUGCUCUCUACUGACAGCUGUAAG |
| | | GAGGCCCCUGACCAUCCGAGUUGUAGCGGCAGAGGCGACUGCUACUGCGGCCAGUG |
| | | CAUCUGCCAUCUAUCUCCAUACGGAAAUAUCUACGGCCCUUACUGCCAGUGCGACA |
| | | AUUUCAGCUGCGUUAGACACAAGGGUCUUCUGUGUGGAGGAAACGGCGAUUGUGAU |
| | | UGCGGUGAGUGCGUGUGCAGAAGCGGCUGGACCGGCGAGUACUGCAAUUGCACCAC |
| | | CUCCACUGACAGCUGCGUGAGCGAGGACGGCGUGCUGUGCAGCGGACGGGGCGACU |
| | | GUGUUUGCGGCAAGUGUGUGUGCACCAAUCCUGGUGCUAGCGGCCCUACUUGCGAA |
| | | AGGUGCCCAACCUGUGGCGACCCGUGCAACUCCAAGAGAAGCUGCAUCGAGUGUCA |
| | | CCUAUCAGCUGCCGGCCAGGCCAGAGAGGAGUGUGUUGACAAGUGCAAGUUAGCUG |
| | | GAGCAACAAUCAGCGAGGAGGAGGACUUCAGCAAGGACGGCAGCGUGAGCUGUAGU |
| | | CUCCAAGGCGAGAACGAGUGCCUGAUCACCUUCCUGAUUACUACCGACAACGAGGG |
| | | CAAGACCAUCAUCCACUCCAUCAACGAGAAGGACUGUCCUAAGCCUCCUAAUAUCC |
| | | CUAUGAUCAUGCUUGGCGUGAGCCUGGCCAUUCUUCUGAUUGGAGUGGUGCUGCUC |
| | | UGUAUUUGGAAGCUACUGGUGAGCUUCCACGACAGAAAGGAAGUUGCCAAGUUCGA |
| | | GGCGGAAAGGAGCAAGGCCAAGUGGCAGACCGGCACUAACCCUCUGUACAGAGGAA |
| | | GUACCAGUACAUUCAAGAACGUGACCUACAAGCACAGAGAGAAGCAGAAGGUGGAU |
| | | CUUUCUACAGACUGCGGUAAGCCUAUCCCUAAUCCACUGUUGGGUUUGGAUUCCAC |
| | | C |
| 3 | Human ITB6 | AUGGGCAUCGAGCUGCUGUGCCUGUUCUUCCUGUUCCUGGGCAGAAACGACCACGU |
| | nucleotide 2, | GCAGGGAGGUUGCGCCCUCGGAGGUGCCGAGACAUGCGAGGACUGCCUGCUGAUCG |
| | variant 1 | GCCCUCAGUGCGCCUGGUGCGCCCAGGAGAACUUCACCCACCCUAGCGGCGUGGGC |
| | | GAGAGGUGCGACACCCCUGCCAACCUGCUGGCCAAGGGCUGCCAGCUGAACUUCAU |
| | | CGAGAACCCUGUGAGCCAGGUGGAGAUCCUGAAGAACAAGCCUCUGAGCGUGGGCA |
| | | GACAGAAGAACAGCAGCGACAUCGUGCAGAUCGCCCCUCAGAGCCUGAUUUUGAAG |
| | | CUCCGGCCUGGAGGAGCACAGACCCUGCAGGUGCACGUGAGACAGACCGAGGACUA |
| | | CCCUGUGGACCUGUACUACCUGAUGGACCUGAGCGCCAGCAUGGACGACGACCUGA |
| | | ACACCAUCAAGGAGCUGGGCAGCAGACUGAGCAAGGAGAUGAGCAAGCUGACCAGC |
| | | AACUUCAGACUGGGCUUCGGCAGCUUCGUGGAGAAGCCUGUAUCGCCUUUCGUGAA |
| | | GACCACCCCUGAGGAGAUCGCCAACCCUUGCAGCAGCAUCCCUUACUUCUGCCUGC |
| | | CUACCUUCGGCUUCAAGCACAUCCUGCCUCUGACCAACGACGCCGAGAGAUUCAAC |
| | | GAGAUAGUUAAGAACCAGAAGAUCAGCGCCAACAUAGAUACUCCAGAAGGAGGUUU |
| | | CGACGCCAUCAUGCAGGCCGCCGUGUGUAAGGAGAAGAUCGGCUGGCGGAACGACA |
| | | GCCUGCAUCUUUUGGUGUUCGUGAGUGACGCAGACAGCCACUUCGGCAUGGACUCU |
| | | AAGUUAGCCGGCAUCGUGAUCCCUAACGACGGCCUGUGCCACCUGGACAGCAAGAA |
| | | CGAGUACAGCAUGAGCACCGUGCUGGAGUACCCUACCAUCGGUCAGCUGAUCGACA |
| | | AGCUGGUGCAGAACAACGUGCUGCUAAUCUUCGCCGUGACCCAGGAACAAGUCCAC |
| | | CUGUACGAGAACUACGCCAAGCUGAUCCCUGGCGCCACCGUGGGCCUGCUGCAGAA |
| | | GGACAGCGGCAAUAUCUUGCAGUUGAUUAUUUCCGCAUACGAGGAGCUGAGAAGCG |
| | | AGGUGGAGCUGGAGGUGCUGGGCGACACCGAGGGCCUGAACCUGAGCUUCACCGCC |
| | | AUCUGCAACAACGGCACCCUGUUCCAGCAUCAGAAGAAGUGCAGCCACAUGAAGGU |
| | | GGGCGAUACAGCCAGCUUCAGCGUGACCGUGAACAUCCCUCACUGCGAGAGAAGAA |
| | | GCAGACACAUCAUCAUCAAGCCUGUGGGCCUUGGUGACGCCCUGGAACUGCUGGUA |
| | | UCACCAGAGUGCAACUGCGACUGUCAGAAGGAAGUGGAGGUUAACUCUUCUAAGUG |
| | | CCACCACGGCAACGGAAGUUUCCAGUGCGGCGUCUGUGCGUGCCACCCUGGCCACA |
| | | UGGGCCCUCGAUGCGAGUGCGGAGAGGACAUGCUCUCUACUGACAGCUGUAAGGAG |
| | | GCCCCUGACCAUCCGAGUUGUAGCGGCAGAGGCGACUGCUACUGCGGCCAGUGCAU |
| | | CUGCCAUCUAUCUCCAUACGGAAAUAUCUACGGCCCUUACUGCCAGUGCGACAAUU |
| | | UCAGCUGCGUUAGACACAAGGGUCUUCUGUGUGGAGGAAACGGCGAUUGUGAUUGC |
| | | GGUGAGUGCGUGUGCAGAAGCGGCUGGACCGGCGAGUACUGCAAUUGCACCACCUC |
| | | CACUGACAGCUGCGUGAGCGAGGACGGCGUGCUGUGCAGCGGACGGGGCGACUGUG |
| | | UUUGCGGCAAGUGUGUGUGCACCAAUCCUGGUGCUAGCGGCCCUACUUGCGAAAGG |
| | | UGCCCAACCUGUGGCGACCCGUGCAACUCCAAGAGAAGCUGCAUCGAGUGUCACCU |
| | | AUCAGCUGCCGGCCAGGCCAGAGAGGAGUGUGUUGACAAGUGCAAGUUAGCUGGAG |
| | | CAACAAUCAGCGAGGAGGAGGACUUCAGCAAGGACGGCAGCGUGAGCUGUAGUCUC |
| | | CAAGGCGAGAACGAGUGCCUGAUCACCUUCCUGAUUACUACCGACAACGAGGGCAA |
| | | GACCAUCAUCCACUCCAUCAACGAGAAGGACUGUCCUAAGCCUCCUAAUAUCCCUA |
| | | UGAUCAUGCUUGGCGUGAGCCUGGCCAUUCUUCUGAUUGGAGUGGUGCUGCUCUGU |
| | | AUUUGGAAGCUACUGGUGAGCUUCCACGACAGAAAGGAAGUUGCCAAGUUCGAGGC |
| | | GGAAAGGAGCAAGGCCAAGUGGCAGACCGGCACUAACCCUCUGUACAGAGGAAGUA |
| | | CCAGUACAUUCAAGAACGUGACCUACAAGCACAGAGAGAAGCAGAAGGUGGAUCUU |
| | | UCUACAGACUGCGGUAAGCCUAUCCCUAAUCCACUGUUGGGUUUGGAUUCCACC |
| 4 | Human ITB6 | AUGGGCAUCGAGCUGCUGUGCCUGUUCUUCCUGUUCCUGGGCAGAAACGACCACGU |
| | nucleotide 3, | GCAGGGAGGUUGCGCCCUCGGAGGUGCCGAGACAUGCGAGGACUGCCUGCUGAUCG |
| | variant 1 | GCCCUCAGUGCGCCUGGUGCGCCCAGGAGAACUUCACCCACCCUAGCGGCGUGGGC |
| | | GAGAGGUGCGACACCCCUGCCAACCUGCUGGCCAAGGGCUGCCAGCUGAACUUCAU |
| | | CGAGAACCCUGUGAGCCAGGUGGAGAUCCUGAAGAACAAGCCUCUGAGCGUGGGCA |
| | | GACAGAAGAACAGCAGCGACAUCGUGCAGAUCGCCCCUCAGAGCCUGAUUUUGAAG |
| | | CUCCGGCCUGGAGGAGCACAGACCCUGCAGGUGCACGUGAGACAGACCGAGGACUA |
| | | CCCUGUGGACCUGUACUACCUGAUGGACCUGAGCGCCAGCAUGGACGACGACCUGA |
| | | ACACCAUCAAGGAGCUGGGCAGCAGACUGAGCAAGGAGAUGAGCAAGCUGACCAGC |
| | | AACUUCAGACUGGGCUUCGGCAGCUUCGUGGAGAAGCCUGUAUCGCCUUUCGUGAA |
| | | GACCACCCCUGAGGAGAUCGCCAACCCUUGCAGCAGCAUCCCUUACUUCUGCCUGC |
| | | CUACCUUCGGCUUCAAGCACAUCCUGCCUCUGACCAACGACGCCGAGAGAUUCAAC |
| | | GAGAUAGUUAAGAACCAGAAGAUCAGCGCCAACAUAGAUACUCCAGAAGGAGGUUU |
| | | CGACGCCAUCAUGCAGGCCGCCGUGUGUAAGGAGAAGAUCGGCUGGCGGAACGACA |
| | | GCCUGCAUCUUUUGGUGUUCGUGAGUGACGCAGACAGCCACUUCGGCAUGGACUCU |
| | | AAGUUAGCCGGCAUCGUGAUCCCUAACGACGGCCUGUGCCACCUGGACAGCAAGAA |
| | | CGAGUACAGCAUGAGCACCGUGCUGGAGUACCCUACCAUCGGUCAGCUGAUCGACA |
| | | AGCUGGUGCAGAACAACGUGCUGCUAAUCUUCGCCGUGACCCAGGAACAAGUCCAC |
| | | CUGUACGAGAACUACGCCAAGCUGAUCCCUGGCGCCACCGUGGGCCUGCUGCAGAA |
| | | GGACAGCGGCAAUAUCUUGCAGUUGAUUAUUUCCGCAUACGAGGAGCUGAGAAGCG |
| | | AGGUGGAGCUGGAGGUGCUGGGCGACACCGAGGGCCUGAACCUGAGCUUCACCGCC |
| | | AUCUGCAACAACGGCACCCUGUUCCAGCAUCAGAAGAAGUGCAGCCACAUGAAGGU |
| | | GGGCGAUACAGCCAGCUUCAGCGUGACCGUGAACAUCCCUCACUGCGAGAGAAGAA |
| | | GCAGACACAUCAUCAUCAAGCCUGUGGGCCUUGGUGACGCCCUGGAACUGCUGGUA |
| | | UCACCAGAGUGCAACUGCGACUGUCAGAAGGAAGUGGAGGUUAACUCUUCUAAGUG |
| | | CCACCACGGCAACGGAAGUUUCCAGUGCGGCGUCUGUGCGUGCCACCCUGGCCACA |
| | | UGGGCCCUCGAUGCGAGUGCGGAGAGGACAUGCUCUCUACUGACAGCUGUAAGGAG |
| | | GCCCCUGACCAUCCGAGUUGUAGCGGCAGAGGCGACUGCUACUGCGGCCAGUGCAU |
| | | CUGCCAUCUAUCUCCAUACGGAAAUAUCUACGGCCCUUACUGCCAGUGCGACAAUU |
| | | UCAGCUGCGUUAGACACAAGGGUCUUCUGUGUGGAGGAAACGGCGAUUGUGAUUGC |
| | | GGUGAGUGCGUGUGCAGAAGCGGCUGGACCGGCGAGUACUGCAAUUGCACCACCUC |
| | | CACUGACAGCUGCGUGAGCGAGGACGGCGUGCUGUGCAGCGGACGGGGCGACUGUG |
| | | UUUGCGGCAAGUGUGUGUGCACCAAUCCUGGUGCUAGCGGCCCUACUUGCGAAAGG |
| | | UGCCCAACCUGUGGCGACCCGUGCAACUCCAAGAGAAGCUGCAUCGAGUGUCACCU |
| | | AUCAGCUGCCGGCCAGGCCAGAGAGGAGUGUGUUGACAAGUGCAAGUUAGCUGGAG |
| | | CAACAAUCAGCGAGGAGGAGGACUUCAGCAAGGACGGCAGCGUGAGCUGUAGUCUC |
| | | CAAGGCGAGAACGAGUGCCUGAUCACCUUCCUGAUUACUACCGACAACGAGGGCAA |
| | | GACCAUCAUCCACUCCAUCAACGAGAAGGACUGUCCUAAGCCUCCUAAUAUCCCUA |
| | | UGAUCAUGCUUGGCGUGAGCCUGGCCAUUCUUCUGAUUGGAGUGGUGCUGCUCUGU |
| | | AUUUGGAAGCUACUGGUGAGCUUCCACGACAGAAAGGAAGUUGCCAAGUUCGAGGC |
| | | GGAAAGGAGCAAGGCCAAGUGGCAGACCGGCACUAACCCUCUGUACAGAGGAAGUA |
| | | CCAGUACAUUCAAGAACGUGACCUACAAGCACAGAGAGAAGCAGAAGGUGGAUCUU |
| | | UCUACAGACUGCGGUAAGCCUAUCCCUAAUCCACUGUUGGGUUUGGAUUCCACC |
| 5 | Human ITB6 | AUGGGGAUCGAGCUGCUGUGCCUGUUCUUCCUGUUCCUGGGACGGAACGACCACGU |
| | nucleotide 4, | GCAGGGUGGCUGCGCCCUUGGUGGGGCAGAGACCUGCGAGGACUGCCUGCUCAUCG |
| | variant 1 | GCCCACAGUGCGCCUGGUGCGCUCAGGAGAACUUCACCCACCCCAGCGGAGUGGGC |
| | | GAGAGGUGCGACACCCCGGCCAACCUGCUGGCCAAGGGGUGCCAGCUGAACUUCAU |
| | | CGAGAACCCCGUCUCUCAGGUGGAGAUCCUGAAGAACAAGCCCCUCAGCGUUGGGC |
| | | GGCAGAAGAACAGCUCUGACAUCGUGCAGAUCGCGCCCCAGAGCCUGAUCCUGAAA |
| | | CUACGGCCUGGCGGUGCGCAAACCCUGCAGGUCCACGUGCGGCAGACCGAGGACUA |
| | | CCCUGUGGACCUGUACUACCUGAUGGACCUCUCCGCCAGCAUGGACGACGAUCUGA |
| | | ACACCAUCAAGGAGCUGGGCAGCCGCCUGAGCAAGGAGAUGUCCAAGCUGACCUCC |
| | | AACUUCCGCCUGGGGUUCGGAUCCUUCGUGGAGAAGCCCGUGAGCCCCUUCGUGAA |
| | | GACCACCCCUGAGGAGAUCGCCAACCCCUGCAGCAGCAUCCCUUACUUCUGCCUGC |
| | | CCACCUUCGGCUUCAAGCACAUCCUGCCACUGACCAACGACGCCGAGCGGUUCAAC |
| | | GAGAUCGUGAAGAACCAGAAGAUCAGCGCCAACAUCGACACCCCGGAGGGUGGGUU |
| | | CGACGCUAUCAUGCAGGCCGCCGUGUGCAAGGAGAAGAUCGGCUGGCGCAACGACA |
| | | GCCUGCACCUGCUGGUGUUCGUGUCCGACGCCGACUCCCACUUCGGCAUGGACAGC |
| | | AAGCUGGCCGGUAUCGUGAUCCCUAACGACGGCCUGUGCCACCUGGACAGCAAGAA |
| | | CGAGUACAGCAUGAGCACCGUGCUGGAGUACCCCACCAUCGGCCAGCUGAUCGACA |
| | | AGCUGGUGCAGAACAACGUGCUGCUGAUCUUCGCCGUGACCCAGGAGCAGGUGCAC |
| | | CUCUACGAGAACUACGCCAAGCUGAUUCCAGGCGCCACCGUCGGUCUGCUGCAGAA |
| | | GGACAGCGGCAACAUCCUGCAGCUGAUCAUCUCCGCCUACGAGGAGCUGAGGAGCG |
| | | AGGUGGAGCUGGAGGUGCUCGGGGACACCGAGGGCCUGAACCUGUCCUUCACCGCC |
| | | AUCUGCAACAACGGGACCCUCUUCCAGCACCAGAAGAAGUGCAGCCACAUGAAGGU |
| | | GGGCGACACCGCCAGCUUCAGCGUCACCGUGAACAUACCUCACUGUGAGCGGCGGA |
| | | GCAGGCACAUCAUCAUCAAGCCCGUGGGCCUGGGAGACGCCCUGGAGCUGCUGGUG |
| | | UCUCCCGAGUGCAACUGCGACUGCCAGAAGGAGGUGGAGGUGAACUCUAGCAAGUG |
| | | CCACCACGGCAACGGGAGUUUCCAGUGUGGCGUGUGUGCCUGCCACCCCGGUCACA |
| | | UGGGACCCCGGUGUGAGUGCGGGGAGGACAUGCUGAGCACCGACAGCUGCAAGGAG |
| | | GCCCCAGACCACCCGAGCUGUUCCGGUCGGGGCGACUGCUACUGCGGCCAGUGCAU |
| | | CUGUCACCUGAGCCCCUACGGCAACAUCUACGGGCCCUACUGCCAGUGCGACAACU |
| | | UCUCCUGCGUGCGCCACAAAGGCCUGCUGUGUGGCGGCAACGGCGACUGCGACUGC |
| | | GGGGAGUGCGUGUGCCGCAGUGGGUGGACCGGCGAGUACUGCAACUGCACCACCUC |
| | | CACCGACUCUUGCGUGUCCGAGGACGGGGUGCUGUGUUCCGGGCGCGGAGAUUGCG |
| | | UGUGCGGGAAGUGCGUGUGCACCAACCCUGGCGCAUCGGGUCCUACCUGCGAGAGG |
| | | UGCCCCACCUGUGGCGACCCCUGCAACAGCAAGCGCUCUUGCAUCGAGUGCCACCU |
| | | CUCAGCAGCAGGGCAGGCUCGGGAGGAGUGCGUGGACAAGUGCAAGCUCGCCGGCG |
| | | CCACCAUCAGUGAGGAGGAGGACUUCUCCAAGGACGGCAGCGUGUCCUGCUCCCUC |
| | | CAGGGCGAGAACGAGUGCCUCAUCACCUUCCUGAUCACCACCGACAACGAGGGCAA |
| | | GACCAUCAUCCACUCCAUCAACGAGAAGGACUGCCCCAAGCCGCCUAACAUCCCCA |
| | | UGAUCAUGCUGGGCGUGAGCCUGGCCAUCCUGCUGAUCGGCGUGGUGCUGCUGUGC |
| | | AUCUGGAAGCUGCUGGUCUCCUUCCACGACCGCAAGGAGGUGGCCAAGUUCGAGGC |
| | | CGAGCGCUCUAAGGCCAAGUGGCAGACCGGCACCAACCCUCUGUACCGGGGCUCCA |
| | | CCAGCACCUUCAAGAACGUGACCUACAAGCACCGCGAGAAGCAGAAGGUGGACCUG |
| | | UCCACCGACUGUGGCAAGCCCAUCCCCAAUCCCCUGCUGGGGCUGGACUCCACC |
| 6 | Human ITB6 | AUGGGUAUCGAGCUGCUGUGUCUGUUCUUUCUGUUUCUGGGCCGCAACGACCACGU |
| | nucleotide 6, | CCAGGGCGGCUGCGCCCUGGGGGGGGCCGAGACCUGCGAGGACUGCCUGCUCAUCG |
| | variant 1 | GGCCACAGUGUGCCUGGUGUGCACAGGAGAACUUCACCCACCCCAGCGGGGUGGGC |
| | | GAGAGGUGCGACACCCCUGCAAAUCUGCUGGCAAAGGGCUGCCAGCUGAAUUUCAU |
| | | CGAGAAUCCCGUCAGCCAGGUGGAGAUUCUCAAGAACAAGCCUCUGAGCGUCGGAC |
| | | GGCAGAAGAACUCUUCUGACAUCGUCCAGAUCGCACCUCAGAGCCUGAUUCUGAAG |
| | | CUCCGCCCAGGCGGAGCUCAGACCCUCCAGGUCCACGUCAGGCAGACUGAGGACUA |
| | | CCCCGUGGACCUGUACUACCUGAUGGAUCUGUCCGCCAGCAUGGACGACGAUCUGA |
| | | ACACCAUUAAGGAGCUGGGCAGCCGGCUCUCUAAGGAGAUGUCCAAGCUCACCAGC |
| | | AACUUCAGGCUGGGAUUCGGCAGCUUUGUCGAGAAGCCUGUGUCUCCUUUCGUGAA |
| | | GACCACACCAGAGGAGAUCGCAAACCCCUGUAGCAGCAUCCCCUACUUCUGCCUGC |
| | | CUACAUUCGGGUUCAAGCACAUUCUGCCUCUGACCAACGACGCAGAGCGCUUUAAC |
| | | GAGAUCGUGAAGAAUCAGAAGAUCAGCGCCAACAUUGAUACUCCUGAGGGGGGCUU |
| | | CGACGCCAUCAUGCAGGCUGCCGUCUGCAAGGAGAAGAUCGGGUGGAGGAACGACA |
| | | GCCUCCACCUGCUCGUGUUCGUGAGCGACGCCGACAGCCACUUUGGGAUGGACUCC |
| | | AAGCUGGCCGGCAUCGUGAUCCCCAACGACGGGCUGUGUCACCUCGAUUCCAAGAA |
| | | CGAGUACAGCAUGAGCACCGUGCUGGAGUACCCCACCAUUGGGCAGCUCAUUGAUA |
| | | AGCUCGUGCAGAACAACGUCCUGCUGAUCUUUGCAGUGACACAGGAGCAGGUGCAC |
| | | CUGUACGAGAAUUACGCAAAGCUCAUCCCCGGAGCCACUGUGGGCCUCCUGCAGAA |
| | | GGACUCCGGGAACAUCCUCCAGCUCAUCAUCUCCGCCUACGAGGAGCUGAGGAGCG |
| | | AGGUGGAGCUCGAGGUCCUCGGGGACACCGAGGGCCUCAACCUGAGCUUUACCGCC |
| | | AUCUGUAACAACGGAACUCUCUUCCAGCACCAGAAGAAGUGCUCUCACAUGAAGGU |
| | | GGGCGACACCGCCUCCUUCAGCGUGACCGUGAACAUCCCUCACUGUGAGCGGCGGA |
| | | GCCGCCACAUCAUCAUCAAGCCCGUGGGGCUCGGGGACGCCCUGGAGCUCCUGGUG |
| | | UCCCCUGAGUGUAACUGCGACUGCCAGAAGGAGGUCGAGGUGAACAGCUCUAAGUG |
| | | CCACCACGGCAACGGGAGCUUCCAGUGCGGGGUGUGCGCAUGCCACCCCGGCCACA |
| | | UGGGGCCUCGCUGCGAGUGCGGCGAGGACAUGCUGUCUACCGACAGCUGCAAGGAG |
| | | GCCCCUGACCACCCUAGCUGCUCUGGCCGCGGAGACUGCUACUGCGGCCAGUGCAU |
| | | CUGCCACCUGUCCCCUUACGGGAACAUUUACGGGCCCUACUGCCAGUGUGAUAACU |
| | | UUUCCUGCGUGCGGCACAAGGGCCUGCUGUGCGGAGGAAAUGGCGACUGCGACUGC |
| | | GGCGAGUGCGUCUGUCGCAGCGGCUGGACCGGGGAGUACUGCAACUGCACUACUUC |
| | | CACUGACAGCUGUGUCAGCGAGGACGGGGUGCUCUGUUCUGGCCGCGGGGACUGCG |
| | | UCUGCGGCAAGUGCGUGUGCACUAAUCCAGGGGCCUCCGGGCCCACCUGCGAGCGG |
| | | UGUCCCACCUGCGGGGACCCUUGCAACAGCAAGAGGUCCUGCAUCGAGUGCCACCU |
| | | GUCCGCCGCCGGACAGGCACGCGAGGAGUGCGUCGAUAAGUGUAAGCUCGCCGGAG |
| | | CUACAAUUAGCGAGGAGGAGGACUUCUCCAAGGACGGGAGCGUGAGCUGCUCCCUC |
| | | CAGGGAGAGAACGAGUGCCUGAUCACUUUCCUCAUCACCACUGAUAACGAGGGAAA |
| | | GACAAUCAUUCACUCUAUCAACGAGAAGGAUUGUCCCAAGCCCCCAAACAUUCCCA |
| | | UGAUCAUGCUGGGAGUGUCCCUGGCAAUCCUGCUGAUCGGAGUCGUGCUGCUCUGC |
| | | AUCUGGAAGCUGCUCGUCUCCUUCCACGACCGCAAGGAGGUCGCAAAGUUUGAGGC |
| | | CGAGCGGUCUAAGGCCAAGUGGCAGACAGGGACCAAUCCUCUCUACCGCGGGUCUA |
| | | CUUCUACCUUCAAGAACGUGACUUACAAGCACAGGGAGAAGCAGAAGGUGGACCUC |
| | | UCCACCGAUUGCGGGAAGCCCAUCCCCAAUCCCCUGCUGGGCCUGGACAGCACC |
| 7 | Human ITB6 | MGIELLCLFFLFLGRNDHVQGGCALGGAETCEDCLLIGPQCAWCAQENFTHPSGVG |
| | amino acid, | ERCDTPANLLAKGCQLNFIENPVSQVEILKNKPLSVGRQKNSSDIVQIAPQSLILK |
| | variant 2 | LRPGGAQTLQVHVRQTEDYPVDLYYLMDLSASMDDDLNTIKELGSRLSKEMSKLTS |
| | | NFRLGFGSFVEKPVSPFVKTTPEEIANPCSSIPYFCLPTFGFKHILPLINDAERFN |
| | | EIVKNQKISANIDAPEGGFDAIMQAAVCKEKIGWRNDSLHLLVFVSDADSHFGMDS |
| | | KLAGIVIPNDGLCHLDSKNEYSMSTVLEYPTIGQLIDKLVQNNVLLIFAVTQEQVH |
| | | LYENYAKLIPGATVGLLQKDSGNILQLIISAYEELRSEVELEVLGDTEGLNLSFTA |
| | | ICNNGTLFQHQKKCSHMKVGDTASFSVTVNIPHCERRSRHIIIKPVGLGDALELLV |
| | | SPECNCDCQKEVEVNSSKCHHGNGSFQCGVCACHPGHMGPRCECGEDMLSTDSCKE |
| | | APDHPSCSGRGDCYCGQCICHLSPYGNIYGPYCQCDNFSCVRHKGLLCGGNGDCDC |
| | | GECVCRSGWTGEYCNCTTSTDSCVSEDGVLCSGRGDCVCGKCVCTNPGASGPTCER |
| | | CPTCGDPCNSKRSCIECHLSAAGQAREECVDKCKLAGATISEEEDFSKDGSVSCSL |
| | | QGENECLITFLITTDNEGKTIIHSINEKDCPKPPNIPMIMLGVSLAILLIGVVLLC |
| | | IWKLLVSFHDRKEVAKFEAERSKAKWQTGTNPLYRGSTSTFKNVTYKHREKQKVDL |
| | | STDCGKPIPNPLLGLDST |
| 8 | Human ITB6 | AUGGGGAUCGAGCUGCUGUGCCUGUUCUUCCUGUUCCUGGGACGGAACGACCACGU |
| | nucleotide, | GCAGGGUGGCUGCGCCCUUGGUGGGGCAGAGACCUGCGAGGACUGCCUGCUCAUCG |
| | variant 2 | GCCCACAGUGCGCCUGGUGCGCUCAGGAGAACUUCACCCACCCCAGCGGAGUGGGC |
| | | GAGAGGUGCGACACCCCGGCCAACCUGCUGGCCAAGGGGUGCCAGCUGAACUUCAU |
| | | CGAGAACCCCGUCUCUCAGGUGGAGAUCCUGAAGAACAAGCCCCUCAGCGUUGGGC |
| | | GGCAGAAGAACAGCUCUGACAUCGUGCAGAUCGCGCCCCAGAGCCUGAUCCUGAAA |
| | | CUACGGCCUGGCGGUGCGCAAACCCUGCAGGUCCACGUGCGGCAGACCGAGGACUA |
| | | CCCUGUGGACCUGUACUACCUGAUGGACCUCUCCGCCAGCAUGGACGACGAUCUGA |
| | | ACACCAUCAAGGAGCUGGGCAGCCGCCUGAGCAAGGAGAUGUCCAAGCUGACCUCC |
| | | AACUUCCGCCUGGGGUUCGGAUCCUUCGUGGAGAAGCCCGUGAGCCCCUUCGUGAA |
| | | GACCACCCCUGAGGAGAUCGCCAACCCCUGCAGCAGCAUCCCUUACUUCUGCCUGC |
| | | CCACCUUCGGCUUCAAGCACAUCCUGCCACUGACCAACGACGCCGAGCGGUUCAAC |
| | | GAGAUCGUGAAGAACCAGAAGAUCAGCGCCAACAUCGACGCCCCGGAGGGUGGGUU |
| | | CGACGCUAUCAUGCAGGCCGCCGUGUGCAAGGAGAAGAUCGGCUGGCGCAACGACA |
| | | GCCUGCACCUGCUGGUGUUCGUGUCCGACGCCGACUCCCACUUCGGCAUGGACAGC |
| | | AAGCUGGCCGGUAUCGUGAUCCCUAACGACGGCCUGUGCCACCUGGACAGCAAGAA |
| | | CGAGUACAGCAUGAGCACCGUGCUGGAGUACCCCACCAUCGGCCAGCUGAUCGACA |
| | | AGCUGGUGCAGAACAACGUGCUGCUGAUCUUCGCCGUGACCCAGGAGCAGGUGCAC |
| | | CUCUACGAGAACUACGCCAAGCUGAUUCCAGGCGCCACCGUCGGUCUGCUGCAGAA |
| | | GGACAGCGGCAACAUCCUGCAGCUGAUCAUCUCCGCCUACGAGGAGCUGAGGAGCG |
| | | AGGUGGAGCUGGAGGUGCUCGGGGACACCGAGGGCCUGAACCUGUCCUUCACCGCC |
| | | AUCUGCAACAACGGGACCCUCUUCCAGCACCAGAAGAAGUGCAGCCACAUGAAGGU |
| | | GGGCGACACCGCCAGCUUCAGCGUCACCGUGAACAUACCUCACUGUGAGCGGCGGA |
| | | GCAGGCACAUCAUCAUCAAGCCCGUGGGCCUGGGAGACGCCCUGGAGCUGCUGGUG |
| | | UCUCCCGAGUGCAACUGCGACUGCCAGAAGGAGGUGGAGGUGAACUCUAGCAAGUG |
| | | CCACCACGGCAACGGGAGUUUCCAGUGUGGCGUGUGUGCCUGCCACCCCGGUCACA |
| | | UGGGACCCCGGUGUGAGUGCGGGGAGGACAUGCUGAGCACCGACAGCUGCAAGGAG |
| | | GCCCCAGACCACCCGAGCUGUUCCGGUCGGGGCGACUGCUACUGCGGCCAGUGCAU |
| | | CUGUCACCUGAGCCCCUACGGCAACAUCUACGGGCCCUACUGCCAGUGCGACAACU |
| | | UCUCCUGCGUGCGCCACAAAGGCCUGCUGUGUGGCGGCAACGGCGACUGCGACUGC |
| | | GGGGAGUGCGUGUGCCGCAGUGGGUGGACCGGCGAGUACUGCAACUGCACCACCUC |
| | | CACCGACUCUUGCGUGUCCGAGGACGGGGUGCUGUGUUCCGGGCGCGGAGAUUGCG |
| | | UGUGCGGGAAGUGCGUGUGCACCAACCCUGGCGCAUCGGGUCCUACCUGCGAGAGG |
| | | UGCCCCACCUGUGGCGACCCCUGCAACAGCAAGCGCUCUUGCAUCGAGUGCCACCU |
| | | CUCAGCAGCAGGGCAGGCUCGGGAGGAGUGCGUGGACAAGUGCAAGCUCGCCGGCG |
| | | CCACCAUCAGUGAGGAGGAGGACUUCUCCAAGGACGGCAGCGUGUCCUGCUCCCUC |
| | | CAGGGCGAGAACGAGUGCCUCAUCACCUUCCUGAUCACCACCGACAACGAGGGCAA |
| | | GACCAUCAUCCACUCCAUCAACGAGAAGGACUGCCCCAAGCCGCCUAACAUCCCCA |
| | | UGAUCAUGCUGGGCGUGAGCCUGGCCAUCCUGCUGAUCGGCGUGGUGCUGCUGUGC |
| | | AUCUGGAAGCUGCUGGUCUCCUUCCACGACCGCAAGGAGGUGGCCAAGUUCGAGGC |
| | | CGAGCGCUCUAAGGCCAAGUGGCAGACCGGCACCAACCCUCUGUACCGGGGCUCCA |
| | | CCAGCACCUUCAAGAACGUGACCUACAAGCACCGCGAGAAGCAGAAGGUGGACCUG |
| | | UCCACCGACUGUGGCAAGCCCAUCCCCAAUCCCCUGCUGGGGCUGGACUCCACC |
| 9 | Human ITB6 | MGIELLCLFFLFLGRNDHVQGGCALGGAETCEDCLLIGPQCAWCAQENFTHPSGVG |
| | amino acid, | ERCDTPANLLAKGCQLNFIENPVSQVEILKNKPLSVGRQKNSSDIVQIAPQSLILK |
| | variant 3 | LRPGGAQTLQVHVRQTEDYPVDLYYLMDLSYSMDDDLNTIKELGSRLSKEMSKLTS |
| | | NFRLGFGSFVEKPVSPFVKTTPEEIANPCSSIPYFCLPTFGFKHILPLINDAERFN |
| | | EIVKNQKISANIDAPEGGFDAIMQAAVCKEKIGWRNDSLHLLVFVSDADSHFGMDS |
| | | KLAGIVIPNDGLCHLDSKNEYSMSTVLEYPTIGQLIDKLVQNNVLLIFAVTQEQVH |
| | | LYENYAKLIPGATVGLLQKDSGNILQLIISAYEELRSEVELEVLGDTEGLNLSFTA |
| | | ICNNGTLFQHQKKCSHMKVGDTASFSVTVNIPHCERRSRHIIIKPVGLGDALELLV |
| | | SPECNCDCQKEVEVNSSKCHHGNGSFQCGVCACHPGHMGPRCECGEDMLSTDSCKE |
| | | APDHPSCSGRGDCYCGQCICHLSPYGNIYGPYCQCDNFSCVRHKGLLCGGNGDCDC |
| | | GECVCRSGWTGEYCNCTTSTDSCVSEDGVLCSGRGDCVCGKCVCTNPGASGPTCER |
| | | CPTCGDPCNSKRSCIECHLSAAGQAREECVDKCKLAGATISEEEDFSKDGSVSCSL |
| | | QGENECLITFLITTDNEGKTIIHSINEKDCPKPPNIPMIMLGVSLAILLIGVVLLC |
| | | IWKLLVSFHDRKEVAKFEAERSKAKWQTGTNPLYRGSTSTFKNVTYKHREKQKVDL |
| | | STDCGKPIPNPLLGLDST |
| 10 | Human ITB6 | AUGGGGAUCGAGCUGCUGUGCCUGUUCUUCCUGUUCCUGGGACGGAACGACCACGU |
| | nucleotide, | GCAGGGUGGCUGCGCCCUUGGUGGGGCAGAGACCUGCGAGGACUGCCUGCUCAUCG |
| | variant 3 | GCCCACAGUGCGCCUGGUGCGCUCAGGAGAACUUCACCCACCCCAGCGGAGUGGGC |
| | | GAGAGGUGCGACACCCCGGCCAACCUGCUGGCCAAGGGGUGCCAGCUGAACUUCAU |
| | | CGAGAACCCCGUCUCUCAGGUGGAGAUCCUGAAGAACAAGCCCCUCAGCGUUGGGC |
| | | GGCAGAAGAACAGCUCUGACAUCGUGCAGAUCGCGCCCCAGAGCCUGAUCCUGAAA |
| | | CUACGGCCUGGCGGUGCGCAAACCCUGCAGGUCCACGUGCGGCAGACCGAGGACUA |
| | | CCCUGUGGACCUGUACUACCUGAUGGACCUCUCCUACAGCAUGGACGACGAUCUGA |
| | | ACACCAUCAAGGAGCUGGGCAGCCGCCUGAGCAAGGAGAUGUCCAAGCUGACCUCC |
| | | AACUUCCGCCUGGGGUUCGGAUCCUUCGUGGAGAAGCCCGUGAGCCCCUUCGUGAA |
| | | GACCACCCCUGAGGAGAUCGCCAACCCCUGCAGCAGCAUCCCUUACUUCUGCCUGC |
| | | CCACCUUCGGCUUCAAGCACAUCCUGCCACUGACCAACGACGCCGAGCGGUUCAAC |
| | | GAGAUCGUGAAGAACCAGAAGAUCAGCGCCAACAUCGACGCCCCGGAGGGUGGGUU |
| | | CGACGCUAUCAUGCAGGCCGCCGUGUGCAAGGAGAAGAUCGGCUGGCGCAACGACA |
| | | GCCUGCACCUGCUGGUGUUCGUGUCCGACGCCGACUCCCACUUCGGCAUGGACAGC |
| | | AAGCUGGCCGGUAUCGUGAUCCCUAACGACGGCCUGUGCCACCUGGACAGCAAGAA |
| | | CGAGUACAGCAUGAGCACCGUGCUGGAGUACCCCACCAUCGGCCAGCUGAUCGACA |
| | | AGCUGGUGCAGAACAACGUGCUGCUGAUCUUCGCCGUGACCCAGGAGCAGGUGCAC |
| | | CUCUACGAGAACUACGCCAAGCUGAUUCCAGGCGCCACCGUCGGUCUGCUGCAGAA |
| | | GGACAGCGGCAACAUCCUGCAGCUGAUCAUCUCCGCCUACGAGGAGCUGAGGAGCG |
| | | AGGUGGAGCUGGAGGUGCUCGGGGACACCGAGGGCCUGAACCUGUCCUUCACCGCC |
| | | AUCUGCAACAACGGGACCCUCUUCCAGCACCAGAAGAAGUGCAGCCACAUGAAGGU |
| | | GGGCGACACCGCCAGCUUCAGCGUCACCGUGAACAUACCUCACUGUGAGCGGCGGA |
| | | GCAGGCACAUCAUCAUCAAGCCCGUGGGCCUGGGAGACGCCCUGGAGCUGCUGGUG |
| | | UCUCCCGAGUGCAACUGCGACUGCCAGAAGGAGGUGGAGGUGAACUCUAGCAAGUG |
| | | CCACCACGGCAACGGGAGUUUCCAGUGUGGCGUGUGUGCCUGCCACCCCGGUCACA |
| | | UGGGACCCCGGUGUGAGUGCGGGGAGGACAUGCUGAGCACCGACAGCUGCAAGGAG |
| | | GCCCCAGACCACCCGAGCUGUUCCGGUCGGGGCGACUGCUACUGCGGCCAGUGCAU |
| | | CUGUCACCUGAGCCCCUACGGCAACAUCUACGGGCCCUACUGCCAGUGCGACAACU |
| | | UCUCCUGCGUGCGCCACAAAGGCCUGCUGUGUGGCGGCAACGGCGACUGCGACUGC |
| | | GGGGAGUGCGUGUGCCGCAGUGGGUGGACCGGCGAGUACUGCAACUGCACCACCUC |
| | | CACCGACUCUUGCGUGUCCGAGGACGGGGUGCUGUGUUCCGGGCGCGGAGAUUGCG |
| | | UGUGCGGGAAGUGCGUGUGCACCAACCCUGGCGCAUCGGGUCCUACCUGCGAGAGG |
| | | UGCCCCACCUGUGGCGACCCCUGCAACAGCAAGCGCUCUUGCAUCGAGUGCCACCU |
| | | CUCAGCAGCAGGGCAGGCUCGGGAGGAGUGCGUGGACAAGUGCAAGCUCGCCGGCG |
| | | CCACCAUCAGUGAGGAGGAGGACUUCUCCAAGGACGGCAGCGUGUCCUGCUCCCUC |
| | | CAGGGCGAGAACGAGUGCCUCAUCACCUUCCUGAUCACCACCGACAACGAGGGCAA |
| | | GACCAUCAUCCACUCCAUCAACGAGAAGGACUGCCCCAAGCCGCCUAACAUCCCCA |
| | | UGAUCAUGCUGGGCGUGAGCCUGGCCAUCCUGCUGAUCGGCGUGGUGCUGCUGUGC |
| | | AUCUGGAAGCUGCUGGUCUCCUUCCACGACCGCAAGGAGGUGGCCAAGUUCGAGGC |
| | | CGAGCGCUCUAAGGCCAAGUGGCAGACCGGCACCAACCCUCUGUACCGGGGCUCCA |
| | | CCAGCACCUUCAAGAACGUGACCUACAAGCACCGCGAGAAGCAGAAGGUGGACCUG |
| | | UCCACCGACUGUGGCAAGCCCAUCCCCAAUCCCCUGCUGGGGCUGGACUCCACC |
| 11 | Human ITB6 | MGIELLCLFFLFLGRNDHVQGGCALGGAETCEDCLLIGPQCAWCAQENFTHPSGVG |
| | amino acid, | ERCDTPANLLAKGCQLNFIENPVSQVEILKNKPLSVGRQKNSSDIVQIAPQSLILK |
| | variant 4 | LRPGGAQTLQVHVRQTEDYPVDLYYLMDLSYSMDDDLNTIKELGSRLSKEMSKLTS |
| | | NFRLGFGSFVEKPVSPFVKTTPEEIANPCSSIPYFCLPTFGFKHILPLINDAERFN |
| | | EIVKNQKISRNIDAPEGGFDAIMQAAVCKEKIGWRNDSLHLLVFVSDADSHFGMDS |
| | | KLAGIVIPNDGLCHLDSKNEYSMSTVLEYPTIGQLIDKLVQNNVLLIFAVTQEQVH |
| | | LYENYAKLIPGATVGLLQKDSGNILQLIISAYEELRSEVELEVLGDTEGLNLSFTA |
| | | ICNNGTLFQHQKKCSHMKVGDTASFSVTVNIPHCERRSRHIIIKPVGLGDALELLV |
| | | SPECNCDCQKEVEVNSSKCHHGNGSFQCGVCACHPGHMGPRCECGEDMLSTDSCKE |
| | | APDHPSCSGRGDCYCGQCICHLSPYGNIYGPYCQCDNFSCVRHKGLLCGGNGDCDC |
| | | GECVCRSGWTGEYCNCTTSTDSCVSEDGVLCSGRGDCVCGKCVCTNPGASGPTCER |
| | | CPTCGDPCNSKRSCIECHLSAAGQAREECVDKCKLAGATISEEEDFSKDGSVSCSL |
| | | QGENECLITFLITTDNEGKTIIHSINEKDCPKPPNIPMIMLGVSLAILLIGVVLLC |
| | | IWKLLVSFHDRKEVAKFEAERSKAKWQTGTNPLYRGSTSTFKNVTYKHREKQKVDL |
| | | STDCGKPIPNPLLGLDST |
| 12 | Human ITB6 | AUGGGGAUCGAGCUGCUGUGCCUGUUCUUCCUGUUCCUGGGACGGAACGACCACGU |
| | nucleotide, | GCAGGGUGGCUGCGCCCUUGGUGGGGCAGAGACCUGCGAGGACUGCCUGCUCAUCG |
| | variant 4 | GCCCACAGUGCGCCUGGUGCGCUCAGGAGAACUUCACCCACCCCAGCGGAGUGGGC |
| | | GAGAGGUGCGACACCCCGGCCAACCUGCUGGCCAAGGGGUGCCAGCUGAACUUCAU |
| | | CGAGAACCCCGUCUCUCAGGUGGAGAUCCUGAAGAACAAGCCCCUCAGCGUUGGGC |
| | | GGCAGAAGAACAGCUCUGACAUCGUGCAGAUCGCGCCCCAGAGCCUGAUCCUGAAA |
| | | CUACGGCCUGGCGGUGCGCAAACCCUGCAGGUCCACGUGCGGCAGACCGAGGACUA |
| | | CCCUGUGGACCUGUACUACCUGAUGGACCUCUCCUACAGCAUGGACGACGAUCUGA |
| | | ACACCAUCAAGGAGCUGGGCAGCCGCCUGAGCAAGGAGAUGUCCAAGCUGACCUCC |
| | | AACUUCCGCCUGGGGUUCGGAUCCUUCGUGGAGAAGCCCGUGAGCCCCUUCGUGAA |
| | | GACCACCCCUGAGGAGAUCGCCAACCCCUGCAGCAGCAUCCCUUACUUCUGCCUGC |
| | | CCACCUUCGGCUUCAAGCACAUCCUGCCACUGACCAACGACGCCGAGCGGUUCAAC |
| | | GAGAUCGUGAAGAACCAGAAGAUCAGCAGAAACAUCGACGCCCCGGAGGGUGGGUU |
| | | CGACGCUAUCAUGCAGGCCGCCGUGUGCAAGGAGAAGAUCGGCUGGCGCAACGACA |
| | | GCCUGCACCUGCUGGUGUUCGUGUCCGACGCCGACUCCCACUUCGGCAUGGACAGC |
| | | AAGCUGGCCGGUAUCGUGAUCCCUAACGACGGCCUGUGCCACCUGGACAGCAAGAA |
| | | CGAGUACAGCAUGAGCACCGUGCUGGAGUACCCCACCAUCGGCCAGCUGAUCGACA |
| | | AGCUGGUGCAGAACAACGUGCUGCUGAUCUUCGCCGUGACCCAGGAGCAGGUGCAC |
| | | CUCUACGAGAACUACGCCAAGCUGAUUCCAGGCGCCACCGUCGGUCUGCUGCAGAA |
| | | GGACAGCGGCAACAUCCUGCAGCUGAUCAUCUCCGCCUACGAGGAGCUGAGGAGCG |
| | | AGGUGGAGCUGGAGGUGCUCGGGGACACCGAGGGCCUGAACCUGUCCUUCACCGCC |
| | | AUCUGCAACAACGGGACCCUCUUCCAGCACCAGAAGAAGUGCAGCCACAUGAAGGU |
| | | GGGCGACACCGCCAGCUUCAGCGUCACCGUGAACAUACCUCACUGUGAGCGGCGGA |
| | | GCAGGCACAUCAUCAUCAAGCCCGUGGGCCUGGGAGACGCCCUGGAGCUGCUGGUG |
| | | UCUCCCGAGUGCAACUGCGACUGCCAGAAGGAGGUGGAGGUGAACUCUAGCAAGUG |
| | | CCACCACGGCAACGGGAGUUUCCAGUGUGGCGUGUGUGCCUGCCACCCCGGUCACA |
| | | UGGGACCCCGGUGUGAGUGCGGGGAGGACAUGCUGAGCACCGACAGCUGCAAGGAG |
| | | GCCCCAGACCACCCGAGCUGUUCCGGUCGGGGCGACUGCUACUGCGGCCAGUGCAU |
| | | CUGUCACCUGAGCCCCUACGGCAACAUCUACGGGCCCUACUGCCAGUGCGACAACU |
| | | UCUCCUGCGUGCGCCACAAAGGCCUGCUGUGUGGCGGCAACGGCGACUGCGACUGC |
| | | GGGGAGUGCGUGUGCCGCAGUGGGUGGACCGGCGAGUACUGCAACUGCACCACCUC |
| | | CACCGACUCUUGCGUGUCCGAGGACGGGGUGCUGUGUUCCGGGCGCGGAGAUUGCG |
| | | UGUGCGGGAAGUGCGUGUGCACCAACCCUGGCGCAUCGGGUCCUACCUGCGAGAGG |
| | | UGCCCCACCUGUGGCGACCCCUGCAACAGCAAGCGCUCUUGCAUCGAGUGCCACCU |
| | | CUCAGCAGCAGGGCAGGCUCGGGAGGAGUGCGUGGACAAGUGCAAGCUCGCCGGCG |
| | | CCACCAUCAGUGAGGAGGAGGACUUCUCCAAGGACGGCAGCGUGUCCUGCUCCCUC |
| | | CAGGGCGAGAACGAGUGCCUCAUCACCUUCCUGAUCACCACCGACAACGAGGGCAA |
| | | GACCAUCAUCCACUCCAUCAACGAGAAGGACUGCCCCAAGCCGCCUAACAUCCCCA |
| | | UGAUCAUGCUGGGCGUGAGCCUGGCCAUCCUGCUGAUCGGCGUGGUGCUGCUGUGC |
| | | AUCUGGAAGCUGCUGGUCUCCUUCCACGACCGCAAGGAGGUGGCCAAGUUCGAGGC |
| | | CGAGCGCUCUAAGGCCAAGUGGCAGACCGGCACCAACCCUCUGUACCGGGGCUCCA |
| | | CCAGCACCUUCAAGAACGUGACCUACAAGCACCGCGAGAAGCAGAAGGUGGACCUG |
| | | UCCACCGACUGUGGCAAGCCCAUCCCCAAUCCCCUGCUGGGGCUGGACUCCACC |
| 17 | Human ITB6 | MGIELLCLFFLFLGRNDHVQGGCALGGAETCEDCLLIGPQCAWCAQENFTH |
| | amino acid, | PSGVGERCDTPANLLAKGCQLNFIENPVSQVEILKNKPLSVGRQKNSSDIV |
| | variant 5 | QIAPQSLILKLRPGGAQTLQVHVRQTEDYPVDLYYLMDLSASMDDDLNTIK |
| | | ELGSRLSKEMSKLTSNFRLGFGSFVEKPVSPFVKTTPEEIANPCSSIPYFC |
| | | LPTFGFKHILPLTNDAERFNEIVKNQKISANIDTPEGGFDAIMQAAVCKEK |
| | | IGWRNDSLHLLVFVSDADSHFGMDSKLAGIVIPNDGLCHLDSKNEYSMSTV |
| | | LEYPTIGQLIDKLVQNNVLLIFAVTQEQVHLYENYAKLIPGATVGLLQKDS |
| | | GNILQLIISAYEELRSEVELEVLGDTEGLNLSFTAICNNGTLFQHQKKCSH |
| | | MKVGDTASFSVTVNIPHCERRSRHIIIKPVGLGDALELLVSPECNCDCQKE |
| | | VEVNSSKCHHGNGSFQCGVCACHPGHMGPRCECGEDMLSTDSCKEAPDHPS |
| | | CSGRGDCYCGQCICHLSPYGNIYGPYCQCDNFSCVRHKGLLCGGNGDCDCG |
| | | ECVCRSGWTGEYCNCTTSTDSCVSEDGVLCSGRGDCVCGKCVCTNPGASGP |
| | | TCERCPTCGDPCNSKRSCIECHLSAAGQAREECVDKCKLAGATISEEEDFS |
| | | KDGSVSCSLQGENECLITFLITTDNEGKTIIHSINEKDCPKPPNIPMIMLG |
| | | VSLAILLIGVVLLCIWKLLVSFHDRKEVAKFEAERSKAKWQTGTNPLYRGS |
| | | TSTFKNVTYKHREKQKVDLSTDC |
| 18 | Human ITB6, | AUGGGUAUCGAGCUGCUGUGUCUGUUCUUUCUGUUUCUGGGCCGCAAC |
| | nucleotide, | GACCACGUCCAGGGCGGCUGCGCCCUGGGCGGGGCCGAGACCUGCGAG |
| | variant 5 (no | GACUGCCUGCUCAUCGGGCCACAGUGUGCCUGGUGUGCACAGGAGAAC |
| | stop codon) | UUCACCCACCCCAGCGGGGUGGGCGAGAGGUGCGACACCCCUGCAAAU |
| | | CUGCUGGCAAAGGGCUGCCAGCUGAAUUUCAUCGAGAAUCCCGUCAGC |
| | | CAGGUGGAGAUUCUCAAGAACAAGCCUCUGAGCGUCGGACGGCAGAAG |
| | | AACUCUUCUGACAUCGUCCAGAUCGCACCUCAGAGCCUGAUUCUGAAG |
| | | CUCCGCCCAGGCGGAGCUCAGACCCUCCAGGUCCACGUCAGGCAGACU |
| | | GAGGACUACCCCGUGGACCUGUACUACCUGAUGGAUCUGUCCGCCAGC |
| | | AUGGACGACGAUCUGAACACCAUUAAGGAGCUGGGCAGCCGGCUCUCU |
| | | AAGGAGAUGUCCAAGCUCACCAGCAACUUCAGGCUGGGAUUCGGCAGC |
| | | UUUGUCGAGAAGCCUGUGUCUCCUUUCGUGAAGACCACACCAGAGGAG |
| | | AUCGCAAACCCCUGUAGCAGCAUCCCCUACUUCUGCCUGCCUACAUUC |
| | | GGGUUCAAGCACAUUCUGCCUCUGACCAACGACGCAGAGCGCUUUAAC |
| | | GAGAUCGUGAAGAAUCAGAAGAUCAGCGCCAACAUUGAUACUCCUGA |
| | | GGGCGGCUUCGACGCCAUCAUGCAGGCUGCCGUCUGCAAGGAGAAGAU |
| | | CGGGUGGAGGAACGACAGCCUCCACCUGCUCGUGUUCGUGAGCGACGC |
| | | CGACAGCCACUUUGGGAUGGACUCCAAGCUGGCCGGCAUCGUGAUCCC |
| | | CAACGACGGGCUGUGUCACCUCGAUUCCAAGAACGAGUACAGCAUGAG |
| | | CACCGUGCUGGAGUACCCCACCAUUGGGCAGCUCAUUGAUAAGCUCGU |
| | | GCAGAACAACGUCCUGCUGAUCUUUGCAGUGACACAGGAGCAGGUGCA |
| | | CCUGUACGAGAAUUACGCAAAGCUCAUCCCCGGAGCCACUGUGGGCCU |
| | | CCUGCAGAAGGACUCCGGGAACAUCCUCCAGCUCAUCAUCUCCGCCUA |
| | | CGAGGAGCUGAGGAGCGAGGUGGAGCUCGAGGUCCUCGGGGACACCGA |
| | | GGGCCUCAACCUGAGCUUUACCGCCAUCUGCAACAACGGAACUCUCUU |
| | | CCAGCACCAGAAGAAGUGCUCUCACAUGAAGGUGGGCGACACCGCCUC |
| | | CUUCAGCGUGACCGUGAACAUCCCUCACUGUGAGCGGCGGAGCCGCCA |
| | | CAUCAUCAUCAAGCCCGUGGGGCUCGGGGACGCCCUGGAGCUCCUGGU |
| | | GUCCCCUGAGUGUAACUGCGACUGCCAGAAGGAGGUCGAGGUGAACAG |
| | | CUCUAAGUGCCACCACGGCAACGGGAGCUUCCAGUGCGGGGUGUGCGC |
| | | AUGCCACCCCGGCCACAUGGGGCCUCGCUGCGAGUGCGGCGAGGACAU |
| | | GCUGUCUACCGACAGCUGCAAGGAGGCCCCUGACCACCCUAGCUGCUC |
| | | UGGCCGCGGAGACUGCUACUGCGGCCAGUGCAUCUGCCACCUGUCCCC |
| | | UUACGGGAACAUUUACGGGCCCUACUGCCAGUGUGAUAACUUUUCCUG |
| | | CGUGCGGCACAAGGGCCUGCUGUGCGGAGGAAAUGGCGACUGCGACUG |
| | | CGGCGAGUGCGUCUGUCGCAGCGGCUGGACCGGGGAGUACUGCAACUG |
| | | CACUACUUCCACUGACAGCUGUGUCAGCGAGGACGGGGUGCUCUGUUC |
| | | UGGCCGCGGGGACUGCGUCUGCGGCAAGUGCGUGUGCACUAAUCCAGG |
| | | GGCCUCCGGGCCCACCUGCGAGCGGUGUCCCACCUGCGGGGACCCUUG |
| | | CAACAGCAAGAGGUCCUGCAUCGAGUGCCACCUGUCCGCCGCCGGACA |
| | | GGCACGCGAGGAGUGCGUCGAUAAGUGUAAGCUCGCCGGAGCUACAAU |
| | | UAGCGAGGAGGAGGACUUCUCCAAGGACGGGAGCGUGAGCUGCUCCCU |
| | | CCAGGGAGAGAACGAGUGCCUGAUCACUUUCCUCAUCACCACUGAUAA |
| | | CGAGGGAAAGACAAUCAUUCACUCUAUCAACGAGAAGGAUUGUCCCAA |
| | | GCCCCCAAACAUUCCCAUGAUCAUGCUGGGAGUGUCCCUGGCAAUCCU |
| | | GCUGAUCGGAGUCGUGCUGCUCUGCAUCUGGAAGCUGCUCGUCUCCUU |
| | | CCACGACCGCAAGGAGGUCGCAAAGUUUGAGGCCGAGCGGUCUAAGGC |
| | | CAAGUGGCAGACAGGGACCAAUCCUCUCUACCGCGGGUCUACUUCUAC |
| | | CUUCAAGAACGUGACUUACAAGCACAGGGAGAAGCAGAAGGUGGACC |
| | | UCUCCACCGAUUGC |
| 13 | Rat Variant 1 | MGIELVCLFLLLLGRNDHVQGGCAWSGAETCSDCLLTGPHCAWCSQENFTHLSGAG |
| | Amino Acid | ERCDTPENLLAKGCQLPFIENPVSQVEILQNKPLSVGRQKNSSDIVQIAPQSLVLK |
| | | LRPGGEQTLQVQVRQTEDYPVDLYYLMDLSASMDDDLNTIKELGSRLAKEMSKLTS |
| | | NFRLGFGSFVEKPVSPFMKTTPEEITNPCSSIPYFCLPTFGFKHILPLTDDAERFN |
| | | EIVRKQKISANIDTPEGGFDAIMQAAVCKEKIGWRNDSLHLLVFVSDADSHFGMDS |
| | | KLAGIVIPNDGLCHLDNRNEYSMSTVLEYPTIGQLIDKLVQNNVLLIFAVTQEQVH |
| | | LYENYAKLIPGATVGLLQKDSGNILQLIISAYEELRSEVELEVLGDTEGLNLSFTA |
| | | LCSNGILFPHQKKCSHMKVGDTASFNVSVSITNCEKRSRKLIIKPVGLGDTLEILV |
| | | SAECDCDCQREVEANSSKCHHGNGSFQCGVCACNPGHMGPRCECGEDMVSTDSCKE |
| | | SPGHPSCSGRGDCYCGQCVCHLSPYGSIYGPYCQCDNFSCLRHKGLLCGDNGDCDC |
| | | GECVCRDGWTGEYCNCTTSRDACASEDGVLCSGRGDCVCGKCVCRNPGASGPTCER |
| | | CPTCGDPCNSRRSCIECYLSADGQAQEECEDKCKATGATISEEEFSKDTSVPCSLQ |
| | | GENECLITFLITADNEGKTIIHNISEKDCPKPPNIPMIMLGVSLAILLIGVVLLCI |
| | | WKLLVSFHDRKEVAKFEAERSKAKWQTGTNPLYRGSTSTFKNVTYKHREKHKVGLS |
| | | SDGGKPIPNPLLGLDST |
| 14 | Rat Variant 1 | AUGGGCAUCGAGCUGGUGUGCCUGUUCCUGUUACUACUGGGCAGAAACGACCACGU |
| | Nucleotide | GCAGGGCGGCUGCGCCUGGAGCGGCGCCGAAACCUGCAGCGACUGCCUGCUGACCG |
| | | GCCCUCACUGCGCUUGGUGCAGCCAGGAGAACUUCACCCACCUCUCAGGCGCUGGC |
| | | GAGCGGUGCGACACCCCUGAGAACCUGCUGGCCAAGGGCUGCCAGCUGCCUUUCAU |
| | | CGAGAACCCUGUGAGCCAGGUGGAGAUCCUGCAGAACAAGCCUCUGAGCGUGGGCA |
| | | GACAGAAGAACAGCAGCGACAUCGUGCAGAUCGCCCCUCAGAGUCUCGUUCUGAAG |
| | | CUUCGGCCUGGCGGCGAGCAGACCCUGCAGGUGCAGGUGAGACAGACCGAGGACUA |
| | | CCCUGUGGACCUGUACUACCUGAUGGACUUAUCCGCCAGCAUGGACGACGACCUGA |
| | | ACACCAUCAAGGAGCUGGGCAGCAGACUUGCAAAGGAGAUGAGCAAGCUGACCAGC |
| | | AACUUCAGACUGGGCUUCGGCAGCUUCGUGGAGAAGCCAGUUAGUCCUUUCAUGAA |
| | | GACGACCCCAGAGGAGAUCACCAACCCUUGCAGCAGCAUCCCUUACUUCUGCCUGC |
| | | CUACCUUCGGCUUCAAGCACAUCCUGCCUCUGACCGACGACGCCGAGAGAUUCAAC |
| | | GAGAUCGUGAGAAAGCAGAAGAUCAGCGCCAACAUCGAUACACCUGAGGGAGGUUU |
| | | CGACGCCAUCAUGCAGGCCGCCGUGUGCAAGGAGAAGAUCGGCUGGCGAAACGACA |
| | | GCCUGCACCUUCUAGUGUUCGUGAGCGACGCAGACAGCCACUUCGGCAUGGACUCU |
| | | AAGCUAGCCGGCAUCGUGAUCCCUAACGACGGCCUGUGCCACCUGGACAAUCGUAA |
| | | CGAGUACAGCAUGAGCACCGUGCUGGAGUACCCUACCAUCGGCCAGCUGAUCGACA |
| | | AGCUUGUGCAGAAUAACGUGCUGCUGAUCUUCGCCGUGACCCAGGAGCAGGUCCAC |
| | | CUGUACGAGAACUACGCCAAGCUGAUCCCUGGCGCCACCGUGGGCCUGCUGCAGAA |
| | | GGACAGCGGCAACAUCUUACAGCUGAUUAUAUCAGCCUACGAGGAGCUGAGAAGCG |
| | | AGGUGGAGCUGGAGGUGCUGGGCGACACCGAGGGCCUGAACCUGAGCUUCACCGCC |
| | | CUGUGCAGCAACGGCAUCCUGUUCCCUCACCAGAAGAAGUGUUCCCACAUGAAGGU |
| | | CGGUGAUACAGCAUCAUUCAACGUGAGCGUGAGCAUUACCAAUUGCGAGAAGAGAA |
| | | GCAGAAAGUUAAUAAUUAAGCCUGUCGGACUGGGCGAUACUCUUGAAAUCCUAGUG |
| | | AGCGCCGAGUGCGACUGUGAUUGCCAGAGAGAGGUGGAAGCCAAUUCUUCCAAGUG |
| | | CCACCACGGCAACGGAUCCUUCCAGUGCGGCGUGUGCGCCUGCAACCCUGGCCACA |
| | | UGGGCCCUCGAUGCGAGUGUGGAGAGGACAUGGUUUCUACCGACAGCUGCAAGGAA |
| | | AGCCCAGGCCAUCCUAGCUGCAGCGGCAGAGGCGACUGCUACUGCGGCCAGUGCGU |
| | | GUGCCAUCUGAGUCCUUACGGCAGCAUCUACGGCCCUUACUGUCAGUGCGAUAACU |
| | | UCUCGUGCCUGAGACACAAGGGUUUGCUGUGCGGUGACAACGGAGAUUGUGACUGC |
| | | GGAGAGUGCGUGUGUAGAGACGGCUGGACCGGCGAGUACUGCAACUGCACCACCAG |
| | | CAGGGACGCCUGCGCCAGCGAGGACGGCGUGUUGUGUUCUGGACGUGGUGAUUGUG |
| | | UGUGUGGCAAGUGCGUCUGCAGAAACCCGGGAGCCAGCGGCCCUACCUGUGAGAGG |
| | | UGCCCAACUUGUGGCGAUCCUUGCAAUUCACGUAGGAGCUGCAUCGAGUGCUACUU |
| | | GAGCGCUGACGGCCAGGCCCAGGAGGAGUGCGAGGAUAAGUGCAAGGCCACCGGAG |
| | | CAACUAUCUCUGAGGAAGAAUUCAGCAAGGACACCAGCGUGCCUUGCAGUUUGCAG |
| | | GGAGAGAACGAGUGCCUGAUCACCUUCCUCAUCACCGCCGACAACGAGGGCAAGAC |
| | | CAUCAUCCACAAUAUUAGCGAGAAGGACUGCCCUAAGCCUCCUAACAUCCCUAUGA |
| | | UCAUGCUGGGAGUGAGCUUGGCUAUCCUGCUUAUCGGCGUGGUCUUGCUGUGUAUC |
| | | UGGAAGCUCCUCGUCAGUUUCCACGACAGAAAGGAGGUGGCCAAGUUCGAGGCUGA |
| | | AAGAUCUAAGGCCAAGUGGCAGACCGGAACCAAUCCUCUGUACAGAGGCAGCACCA |
| | | GCACCUUCAAGAACGUGACCUACAAGCACCGGGAGAAGCACAAGGUCGGCCUGAGU |
| | | AGCGACGGCGGCAAGCCUAUUCCAAACCCACUGCUCGGUCUGGACUCAACC |
| 15 | Mouse ITB6 | MGIELVCLFLLLLGRNDHVQGGCAWGGAESCSDCLLTGPHCAWCSQENFTHLS |
| | amino acid | GAGERCDTPANLLAKGCQLPFIENPVSRIEVLQNKPLSVGRQKNSSDIVQIAPQSL |
| | | VLKLRPGREQTLQVQVRQTEDYPVDLYYLMDLSASMDDDLNTIKELGSRLAKEMSK |
| | | LTSNFRLGFGSFVEKPVSPFMKTTPEEITNPCSSIPYFCLPTFGFKHILPLTDDAE |
| | | RFNEIVRKQKISANIDTPEGGFDAIMQAAVCKEKIGWRNDSLHLLVFVSDADSHFG |
| | | MDSKLAGIVIPNDGLCHLDHRNEYSMSTVLEYPTIGQLIDKLVQNNVLLIFAVTQE |
| | | QVHLYENYAKLIPGATVGLLQKDSGNILQLIISAYEELRSEVELEVLGDTEGLNLS |
| | | FTALCNNGVLFPHQKKCSHMKVGDTASFNVTVSVSNCEKRSRNLIIKPVGLGDTLE |
| | | ILVSAECDCDCQREIETNSSKCHNGNGSFQCGVCTCNPGHMGPHCECGEDMVSTDS |
| | | CKESPGHPSCSGRGDCYCGQCICHLSPYGSIYGPYCQCDNFSCLRHKGLLCGDNGD |
| | | CDCGECVCRDGWTGEYCNCTTNRDSCTSEDGVLCSGRGDCVCGKCVCRNPGASGPT |
| | | CERCPTCGDPCNSKRSCIECYLSADGQAQEECADKCKAIGATISEEDFSKDTSVSC |
| | | SLQGENECLITFLITTDNEGKTIIHNINEKDCPKPPNIPMIMLGVSLAILLIGVVL |
| | | LCIWKLLVSFHDRKEVAKFEAERSKAKWQTGTNPLYRGSTSTFKNVTYKHREKHKA |
| | | GLSSDGGKPIPNPLLGLDST |
| 16 | Mouse ITB6 | AUGGGCAUCGAGCUGGUGUGCCUGUUCCUGCUACUCUUAGGCAGAAACGACCA |
| | nucleotide | CGUGCAAGGCGGCUGCGCCUGGGGAGGUGCCGAGAGCUGCAGCGACUGCCUGCUGA |
| | | CCGGCCCUCACUGUGCGUGGUGCAGCCAGGAGAACUUCACCCACCUGAGUGGCGCC |
| | | GGCGAGCGAUGCGACACCCCUGCCAACCUGCUGGCCAAGGGCUGCCAGCUGCCUUU |
| | | CAUCGAGAACCCUGUGAGCAGAAUCGAGGUGCUGCAGAACAAGCCUCUGAGCGUGG |
| | | GCAGACAGAAGAACAGCAGCGACAUCGUGCAGAUCGCCCCUCAGUCACUUGUACUG |
| | | AAGUUGCGGCCUGGCAGAGAGCAGACCCUGCAGGUGCAGGUGAGACAGACCGAGGA |
| | | CUACCCUGUGGACCUGUACUACCUGAUGGAUCUGAGCGCCAGCAUGGACGACGACC |
| | | UGAACACCAUCAAGGAACUGGGUAGCAGACUGGCUAAGGAGAUGAGCAAGCUGACC |
| | | AGCAACUUCAGACUGGGCUUCGGCAGCUUCGUGGAGAAGCCUGUUUCACCUUUCAU |
| | | GAAGACCACCCCUGAGGAGAUCACCAACCCUUGCAGCAGCAUCCCUUACUUCUGCC |
| | | UGCCUACCUUCGGCUUCAAGCACAUCCUGCCUCUGACCGACGACGCGGAGAGAUUC |
| | | AACGAGAUCGUGAGAAAGCAGAAGAUCAGCGCCAACAUUGACACCCCGGAAGGCGG |
| | | AUUCGACGCCAUCAUGCAGGCCGCCGUGUGCAAGGAGAAGAUCGGCUGGAGAAACG |
| | | AUAGCCUGCAUUUGUUAGUGUUCGUGAGUGACGCAGACAGCCACUUCGGCAUGGAC |
| | | UCCAAGCUUGCCGGCAUCGUGAUCCCUAACGACGGCCUGUGCCACCUGGACCACAG |
| | | GAACGAGUACAGCAUGAGCACCGUGCUGGAGUACCCUACCAUCGGCCAGCUGAUCG |
| | | ACAAGCUGGUCCAGAAUAACGUGCUGCUGAUCUUCGCCGUGACCCAGGAACAGGUU |
| | | CACCUGUACGAGAACUACGCCAAGCUGAUCCCUGGCGCCACCGUGGGCCUCCUGCA |
| | | GAAGGACAGCGGCAACAUCCUCCAACUGAUCAUAUCUGCUUACGAGGAGCUGAGAA |
| | | GCGAGGUGGAGCUGGAGGUGCUCGGCGACACCGAGGGCCUGAACCUGAGCUUCACC |
| | | GCCCUGUGCAACAACGGCGUGCUGUUCCCUCACCAGAAGAAGUGUAGUCACAUGAA |
| | | GGUUGGCGAUACCGCCAGCUUCAACGUGACCGUGAGCGUGAGCAACUGCGAGAAGA |
| | | GAAGCAGAAACCUUAUAAUAAAGCCAGUUGGUCUGGGCGACACACUGGAGAUCCUG |
| | | GUGAGCGCCGAGUGCGACUGCGAUUGUCAGAGAGAGAUCGAGACAAAUUCGUCUAA |
| | | GUGCCACAACGGCAACGGAUCUUUCCAGUGCGGCGUGUGCACCUGCAACCCUGGCC |
| | | ACAUGGGACCACACUGCGAGUGCGGAGAGGACAUGGUGUCAACCGACAGCUGUAAG |
| | | GAGAGCCCAGGCCAUCCUUCUUGCUCCGGCAGAGGCGACUGCUACUGCGGCCAGUG |
| | | CAUCUGCCAUCUCUCCCCUUACGGCAGCAUCUACGGCCCUUACUGCCAGUGCGAUA |
| | | ACUUCUCGUGCCUGAGACACAAGGGCCUGCUCUGCGGCGACAACGGCGACUGUGAU |
| | | UGUGGUGAGUGUGUGUGCAGAGACGGCUGGACCGGCGAGUACUGCAACUGCACCAC |
| | | CAACAGAGAUAGCUGUACCAGCGAGGACGGUGUCCUCUGUUCCGGACGGGGUGAUU |
| | | GCGUUUGUGGCAAGUGUGUUUGCCGUAACCCAGGAGCCAGCGGCCCUACCUGCGAA |
| | | AGGUGCCCGACCUGUGGAGAUCCGUGUAACAGCAAGCGAAGUUGCAUCGAGUGCUA |
| | | UCUCUCCGCAGACGGCCAGGCCCAGGAGGAGUGCGCCGACAAGUGCAAGGCCAUCG |
| | | GCGCUACUAUUUCGGAAGAGGACUUCAGCAAGGACACCAGCGUUAGCUGUUCGCUG |
| | | CAGGGUGAGAACGAGUGCCUGAUCACCUUCCUCAUCACGACCGACAACGAGGGCAA |
| | | GACCAUCAUCCACAACAUCAACGAGAAGGACUGCCCUAAGCCUCCUAACAUCCCUA |
| | | UGAUCAUGCUGGGAGUGAGCCUGGCCAUACUGCUUAUCGGCGUGGUUCUGCUGUGC |
| | | AUUUGGAAGUUAUUAGUGAGUUUCCACGACAGAAAGGAGGUGGCCAAGUUCGAGGC |
| | | CGAGAGGUCCAAGGCCAAGUGGCAGACCGGCACGAACCCACUGUACAGAGGCAGCA |
| | | CCAGCACCUUCAAGAACGUCACAUACAAGCACCGGGAGAAGCACAAGGCCGGCCUG |
| | | UCAUCUGACGGCGGCAAGCCUAUCCCGAAUCCACUCCUAGGACUGGAUAGUACC |

Signal sequence underlined;
tag sequence italicized and underlined

In some embodiments, a polynucleotide of the present disclosure, for example a polynucleotide comprising an mRNA nucleotide sequence encoding an ITB6 polypeptide, comprises (1) a 5′ cap, e.g., as disclosed herein, e.g., as provided in Table 2A, (2) a 5′ UTR, e.g., as provided in Table 2A, (3) a nucleotide sequence ORF provided in Table 2A, (4) a stop codon, (5) a 3′UTR, e.g., as provided in Table 2A, and (6) a tail (e.g., poly-A tail), e.g., as disclosed herein, e.g., a poly-A tail of about 100 residues (e.g., SEQ ID NO:502).

In some embodiments, the polynucleotide comprises an mRNA nucleotide sequence encoding an ITB6 polypeptide.

In some embodiments, the polynucleotide (e.g., mRNA) encoding the ITB6 molecule comprises the nucleotide sequence of SEQ ID NO: 175, which comprises from 5′ to 3′ end: the 5′ UTR sequence of SEQ ID NO: 50, the ORF sequence of SEQ ID NO: 18, and the 3′ UTR sequence of SEQ ID NO: 142.

In some embodiments, the polynucleotide (e.g., mRNA) encoding the ITB6 molecule comprises the nucleotide sequence of SEQ ID NO: 160, which comprises from 5′ to 3′ end: the 5′ UTR sequence of SEQ ID NO: 56, the ORF sequence of SEQ ID NO: 2, and the 3′ UTR sequence of SEQ ID NO: 110.

In some embodiments, the polynucleotide (e.g., mRNA) encoding the ITB6 molecule comprises the nucleotide sequence of SEQ ID NO: 161, which comprises from 5′ to 3′ end: the 5′ UTR sequence of SEQ ID NO: 50, the ORF sequence of SEQ ID NO: 3, and the 3′ UTR sequence of SEQ ID NO: 110.

In some embodiments, the polynucleotide (e.g., mRNA) encoding the ITB6 molecule comprises the nucleotide sequence of SEQ ID NO: 162, which comprises from 5′ to 3′ end: the 5′ UTR sequence of SEQ ID NO: 50, ORF sequence of SEQ ID NO: 4, and the 3′ UTR sequence of SEQ ID NO: 143.

In some embodiments, the polynucleotide (e.g., mRNA) encoding the ITB6 molecule comprises the nucleotide sequence of SEQ ID NO: 163, which comprises from 5′ to 3′ end: the 5′ UTR sequence of SEQ ID NO: 50, the ORF sequence of SEQ ID NO: 5, and the 3′ UTR sequence of SEQ ID NO: 110.

In some embodiments, the polynucleotide (e.g., mRNA) encoding the ITB6 molecule comprises the nucleotide sequence of SEQ ID NO: 164, which comprises from 5′ to 3′ end: the 5′ UTR sequence of SEQ ID NO: 50, the ORF sequence of SEQ ID NO: 5, and the 3′ UTR sequence of SEQ ID NO: 144.

In some embodiments, the polynucleotide (e.g., mRNA) encoding the ITB6 molecule comprises the nucleotide sequence of SEQ ID NO: 169, which comprises from 5′ to 3′ end: the 5′ UTR sequence of SEQ ID NO: 50, the ORF sequence of SEQ ID NO: 5, and the 3′ UTR sequence of SEQ ID NO: 145.

In some embodiments, the polynucleotide (e.g., mRNA) encoding the ITB6 molecule comprises the nucleotide sequence of SEQ ID NO: 165, which comprises from 5′ to 3′ end: the 5′ UTR sequence of SEQ ID NO: 50, the ORF sequence of SEQ ID NO: 6, and the 3′ UTR sequence of SEQ ID NO: 110.

In some embodiments, the polynucleotide (e.g., mRNA) encoding the ITB6 molecule comprises the nucleotide sequence of SEQ ID NO: 166, which comprises from 5′ to 3′ end: the 5′ UTR sequence of SEQ ID NO: 50, the ORF sequence of SEQ ID NO: 6, and the 3′ UTR sequence of SEQ ID NO: 110.

In some embodiments, the polynucleotide (e.g., mRNA) encoding the ITB6 molecule comprises the nucleotide sequence of SEQ ID NO: 167, which comprises from 5′ to 3′ end: the 5′ UTR sequence of SEQ ID NO: 50, the ORF sequence of SEQ ID NO: 6, and the 3′ UTR sequence of SEQ ID NO: 145.

In some embodiments, the polynucleotide (e.g., mRNA) encoding the ITB6 molecule comprises the nucleotide sequence of SEQ ID NO: 168, which comprises from 5′ to 3′ end: the 5′ UTR sequence of SEQ ID NO: 50, the ORF sequence of SEQ ID NO: 6, and the 3′ UTR sequence of SEQ ID NO: 143.

In some embodiments, the polynucleotide (e.g., mRNA) encoding the ITB6 molecule comprises the nucleotide sequence of SEQ ID NO: 170, which comprises from 5′ to 3′ end: the 5′ UTR sequence of SEQ ID NO: 50, the ORF sequence of SEQ ID NO: 8, and the 3′ UTR sequence of SEQ ID NO: 110.

In some embodiments, the polynucleotide (e.g., mRNA) encoding the ITB6 molecule comprises the nucleotide sequence of SEQ ID NO: 171, which comprises from 5′ to 3′ end: the 5′ UTR sequence of SEQ ID NO: 50, the ORF sequence of SEQ ID NO: 10, and the 3′ UTR sequence of SEQ ID NO: 110.

In some embodiments, the polynucleotide (e.g., mRNA) encoding the ITB6 molecule comprises the nucleotide sequence of SEQ ID NO: 172, which comprises from 5′ to 3′ end: the 5′ UTR sequence of SEQ ID NO: 50, the ORF sequence of SEQ ID NO: 12, and the 3′ UTR sequence of SEQ ID NO: 110.

In some embodiments, the polynucleotide (e.g., mRNA) encoding the ITB6 molecule comprises the nucleotide sequence of SEQ ID NO: 173, which comprises from 5′ to 3′ end: the 5′ UTR sequence of SEQ ID NO: 50, the ORF sequence of SEQ ID NO: 14, and the 3′ UTR sequence of SEQ ID NO: 110.

In some embodiments, the polynucleotide (e.g., mRNA) encoding the ITB6 molecule comprises the nucleotide sequence of SEQ ID NO: 174, which comprises from 5′ to 3′ end: the 5′ UTR sequence of SEQ ID NO: 56, the ORF sequence of SEQ ID NO: 16, and the 3′ UTR sequence of SEQ ID NO: 110.

In some embodiments, all of the 5′ UTR, ORF, and/or 3′ UTR sequences include the modification(s) described in Table 2A. In some embodiments, one, two, or all of the 5′ UTR, ORF, and/or 3′ UTR sequences do not include the modification(s) described in Table 2A. In some embodiments, the 5′ UTRs described in Table 2A additionally comprise a first nucleotide that is an “A” or a “G.”

TABLE 2A
Exemplary ITB6 construct sequences

| | | ORF | | | | |
|---|---|---|---|---|---|---|
| | mRNA | Sequence | | | | |
| | Descrip- | (Amino | ORF Sequence | 5′ UTR | 3′ UTR | Construct |
| | tion | Acid) | (Nucleotide) | Sequence | Sequence | Sequence |
| SEQ ID | | | | | | |
| NO | | 17 | 18 | 50 | 142 | 175 |
| Human | ITB6 | MGIELLCLF | AUGGGUAUCGAGCUGCUGUGUCUGUUCUUUCUGUUUCUG | GGAAAUCG | UAAGCCCC | SEQ ID |
| Var- | G5 | FLFLGRNDH | GGCCGCAACGACCACGUCCAGGGCGGCUGCGCCCUGGGC | CAAAAUUU | UCCGGGGC | NO: 175 |
| iant | Cap: | VQGGCALGG | GGGGCCGAGACCUGCGAGGACUGCCUGCUCAUCGGGCCA | GCUCUUCG | AAACACCA | consists |
| 5.1 | m⁷GpppG_{2′O} | AETCEDCLL | CAGUGUGCCUGGUGUGCACAGGAGAACUUCACCCACCCC | CGUUAGAU | UUGUCACA | from 5′ |
| | MepA | IGPQCAWCA | AGCGGGGUGGGCGAGAGGUGCGACACCCCUGCAAAUCUG | UUCUUUUA | CUCCAGCC | to 3′ |
| | Poly A | QENFTHPSG | CUGGCAAAGGGCUGCCAGCUGAAUUUCAUCGAGAAUCCC | GUUUUCUC | UCGGUGGC | end: 5′ |
| | tail: | VGERCDTPA | GUCAGCCAGGUGGAGAUUCUCAAGAACAAGCCUCUGAGC | GCAACUAG | CUAGCUUC | UTR of |
| | 100 nt | NLLAKGCQL | GUCGGACGGCAGAAGAACUCUUCUGACAUCGUCCAGAUC | CAAGCUUU | UUGCCCCU | SEQ ID |
| | (SEQ ID | NFIENPVSQ | GCACCUCAGAGCCUGAUUCUGAAGCUCCGCCCAGGCGGA | UUGUUCUC | UGGGCCCA | NO: 50, |
| | NO: | VEILKNKPL | GCUCAGACCCUCCAGGUCCACGUCAGGCAGACUGAGGAC | GCC | AACACCAU | ORF |
| | 502) | SVGRQKNSS | UACCCCGUGGACCUGUACUACCUGAUGGAUCUGUCCGCC | | UGUCACAC | sequence |
| | | DIVQIAPQS | AGCAUGGACGACGAUCUGAACACCAUUAAGGAGCUGGGC | | UCCAUCCC | of SEQ |
| | | LILKLRPGG | AGCCGGCUCUCUAAGGAGAUGUCCAAGCUCACCAGCAAC | | CCCAGCCC | ID NO: |
| | | AQTLQVHVR | UUCAGGCUGGGAUUCGGCAGCUUUGUCGAGAAGCCUGUG | | CUCCUCCC | 18, and |
| | | QTEDYPVDL | UCUCCUUUCGUGAAGACCACACCAGAGGAGAUCGCAAAC | | CUUCCUGC | 3′ UTR |
| | | YYLMDLSAS | CCCUGUAGCAGCAUCCCCUACUUCUGCCUGCCUACAUUC | | ACCCGUAC | sequence |
| | | MDDDLNTIK | GGGUUCAAGCACAUUCUGCCUCUGACCAACGACGCAGAG | | CCCCCAAA | of SEQ |
| | | ELGSRLSKE | CGCUUUAACGAGAUCGUGAAGAAUCAGAAGAUCAGCGCC | | CACCAUUG | ID NO: |
| | | MSKLTSNFR | AACAUUGAUACUCCUGAGGGCGGCUUCGACGCCAUCAUG | | UCACACUC | 142. |
| | | LGFGSFVEK | CAGGCUGCCGUCUGCAAGGAGAAGAUCGGGUGGAGGAAC | | CAGUGGUC | |
| | | PVSPFVKTT | GACAGCCUCCACCUGCUCGUGUUCGUGAGCGACGCCGAC | | UUUGAAUA | |
| | | PEEIANPCS | AGCCACUUUGGGAUGGACUCCAAGCUGGCCGGCAUCGUG | | AAGUCUGA | |
| | | SIPYFCLPT | AUCCCCAACGACGGGCUGUGUCACCUCGAUUCCAAGAAC | | GUGGGCGG | |
| | | FGFKHILPL | GAGUACAGCAUGAGCACCGUGCUGGAGUACCCCACCAUU | | C | |
| | | TNDAERFNE | GGGCAGCUCAUUGAUAAGCUCGUGCAGAACAACGUCCUG | | | |
| | | IVKNQKISA | CUGAUCUUUGCAGUGACACAGGAGCAGGUGCACCUGUAC | | | |
| | | NIDTPEGGF | GAGAAUUACGCAAAGCUCAUCCCCGGAGCCACUGUGGGC | | | |
| | | DAIMQAAVC | CUCCUGCAGAAGGACUCCGGGAACAUCCUCCAGCUCAUC | | | |
| | | KEKIGWRND | AUCUCCGCCUACGAGGAGCUGAGGAGCGAGGUGGAGCUC | | | |
| | | SLHLLVFVS | GAGGUCCUCGGGGACACCGAGGGCCUCAACCUGAGCUUU | | | |
| | | DADSHFGMD | ACCGCCAUCUGCAACAACGGAACUCUCUUCCAGCACCAG | | | |
| | | SKLAGIVIP | AAGAAGUGCUCUCACAUGAAGGUGGGCGACACCGCCUCC | | | |
| | | NDGLCHLDS | UUCAGCGUGACCGUGAACAUCCCUCACUGUGAGCGGCGG | | | |
| | | KNEYSMSTV | AGCCGCCACAUCAUCAUCAAGCCCGUGGGGCUCGGGGAC | | | |
| | | LEYPTIGQL | GCCCUGGAGCUCCUGGUGUCCCCUGAGUGUAACUGCGAC | | | |
| | | IDKLVQNNV | UGCCAGAAGGAGGUCGAGGUGAACAGCUCUAAGUGCCAC | | | |
| | | LLIFAVTQE | CACGGCAACGGGAGCUUCCAGUGCGGGGUGUGCGCAUGC | | | |
| | | QVHLYENYA | CACCCCGGCCACAUGGGGCCUCGCUGCGAGUGCGGCGAG | | | |
| | | KLIPGATVG | GACAUGCUGUCUACCGACAGCUGCAAGGAGGCCCCUGAC | | | |
| | | LLQKDSGNI | CACCCUAGCUGCUCUGGCCGCGGAGACUGCUACUGCGGC | | | |
| | | LQLIISAYE | CAGUGCAUCUGCCACCUGUCCCCUUACGGGAACAUUUAC | | | |
| | | ELRSEVELE | GGGCCCUACUGCCAGUGUGAUAACUUUUCCUGCGUGCGG | | | |
| | | VLGDTEGLN | CACAAGGGCCUGCUGUGCGGAGGAAAUGGCGACUGCGAC | | | |
| | | LSFTAICNN | UGCGGCGAGUGCGUCUGUCGCAGCGGCUGGACCGGGGAG | | | |
| | | GTLFQHQKK | UACUGCAACUGCACUACUUCCACUGACAGCUGUGUCAGC | | | |
| | | CSHMKVGDT | GAGGACGGGGUGCUCUGUUCUGGCCGCGGGGACUGCGUC | | | |
| | | ASFSVTVNI | UGCGGCAAGUGCGUGUGCACUAAUCCAGGGGCCUCCGGG | | | |
| | | PHCERRSRH | CCCACCUGCGAGCGGUGUCCCACCUGCGGGGACCCUUGC | | | |
| | | IIIKPVGLG | AACAGCAAGAGGUCCUGCAUCGAGUGCCACCUGUCCGCC | | | |
| | | DALELLVSP | GCCGGACAGGCACGCGAGGAGUGCGUCGAUAAGUGUAAG | | | |
| | | ECNCDCQKE | CUCGCCGGAGCUACAAUUAGCGAGGAGGAGGACUUCUCC | | | |
| | | VEVNSSKCH | AAGGACGGGAGCGUGAGCUGCUCCCUCCAGGGAGAGAAC | | | |
| | | HGNGSFQCG | GAGUGCCUGAUCACUUUCCUCAUCACCACUGAUAACGAG | | | |
| | | VCACHPGHM | GGAAAGACAAUCAUUCACUCUAUCAACGAGAAGGAUUGU | | | |
| | | GPRCECGED | CCCAAGCCCCCAAACAUUCCCAUGAUCAUGCUGGGAGUG | | | |
| | | MLSTDSCKE | UCCCUGGCAAUCCUGCUGAUCGGAGUCGUGCUGCUCUGC | | | |
| | | APDHPSCSG | AUCUGGAAGCUGCUCGUCUCCUUCCACGACCGCAAGGAG | | | |
| | | RGDCYCGQC | GUCGCAAAGUUUGAGGCCGAGCGGUCUAAGGCCAAGUGG | | | |
| | | ICHLSPYGN | CAGACAGGGACCAAUCCUCUCUACCGCGGGUCUACUUCU | | | |
| | | IYGPYCQCD | ACCUUCAAGAACGUGACUUACAAGCACAGGGAGAAGCAG | | | |
| | | NFSCVRHKG | AAGGUGGACCUCUCCACCGAUUGC | | | |
| | | LLCGGNGDC | | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDC | | | | |
| SEQ ID | | | | | | |
| NO: | | 1 | 2 | 56 | 110 | 160 |
| Human | ITB6 | MGIELLCLF | AUGGGCAUCGAGCUGCUGUGCCUGUUCUUCCUGUUCCUG | GGAAAUAA | UGAUAAUA | SEQ ID |
| Var- | G5 | FLFLGRNDH | GGCAGAAACGACCACGUGCAGGGAGGUUGCGCCCUCGGA | GAGAGAAA | GGCUGGAG | NO: 160 |
| iant | Cap C1 | VQGGCALGG | GGUGCCGAGACAUGCGAGGACUGCCUGCUGAUCGGCCCU | AGAAGAGU | CCUCGGUG | consists |
| 1.1 | Poly A | AETCEDCLL | CAGUGCGCCUGGUGCGCCCAGGAGAACUUCACCCACCCU | AAGAAGAA | GCCUAGCU | from 5′ |
| | tail: | IGPQCAWCA | AGCGGCGUGGGCGAGAGGUGCGACACCCCUGCCAACCUG | AUAUAAGA | UCUUGCCC | to 3′ |
| | 100 nt | QENFTHPSG | CUGGCCAAGGGCUGCCAGCUGAACUUCAUCGAGAACCCU | CCCCGGCG | CUUGGGCC | end: 5′ |
| | (SEQ ID | VGERCDTPA | GUGAGCCAGGUGGAGAUCCUGAAGAACAAGCCUCUGAGC | CCGCCACC | UCCCCCCA | UTR of |
| | NO: 502) | NLLAKGCQL | GUGGGCAGACAGAAGAACAGCAGCGACAUCGUGCAGAUC | | GCCCCUCC | SEQ ID |
| | | NFIENPVSQ | GCCCCUCAGAGCCUGAUUUUGAAGCUCCGGCCUGGAGGA | | UCCCCUUC | NO: 56, |
| | | VEILKNKPL | GCACAGACCCUGCAGGUGCACGUGAGACAGACCGAGGAC | | CUGCACCC | ORF |
| | | SVGRQKNSS | UACCCUGUGGACCUGUACUACCUGAUGGACCUGAGCGCC | | GUACCCCC | sequence |
| | | DIVQIAPQS | AGCAUGGACGACGACCUGAACACCAUCAAGGAGCUGGGC | | CAAACACC | of SEQ |
| | | LILKLRPGG | AGCAGACUGAGCAAGGAGAUGAGCAAGCUGACCAGCAAC | | AUUGUCAC | ID NO: |
| | | AQTLQVHVR | UUCAGACUGGGCUUCGGCAGCUUCGUGGAGAAGCCUGUA | | ACUCCAGU | 2, and |
| | | QTEDYPVDL | UCGCCUUUCGUGAAGACCACCCCUGAGGAGAUCGCCAAC | | GGUCUUUG | 3′ UTR |
| | | YYLMDLSAS | CCUUGCAGCAGCAUCCCUUACUUCUGCCUGCCUACCUUC | | AAUAAAGU | sequence |
| | | MDDDLNTIK | GGCUUCAAGCACAUCCUGCCUCUGACCAACGACGCCGAG | | CUGAGUGG | of SEQ |
| | | ELGSRLSKE | AGAUUCAACGAGAUAGUUAAGAACCAGAAGAUCAGCGCC | | GCGGC | ID NO: |
| | | MSKLTSNFR | AACAUAGAUACUCCAGAAGGAGGUUUCGACGCCAUCAUG | | | 110. |
| | | LGFGSFVEK | CAGGCCGCCGUGUGUAAGGAGAAGAUCGGCUGGCGGAAC | | | |
| | | PVSPFVKTT | GACAGCCUGCAUCUUUUGGUGUUCGUGAGUGACGCAGAC | | | |
| | | PEEIANPCS | AGCCACUUCGGCAUGGACUCUAAGUUAGCCGGCAUCGUG | | | |
| | | SIPYFCLPT | AUCCCUAACGACGGCCUGUGCCACCUGGACAGCAAGAAC | | | |
| | | FGFKHILPL | GAGUACAGCAUGAGCACCGUGCUGGAGUACCCUACCAUC | | | |
| | | TNDAERFNE | GGUCAGCUGAUCGACAAGCUGGUGCAGAACAACGUGCUG | | | |
| | | IVKNQKISA | CUAAUCUUCGCCGUGACCCAGGAACAAGUCCACCUGUAC | | | |
| | | NIDTPEGGF | GAGAACUACGCCAAGCUGAUCCCUGGCGCCACCGUGGGC | | | |
| | | DAIMQAAVC | CUGCUGCAGAAGGACAGCGGCAAUAUCUUGCAGUUGAUU | | | |
| | | KEKIGWRND | AUUUCCGCAUACGAGGAGCUGAGAAGCGAGGUGGAGCUG | | | |
| | | SLHLLVFVS | GAGGUGCUGGGCGACACCGAGGGCCUGAACCUGAGCUUC | | | |
| | | DADSHFGMD | ACCGCCAUCUGCAACAACGGCACCCUGUUCCAGCAUCAG | | | |
| | | SKLAGIVIP | AAGAAGUGCAGCCACAUGAAGGUGGGCGAUACAGCCAGC | | | |
| | | NDGLCHLDS | UUCAGCGUGACCGUGAACAUCCCUCACUGCGAGAGAAGA | | | |
| | | KNEYSMSTV | AGCAGACACAUCAUCAUCAAGCCUGUGGGCCUUGGUGAC | | | |
| | | LEYPTIGOL | GCCCUGGAACUGCUGGUAUCACCAGAGUGCAACUGCGAC | | | |
| | | IDKLVQNNV | UGUCAGAAGGAAGUGGAGGUUAACUCUUCUAAGUGCCAC | | | |
| | | LLIFAVTQE | CACGGCAACGGAAGUUUCCAGUGCGGCGUCUGUGCGUGC | | | |
| | | QVHLYENYA | CACCCUGGCCACAUGGGCCCUCGAUGCGAGUGCGGAGAG | | | |
| | | KLIPGATVG | GACAUGCUCUCUACUGACAGCUGUAAGGAGGCCCCUGAC | | | |
| | | LLQKDSGNI | CAUCCGAGUUGUAGCGGCAGAGGCGACUGCUACUGCGGC | | | |
| | | LQLIISAYE | CAGUGCAUCUGCCAUCUAUCUCCAUACGGAAAUAUCUAC | | | |
| | | ELRSEVELE | GGCCCUUACUGCCAGUGCGACAAUUUCAGCUGCGUUAGA | | | |
| | | VLGDTEGLN | CACAAGGGUCUUCUGUGUGGAGGAAACGGCGAUUGUGAU | | | |
| | | LSFTAICNN | UGCGGUGAGUGCGUGUGCAGAAGCGGCUGGACCGGCGAG | | | |
| | | GTLFQHQKK | UACUGCAAUUGCACCACCUCCACUGACAGCUGCGUGAGC | | | |
| | | CSHMKVGDT | GAGGACGGCGUGCUGUGCAGCGGACGGGGCGACUGUGUU | | | |
| | | ASFSVTVNI | UGCGGCAAGUGUGUGUGCACCAAUCCUGGUGCUAGCGGC | | | |
| | | PHCERRSRH | CCUACUUGCGAAAGGUGCCCAACCUGUGGCGACCCGUGC | | | |
| | | IIIKPVGLG | AACUCCAAGAGAAGCUGCAUCGAGUGUCACCUAUCAGCU | | | |
| | | DALELLVSP | GCCGGCCAGGCCAGAGAGGAGUGUGUUGACAAGUGCAAG | | | |
| | | ECNCDCQKE | UUAGCUGGAGCAACAAUCAGCGAGGAGGAGGACUUCAGC | | | |
| | | VEVNSSKCH | AAGGACGGCAGCGUGAGCUGUAGUCUCCAAGGCGAGAAC | | | |
| | | HGNGSFQCG | GAGUGCCUGAUCACCUUCCUGAUUACUACCGACAACGAG | | | |
| | | VCACHPGHM | GGCAAGACCAUCAUCCACUCCAUCAACGAGAAGGACUGU | | | |
| | | GPRCECGED | CCUAAGCCUCCUAAUAUCCCUAUGAUCAUGCUUGGCGUG | | | |
| | | MLSTDSCKE | AGCCUGGCCAUUCUUCUGAUUGGAGUGGUGCUGCUCUGU | | | |
| | | APDHPSCSG | AUUUGGAAGCUACUGGUGAGCUUCCACGACAGAAAGGAA | | | |
| | | RGDCYCGQC | GUUGCCAAGUUCGAGGCGGAAAGGAGCAAGGCCAAGUGG | | | |
| | | ICHLSPYGN | CAGACCGGCACUAACCCUCUGUACAGAGGAAGUACCAGU | | | |
| | | IYGPYCQCD | ACAUUCAAGAACGUGACCUACAAGCACAGAGAGAAGCAG | | | |
| | | NFSCVRHKG | AAGGUGGAUCUUUCUACAGACUGCGGUAAGCCUAUCCCU | | | |
| | | LLCGGNGDC | AAUCCACUGUUGGGUUUGGAUUCCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 1 | 3 | 50 | 110 | 161 |
| Human | ITB6 | MGIELLCLF | AUGGGCAUCGAGCUGCUGUGCCUGUUCUUCCUGUUCCUGG | GGAAAUCG | UGAUAAUA | SEQ ID |
| Var- | G5 | FLFLGRNDH | GCAGAAACGACCACGUGCAGGGAGGUUGCGCCCUCGGAG | CAAAAUUU | GGCUGGAG | NO: 161 |
| iant | Cap C1 | VQGGCALGG | GUGCCGAGACAUGCGAGGACUGCCUGCUGAUCGGCCCUC | GCUCUUCG | CCUCGGUG | consists |
| 1.2 | Poly A | AETCEDCLL | AGUGCGCCUGGUGCGCCCAGGAGAACUUCACCCACCCUA | CGUUAGAU | GCCUAGCU | from 5′ |
| | tail: | IGPQCAWCA | GCGGCGUGGGCGAGAGGUGCGACACCCCUGCCAACCUGC | UUCUUUUA | UCUUGCCC | to 3′ |
| | 100 nt | QENFTHPSG | UGGCCAAGGGCUGCCAGCUGAACUUCAUCGAGAACCCUG | GUUUUCUC | CUUGGGCC | end: 5′ |
| | (SEQ ID | VGERCDTPA | UGAGCCAGGUGGAGAUCCUGAAGAACAAGCCUCUGAGCG | GCAACUAG | UCCCCCCA | UTR of |
| | NO: 502) | NLLAKGCQL | UGGGCAGACAGAAGAACAGCAGCGACAUCGUGCAGAUCG | CAAGCUUU | GCCCCUCC | SEQ ID |
| | | NFIENPVSQ | CCCCUCAGAGCCUGAUUUUGAAGCUCCGGCCUGGAGGAG | UUGUUCUC | UCCCCUUC | NO: 50, |
| | | VEILKNKPL | CACAGACCCUGCAGGUGCACGUGAGACAGACCGAGGACU | GCC | CUGCACCC | ORF |
| | | SVGRQKNSS | ACCCUGUGGACCUGUACUACCUGAUGGACCUGAGCGCCA | | GUACCCCC | sequence |
| | | DIVQIAPQS | GCAUGGACGACGACCUGAACACCAUCAAGGAGCUGGGCA | | CAAACACC | of SEQ |
| | | LILKLRPGG | GCAGACUGAGCAAGGAGAUGAGCAAGCUGACCAGCAACU | | AUUGUCAC | ID NO: |
| | | AQTLQVHVR | UCAGACUGGGCUUCGGCAGCUUCGUGGAGAAGCCUGUAU | | ACUCCAGU | 3, and |
| | | QTEDYPVDL | CGCCUUUCGUGAAGACCACCCCUGAGGAGAUCGCCAACC | | GGUCUUUG | 3′ UTR |
| | | YYLMDLSAS | CUUGCAGCAGCAUCCCUUACUUCUGCCUGCCUACCUUCG | | AAUAAAGU | sequence |
| | | MDDDLNTIK | GCUUCAAGCACAUCCUGCCUCUGACCAACGACGCCGAGA | | CUGAGUGG | of SEQ |
| | | ELGSRLSKE | GAUUCAACGAGAUAGUUAAGAACCAGAAGAUCAGCGCCA | | GCGGC | ID NO: |
| | | MSKLTSNFR | ACAUAGAUACUCCAGAAGGAGGUUUCGACGCCAUCAUGC | | | 110. |
| | | LGFGSFVEK | AGGCCGCCGUGUGUAAGGAGAAGAUCGGCUGGCGGAACG | | | |
| | | PVSPFVKTT | ACAGCCUGCAUCUUUUGGUGUUCGUGAGUGACGCAGACA | | | |
| | | PEEIANPCS | GCCACUUCGGCAUGGACUCUAAGUUAGCCGGCAUCGUGA | | | |
| | | SIPYFCLPT | UCCCUAACGACGGCCUGUGCCACCUGGACAGCAAGAACG | | | |
| | | FGFKHILPL | AGUACAGCAUGAGCACCGUGCUGGAGUACCCUACCAUCG | | | |
| | | TNDAERFNE | GUCAGCUGAUCGACAAGCUGGUGCAGAACAACGUGCUGC | | | |
| | | IVKNQKISA | UAAUCUUCGCCGUGACCCAGGAACAAGUCCACCUGUACG | | | |
| | | NIDTPEGGF | AGAACUACGCCAAGCUGAUCCCUGGCGCCACCGUGGGCC | | | |
| | | DAIMQAAVC | UGCUGCAGAAGGACAGCGGCAAUAUCUUGCAGUUGAUUA | | | |
| | | KEKIGWRND | UUUCCGCAUACGAGGAGCUGAGAAGCGAGGUGGAGCUGG | | | |
| | | SLHLLVFVS | AGGUGCUGGGCGACACCGAGGGCCUGAACCUGAGCUUCA | | | |
| | | DADSHFGMD | CCGCCAUCUGCAACAACGGCACCCUGUUCCAGCAUCAGA | | | |
| | | SKLAGIVIP | AGAAGUGCAGCCACAUGAAGGUGGGCGAUACAGCCAGCU | | | |
| | | NDGLCHLDS | UCAGCGUGACCGUGAACAUCCCUCACUGCGAGAGAAGAA | | | |
| | | KNEYSMSTV | GCAGACACAUCAUCAUCAAGCCUGUGGGCCUUGGUGACG | | | |
| | | LEYPTIGQL | CCCUGGAACUGCUGGUAUCACCAGAGUGCAACUGCGACU | | | |
| | | IDKLVQNNV | GUCAGAAGGAAGUGGAGGUUAACUCUUCUAAGUGCCACC | | | |
| | | LLIFAVTQE | ACGGCAACGGAAGUUUCCAGUGCGGCGUCUGUGCGUGCC | | | |
| | | QVHLYENYA | ACCCUGGCCACAUGGGCCCUCGAUGCGAGUGCGGAGAGG | | | |
| | | KLIPGATVG | ACAUGCUCUCUACUGACAGCUGUAAGGAGGCCCCUGACC | | | |
| | | LLQKDSGNI | AUCCGAGUUGUAGCGGCAGAGGCGACUGCUACUGCGGCC | | | |
| | | LQLIISAYE | AGUGCAUCUGCCAUCUAUCUCCAUACGGAAAUAUCUACG | | | |
| | | ELRSEVELE | GCCCUUACUGCCAGUGCGACAAUUUCAGCUGCGUUAGAC | | | |
| | | VLGDTEGLN | ACAAGGGUCUUCUGUGUGGAGGAAACGGCGAUUGUGAUU | | | |
| | | LSFTAICNN | GCGGUGAGUGCGUGUGCAGAAGCGGCUGGACCGGCGAGU | | | |
| | | GTLFQHQKK | ACUGCAAUUGCACCACCUCCACUGACAGCUGCGUGAGCG | | | |
| | | CSHMKVGDT | AGGACGGCGUGCUGUGCAGCGGACGGGGCGACUGUGUUU | | | |
| | | ASFSVTVNI | GCGGCAAGUGUGUGUGCACCAAUCCUGGUGCUAGCGGCC | | | |
| | | PHCERRSRH | CUACUUGCGAAAGGUGCCCAACCUGUGGCGACCCGUGCA | | | |
| | | IIIKPVGLG | ACUCCAAGAGAAGCUGCAUCGAGUGUCACCUAUCAGCUG | | | |
| | | DALELLVSP | CCGGCCAGGCCAGAGAGGAGUGUGUUGACAAGUGCAAGU | | | |
| | | ECNCDCQKE | UAGCUGGAGCAACAAUCAGCGAGGAGGAGGACUUCAGCA | | | |
| | | VEVNSSKCH | AGGACGGCAGCGUGAGCUGUAGUCUCCAAGGCGAGAACG | | | |
| | | HGNGSFQCG | AGUGCCUGAUCACCUUCCUGAUUACUACCGACAACGAGG | | | |
| | | VCACHPGHM | GCAAGACCAUCAUCCACUCCAUCAACGAGAAGGACUGUC | | | |
| | | GPRCECGED | CUAAGCCUCCUAAUAUCCCUAUGAUCAUGCUUGGCGUGA | | | |
| | | MLSTDSCKE | GCCUGGCCAUUCUUCUGAUUGGAGUGGUGCUGCUCUGUA | | | |
| | | APDHPSCSG | UUUGGAAGCUACUGGUGAGCUUCCACGACAGAAAGGAAG | | | |
| | | RGDCYCGQC | UUGCCAAGUUCGAGGCGGAAAGGAGCAAGGCCAAGUGGC | | | |
| | | ICHLSPYGN | AGACCGGCACUAACCCUCUGUACAGAGGAAGUACCAGUA | | | |
| | | IYGPYCQCD | CAUUCAAGAACGUGACCUACAAGCACAGAGAGAAGCAGA | | | |
| | | NFSCVRHKG | AGGUGGAUCUUUCUACAGACUGCGGUAAGCCUAUCCCUA | | | |
| | | LLCGGNGDC | AUCCACUGUUGGGUUUGGAUUCCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 1 | 4 | 50 | 143 | 162 |
| Human | ITB6 | MGIELLCLF | AUGGGCAUCGAGCUGCUGUGCCUGUUCUUCCUGUUCCUGG | GGAAAUCG | UAAGCCCC | SEQ ID |
| Var- | G5 | FLFLGRNDH | GCAGAAACGACCACGUGCAGGGAGGUUGCGCCCUCGGAG | CAAAAUUU | UCCGGGGG | NO: 162 |
| iant | Cap C1 | VQGGCALGG | GUGCCGAGACAUGCGAGGACUGCCUGCUGAUCGGCCCUC | GCUCUUCG | CCUCGGUG | consists |
| 1.3 | Poly A | AETCEDCLL | AGUGCGCCUGGUGCGCCCAGGAGAACUUCACCCACCCUA | CGUUAGAU | GCCUAGCU | from 5′ |
| | Tail: | IGPQCAWCA | GCGGCGUGGGCGAGAGGUGCGACACCCCUGCCAACCUGC | UUCUUUUA | UCUUGCCC | to 3′ |
| | 100 nt | QENFTHPSG | UGGCCAAGGGCUGCCAGCUGAACUUCAUCGAGAACCCUG | GUUUUCUC | CUUGGGCC | end: 5′ |
| | (SEQ ID | VGERCDTPA | UGAGCCAGGUGGAGAUCCUGAAGAACAAGCCUCUGAGCG | GCAACUAG | UCCCCCCA | UTR of |
| | NO: 502) | NLLAKGCQL | UGGGCAGACAGAAGAACAGCAGCGACAUCGUGCAGAUCG | CAAGCUUU | GCCCCUCC | SEQ ID |
| | | NFIENPVSQ | CCCCUCAGAGCCUGAUUUUGAAGCUCCGGCCUGGAGGAG | UUGUUCUC | UCCCCUUC | NO: 50, |
| | | VEILKNKPL | CACAGACCCUGCAGGUGCACGUGAGACAGACCGAGGACU | GCC | CUGCACCC | ORF |
| | | SVGRQKNSS | ACCCUGUGGACCUGUACUACCUGAUGGACCUGAGCGCCA | | GUACCCCC | sequence |
| | | DIVQIAPQS | GCAUGGACGACGACCUGAACACCAUCAAGGAGCUGGGCA | | CAAACACC | of SEQ |
| | | LILKLRPGG | GCAGACUGAGCAAGGAGAUGAGCAAGCUGACCAGCAACU | | AUUGUCAC | ID NO: |
| | | AQTLQVHVR | UCAGACUGGGCUUCGGCAGCUUCGUGGAGAAGCCUGUAU | | ACUCCAGU | 4, and |
| | | QTEDYPVDL | CGCCUUUCGUGAAGACCACCCCUGAGGAGAUCGCCAACC | | GGUCUUUG | 3′ UTR |
| | | YYLMDLSAS | CUUGCAGCAGCAUCCCUUACUUCUGCCUGCCUACCUUCG | | AAUAAAGU | sequence |
| | | MDDDLNTIK | GCUUCAAGCACAUCCUGCCUCUGACCAACGACGCCGAGA | | CUGAGUGG | of SEQ |
| | | ELGSRLSKE | GAUUCAACGAGAUAGUUAAGAACCAGAAGAUCAGCGCCA | | GCGGC | ID NO: |
| | | MSKLTSNFR | ACAUAGAUACUCCAGAAGGAGGUUUCGACGCCAUCAUGC | | | 143. |
| | | LGFGSFVEK | AGGCCGCCGUGUGUAAGGAGAAGAUCGGCUGGCGGAACG | | | |
| | | PVSPFVKTT | ACAGCCUGCAUCUUUUGGUGUUCGUGAGUGACGCAGACA | | | |
| | | PEEIANPCS | GCCACUUCGGCAUGGACUCUAAGUUAGCCGGCAUCGUGA | | | |
| | | SIPYFCLPT | UCCCUAACGACGGCCUGUGCCACCUGGACAGCAAGAACG | | | |
| | | FGFKHILPL | AGUACAGCAUGAGCACCGUGCUGGAGUACCCUACCAUCG | | | |
| | | TNDAERFNE | GUCAGCUGAUCGACAAGCUGGUGCAGAACAACGUGCUGC | | | |
| | | IVKNQKISA | UAAUCUUCGCCGUGACCCAGGAACAAGUCCACCUGUACG | | | |
| | | NIDTPEGGF | AGAACUACGCCAAGCUGAUCCCUGGCGCCACCGUGGGCC | | | |
| | | DAIMQAAVC | UGCUGCAGAAGGACAGCGGCAAUAUCUUGCAGUUGAUUA | | | |
| | | KEKIGWRND | UUUCCGCAUACGAGGAGCUGAGAAGCGAGGUGGAGCUGG | | | |
| | | SLHLLVFVS | AGGUGCUGGGCGACACCGAGGGCCUGAACCUGAGCUUCA | | | |
| | | DADSHFGMD | CCGCCAUCUGCAACAACGGCACCCUGUUCCAGCAUCAGA | | | |
| | | SKLAGIVIP | AGAAGUGCAGCCACAUGAAGGUGGGCGAUACAGCCAGCU | | | |
| | | NDGLCHLDS | UCAGCGUGACCGUGAACAUCCCUCACUGCGAGAGAAGAA | | | |
| | | KNEYSMSTV | GCAGACACAUCAUCAUCAAGCCUGUGGGCCUUGGUGACG | | | |
| | | LEYPTIGQL | CCCUGGAACUGCUGGUAUCACCAGAGUGCAACUGCGACU | | | |
| | | IDKLVQNNV | GUCAGAAGGAAGUGGAGGUUAACUCUUCUAAGUGCCACC | | | |
| | | LLIFAVTQE | ACGGCAACGGAAGUUUCCAGUGCGGCGUCUGUGCGUGCC | | | |
| | | QVHLYENYA | ACCCUGGCCACAUGGGCCCUCGAUGCGAGUGCGGAGAGG | | | |
| | | KLIPGATVG | ACAUGCUCUCUACUGACAGCUGUAAGGAGGCCCCUGACC | | | |
| | | LLQKDSGNI | AUCCGAGUUGUAGCGGCAGAGGCGACUGCUACUGCGGCC | | | |
| | | LQLIISAYE | AGUGCAUCUGCCAUCUAUCUCCAUACGGAAAUAUCUACG | | | |
| | | ELRSEVELE | GCCCUUACUGCCAGUGCGACAAUUUCAGCUGCGUUAGAC | | | |
| | | VLGDTEGLN | ACAAGGGUCUUCUGUGUGGAGGAAACGGCGAUUGUGAUU | | | |
| | | LSFTAICNN | GCGGUGAGUGCGUGUGCAGAAGCGGCUGGACCGGCGAGU | | | |
| | | GTLFQHQKK | ACUGCAAUUGCACCACCUCCACUGACAGCUGCGUGAGCG | | | |
| | | CSHMKVGDT | AGGACGGCGUGCUGUGCAGCGGACGGGGCGACUGUGUUU | | | |
| | | ASFSVTVNI | GCGGCAAGUGUGUGUGCACCAAUCCUGGUGCUAGCGGCC | | | |
| | | PHCERRSRH | CUACUUGCGAAAGGUGCCCAACCUGUGGCGACCCGUGCA | | | |
| | | IIIKPVGLG | ACUCCAAGAGAAGCUGCAUCGAGUGUCACCUAUCAGCUG | | | |
| | | DALELLVSP | CCGGCCAGGCCAGAGAGGAGUGUGUUGACAAGUGCAAGU | | | |
| | | ECNCDCQKE | UAGCUGGAGCAACAAUCAGCGAGGAGGAGGACUUCAGCA | | | |
| | | VEVNSSKCH | AGGACGGCAGCGUGAGCUGUAGUCUCCAAGGCGAGAACG | | | |
| | | HGNGSFQCG | AGUGCCUGAUCACCUUCCUGAUUACUACCGACAACGAGG | | | |
| | | VCACHPGHM | GCAAGACCAUCAUCCACUCCAUCAACGAGAAGGACUGUC | | | |
| | | GPRCECGED | CUAAGCCUCCUAAUAUCCCUAUGAUCAUGCUUGGCGUGA | | | |
| | | MLSTDSCKE | GCCUGGCCAUUCUUCUGAUUGGAGUGGUGCUGCUCUGUA | | | |
| | | APDHPSCSG | UUUGGAAGCUACUGGUGAGCUUCCACGACAGAAAGGAAG | | | |
| | | RGDCYCGQC | UUGCCAAGUUCGAGGCGGAAAGGAGCAAGGCCAAGUGGC | | | |
| | | ICHLSPYGN | AGACCGGCACUAACCCUCUGUACAGAGGAAGUACCAGUA | | | |
| | | IYGPYCQCD | CAUUCAAGAACGUGACCUACAAGCACAGAGAGAAGCAGA | | | |
| | | NFSCVRHKG | AGGUGGAUCUUUCUACAGACUGCGGUAAGCCUAUCCCUA | | | |
| | | LLCGGNGDC | AUCCACUGUUGGGUUUGGAUUCCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 1 | 5 | 50 | 110 | 163 |
| Human | ITB6 | MGIELLCLF | AUGGGGAUCGAGCUGCUGUGCCUGUUCUUCCUGUUCCUGG | GGAAAUCG | UGAUAAUA | SEQ ID |
| Var- | G5 | FLFLGRNDH | GACGGAACGACCACGUGCAGGGUGGCUGCGCCCUUGGUG | CAAAAUUU | GGCUGGAG | NO: 163 |
| iant | Cap C1 | VQGGCALGG | GGGCAGAGACCUGCGAGGACUGCCUGCUCAUCGGCCCAC | GCUCUUCG | CCUCGGUG | consists |
| 1.4 | Poly A | AETCEDCLL | AGUGCGCCUGGUGCGCUCAGGAGAACUUCACCCACCCCA | CGUUAGAU | GCCUAGCU | from 5′ |
| | Tail: | IGPQCAWCA | GCGGAGUGGGCGAGAGGUGCGACACCCCGGCCAACCUGC | UUCUUUUA | UCUUGCCC | to 3′ |
| | 100 nt | QENFTHPSG | UGGCCAAGGGGUGCCAGCUGAACUUCAUCGAGAACCCCG | GUUUUCUC | CUUGGGCC | end: 5′ |
| | (SEQ ID | VGERCDTPA | UCUCUCAGGUGGAGAUCCUGAAGAACAAGCCCCUCAGCG | GCAACUAG | UCCCCCCA | UTR of |
| | NO: 502) | NLLAKGCQL | UUGGGCGGCAGAAGAACAGCUCUGACAUCGUGCAGAUCG | CAAGCUUU | GCCCCUCC | SEQ ID |
| | | NFIENPVSQ | CGCCCCAGAGCCUGAUCCUGAAACUACGGCCUGGCGGUG | UUGUUCUC | UCCCCUUC | NO: 50, |
| | | VEILKNKPL | CGCAAACCCUGCAGGUCCACGUGCGGCAGACCGAGGACU | GCC | CUGCACCC | ORF |
| | | SVGRQKNSS | ACCCUGUGGACCUGUACUACCUGAUGGACCUCUCCGCCA | | GUACCCCC | sequence |
| | | DIVQIAPQS | GCAUGGACGACGAUCUGAACACCAUCAAGGAGCUGGGCA | | CAAACACC | of SEQ |
| | | LILKLRPGG | GCCGCCUGAGCAAGGAGAUGUCCAAGCUGACCUCCAACU | | AUUGUCAC | ID NO: |
| | | AQTLQVHVR | UCCGCCUGGGGUUCGGAUCCUUCGUGGAGAAGCCCGUGA | | ACUCCAGU | 5, and |
| | | QTEDYPVDL | GCCCCUUCGUGAAGACCACCCCUGAGGAGAUCGCCAACC | | GGUCUUUG | 3′ UTR |
| | | YYLMDLSAS | CCUGCAGCAGCAUCCCUUACUUCUGCCUGCCCACCUUCG | | AAUAAAGU | sequence |
| | | MDDDLNTIK | GCUUCAAGCACAUCCUGCCACUGACCAACGACGCCGAGC | | CUGAGUGG | of SEQ |
| | | ELGSRLSKE | GGUUCAACGAGAUCGUGAAGAACCAGAAGAUCAGCGCCA | | GCGGC | ID NO: |
| | | MSKLTSNFR | ACAUCGACACCCCGGAGGGUGGGUUCGACGCUAUCAUGC | | | 110. |
| | | LGFGSFVEK | AGGCCGCCGUGUGCAAGGAGAAGAUCGGCUGGCGCAACG | | | |
| | | PVSPFVKTT | ACAGCCUGCACCUGCUGGUGUUCGUGUCCGACGCCGACU | | | |
| | | PEEIANPCS | CCCACUUCGGCAUGGACAGCAAGCUGGCCGGUAUCGUGA | | | |
| | | SIPYFCLPT | UCCCUAACGACGGCCUGUGCCACCUGGACAGCAAGAACG | | | |
| | | FGFKHILPL | AGUACAGCAUGAGCACCGUGCUGGAGUACCCCACCAUCG | | | |
| | | TNDAERFNE | GCCAGCUGAUCGACAAGCUGGUGCAGAACAACGUGCUGC | | | |
| | | IVKNQKISA | UGAUCUUCGCCGUGACCCAGGAGCAGGUGCACCUCUACG | | | |
| | | NIDTPEGGF | AGAACUACGCCAAGCUGAUUCCAGGCGCCACCGUCGGUC | | | |
| | | DAIMQAAVC | UGCUGCAGAAGGACAGCGGCAACAUCCUGCAGCUGAUCA | | | |
| | | KEKIGWRND | UCUCCGCCUACGAGGAGCUGAGGAGCGAGGUGGAGCUGG | | | |
| | | SLHLLVFVS | AGGUGCUCGGGGACACCGAGGGCCUGAACCUGUCCUUCA | | | |
| | | DADSHFGMD | CCGCCAUCUGCAACAACGGGACCCUCUUCCAGCACCAGA | | | |
| | | SKLAGIVIP | AGAAGUGCAGCCACAUGAAGGUGGGCGACACCGCCAGCU | | | |
| | | NDGLCHLDS | UCAGCGUCACCGUGAACAUACCUCACUGUGAGCGGCGGA | | | |
| | | KNEYSMSTV | GCAGGCACAUCAUCAUCAAGCCCGUGGGCCUGGGAGACG | | | |
| | | LEYPTIGQL | CCCUGGAGCUGCUGGUGUCUCCCGAGUGCAACUGCGACU | | | |
| | | IDKLVQNNV | GCCAGAAGGAGGUGGAGGUGAACUCUAGCAAGUGCCACC | | | |
| | | LLIFAVTQE | ACGGCAACGGGAGUUUCCAGUGUGGCGUGUGUGCCUGCC | | | |
| | | QVHLYENYA | ACCCCGGUCACAUGGGACCCCGGUGUGAGUGCGGGGAGG | | | |
| | | KLIPGATVG | ACAUGCUGAGCACCGACAGCUGCAAGGAGGCCCCAGACC | | | |
| | | LLQKDSGNI | ACCCGAGCUGUUCCGGUCGGGGCGACUGCUACUGCGGCC | | | |
| | | LQLIISAYE | AGUGCAUCUGUCACCUGAGCCCCUACGGCAACAUCUACG | | | |
| | | ELRSEVELE | GGCCCUACUGCCAGUGCGACAACUUCUCCUGCGUGCGCC | | | |
| | | VLGDTEGLN | ACAAAGGCCUGCUGUGUGGCGGCAACGGCGACUGCGACU | | | |
| | | LSFTAICNN | GCGGGGAGUGCGUGUGCCGCAGUGGGUGGACCGGCGAGU | | | |
| | | GTLFQHQKK | ACUGCAACUGCACCACCUCCACCGACUCUUGCGUGUCCG | | | |
| | | CSHMKVGDT | AGGACGGGGUGCUGUGUUCCGGGCGCGGAGAUUGCGUGU | | | |
| | | ASFSVTVNI | GCGGGAAGUGCGUGUGCACCAACCCUGGCGCAUCGGGUC | | | |
| | | PHCERRSRH | CUACCUGCGAGAGGUGCCCCACCUGUGGCGACCCCUGCA | | | |
| | | IIIKPVGLG | ACAGCAAGCGCUCUUGCAUCGAGUGCCACCUCUCAGCAG | | | |
| | | DALELLVSP | CAGGGCAGGCUCGGGAGGAGUGCGUGGACAAGUGCAAGC | | | |
| | | ECNCDCQKE | UCGCCGGCGCCACCAUCAGUGAGGAGGAGGACUUCUCCA | | | |
| | | VEVNSSKCH | AGGACGGCAGCGUGUCCUGCUCCCUCCAGGGCGAGAACG | | | |
| | | HGNGSFQCG | AGUGCCUCAUCACCUUCCUGAUCACCACCGACAACGAGG | | | |
| | | VCACHPGHM | GCAAGACCAUCAUCCACUCCAUCAACGAGAAGGACUGCC | | | |
| | | GPRCECGED | CCAAGCCGCCUAACAUCCCCAUGAUCAUGCUGGGCGUGA | | | |
| | | MLSTDSCKE | GCCUGGCCAUCCUGCUGAUCGGCGUGGUGCUGCUGUGCA | | | |
| | | APDHPSCSG | UCUGGAAGCUGCUGGUCUCCUUCCACGACCGCAAGGAGG | | | |
| | | RGDCYCGQC | UGGCCAAGUUCGAGGCCGAGCGCUCUAAGGCCAAGUGGC | | | |
| | | ICHLSPYGN | AGACCGGCACCAACCCUCUGUACCGGGGCUCCACCAGCA | | | |
| | | IYGPYCQCD | CCUUCAAGAACGUGACCUACAAGCACCGCGAGAAGCAGA | | | |
| | | NFSCVRHKG | AGGUGGACCUGUCCACCGACUGUGGCAAGCCCAUCCCCA | | | |
| | | LLCGGNGDC | AUCCCCUGCUGGGGCUGGACUCCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 1 | 5 | 50 | 144 | 164 |
| Human | ITB6 | MGIELLCLF | AUGGGGAUCGAGCUGCUGUGCCUGUUCUUCCUGUUCCUGG | GGAAAUCG | UAAGCCCC | SEQ ID |
| Var- | G5 | FLFLGRNDH | GACGGAACGACCACGUGCAGGGUGGCUGCGCCCUUGGUG | CAAAAUUU | UCCGGGGG | NO: 164 |
| iant | Cap C1 | VQGGCALGG | GGGCAGAGACCUGCGAGGACUGCCUGCUCAUCGGCCCAC | GCUCUUCG | CCUCCUGA | consists |
| 1.5 | Poly A | AETCEDCLL | AGUGCGCCUGGUGCGCUCAGGAGAACUUCACCCACCCCA | CGUUAGAU | GAGACCUG | from 5′ |
| | Tail: | IGPQCAWCA | GCGGAGUGGGCGAGAGGUGCGACACCCCGGCCAACCUGC | UUCUUUUA | UGUGAACU | to 3′ |
| | 100 nt | QENFTHPSG | UGGCCAAGGGGUGCCAGCUGAACUUCAUCGAGAACCCCG | GUUUUCUC | AUUGAGAA | end: 5′ |
| | (SEQ ID | VGERCDTPA | UCUCUCAGGUGGAGAUCCUGAAGAACAAGCCCCUCAGCG | GCAACUAG | GAUCGGAA | UTR of |
| | NO: 502) | NLLAKGCQL | UUGGGCGGCAGAAGAACAGCUCUGACAUCGUGCAGAUCG | CAAGCUUU | CAGCUCCU | SEQ ID |
| | | NFIENPVSQ | CGCCCCAGAGCCUGAUCCUGAAACUACGGCCUGGCGGUG | UUGUUCUC | UACUCUGA | NO: 50, |
| | | VEILKNKPL | CGCAAACCCUGCAGGUCCACGUGCGGCAGACCGAGGACU | GCC | GGAAGUUG | ORF |
| | | SVGRQKNSS | ACCCUGUGGACCUGUACUACCUGAUGGACCUCUCCGCCA | | GUACCCCC | sequence |
| | | DIVQIAPQS | GCAUGGACGACGAUCUGAACACCAUCAAGGAGCUGGGCA | | CAAACACC | of SEQ |
| | | LILKLRPGG | GCCGCCUGAGCAAGGAGAUGUCCAAGCUGACCUCCAACU | | AUUGUCAC | ID NO: |
| | | AQTLQVHVR | UCCGCCUGGGGUUCGGAUCCUUCGUGGAGAAGCCCGUGA | | ACUCCAGU | 5, and |
| | | QTEDYPVDL | GCCCCUUCGUGAAGACCACCCCUGAGGAGAUCGCCAACC | | GGUCUUUG | 3′ UTR |
| | | YYLMDLSAS | CCUGCAGCAGCAUCCCUUACUUCUGCCUGCCCACCUUCG | | AAUAAAGU | sequence |
| | | MDDDLNTIK | GCUUCAAGCACAUCCUGCCACUGACCAACGACGCCGAGC | | CUGAGUGG | of SEQ |
| | | ELGSRLSKE | GGUUCAACGAGAUCGUGAAGAACCAGAAGAUCAGCGCCA | | GCGGC | ID NO: |
| | | MSKLTSNFR | ACAUCGACACCCCGGAGGGUGGGUUCGACGCUAUCAUGC | | | 144. |
| | | LGFGSFVEK | AGGCCGCCGUGUGCAAGGAGAAGAUCGGCUGGCGCAACG | | | |
| | | PVSPFVKTT | ACAGCCUGCACCUGCUGGUGUUCGUGUCCGACGCCGACU | | | |
| | | PEEIANPCS | CCCACUUCGGCAUGGACAGCAAGCUGGCCGGUAUCGUGA | | | |
| | | SIPYFCLPT | UCCCUAACGACGGCCUGUGCCACCUGGACAGCAAGAACG | | | |
| | | FGFKHILPL | AGUACAGCAUGAGCACCGUGCUGGAGUACCCCACCAUCG | | | |
| | | TNDAERFNE | GCCAGCUGAUCGACAAGCUGGUGCAGAACAACGUGCUGC | | | |
| | | IVKNQKISA | UGAUCUUCGCCGUGACCCAGGAGCAGGUGCACCUCUACG | | | |
| | | NIDTPEGGF | AGAACUACGCCAAGCUGAUUCCAGGCGCCACCGUCGGUC | | | |
| | | DAIMQAAVC | UGCUGCAGAAGGACAGCGGCAACAUCCUGCAGCUGAUCA | | | |
| | | KEKIGWRND | UCUCCGCCUACGAGGAGCUGAGGAGCGAGGUGGAGCUGG | | | |
| | | SLHLLVFVS | AGGUGCUCGGGGACACCGAGGGCCUGAACCUGUCCUUCA | | | |
| | | DADSHFGMD | CCGCCAUCUGCAACAACGGGACCCUCUUCCAGCACCAGA | | | |
| | | SKLAGIVIP | AGAAGUGCAGCCACAUGAAGGUGGGCGACACCGCCAGCU | | | |
| | | NDGLCHLDS | UCAGCGUCACCGUGAACAUACCUCACUGUGAGCGGCGGA | | | |
| | | KNEYSMSTV | GCAGGCACAUCAUCAUCAAGCCCGUGGGCCUGGGAGACG | | | |
| | | LEYPTIGQL | CCCUGGAGCUGCUGGUGUCUCCCGAGUGCAACUGCGACU | | | |
| | | IDKLVQNNV | GCCAGAAGGAGGUGGAGGUGAACUCUAGCAAGUGCCACC | | | |
| | | LLIFAVTQE | ACGGCAACGGGAGUUUCCAGUGUGGCGUGUGUGCCUGCC | | | |
| | | QVHLYENYA | ACCCCGGUCACAUGGGACCCCGGUGUGAGUGCGGGGAGG | | | |
| | | KLIPGATVG | ACAUGCUGAGCACCGACAGCUGCAAGGAGGCCCCAGACC | | | |
| | | LLQKDSGNI | ACCCGAGCUGUUCCGGUCGGGGCGACUGCUACUGCGGCC | | | |
| | | LQLIISAYE | AGUGCAUCUGUCACCUGAGCCCCUACGGCAACAUCUACG | | | |
| | | ELRSEVELE | GGCCCUACUGCCAGUGCGACAACUUCUCCUGCGUGCGCC | | | |
| | | VLGDTEGLN | ACAAAGGCCUGCUGUGUGGCGGCAACGGCGACUGCGACU | | | |
| | | LSFTAICNN | GCGGGGAGUGCGUGUGCCGCAGUGGGUGGACCGGCGAGU | | | |
| | | GTLFQHQKK | ACUGCAACUGCACCACCUCCACCGACUCUUGCGUGUCCG | | | |
| | | CSHMKVGDT | AGGACGGGGUGCUGUGUUCCGGGCGCGGAGAUUGCGUGU | | | |
| | | ASFSVTVNI | GCGGGAAGUGCGUGUGCACCAACCCUGGCGCAUCGGGUC | | | |
| | | PHCERRSRH | CUACCUGCGAGAGGUGCCCCACCUGUGGCGACCCCUGCA | | | |
| | | IIIKPVGLG | ACAGCAAGCGCUCUUGCAUCGAGUGCCACCUCUCAGCAG | | | |
| | | DALELLVSP | CAGGGCAGGCUCGGGAGGAGUGCGUGGACAAGUGCAAGC | | | |
| | | ECNCDCQKE | UCGCCGGCGCCACCAUCAGUGAGGAGGAGGACUUCUCCA | | | |
| | | VEVNSSKCH | AGGACGGCAGCGUGUCCUGCUCCCUCCAGGGCGAGAACG | | | |
| | | HGNGSFQCG | AGUGCCUCAUCACCUUCCUGAUCACCACCGACAACGAGG | | | |
| | | VCACHPGHM | GCAAGACCAUCAUCCACUCCAUCAACGAGAAGGACUGCC | | | |
| | | GPRCECGED | CCAAGCCGCCUAACAUCCCCAUGAUCAUGCUGGGCGUGA | | | |
| | | MLSTDSCKE | GCCUGGCCAUCCUGCUGAUCGGCGUGGUGCUGCUGUGCA | | | |
| | | APDHPSCSG | UCUGGAAGCUGCUGGUCUCCUUCCACGACCGCAAGGAGG | | | |
| | | RGDCYCGQC | UGGCCAAGUUCGAGGCCGAGCGCUCUAAGGCCAAGUGGC | | | |
| | | ICHLSPYGN | AGACCGGCACCAACCCUCUGUACCGGGGCUCCACCAGCA | | | |
| | | IYGPYCQCD | CCUUCAAGAACGUGACCUACAAGCACCGCGAGAAGCAGA | | | |
| | | NFSCVRHKG | AGGUGGACCUGUCCACCGACUGUGGCAAGCCCAUCCCCA | | | |
| | | LLCGGNGDC | AUCCCCUGCUGGGGCUGGACUCCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 1 | 6 | 50 | 110 | 165 |
| Human | ITB6 | MGIELLCLF | AUGGGUAUCGAGCUGCUGUGUCUGUUCUUUCUGUUUCUGG | GGAAAUCG | UGAUAAUA | SEQ ID |
| Var- | G5 | FLFLGRNDH | GCCGCAACGACCACGUCCAGGGCGGCUGCGCCCUGGGCG | CAAAAUUU | GGCUGGAG | NO: 165 |
| iant | Cap C1 | VQGGCALGG | GGGCCGAGACCUGCGAGGACUGCCUGCUCAUCGGGCCAC | GCUCUUCG | CCUCGGUG | consists |
| 1.6 | Poly A | AETCEDCLL | AGUGUGCCUGGUGUGCACAGGAGAACUUCACCCACCCCA | CGUUAGAU | GCCUAGCU | from 5′ |
| | Tail: | IGPQCAWCA | GCGGGGUGGGCGAGAGGUGCGACACCCCUGCAAAUCUGC | UUCUUUUA | UCUUGCCC | to 3′ |
| | 100 nt | QENFTHPSG | UGGCAAAGGGCUGCCAGCUGAAUUUCAUCGAGAAUCCCG | GUUUUCUC | CUUGGGCC | end: 5′ |
| | (SEQ ID | VGERCDTPA | UCAGCCAGGUGGAGAUUCUCAAGAACAAGCCUCUGAGCG | GCAACUAG | UCCCCCCA | UTR of |
| | NO: 502) | NLLAKGCOL | UCGGACGGCAGAAGAACUCUUCUGACAUCGUCCAGAUCG | CAAGCUUU | GCCCCUCC | SEQ ID |
| | | NFIENPVSQ | CACCUCAGAGCCUGAUUCUGAAGCUCCGCCCAGGCGGAG | UUGUUCUC | UCCCCUUC | NO: 50, |
| | | VEILKNKPL | CUCAGACCCUCCAGGUCCACGUCAGGCAGACUGAGGACU | GCC | CUGCACCC | ORF |
| | | SVGRQKNSS | ACCCCGUGGACCUGUACUACCUGAUGGAUCUGUCCGCCA | | GUACCCCC | sequence |
| | | DIVQIAPQS | GCAUGGACGACGAUCUGAACACCAUUAAGGAGCUGGGCA | | CAAACACC | of SEQ |
| | | LILKLRPGG | GCCGGCUCUCUAAGGAGAUGUCCAAGCUCACCAGCAACU | | AUUGUCAC | ID NO: |
| | | AQTLQVHVR | UCAGGCUGGGAUUCGGCAGCUUUGUCGAGAAGCCUGUGU | | ACUCCAGU | 6, and |
| | | QTEDYPVDL | CUCCUUUCGUGAAGACCACACCAGAGGAGAUCGCAAACC | | GGUCUUUG | 3′ UTR |
| | | YYLMDLSAS | CCUGUAGCAGCAUCCCCUACUUCUGCCUGCCUACAUUCG | | AAUAAAGU | sequence |
| | | MDDDLNTIK | GGUUCAAGCACAUUCUGCCUCUGACCAACGACGCAGAGC | | CUGAGUGG | of SEQ |
| | | ELGSRLSKE | GCUUUAACGAGAUCGUGAAGAAUCAGAAGAUCAGCGCCA | | GCGGC | ID NO: |
| | | MSKLTSNFR | ACAUUGAUACUCCUGAGGGCGGCUUCGACGCCAUCAUGC | | | 110. |
| | | LGFGSFVEK | AGGCUGCCGUCUGCAAGGAGAAGAUCGGGUGGAGGAACG | | | |
| | | PVSPFVKTT | ACAGCCUCCACCUGCUCGUGUUCGUGAGCGACGCCGACA | | | |
| | | PEEIANPCS | GCCACUUUGGGAUGGACUCCAAGCUGGCCGGCAUCGUGA | | | |
| | | SIPYFCLPT | UCCCCAACGACGGGCUGUGUCACCUCGAUUCCAAGAACG | | | |
| | | FGFKHILPL | AGUACAGCAUGAGCACCGUGCUGGAGUACCCCACCAUUG | | | |
| | | TNDAERFNE | GGCAGCUCAUUGAUAAGCUCGUGCAGAACAACGUCCUGC | | | |
| | | IVKNQKISA | UGAUCUUUGCAGUGACACAGGAGCAGGUGCACCUGUACG | | | |
| | | NIDTPEGGF | AGAAUUACGCAAAGCUCAUCCCCGGAGCCACUGUGGGCC | | | |
| | | DAIMQAAVC | UCCUGCAGAAGGACUCCGGGAACAUCCUCCAGCUCAUCA | | | |
| | | KEKIGWRND | UCUCCGCCUACGAGGAGCUGAGGAGCGAGGUGGAGCUCG | | | |
| | | SLHLLVFVS | AGGUCCUCGGGGACACCGAGGGCCUCAACCUGAGCUUUA | | | |
| | | DADSHFGMD | CCGCCAUCUGUAACAACGGAACUCUCUUCCAGCACCAGA | | | |
| | | SKLAGIVIP | AGAAGUGCUCUCACAUGAAGGUGGGCGACACCGCCUCCU | | | |
| | | NDGLCHLDS | UCAGCGUGACCGUGAACAUCCCUCACUGUGAGCGGCGGA | | | |
| | | KNEYSMSTV | GCCGCCACAUCAUCAUCAAGCCCGUGGGGCUCGGGGACG | | | |
| | | LEYPTIGQL | CCCUGGAGCUCCUGGUGUCCCCUGAGUGUAACUGCGACU | | | |
| | | IDKLVQNNV | GCCAGAAGGAGGUCGAGGUGAACAGCUCUAAGUGCCACC | | | |
| | | LLIFAVTQE | ACGGCAACGGGAGCUUCCAGUGCGGGGUGUGCGCAUGCC | | | |
| | | QVHLYENYA | ACCCCGGCCACAUGGGGCCUCGCUGCGAGUGCGGCGAGG | | | |
| | | KLIPGATVG | ACAUGCUGUCUACCGACAGCUGCAAGGAGGCCCCUGACC | | | |
| | | LLQKDSGNI | ACCCUAGCUGCUCUGGCCGCGGAGACUGCUACUGCGGCC | | | |
| | | LQLIISAYE | AGUGCAUCUGCCACCUGUCCCCUUACGGGAACAUUUACG | | | |
| | | ELRSEVELE | GGCCCUACUGCCAGUGUGAUAACUUUUCCUGCGUGCGGC | | | |
| | | VLGDTEGLN | ACAAGGGCCUGCUGUGCGGAGGAAAUGGCGACUGCGACU | | | |
| | | LSFTAICNN | GCGGCGAGUGCGUCUGUCGCAGCGGCUGGACCGGGGAGU | | | |
| | | GTLFQHQKK | ACUGCAACUGCACUACUUCCACUGACAGCUGUGUCAGCG | | | |
| | | CSHMKVGDT | AGGACGGGGUGCUCUGUUCUGGCCGCGGGGACUGCGUCU | | | |
| | | ASFSVTVNI | GCGGCAAGUGCGUGUGCACUAAUCCAGGGGCCUCCGGGC | | | |
| | | PHCERRSRH | CCACCUGCGAGCGGUGUCCCACCUGCGGGGACCCUUGCA | | | |
| | | IIIKPVGLG | ACAGCAAGAGGUCCUGCAUCGAGUGCCACCUGUCCGCCG | | | |
| | | DALELLVSP | CCGGACAGGCACGCGAGGAGUGCGUCGAUAAGUGUAAGC | | | |
| | | ECNCDCQKE | UCGCCGGAGCUACAAUUAGCGAGGAGGAGGACUUCUCCA | | | |
| | | VEVNSSKCH | AGGACGGGAGCGUGAGCUGCUCCCUCCAGGGAGAGAACG | | | |
| | | HGNGSFQCG | AGUGCCUGAUCACUUUCCUCAUCACCACUGAUAACGAGG | | | |
| | | VCACHPGHM | GAAAGACAAUCAUUCACUCUAUCAACGAGAAGGAUUGUC | | | |
| | | GPRCECGED | CCAAGCCCCCAAACAUUCCCAUGAUCAUGCUGGGAGUGU | | | |
| | | MLSTDSCKE | CCCUGGCAAUCCUGCUGAUCGGAGUCGUGCUGCUCUGCA | | | |
| | | APDHPSCSG | UCUGGAAGCUGCUCGUCUCCUUCCACGACCGCAAGGAGG | | | |
| | | RGDCYCGQC | UCGCAAAGUUUGAGGCCGAGCGGUCUAAGGCCAAGUGGC | | | |
| | | ICHLSPYGN | AGACAGGGACCAAUCCUCUCUACCGCGGGUCUACUUCUA | | | |
| | | IYGPYCQCD | CCUUCAAGAACGUGACUUACAAGCACAGGGAGAAGCAGA | | | |
| | | NFSCVRHKG | AGGUGGACCUCUCCACCGAUUGCGGGAAGCCCAUCCCCA | | | |
| | | LLCGGNGDC | AUCCCCUGCUGGGCCUGGACAGCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 1 | 6 | 50 | 143 | 166 |
| Human | ITB6 | MGIELLCLF | AUGGGUAUCGAGCUGCUGUGUCUGUUCUUUCUGUUUCUGG | GGAAAUCG | UAAGCCCC | SEQ ID |
| Var- | G5 | FLFLGRNDH | GCCGCAACGACCACGUCCAGGGCGGCUGCGCCCUGGGCG | CAAAAUUU | UCCGGGGG | NO: 166 |
| iant | Cap II-b | VQGGCALGG | GGGCCGAGACCUGCGAGGACUGCCUGCUCAUCGGGCCAC | GCUCUUCG | CCUCGGUG | consists |
| 1.7 | Poly A | AETCEDCLL | AGUGUGCCUGGUGUGCACAGGAGAACUUCACCCACCCCA | CGUUAGAU | GCCUAGCU | from 5′ |
| | Tail: | IGPQCAWCA | GCGGGGUGGGCGAGAGGUGCGACACCCCUGCAAAUCUGC | UUCUUUUA | UCUUGCCC | 3′ |
| | 100 nt | QENFTHPSG | UGGCAAAGGGCUGCCAGCUGAAUUUCAUCGAGAAUCCCG | GUUUUCUC | CUUGGGCC | end: 5′ |
| | (SEQ ID | VGERCDTPA | UCAGCCAGGUGGAGAUUCUCAAGAACAAGCCUCUGAGCG | GCAACUAG | UCCCCCCA | UTR of |
| | NO: 502) | NLLAKGCQL | UCGGACGGCAGAAGAACUCUUCUGACAUCGUCCAGAUCG | CAAGCUUU | GCCCCUCC | SEQ ID |
| | | NFIENPVSQ | CACCUCAGAGCCUGAUUCUGAAGCUCCGCCCAGGCGGAG | UUGUUCUC | UCCCCUUC | NO: 50, |
| | | VEILKNKPL | CUCAGACCCUCCAGGUCCACGUCAGGCAGACUGAGGACU | GCC | CUGCACCC | ORF |
| | | SVGRQKNSS | ACCCCGUGGACCUGUACUACCUGAUGGAUCUGUCCGCCA | | GUACCCCC | sequence |
| | | DIVQIAPQS | GCAUGGACGACGAUCUGAACACCAUUAAGGAGCUGGGCA | | CAAACACC | of SEQ |
| | | LILKLRPGG | GCCGGCUCUCUAAGGAGAUGUCCAAGCUCACCAGCAACU | | AUUGUCAC | ID NO: |
| | | AQTLQVHVR | UCAGGCUGGGAUUCGGCAGCUUUGUCGAGAAGCCUGUGU | | ACUCCAGU | 6, and |
| | | QTEDYPVDL | CUCCUUUCGUGAAGACCACACCAGAGGAGAUCGCAAACC | | GGUCUUUG | 3′ UTR |
| | | YYLMDLSAS | CCUGUAGCAGCAUCCCCUACUUCUGCCUGCCUACAUUCG | | AAUAAAGU | sequence |
| | | MDDDLNTIK | GGUUCAAGCACAUUCUGCCUCUGACCAACGACGCAGAGC | | CUGAGUGG | of SEQ |
| | | ELGSRLSKE | GCUUUAACGAGAUCGUGAAGAAUCAGAAGAUCAGCGCCA | | GCGGC | ID NO: |
| | | MSKLTSNFR | ACAUUGAUACUCCUGAGGGCGGCUUCGACGCCAUCAUGC | | | 143. |
| | | LGFGSFVEK | AGGCUGCCGUCUGCAAGGAGAAGAUCGGGUGGAGGAACG | | | |
| | | PVSPFVKTT | ACAGCCUCCACCUGCUCGUGUUCGUGAGCGACGCCGACA | | | |
| | | PEEIANPCS | GCCACUUUGGGAUGGACUCCAAGCUGGCCGGCAUCGUGA | | | |
| | | SIPYFCLPT | UCCCCAACGACGGGCUGUGUCACCUCGAUUCCAAGAACG | | | |
| | | FGFKHILPL | AGUACAGCAUGAGCACCGUGCUGGAGUACCCCACCAUUG | | | |
| | | TNDAERFNE | GGCAGCUCAUUGAUAAGCUCGUGCAGAACAACGUCCUGC | | | |
| | | IVKNQKISA | UGAUCUUUGCAGUGACACAGGAGCAGGUGCACCUGUACG | | | |
| | | NIDTPEGGF | AGAAUUACGCAAAGCUCAUCCCCGGAGCCACUGUGGGCC | | | |
| | | DAIMQAAVC | UCCUGCAGAAGGACUCCGGGAACAUCCUCCAGCUCAUCA | | | |
| | | KEKIGWRND | UCUCCGCCUACGAGGAGCUGAGGAGCGAGGUGGAGCUCG | | | |
| | | SLHLLVFVS | AGGUCCUCGGGGACACCGAGGGCCUCAACCUGAGCUUUA | | | |
| | | DADSHFGMD | CCGCCAUCUGUAACAACGGAACUCUCUUCCAGCACCAGA | | | |
| | | SKLAGIVIP | AGAAGUGCUCUCACAUGAAGGUGGGCGACACCGCCUCCU | | | |
| | | NDGLCHLDS | UCAGCGUGACCGUGAACAUCCCUCACUGUGAGCGGCGGA | | | |
| | | KNEYSMSTV | GCCGCCACAUCAUCAUCAAGCCCGUGGGGCUCGGGGACG | | | |
| | | LEYPTIGQL | CCCUGGAGCUCCUGGUGUCCCCUGAGUGUAACUGCGACU | | | |
| | | IDKLVQNNV | GCCAGAAGGAGGUCGAGGUGAACAGCUCUAAGUGCCACC | | | |
| | | LLIFAVTQE | ACGGCAACGGGAGCUUCCAGUGCGGGGUGUGCGCAUGCC | | | |
| | | QVHLYENYA | ACCCCGGCCACAUGGGGCCUCGCUGCGAGUGCGGCGAGG | | | |
| | | KLIPGATVG | ACAUGCUGUCUACCGACAGCUGCAAGGAGGCCCCUGACC | | | |
| | | LLQKDSGNI | ACCCUAGCUGCUCUGGCCGCGGAGACUGCUACUGCGGCC | | | |
| | | LQLIISAYE | AGUGCAUCUGCCACCUGUCCCCUUACGGGAACAUUUACG | | | |
| | | ELRSEVELE | GGCCCUACUGCCAGUGUGAUAACUUUUCCUGCGUGCGGC | | | |
| | | VLGDTEGLN | ACAAGGGCCUGCUGUGCGGAGGAAAUGGCGACUGCGACU | | | |
| | | LSFTAICNN | GCGGCGAGUGCGUCUGUCGCAGCGGCUGGACCGGGGAGU | | | |
| | | GTLFQHQKK | ACUGCAACUGCACUACUUCCACUGACAGCUGUGUCAGCG | | | |
| | | CSHMKVGDT | AGGACGGGGUGCUCUGUUCUGGCCGCGGGGACUGCGUCU | | | |
| | | ASFSVTVNI | GCGGCAAGUGCGUGUGCACUAAUCCAGGGGCCUCCGGGC | | | |
| | | PHCERRSRH | CCACCUGCGAGCGGUGUCCCACCUGCGGGGACCCUUGCA | | | |
| | | IIIKPVGLG | ACAGCAAGAGGUCCUGCAUCGAGUGCCACCUGUCCGCCG | | | |
| | | DALELLVSP | CCGGACAGGCACGCGAGGAGUGCGUCGAUAAGUGUAAGC | | | |
| | | ECNCDCQKE | UCGCCGGAGCUACAAUUAGCGAGGAGGAGGACUUCUCCA | | | |
| | | VEVNSSKCH | AGGACGGGAGCGUGAGCUGCUCCCUCCAGGGAGAGAACG | | | |
| | | HGNGSFQCG | AGUGCCUGAUCACUUUCCUCAUCACCACUGAUAACGAGG | | | |
| | | VCACHPGHM | GAAAGACAAUCAUUCACUCUAUCAACGAGAAGGAUUGUC | | | |
| | | GPRCECGED | CCAAGCCCCCAAACAUUCCCAUGAUCAUGCUGGGAGUGU | | | |
| | | MLSTDSCKE | CCCUGGCAAUCCUGCUGAUCGGAGUCGUGCUGCUCUGCA | | | |
| | | APDHPSCSG | UCUGGAAGCUGCUCGUCUCCUUCCACGACCGCAAGGAGG | | | |
| | | RGDCYCGQC | UCGCAAAGUUUGAGGCCGAGCGGUCUAAGGCCAAGUGGC | | | |
| | | ICHLSPYGN | AGACAGGGACCAAUCCUCUCUACCGCGGGUCUACUUCUA | | | |
| | | IYGPYCQCD | CCUUCAAGAACGUGACUUACAAGCACAGGGAGAAGCAGA | | | |
| | | NFSCVRHKG | AGGUGGACCUCUCCACCGAUUGCGGGAAGCCCAUCCCCA | | | |
| | | LLCGGNGDC | AUCCCCUGCUGGGCCUGGACAGCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 1 | 6 | 50 | 145 | 167 |
| Human | ITB6 | MGIELLCLF | AUGGGUAUCGAGCUGCUGUGUCUGUUCUUUCUGUUUCUGG | GGAAAUCG | UAAAGCUC | SEQ ID |
| Var- | G5 | FLFLGRNDH | GCCGCAACGACCACGUCCAGGGCGGCUGCGCCCUGGGCG | CAAAAUUU | CCCGGGGG | NO: 167 |
| iant | Cap II-b | VQGGCALGG | GGGCCGAGACCUGCGAGGACUGCCUGCUCAUCGGGCCAC | GCUCUUCG | CCUCGGUG | consists |
| 1.8 | Poly A | AETCEDCLL | AGUGUGCCUGGUGUGCACAGGAGAACUUCACCCACCCCA | CGUUAGAU | GCCUAGCU | from 5′ |
| | Tail: | IGPQCAWCA | GCGGGGUGGGCGAGAGGUGCGACACCCCUGCAAAUCUGC | UUCUUUUA | UCUUGCCC | to 3′ |
| | 100 nt | QENFTHPSG | UGGCAAAGGGCUGCCAGCUGAAUUUCAUCGAGAAUCCCG | GUUUUCUC | CUUGGGCC | end: 5′ |
| | (SEQ ID | VGERCDTPA | UCAGCCAGGUGGAGAUUCUCAAGAACAAGCCUCUGAGCG | GCAACUAG | UCCCCCCA | UTR of |
| | NO: 502) | NLLAKGCQL | UCGGACGGCAGAAGAACUCUUCUGACAUCGUCCAGAUCG | CAAGCUUU | GCCCCUCC | SEQ ID |
| | | NFIENPVSQ | CACCUCAGAGCCUGAUUCUGAAGCUCCGCCCAGGCGGAG | UUGUUCUC | UCCCCUUC | NO: 50, |
| | | VEILKNKPL | CUCAGACCCUCCAGGUCCACGUCAGGCAGACUGAGGACU | GCC | CUGCACCC | ORF |
| | | SVGROKNSS | ACCCCGUGGACCUGUACUACCUGAUGGAUCUGUCCGCCA | | GUACCCCC | sequence |
| | | DIVQIAPQS | GCAUGGACGACGAUCUGAACACCAUUAAGGAGCUGGGCA | | CAAACACC | of SEQ |
| | | LILKLRPGG | GCCGGCUCUCUAAGGAGAUGUCCAAGCUCACCAGCAACU | | AUUGUCAC | ID NO: |
| | | AQTLQVHVR | UCAGGCUGGGAUUCGGCAGCUUUGUCGAGAAGCCUGUGU | | ACUCCAGU | 6, and |
| | | QTEDYPVDL | CUCCUUUCGUGAAGACCACACCAGAGGAGAUCGCAAACC | | GGUCUUUG | 3′ UTR |
| | | YYLMDLSAS | CCUGUAGCAGCAUCCCCUACUUCUGCCUGCCUACAUUCG | | AAUAAAGU | sequence |
| | | MDDDLNTIK | GGUUCAAGCACAUUCUGCCUCUGACCAACGACGCAGAGC | | CUGAGUGG | of SEQ |
| | | ELGSRLSKE | GCUUUAACGAGAUCGUGAAGAAUCAGAAGAUCAGCGCCA | | GCGGC | ID NO: |
| | | MSKLTSNFR | ACAUUGAUACUCCUGAGGGGGCUUCGACGCCAUCAUGC | | | 145. |
| | | LGFGSFVEK | AGGCUGCCGUCUGCAAGGAGAAGAUCGGGUGGAGGAACG | | | |
| | | PVSPFVKTT | ACAGCCUCCACCUGCUCGUGUUCGUGAGCGACGCCGACA | | | |
| | | PEEIANPCS | GCCACUUUGGGAUGGACUCCAAGCUGGCCGGCAUCGUGA | | | |
| | | SIPYFCLPT | UCCCCAACGACGGGCUGUGUCACCUCGAUUCCAAGAACG | | | |
| | | FGFKHILPL | AGUACAGCAUGAGCACCGUGCUGGAGUACCCCACCAUUG | | | |
| | | TNDAERFNE | GGCAGCUCAUUGAUAAGCUCGUGCAGAACAACGUCCUGC | | | |
| | | IVKNQKISA | UGAUCUUUGCAGUGACACAGGAGCAGGUGCACCUGUACG | | | |
| | | NIDTPEGGF | AGAAUUACGCAAAGCUCAUCCCCGGAGCCACUGUGGGCC | | | |
| | | DAIMQAAVC | UCCUGCAGAAGGACUCCGGGAACAUCCUCCAGCUCAUCA | | | |
| | | KEKIGWRND | UCUCCGCCUACGAGGAGCUGAGGAGCGAGGUGGAGCUCG | | | |
| | | SLHLLVFVS | AGGUCCUCGGGGACACCGAGGGCCUCAACCUGAGCUUUA | | | |
| | | DADSHFGMD | CCGCCAUCUGUAACAACGGAACUCUCUUCCAGCACCAGA | | | |
| | | SKLAGIVIP | AGAAGUGCUCUCACAUGAAGGUGGGCGACACCGCCUCCU | | | |
| | | NDGLCHLDS | UCAGCGUGACCGUGAACAUCCCUCACUGUGAGCGGCGGA | | | |
| | | KNEYSMSTV | GCCGCCACAUCAUCAUCAAGCCCGUGGGGCUCGGGGACG | | | |
| | | LEYPTIGQL | CCCUGGAGCUCCUGGUGUCCCCUGAGUGUAACUGCGACU | | | |
| | | IDKLVQNNV | GCCAGAAGGAGGUCGAGGUGAACAGCUCUAAGUGCCACC | | | |
| | | LLIFAVTQE | ACGGCAACGGGAGCUUCCAGUGCGGGGUGUGCGCAUGCC | | | |
| | | QVHLYENYA | ACCCCGGCCACAUGGGGCCUCGCUGCGAGUGCGGCGAGG | | | |
| | | KLIPGATVG | ACAUGCUGUCUACCGACAGCUGCAAGGAGGCCCCUGACC | | | |
| | | LLQKDSGNI | ACCCUAGCUGCUCUGGCCGCGGAGACUGCUACUGCGGCC | | | |
| | | LQLIISAYE | AGUGCAUCUGCCACCUGUCCCCUUACGGGAACAUUUACG | | | |
| | | ELRSEVELE | GGCCCUACUGCCAGUGUGAUAACUUUUCCUGCGUGCGGC | | | |
| | | VLGDTEGLN | ACAAGGGCCUGCUGUGCGGAGGAAAUGGCGACUGCGACU | | | |
| | | LSFTAICNN | GCGGCGAGUGCGUCUGUCGCAGCGGCUGGACCGGGGAGU | | | |
| | | GTLFQHQKK | ACUGCAACUGCACUACUUCCACUGACAGCUGUGUCAGCG | | | |
| | | CSHMKVGDT | AGGACGGGGUGCUCUGUUCUGGCCGCGGGGACUGCGUCU | | | |
| | | ASFSVTVNI | GCGGCAAGUGCGUGUGCACUAAUCCAGGGGCCUCCGGGC | | | |
| | | PHCERRSRH | CCACCUGCGAGCGGUGUCCCACCUGCGGGGACCCUUGCA | | | |
| | | IIIKPVGLG | ACAGCAAGAGGUCCUGCAUCGAGUGCCACCUGUCCGCCG | | | |
| | | DALELLVSP | CCGGACAGGCACGCGAGGAGUGCGUCGAUAAGUGUAAGC | | | |
| | | ECNCDCQKE | UCGCCGGAGCUACAAUUAGCGAGGAGGAGGACUUCUCCA | | | |
| | | VEVNSSKCH | AGGACGGGAGCGUGAGCUGCUCCCUCCAGGGAGAGAACG | | | |
| | | HGNGSFQCG | AGUGCCUGAUCACUUUCCUCAUCACCACUGAUAACGAGG | | | |
| | | VCACHPGHM | GAAAGACAAUCAUUCACUCUAUCAACGAGAAGGAUUGUC | | | |
| | | GPRCECGED | CCAAGCCCCCAAACAUUCCCAUGAUCAUGCUGGGAGUGU | | | |
| | | MLSTDSCKE | CCCUGGCAAUCCUGCUGAUCGGAGUCGUGCUGCUCUGCA | | | |
| | | APDHPSCSG | UCUGGAAGCUGCUCGUCUCCUUCCACGACCGCAAGGAGG | | | |
| | | RGDCYCGQC | UCGCAAAGUUUGAGGCCGAGCGGUCUAAGGCCAAGUGGC | | | |
| | | ICHLSPYGN | AGACAGGGACCAAUCCUCUCUACCGCGGGUCUACUUCUA | | | |
| | | IYGPYCQCD | CCUUCAAGAACGUGACUUACAAGCACAGGGAGAAGCAGA | | | |
| | | NFSCVRHKG | AGGUGGACCUCUCCACCGAUUGCGGGAAGCCCAUCCCCA | | | |
| | | LLCGGNGDC | AUCCCCUGCUGGGCCUGGACAGCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 1 | 6 | 50 | 143 | 168 |
| Human | ITB6 | MGIELLCLF | AUGGGUAUCGAGCUGCUGUGUCUGUUCUUUCUGUUUCUGG | GGAAAUCG | UAAGCCCC | SEQ ID |
| Var- | G5 | FLFLGRNDH | GCCGCAACGACCACGUCCAGGGCGGCUGCGCCCUGGGCG | CAAAAUUU | UCCGGGGG | NO: 168 |
| iant | Cap II-b | VQGGCALGG | GGGCCGAGACCUGCGAGGACUGCCUGCUCAUCGGGCCAC | GCUCUUCG | CCUCGGUG | consists |
| 1.9 | Poly A | AETCEDCLL | AGUGUGCCUGGUGUGCACAGGAGAACUUCACCCACCCCA | CGUUAGAU | GCCUAGCU | from 5′ |
| | Tail: | IGPQCAWCA | GCGGGGUGGGCGAGAGGUGCGACACCCCUGCAAAUCUGC | UUCUUUUA | UCUUGCCC | to 3′ |
| | 100 nt | QENFTHPSG | UGGCAAAGGGCUGCCAGCUGAAUUUCAUCGAGAAUCCCG | GUUUUCUC | CUUGGGCC | end: 5′ |
| | (SEQ ID | VGERCDTPA | UCAGCCAGGUGGAGAUUCUCAAGAACAAGCCUCUGAGCG | GCAACUAG | UCCCCCCA | UTR of |
| | NO: 502) | NLLAKGCQL | UCGGACGGCAGAAGAACUCUUCUGACAUCGUCCAGAUCG | CAAGCUUU | GCCCCUCC | SEQ ID |
| | | NFIENPVSQ | CACCUCAGAGCCUGAUUCUGAAGCUCCGCCCAGGCGGAG | UUGUUCUC | UCCCCUUC | NO: 50, |
| | | VEILKNKPL | CUCAGACCCUCCAGGUCCACGUCAGGCAGACUGAGGACU | GCC | CUGCACCC | ORF |
| | | SVGRQKNSS | ACCCCGUGGACCUGUACUACCUGAUGGAUCUGUCCGCCA | | GUACCCCC | sequence |
| | | DIVQIAPQS | GCAUGGACGACGAUCUGAACACCAUUAAGGAGCUGGGCA | | CAAACACC | of SEQ |
| | | LILKLRPGG | GCCGGCUCUCUAAGGAGAUGUCCAAGCUCACCAGCAACU | | AUUGUCAC | ID NO: |
| | | AQTLQVHVR | UCAGGCUGGGAUUCGGCAGCUUUGUCGAGAAGCCUGUGU | | ACUCCAGU | 6, and |
| | | QTEDYPVDL | CUCCUUUCGUGAAGACCACACCAGAGGAGAUCGCAAACC | | GGUCUUUG | 3′ UTR |
| | | YYLMDLSAS | CCUGUAGCAGCAUCCCCUACUUCUGCCUGCCUACAUUCG | | AAUAAAGU | sequence |
| | | MDDDLNTIK | GGUUCAAGCACAUUCUGCCUCUGACCAACGACGCAGAGC | | CUGAGUGG | of SEQ |
| | | ELGSRLSKE | GCUUUAACGAGAUCGUGAAGAAUCAGAAGAUCAGCGCCA | | GCGGC | ID NO: |
| | | MSKLTSNFR | ACAUUGAUACUCCUGAGGGCGGCUUCGACGCCAUCAUGC | | | 143. |
| | | LGFGSFVEK | AGGCUGCCGUCUGCAAGGAGAAGAUCGGGUGGAGGAACG | | | |
| | | PVSPFVKTT | ACAGCCUCCACCUGCUCGUGUUCGUGAGCGACGCCGACA | | | |
| | | PEEIANPCS | GCCACUUUGGGAUGGACUCCAAGCUGGCCGGCAUCGUGA | | | |
| | | SIPYFCLPT | UCCCCAACGACGGGCUGUGUCACCUCGAUUCCAAGAACG | | | |
| | | FGFKHILPL | AGUACAGCAUGAGCACCGUGCUGGAGUACCCCACCAUUG | | | |
| | | TNDAERFNE | GGCAGCUCAUUGAUAAGCUCGUGCAGAACAACGUCCUGC | | | |
| | | IVKNQKISA | UGAUCUUUGCAGUGACACAGGAGCAGGUGCACCUGUACG | | | |
| | | NIDTPEGGF | AGAAUUACGCAAAGCUCAUCCCCGGAGCCACUGUGGGCC | | | |
| | | DAIMQAAVC | UCCUGCAGAAGGACUCCGGGAACAUCCUCCAGCUCAUCA | | | |
| | | KEKIGWRND | UCUCCGCCUACGAGGAGCUGAGGAGCGAGGUGGAGCUCG | | | |
| | | SLHLLVFVS | AGGUCCUCGGGGACACCGAGGGCCUCAACCUGAGCUUUA | | | |
| | | DADSHFGMD | CCGCCAUCUGUAACAACGGAACUCUCUUCCAGCACCAGA | | | |
| | | SKLAGIVIP | AGAAGUGCUCUCACAUGAAGGUGGGCGACACCGCCUCCU | | | |
| | | NDGLCHLDS | UCAGCGUGACCGUGAACAUCCCUCACUGUGAGCGGCGGA | | | |
| | | KNEYSMSTV | GCCGCCACAUCAUCAUCAAGCCCGUGGGGCUCGGGGACG | | | |
| | | LEYPTIGQL | CCCUGGAGCUCCUGGUGUCCCCUGAGUGUAACUGCGACU | | | |
| | | IDKLVQNNV | GCCAGAAGGAGGUCGAGGUGAACAGCUCUAAGUGCCACC | | | |
| | | LLIFAVTQE | ACGGCAACGGGAGCUUCCAGUGCGGGGUGUGCGCAUGCC | | | |
| | | QVHLYENYA | ACCCCGGCCACAUGGGGCCUCGCUGCGAGUGCGGCGAGG | | | |
| | | KLIPGATVG | ACAUGCUGUCUACCGACAGCUGCAAGGAGGCCCCUGACC | | | |
| | | LLQKDSGNI | ACCCUAGCUGCUCUGGCCGCGGAGACUGCUACUGCGGCC | | | |
| | | LQLIISAYE | AGUGCAUCUGCCACCUGUCCCCUUACGGGAACAUUUACG | | | |
| | | ELRSEVELE | GGCCCUACUGCCAGUGUGAUAACUUUUCCUGCGUGCGGC | | | |
| | | VLGDTEGLN | ACAAGGGCCUGCUGUGCGGAGGAAAUGGCGACUGCGACU | | | |
| | | LSFTAICNN | GCGGCGAGUGCGUCUGUCGCAGCGGCUGGACCGGGGAGU | | | |
| | | GTLFQHQKK | ACUGCAACUGCACUACUUCCACUGACAGCUGUGUCAGCG | | | |
| | | CSHMKVGDT | AGGACGGGGUGCUCUGUUCUGGCCGCGGGGACUGCGUCU | | | |
| | | ASFSVTVNI | GCGGCAAGUGCGUGUGCACUAAUCCAGGGGCCUCCGGGC | | | |
| | | PHCERRSRH | CCACCUGCGAGCGGUGUCCCACCUGCGGGGACCCUUGCA | | | |
| | | IIIKPVGLG | ACAGCAAGAGGUCCUGCAUCGAGUGCCACCUGUCCGCCG | | | |
| | | DALELLVSP | CCGGACAGGCACGCGAGGAGUGCGUCGAUAAGUGUAAGC | | | |
| | | ECNCDCQKE | UCGCCGGAGCUACAAUUAGCGAGGAGGAGGACUUCUCCA | | | |
| | | VEVNSSKCH | AGGACGGGAGCGUGAGCUGCUCCCUCCAGGGAGAGAACG | | | |
| | | HGNGSFQCG | AGUGCCUGAUCACUUUCCUCAUCACCACUGAUAACGAGG | | | |
| | | VCACHPGHM | GAAAGACAAUCAUUCACUCUAUCAACGAGAAGGAUUGUC | | | |
| | | GPRCECGED | CCAAGCCCCCAAACAUUCCCAUGAUCAUGCUGGGAGUGU | | | |
| | | MLSTDSCKE | CCCUGGCAAUCCUGCUGAUCGGAGUCGUGCUGCUCUGCA | | | |
| | | APDHPSCSG | UCUGGAAGCUGCUCGUCUCCUUCCACGACCGCAAGGAGG | | | |
| | | RGDCYCGQC | UCGCAAAGUUUGAGGCCGAGCGGUCUAAGGCCAAGUGGC | | | |
| | | ICHLSPYGN | AGACAGGGACCAAUCCUCUCUACCGCGGGUCUACUUCUA | | | |
| | | IYGPYCQCD | CCUUCAAGAACGUGACUUACAAGCACAGGGAGAAGCAGA | | | |
| | | NFSCVRHKG | AGGUGGACCUCUCCACCGAUUGCGGGAAGCCCAUCCCCA | | | |
| | | LLCGGNGDC | AUCCCCUGCUGGGCCUGGACAGCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 1 | 5 | 50 | 145 | 169 |
| Human | ITB6 | MGIELLCLF | AUGGGGAUCGAGCUGCUGUGCCUGUUCUUCCUGUUCCUGG | GGAAAUCG | UAAAGCUC | SEQ ID |
| Var- | G5 | FLFLGRNDH | GACGGAACGACCACGUGCAGGGUGGCUGCGCCCUUGGUG | CAAAAUUU | CCCGGGGG | NO: 169 |
| iant | Cap II-b | VQGGCALGG | GGGCAGAGACCUGCGAGGACUGCCUGCUCAUCGGCCCAC | GCUCUUCG | CCUCGGUG | consists |
| 1.10 | Poly A | AETCEDCLL | AGUGCGCCUGGUGCGCUCAGGAGAACUUCACCCACCCCA | CGUUAGAU | GCCUAGCU | from 5′ |
| | Tail: | IGPQCAWCA | GCGGAGUGGGCGAGAGGUGCGACACCCCGGCCAACCUGC | UUCUUUUA | UCUUGCCC | to 3′ |
| | 100 nt | QENFTHPSG | UGGCCAAGGGGUGCCAGCUGAACUUCAUCGAGAACCCCG | GUUUUCUC | CUUGGGCC | end: 5′ |
| | (SEQ ID | VGERCDTPA | UCUCUCAGGUGGAGAUCCUGAAGAACAAGCCCCUCAGCG | GCAACUAG | UCCCCCCA | UTR of |
| | NO: 502) | NLLAKGCQL | UUGGGCGGCAGAAGAACAGCUCUGACAUCGUGCAGAUCG | CAAGCUUU | GCCCCUCC | SEQ ID |
| | | NFIENPVSQ | CGCCCCAGAGCCUGAUCCUGAAACUACGGCCUGGCGGUG | UUGUUCUC | UCCCCUUC | NO: 50, |
| | | VEILKNKPL | CGCAAACCCUGCAGGUCCACGUGCGGCAGACCGAGGACU | GCC | CUGCACCC | ORF |
| | | SVGRQKNSS | ACCCUGUGGACCUGUACUACCUGAUGGACCUCUCCGCCA | | GUACCCCC | sequence |
| | | DIVQIAPQS | GCAUGGACGACGAUCUGAACACCAUCAAGGAGCUGGGCA | | CAAACACC | of SEQ |
| | | LILKLRPGG | GCCGCCUGAGCAAGGAGAUGUCCAAGCUGACCUCCAACU | | AUUGUCAC | ID NO: |
| | | AQTLQVHVR | UCCGCCUGGGGUUCGGAUCCUUCGUGGAGAAGCCCGUGA | | ACUCCAGU | 5, and |
| | | QTEDYPVDL | GCCCCUUCGUGAAGACCACCCCUGAGGAGAUCGCCAACC | | GGUCUUUG | 3′ UTR |
| | | YYLMDLSAS | CCUGCAGCAGCAUCCCUUACUUCUGCCUGCCCACCUUCG | | AAUAAAGU | sequence |
| | | MDDDLNTIK | GCUUCAAGCACAUCCUGCCACUGACCAACGACGCCGAGC | | CUGAGUGG | of SEQ |
| | | ELGSRLSKE | GGUUCAACGAGAUCGUGAAGAACCAGAAGAUCAGCGCCA | | GCGGC | ID NO: |
| | | MSKLTSNFR | ACAUCGACACCCCGGAGGGUGGGUUCGACGCUAUCAUGC | | | 145. |
| | | LGFGSFVEK | AGGCCGCCGUGUGCAAGGAGAAGAUCGGCUGGCGCAACG | | | |
| | | PVSPFVKTT | ACAGCCUGCACCUGCUGGUGUUCGUGUCCGACGCCGACU | | | |
| | | PEEIANPCS | CCCACUUCGGCAUGGACAGCAAGCUGGCCGGUAUCGUGA | | | |
| | | SIPYFCLPT | UCCCUAACGACGGCCUGUGCCACCUGGACAGCAAGAACG | | | |
| | | FGFKHILPL | AGUACAGCAUGAGCACCGUGCUGGAGUACCCCACCAUCG | | | |
| | | TNDAERFNE | GCCAGCUGAUCGACAAGCUGGUGCAGAACAACGUGCUGC | | | |
| | | IVKNQKISA | UGAUCUUCGCCGUGACCCAGGAGCAGGUGCACCUCUACG | | | |
| | | NIDTPEGGF | AGAACUACGCCAAGCUGAUUCCAGGCGCCACCGUCGGUC | | | |
| | | DAIMQAAVC | UGCUGCAGAAGGACAGCGGCAACAUCCUGCAGCUGAUCA | | | |
| | | KEKIGWRND | UCUCCGCCUACGAGGAGCUGAGGAGCGAGGUGGAGCUGG | | | |
| | | SLHLLVFVS | AGGUGCUCGGGGACACCGAGGGCCUGAACCUGUCCUUCA | | | |
| | | DADSHFGMD | CCGCCAUCUGCAACAACGGGACCCUCUUCCAGCACCAGA | | | |
| | | SKLAGIVIP | AGAAGUGCAGCCACAUGAAGGUGGGCGACACCGCCAGCU | | | |
| | | NDGLCHLDS | UCAGCGUCACCGUGAACAUACCUCACUGUGAGCGGCGGA | | | |
| | | KNEYSMSTV | GCAGGCACAUCAUCAUCAAGCCCGUGGGCCUGGGAGACG | | | |
| | | LEYPTIGQL | CCCUGGAGCUGCUGGUGUCUCCCGAGUGCAACUGCGACU | | | |
| | | IDKLVQNNV | GCCAGAAGGAGGUGGAGGUGAACUCUAGCAAGUGCCACC | | | |
| | | LLIFAVTQE | ACGGCAACGGGAGUUUCCAGUGUGGCGUGUGUGCCUGCC | | | |
| | | QVHLYENYA | ACCCCGGUCACAUGGGACCCCGGUGUGAGUGCGGGGAGG | | | |
| | | KLIPGATVG | ACAUGCUGAGCACCGACAGCUGCAAGGAGGCCCCAGACC | | | |
| | | LLQKDSGNI | ACCCGAGCUGUUCCGGUCGGGGCGACUGCUACUGCGGCC | | | |
| | | LQLIISAYE | AGUGCAUCUGUCACCUGAGCCCCUACGGCAACAUCUACG | | | |
| | | ELRSEVELE | GGCCCUACUGCCAGUGCGACAACUUCUCCUGCGUGCGCC | | | |
| | | VLGDTEGLN | ACAAAGGCCUGCUGUGUGGCGGCAACGGCGACUGCGACU | | | |
| | | LSFTAICNN | GCGGGGAGUGCGUGUGCCGCAGUGGGUGGACCGGCGAGU | | | |
| | | GTLFQHQKK | ACUGCAACUGCACCACCUCCACCGACUCUUGCGUGUCCG | | | |
| | | CSHMKVGDT | AGGACGGGGUGCUGUGUUCCGGGCGCGGAGAUUGCGUGU | | | |
| | | ASFSVTVNI | GCGGGAAGUGCGUGUGCACCAACCCUGGCGCAUCGGGUC | | | |
| | | PHCERRSRH | CUACCUGCGAGAGGUGCCCCACCUGUGGCGACCCCUGCA | | | |
| | | IIIKPVGLG | ACAGCAAGCGCUCUUGCAUCGAGUGCCACCUCUCAGCAG | | | |
| | | DALELLVSP | CAGGGCAGGCUCGGGAGGAGUGCGUGGACAAGUGCAAGC | | | |
| | | ECNCDCQKE | UCGCCGGCGCCACCAUCAGUGAGGAGGAGGACUUCUCCA | | | |
| | | VEVNSSKCH | AGGACGGCAGCGUGUCCUGCUCCCUCCAGGGCGAGAACG | | | |
| | | HGNGSFQCG | AGUGCCUCAUCACCUUCCUGAUCACCACCGACAACGAGG | | | |
| | | VCACHPGHM | GCAAGACCAUCAUCCACUCCAUCAACGAGAAGGACUGCC | | | |
| | | GPRCECGED | CCAAGCCGCCUAACAUCCCCAUGAUCAUGCUGGGCGUGA | | | |
| | | MLSTDSCKE | GCCUGGCCAUCCUGCUGAUCGGCGUGGUGCUGCUGUGCA | | | |
| | | APDHPSCSG | UCUGGAAGCUGCUGGUCUCCUUCCACGACCGCAAGGAGG | | | |
| | | RGDCYCGQC | UGGCCAAGUUCGAGGCCGAGCGCUCUAAGGCCAAGUGGC | | | |
| | | ICHLSPYGN | AGACCGGCACCAACCCUCUGUACCGGGGCUCCACCAGCA | | | |
| | | IYGPYCQCD | CCUUCAAGAACGUGACCUACAAGCACCGCGAGAAGCAGA | | | |
| | | NFSCVRHKG | AGGUGGACCUGUCCACCGACUGUGGCAAGCCCAUCCCCA | | | |
| | | LLCGGNGDC | AUCCCCUGCUGGGGCUGGACUCCACC | | | |
| | | DCGECVCRS | | | | |
| | | GWTGEYCNC | | | | |
| | | TTSTDSCVS | | | | |
| | | EDGVLCSGR | | | | |
| | | GDCVCGKCV | | | | |
| | | CTNPGASGP | | | | |
| | | TCERCPTCG | | | | |
| | | DPCNSKRSC | | | | |
| | | IECHLSAAG | | | | |
| | | QAREECVDK | | | | |
| | | CKLAGATIS | | | | |
| | | EEEDFSKDG | | | | |
| | | SVSCSLQGE | | | | |
| | | NECLITFLI | | | | |
| | | TTDNEGKTI | | | | |
| | | IHSINEKDC | | | | |
| | | PKPPNIPMI | | | | |
| | | MLGVSLAIL | | | | |
| | | LIGVVLLCI | | | | |
| | | WKLLVSFHD | | | | |
| | | RKEVAKFEA | | | | |
| | | ERSKAKWQT | | | | |
| | | GTNPLYRGS | | | | |
| | | TSTFKNVTY | | | | |
| | | KHREKQKVD | | | | |
| | | LSTDCGKPI | | | | |
| | | PNPLLGLDS | | | | |
| | | T | | | | |
| | | 7 | 8 | 50 | 110 | 170 |
| Human | ITB6 | MGIELLCLFF | AUGGGGAUCGAGCUGCUGUGCCUGUUCUUCCUGUUCCUGG | GGAAAUCG | UGAUAAUA | SEQ ID |
| Var- | G5 | LFLGRNDHV | GACGGAACGACCACGUGCAGGGUGGCUGCGCCCUUGGUG | CAAAAUUU | GGCUGGAG | NO: 170 |
| iant | Cap C1 | QGGCALGGA | GGGCAGAGACCUGCGAGGACUGCCUGCUCAUCGGCCCAC | GCUCUUCG | CCUCGGUG | consists |
| 2.1 | Poly A | ETCEDCLLI | AGUGCGCCUGGUGCGCUCAGGAGAACUUCACCCACCCCA | CGUUAGAU | GCCUAGCU | from 5′ |
| | Tail: | GPQCAWCAQ | GCGGAGUGGGCGAGAGGUGCGACACCCCGGCCAACCUGC | UUCUUUUA | UCUUGCCC | to 3′ |
| | 100 nt | ENFTHPSGV | UGGCCAAGGGGUGCCAGCUGAACUUCAUCGAGAACCCCG | GUUUUCUC | CUUGGGCC | end: 5′ |
| | (SEQ ID | GERCDTPAN | UCUCUCAGGUGGAGAUCCUGAAGAACAAGCCCCUCAGCG | GCAACUAG | UCCCCCCA | UTR of |
| | NO: 502) | LLAKGCQLN | UUGGGCGGCAGAAGAACAGCUCUGACAUCGUGCAGAUCG | CAAGCUUU | GCCCCUCC | SEQ ID |
| | | FIENPVSQV | CGCCCCAGAGCCUGAUCCUGAAACUACGGCCUGGCGGUG | UUGUUCUC | UCCCCUUC | NO: 50, |
| | | EILKNKPLS | CGCAAACCCUGCAGGUCCACGUGCGGCAGACCGAGGACU | GCC | CUGCACCC | ORF |
| | | VGRQKNSSD | ACCCUGUGGACCUGUACUACCUGAUGGACCUCUCCGCCA | | GUACCCCC | sequence |
| | | IVQIAPQSL | GCAUGGACGACGAUCUGAACACCAUCAAGGAGCUGGGCA | | CAAACACC | of SEQ |
| | | ILKLRPGGA | GCCGCCUGAGCAAGGAGAUGUCCAAGCUGACCUCCAACU | | AUUGUCAC | ID NO: |
| | | QTLQVHVRQ | UCCGCCUGGGGUUCGGAUCCUUCGUGGAGAAGCCCGUGA | | ACUCCAGU | 8, and |
| | | TEDYPVDLY | GCCCCUUCGUGAAGACCACCCCUGAGGAGAUCGCCAACC | | GGUCUUUG | 3′ UTR |
| | | YLMDLSASM | CCUGCAGCAGCAUCCCUUACUUCUGCCUGCCCACCUUCG | | AAUAAAGU | sequence |
| | | DDDLNTIKE | GCUUCAAGCACAUCCUGCCACUGACCAACGACGCCGAGC | | CUGAGUGG | of SEQ |
| | | LGSRLSKEM | GGUUCAACGAGAUCGUGAAGAACCAGAAGAUCAGCGCCA | | GCGGC | ID NO: |
| | | SKLTSNFRL | ACAUCGACGCCCCGGAGGGUGGGUUCGACGCUAUCAUGC | | | 110. |
| | | GFGSFVEKP | AGGCCGCCGUGUGCAAGGAGAAGAUCGGCUGGCGCAACG | | | |
| | | VSPFVKTTP | ACAGCCUGCACCUGCUGGUGUUCGUGUCCGACGCCGACU | | | |
| | | EEIANPCSS | CCCACUUCGGCAUGGACAGCAAGCUGGCCGGUAUCGUGA | | | |
| | | IPYFCLPTF | UCCCUAACGACGGCCUGUGCCACCUGGACAGCAAGAACG | | | |
| | | GFKHILPLT | AGUACAGCAUGAGCACCGUGCUGGAGUACCCCACCAUCG | | | |
| | | NDAERFNEI | GCCAGCUGAUCGACAAGCUGGUGCAGAACAACGUGCUGC | | | |
| | | VKNQKISAN | UGAUCUUCGCCGUGACCCAGGAGCAGGUGCACCUCUACG | | | |
| | | IDAPEGGED | AGAACUACGCCAAGCUGAUUCCAGGCGCCACCGUCGGUC | | | |
| | | AIMQAAVCK | UGCUGCAGAAGGACAGCGGCAACAUCCUGCAGCUGAUCA | | | |
| | | EKIGWRNDS | UCUCCGCCUACGAGGAGCUGAGGAGCGAGGUGGAGCUGG | | | |
| | | LHLLVFVSD | AGGUGCUCGGGGACACCGAGGGCCUGAACCUGUCCUUCA | | | |
| | | ADSHFGMDS | CCGCCAUCUGCAACAACGGGACCCUCUUCCAGCACCAGA | | | |
| | | KLAGIVIPN | AGAAGUGCAGCCACAUGAAGGUGGGCGACACCGCCAGCU | | | |
| | | DGLCHLDSK | UCAGCGUCACCGUGAACAUACCUCACUGUGAGCGGCGGA | | | |
| | | NEYSMSTVL | GCAGGCACAUCAUCAUCAAGCCCGUGGGCCUGGGAGACG | | | |
| | | EYPTIGQLI | CCCUGGAGCUGCUGGUGUCUCCCGAGUGCAACUGCGACU | | | |
| | | DKLVQNNVL | GCCAGAAGGAGGUGGAGGUGAACUCUAGCAAGUGCCACC | | | |
| | | LIFAVTQEQ | ACGGCAACGGGAGUUUCCAGUGUGGCGUGUGUGCCUGCC | | | |
| | | VHLYENYAK | ACCCCGGUCACAUGGGACCCCGGUGUGAGUGCGGGGAGG | | | |
| | | LIPGATVGL | ACAUGCUGAGCACCGACAGCUGCAAGGAGGCCCCAGACC | | | |
| | | LQKDSGNIL | ACCCGAGCUGUUCCGGUCGGGGCGACUGCUACUGCGGCC | | | |
| | | QLIISAYEE | AGUGCAUCUGUCACCUGAGCCCCUACGGCAACAUCUACG | | | |
| | | LRSEVELEV | GGCCCUACUGCCAGUGCGACAACUUCUCCUGCGUGCGCC | | | |
| | | LGDTEGLNL | ACAAAGGCCUGCUGUGUGGCGGCAACGGCGACUGCGACU | | | |
| | | SFTAICNNG | GCGGGGAGUGCGUGUGCCGCAGUGGGUGGACCGGCGAGU | | | |
| | | TLFQHQKKC | ACUGCAACUGCACCACCUCCACCGACUCUUGCGUGUCCG | | | |
| | | SHMKVGDTA | AGGACGGGGUGCUGUGUUCCGGGCGCGGAGAUUGCGUGU | | | |
| | | SFSVTVNIP | GCGGGAAGUGCGUGUGCACCAACCCUGGCGCAUCGGGUC | | | |
| | | HCERRSRHI | CUACCUGCGAGAGGUGCCCCACCUGUGGCGACCCCUGCA | | | |
| | | IIKPVGLGD | ACAGCAAGCGCUCUUGCAUCGAGUGCCACCUCUCAGCAG | | | |
| | | ALELLVSPE | CAGGGCAGGCUCGGGAGGAGUGCGUGGACAAGUGCAAGC | | | |
| | | CNCDCQKEV | UCGCCGGCGCCACCAUCAGUGAGGAGGAGGACUUCUCCA | | | |
| | | EVNSSKCHH | AGGACGGCAGCGUGUCCUGCUCCCUCCAGGGCGAGAACG | | | |
| | | GNGSFQCGV | AGUGCCUCAUCACCUUCCUGAUCACCACCGACAACGAGG | | | |
| | | CACHPGHMG | GCAAGACCAUCAUCCACUCCAUCAACGAGAAGGACUGCC | | | |
| | | PRCECGEDM | CCAAGCCGCCUAACAUCCCCAUGAUCAUGCUGGGCGUGA | | | |
| | | LSTDSCKEA | GCCUGGCCAUCCUGCUGAUCGGCGUGGUGCUGCUGUGCA | | | |
| | | PDHPSCSGR | UCUGGAAGCUGCUGGUCUCCUUCCACGACCGCAAGGAGG | | | |
| | | GDCYCGQCI | UGGCCAAGUUCGAGGCCGAGCGCUCUAAGGCCAAGUGGC | | | |
| | | CHLSPYGNI | AGACCGGCACCAACCCUCUGUACCGGGGCUCCACCAGCA | | | |
| | | YGPYCQCDN | CCUUCAAGAACGUGACCUACAAGCACCGCGAGAAGCAGA | | | |
| | | FSCVRHKGL | AGGUGGACCUGUCCACCGACUGUGGCAAGCCCAUCCCCA | | | |
| | | LCGGNGDCD | AUCCCCUGCUGGGGCUGGACUCCACC | | | |
| | | CGECVCRSG | | | | |
| | | WTGEYCNCT | | | | |
| | | TSTDSCVSE | | | | |
| | | DGVLCSGRG | | | | |
| | | DCVCGKCVC | | | | |
| | | TNPGASGPT | | | | |
| | | CERCPTCGD | | | | |
| | | PCNSKRSCI | | | | |
| | | ECHLSAAGQ | | | | |
| | | AREECVDKC | | | | |
| | | KLAGATISE | | | | |
| | | EEDFSKDGS | | | | |
| | | VSCSLQGEN | | | | |
| | | ECLITFLIT | | | | |
| | | TDNEGKTII | | | | |
| | | HSINEKDCP | | | | |
| | | KPPNIPMIM | | | | |
| | | LGVSLAILL | | | | |
| | | IGVVLLCIW | | | | |
| | | KLLVSFHDR | | | | |
| | | KEVAKFEAE | | | | |
| | | RSKAKWQTG | | | | |
| | | TNPLYRGST | | | | |
| | | STFKNVTYK | | | | |
| | | HREKQKVDL | | | | |
| | | STDCGKPIP | | | | |
| | | NPLLGLDST | | | | |
| | | 9 | 10 | 50 | 110 | 171 |
| Human | ITB6 | MGIELLCLFF | AUGGGGAUCGAGCUGCUGUGCCUGUUCUUCCUGUUCCUGG | GGAAAUCG | UGAUAAUA | SEQ ID |
| Var- | G5 | LFLGRNDHV | GACGGAACGACCACGUGCAGGGUGGCUGCGCCCUUGGUG | CAAAAUUU | GGCUGGAG | NO: 171 |
| iant | Cap C1 | QGGCALGGA | GGGCAGAGACCUGCGAGGACUGCCUGCUCAUCGGCCCAC | GCUCUUCG | CCUCGGUG | consists |
| 3.1 | Poly A | ETCEDCLLI | AGUGCGCCUGGUGCGCUCAGGAGAACUUCACCCACCCCA | CGUUAGAU | GCCUAGCU | from 5′ |
| | Tail: | GPQCAWCAQ | GCGGAGUGGGCGAGAGGUGCGACACCCCGGCCAACCUGC | UUCUUUUA | UCUUGCCC | to 3′ |
| | 100 nt | ENFTHPSGV | UGGCCAAGGGGUGCCAGCUGAACUUCAUCGAGAACCCCG | GUUUUCUC | CUUGGGCC | end: 5′ |
| | (SEQ ID | GERCDTPAN | UCUCUCAGGUGGAGAUCCUGAAGAACAAGCCCCUCAGCG | GCAACUAG | UCCCCCCA | UTR of |
| | NO: 502) | LLAKGCQLN | UUGGGCGGCAGAAGAACAGCUCUGACAUCGUGCAGAUCG | CAAGCUUU | GCCCCUCC | SEQ ID |
| | | FIENPVSQV | CGCCCCAGAGCCUGAUCCUGAAACUACGGCCUGGCGGUG | UUGUUCUC | UCCCCUUC | NO: 50, |
| | | EILKNKPLS | CGCAAACCCUGCAGGUCCACGUGCGGCAGACCGAGGACU | GCC | CUGCACCC | ORF |
| | | VGRQKNSSD | ACCCUGUGGACCUGUACUACCUGAUGGACCUCUCCUACA | | GUACCCCC | sequence |
| | | IVQIAPQSL | GCAUGGACGACGAUCUGAACACCAUCAAGGAGCUGGGCA | | CAAACACC | of SEQ |
| | | ILKLRPGGA | GCCGCCUGAGCAAGGAGAUGUCCAAGCUGACCUCCAACU | | AUUGUCAC | ID NO: |
| | | QTLQVHVRQ | UCCGCCUGGGGUUCGGAUCCUUCGUGGAGAAGCCCGUGA | | ACUCCAGU | 10, and |
| | | TEDYPVDLY | GCCCCUUCGUGAAGACCACCCCUGAGGAGAUCGCCAACC | | GGUCUUUG | 3′ UTR |
| | | YLMDLSYSM | CCUGCAGCAGCAUCCCUUACUUCUGCCUGCCCACCUUCG | | AAUAAAGU | sequence |
| | | DDDLNTIKE | GCUUCAAGCACAUCCUGCCACUGACCAACGACGCCGAGC | | CUGAGUGG | of SEQ |
| | | LGSRLSKEM | GGUUCAACGAGAUCGUGAAGAACCAGAAGAUCAGCGCCA | | GCGGC | ID NO: |
| | | SKLTSNFRL | ACAUCGACGCCCCGGAGGGUGGGUUCGACGCUAUCAUGC | | | 110. |
| | | GFGSFVEKP | AGGCCGCCGUGUGCAAGGAGAAGAUCGGCUGGCGCAACG | | | |
| | | VSPFVKTTP | ACAGCCUGCACCUGCUGGUGUUCGUGUCCGACGCCGACU | | | |
| | | EEIANPCSS | CCCACUUCGGCAUGGACAGCAAGCUGGCCGGUAUCGUGA | | | |
| | | IPYFCLPTF | UCCCUAACGACGGCCUGUGCCACCUGGACAGCAAGAACG | | | |
| | | GFKHILPLT | AGUACAGCAUGAGCACCGUGCUGGAGUACCCCACCAUCG | | | |
| | | NDAERFNEI | GCCAGCUGAUCGACAAGCUGGUGCAGAACAACGUGCUGC | | | |
| | | VKNQKISAN | UGAUCUUCGCCGUGACCCAGGAGCAGGUGCACCUCUACG | | | |
| | | IDAPEGGED | AGAACUACGCCAAGCUGAUUCCAGGCGCCACCGUCGGUC | | | |
| | | AIMQAAVCK | UGCUGCAGAAGGACAGCGGCAACAUCCUGCAGCUGAUCA | | | |
| | | EKIGWRNDS | UCUCCGCCUACGAGGAGCUGAGGAGCGAGGUGGAGCUGG | | | |
| | | LHLLVFVSD | AGGUGCUCGGGGACACCGAGGGCCUGAACCUGUCCUUCA | | | |
| | | ADSHFGMDS | CCGCCAUCUGCAACAACGGGACCCUCUUCCAGCACCAGA | | | |
| | | KLAGIVIPN | AGAAGUGCAGCCACAUGAAGGUGGGCGACACCGCCAGCU | | | |
| | | DGLCHLDSK | UCAGCGUCACCGUGAACAUACCUCACUGUGAGCGGCGGA | | | |
| | | NEYSMSTVL | GCAGGCACAUCAUCAUCAAGCCCGUGGGCCUGGGAGACG | | | |
| | | EYPTIGQLI | CCCUGGAGCUGCUGGUGUCUCCCGAGUGCAACUGCGACU | | | |
| | | DKLVQNNVL | GCCAGAAGGAGGUGGAGGUGAACUCUAGCAAGUGCCACC | | | |
| | | LIFAVTQEQ | ACGGCAACGGGAGUUUCCAGUGUGGCGUGUGUGCCUGCC | | | |
| | | VHLYENYAK | ACCCCGGUCACAUGGGACCCCGGUGUGAGUGCGGGGAGG | | | |
| | | LIPGATVGL | ACAUGCUGAGCACCGACAGCUGCAAGGAGGCCCCAGACC | | | |
| | | LQKDSGNIL | ACCCGAGCUGUUCCGGUCGGGGCGACUGCUACUGCGGCC | | | |
| | | QLIISAYEE | AGUGCAUCUGUCACCUGAGCCCCUACGGCAACAUCUACG | | | |
| | | LRSEVELEV | GGCCCUACUGCCAGUGCGACAACUUCUCCUGCGUGCGCC | | | |
| | | LGDTEGLNL | ACAAAGGCCUGCUGUGUGGCGGCAACGGCGACUGCGACU | | | |
| | | SFTAICNNG | GCGGGGAGUGCGUGUGCCGCAGUGGGUGGACCGGCGAGU | | | |
| | | TLFQHQKKC | ACUGCAACUGCACCACCUCCACCGACUCUUGCGUGUCCG | | | |
| | | SHMKVGDTA | AGGACGGGGUGCUGUGUUCCGGGCGCGGAGAUUGCGUGU | | | |
| | | SFSVTVNIP | GCGGGAAGUGCGUGUGCACCAACCCUGGCGCAUCGGGUC | | | |
| | | HCERRSRHI | CUACCUGCGAGAGGUGCCCCACCUGUGGCGACCCCUGCA | | | |
| | | IIKPVGLGD | ACAGCAAGCGCUCUUGCAUCGAGUGCCACCUCUCAGCAG | | | |
| | | ALELLVSPE | CAGGGCAGGCUCGGGAGGAGUGCGUGGACAAGUGCAAGC | | | |
| | | CNCDCQKEV | UCGCCGGCGCCACCAUCAGUGAGGAGGAGGACUUCUCCA | | | |
| | | EVNSSKCHH | AGGACGGCAGCGUGUCCUGCUCCCUCCAGGGCGAGAACG | | | |
| | | GNGSFQCGV | AGUGCCUCAUCACCUUCCUGAUCACCACCGACAACGAGG | | | |
| | | CACHPGHMG | GCAAGACCAUCAUCCACUCCAUCAACGAGAAGGACUGCC | | | |
| | | PRCECGEDM | CCAAGCCGCCUAACAUCCCCAUGAUCAUGCUGGGCGUGA | | | |
| | | LSTDSCKEA | GCCUGGCCAUCCUGCUGAUCGGCGUGGUGCUGCUGUGCA | | | |
| | | PDHPSCSGR | UCUGGAAGCUGCUGGUCUCCUUCCACGACCGCAAGGAGG | | | |
| | | GDCYCGQCI | UGGCCAAGUUCGAGGCCGAGCGCUCUAAGGCCAAGUGGC | | | |
| | | CHLSPYGNI | AGACCGGCACCAACCCUCUGUACCGGGGCUCCACCAGCA | | | |
| | | YGPYCQCDN | CCUUCAAGAACGUGACCUACAAGCACCGCGAGAAGCAGA | | | |
| | | FSCVRHKGL | AGGUGGACCUGUCCACCGACUGUGGCAAGCCCAUCCCCA | | | |
| | | LCGGNGDCD | AUCCCCUGCUGGGGCUGGACUCCACC | | | |
| | | CGECVCRSG | | | | |
| | | WTGEYCNCT | | | | |
| | | TSTDSCVSE | | | | |
| | | DGVLCSGRG | | | | |
| | | DCVCGKCVC | | | | |
| | | TNPGASGPT | | | | |
| | | CERCPTCGD | | | | |
| | | PCNSKRSCI | | | | |
| | | ECHLSAAGQ | | | | |
| | | AREECVDKC | | | | |
| | | KLAGATISE | | | | |
| | | EEDFSKDGS | | | | |
| | | VSCSLQGEN | | | | |
| | | ECLITFLIT | | | | |
| | | TDNEGKTII | | | | |
| | | HSINEKDCP | | | | |
| | | KPPNIPMIM | | | | |
| | | LGVSLAILL | | | | |
| | | IGVVLLCIW | | | | |
| | | KLLVSFHDR | | | | |
| | | KEVAKFEAE | | | | |
| | | RSKAKWQTG | | | | |
| | | TNPLYRGST | | | | |
| | | STFKNVTYK | | | | |
| | | HREKQKVDL | | | | |
| | | STDCGKPIP | | | | |
| | | NPLLGLDST | | | | |
| | | 11 | 12 | 50 | 110 | 172 |
| Human | ITB6 | MGIELLCLFF | AUGGGGAUCGAGCUGCUGUGCCUGUUCUUCCUGUUCCUGG | GGAAAUCG | UGAUAAUA | SEQ ID |
| Var- | G5 | LFLGRNDHV | GACGGAACGACCACGUGCAGGGUGGCUGCGCCCUUGGUG | CAAAAUUU | GGCUGGAG | NO: 172 |
| iant | Cap C1 | QGGCALGGA | GGGCAGAGACCUGCGAGGACUGCCUGCUCAUCGGCCCAC | GCUCUUCG | CCUCGGUG | consists |
| 4.1 | Poly A | ETCEDCLLI | AGUGCGCCUGGUGCGCUCAGGAGAACUUCACCCACCCCA | CGUUAGAU | GCCUAGCU | from 5′ |
| | Tail: | GPQCAWCAQ | GCGGAGUGGGCGAGAGGUGCGACACCCCGGCCAACCUGC | UUCUUUUA | UCUUGCCC | to 3′ |
| | 100 nt | ENFTHPSGV | UGGCCAAGGGGUGCCAGCUGAACUUCAUCGAGAACCCCG | GUUUUCUC | CUUGGGCC | end: 5′ |
| | (SEQ ID | GERCDTPAN | UCUCUCAGGUGGAGAUCCUGAAGAACAAGCCCCUCAGCG | GCAACUAG | UCCCCCCA | UTR of |
| | NO: 502) | LLAKGCQLN | UUGGGCGGCAGAAGAACAGCUCUGACAUCGUGCAGAUCG | CAAGCUUU | GCCCCUCC | SEQ ID |
| | | FIENPVSQV | CGCCCCAGAGCCUGAUCCUGAAACUACGGCCUGGCGGUG | UUGUUCUC | UCCCCUUC | NO: 50, |
| | | EILKNKPLS | CGCAAACCCUGCAGGUCCACGUGCGGCAGACCGAGGACU | GCC | CUGCACCC | ORF |
| | | VGRQKNSSD | ACCCUGUGGACCUGUACUACCUGAUGGACCUCUCCUACA | | GUACCCCC | sequence |
| | | IVQIAPQSL | GCAUGGACGACGAUCUGAACACCAUCAAGGAGCUGGGCA | | CAAACACC | of SEQ |
| | | ILKLRPGGA | GCCGCCUGAGCAAGGAGAUGUCCAAGCUGACCUCCAACU | | AUUGUCAC | ID NO: |
| | | QTLQVHVRQ | UCCGCCUGGGGUUCGGAUCCUUCGUGGAGAAGCCCGUGA | | ACUCCAGU | 12, and |
| | | TEDYPVDLY | GCCCCUUCGUGAAGACCACCCCUGAGGAGAUCGCCAACC | | GGUCUUUG | 3′ UTR |
| | | YLMDLSYSM | CCUGCAGCAGCAUCCCUUACUUCUGCCUGCCCACCUUCG | | AAUAAAGU | sequence |
| | | DDDLNTIKE | GCUUCAAGCACAUCCUGCCACUGACCAACGACGCCGAGC | | CUGAGUGG | of SEQ |
| | | LGSRLSKEM | GGUUCAACGAGAUCGUGAAGAACCAGAAGAUCAGCAGAA | | GCGGC | ID NO: |
| | | SKLTSNFRL | ACAUCGACGCCCCGGAGGGUGGGUUCGACGCUAUCAUGC | | | 110. |
| | | GFGSFVEKP | AGGCCGCCGUGUGCAAGGAGAAGAUCGGCUGGCGCAACG | | | |
| | | VSPFVKTTP | ACAGCCUGCACCUGCUGGUGUUCGUGUCCGACGCCGACU | | | |
| | | EEIANPCSS | CCCACUUCGGCAUGGACAGCAAGCUGGCCGGUAUCGUGA | | | |
| | | IPYFCLPTF | UCCCUAACGACGGCCUGUGCCACCUGGACAGCAAGAACG | | | |
| | | GFKHILPLT | AGUACAGCAUGAGCACCGUGCUGGAGUACCCCACCAUCG | | | |
| | | NDAERFNEI | GCCAGCUGAUCGACAAGCUGGUGCAGAACAACGUGCUGC | | | |
| | | VKNQKISRN | UGAUCUUCGCCGUGACCCAGGAGCAGGUGCACCUCUACG | | | |
| | | IDAPEGGED | AGAACUACGCCAAGCUGAUUCCAGGCGCCACCGUCGGUC | | | |
| | | AIMQAAVCK | UGCUGCAGAAGGACAGCGGCAACAUCCUGCAGCUGAUCA | | | |
| | | EKIGWRNDS | UCUCCGCCUACGAGGAGCUGAGGAGCGAGGUGGAGCUGG | | | |
| | | LHLLVFVSD | AGGUGCUCGGGGACACCGAGGGCCUGAACCUGUCCUUCA | | | |
| | | ADSHFGMDS | CCGCCAUCUGCAACAACGGGACCCUCUUCCAGCACCAGA | | | |
| | | KLAGIVIPN | AGAAGUGCAGCCACAUGAAGGUGGGCGACACCGCCAGCU | | | |
| | | DGLCHLDSK | UCAGCGUCACCGUGAACAUACCUCACUGUGAGCGGCGGA | | | |
| | | NEYSMSTVL | GCAGGCACAUCAUCAUCAAGCCCGUGGGCCUGGGAGACG | | | |
| | | EYPTIGQLI | CCCUGGAGCUGCUGGUGUCUCCCGAGUGCAACUGCGACU | | | |
| | | DKLVQNNVL | GCCAGAAGGAGGUGGAGGUGAACUCUAGCAAGUGCCACC | | | |
| | | LIFAVTQEQ | ACGGCAACGGGAGUUUCCAGUGUGGCGUGUGUGCCUGCC | | | |
| | | VHLYENYAK | ACCCCGGUCACAUGGGACCCCGGUGUGAGUGCGGGGAGG | | | |
| | | LIPGATVGL | ACAUGCUGAGCACCGACAGCUGCAAGGAGGCCCCAGACC | | | |
| | | LQKDSGNIL | ACCCGAGCUGUUCCGGUCGGGGCGACUGCUACUGCGGCC | | | |
| | | QLIISAYEE | AGUGCAUCUGUCACCUGAGCCCCUACGGCAACAUCUACG | | | |
| | | LRSEVELEV | GGCCCUACUGCCAGUGCGACAACUUCUCCUGCGUGCGCC | | | |
| | | LGDTEGLNL | ACAAAGGCCUGCUGUGUGGCGGCAACGGCGACUGCGACU | | | |
| | | SFTAICNNG | GCGGGGAGUGCGUGUGCCGCAGUGGGUGGACCGGCGAGU | | | |
| | | TLFQHQKKC | ACUGCAACUGCACCACCUCCACCGACUCUUGCGUGUCCG | | | |
| | | SHMKVGDTA | AGGACGGGGUGCUGUGUUCCGGGCGCGGAGAUUGCGUGU | | | |
| | | SFSVTVNIP | GCGGGAAGUGCGUGUGCACCAACCCUGGCGCAUCGGGUC | | | |
| | | HCERRSRHI | CUACCUGCGAGAGGUGCCCCACCUGUGGCGACCCCUGCA | | | |
| | | IIKPVGLGD | ACAGCAAGCGCUCUUGCAUCGAGUGCCACCUCUCAGCAG | | | |
| | | ALELLVSPE | CAGGGCAGGCUCGGGAGGAGUGCGUGGACAAGUGCAAGC | | | |
| | | CNCDCQKEV | UCGCCGGCGCCACCAUCAGUGAGGAGGAGGACUUCUCCA | | | |
| | | EVNSSKCHH | AGGACGGCAGCGUGUCCUGCUCCCUCCAGGGCGAGAACG | | | |
| | | GNGSFQCGV | AGUGCCUCAUCACCUUCCUGAUCACCACCGACAACGAGG | | | |
| | | CACHPGHMG | GCAAGACCAUCAUCCACUCCAUCAACGAGAAGGACUGCC | | | |
| | | PRCECGEDM | CCAAGCCGCCUAACAUCCCCAUGAUCAUGCUGGGCGUGA | | | |
| | | LSTDSCKEA | GCCUGGCCAUCCUGCUGAUCGGCGUGGUGCUGCUGUGCA | | | |
| | | PDHPSCSGR | UCUGGAAGCUGCUGGUCUCCUUCCACGACCGCAAGGAGG | | | |
| | | GDCYCGQCI | UGGCCAAGUUCGAGGCCGAGCGCUCUAAGGCCAAGUGGC | | | |
| | | CHLSPYGNI | AGACCGGCACCAACCCUCUGUACCGGGGCUCCACCAGCA | | | |
| | | YGPYCQCDN | CCUUCAAGAACGUGACCUACAAGCACCGCGAGAAGCAGA | | | |
| | | FSCVRHKGL | AGGUGGACCUGUCCACCGACUGUGGCAAGCCCAUCCCCA | | | |
| | | LCGGNGDCD | AUCCCCUGCUGGGGCUGGACUCCACC | | | |
| | | CGECVCRSG | | | | |
| | | WTGEYCNCT | | | | |
| | | TSTDSCVSE | | | | |
| | | DGVLCSGRG | | | | |
| | | DCVCGKCVC | | | | |
| | | TNPGASGPT | | | | |
| | | CERCPTCGD | | | | |
| | | PCNSKRSCI | | | | |
| | | ECHLSAAGQ | | | | |
| | | AREECVDKC | | | | |
| | | KLAGATISE | | | | |
| | | EEDFSKDGS | | | | |
| | | VSCSLQGEN | | | | |
| | | ECLITFLIT | | | | |
| | | TDNEGKTII | | | | |
| | | HSINEKDCP | | | | |
| | | KPPNIPMIM | | | | |
| | | LGVSLAILL | | | | |
| | | IGVVLLCIW | | | | |
| | | KLLVSFHDR | | | | |
| | | KEVAKFEAE | | | | |
| | | RSKAKWQTG | | | | |
| | | TNPLYRGST | | | | |
| | | STFKNVTYK | | | | |
| | | HREKQKVDL | | | | |
| | | STDCGKPIP | | | | |
| | | NPLLGLDST | | | | |
| | | 13 | 14 | 50 | 110 | 173 |
| Rat | ITB6 | MGIELVCLFL | AUGGGCAUCGAGCUGGUGUGCCUGUUCCUGUUACUACUGG | GGAAAUCG | UGAUAAUA | SEQ ID |
| Var- | G5 | LLLGRNDHV | GCAGAAACGACCACGUGCAGGGCGGCUGCGCCUGGAGCG | CAAAAUUU | GGCUGGAG | NO: 173 |
| iant | Cap II-b | QGGCAWSGA | GCGCCGAAACCUGCAGCGACUGCCUGCUGACCGGCCCUC | GCUCUUCG | CCUCGGUG | consists |
| 1.1 | Poly A | ETCSDCLLT | ACUGCGCUUGGUGCAGCCAGGAGAACUUCACCCACCUCU | CGUUAGAU | GCCUAGCU | from 5′ |
| | Tail: | GPHCAWCSQ | CAGGCGCUGGCGAGCGGUGCGACACCCCUGAGAACCUGC | UUCUUUUA | UCUUGCCC | to 3′ |
| | 100 nt | ENFTHLSGA | UGGCCAAGGGCUGCCAGCUGCCUUUCAUCGAGAACCCUG | GUUUUCUC | CUUGGGCC | end: 5′ |
| | (SEQ ID | GERCDTPEN | UGAGCCAGGUGGAGAUCCUGCAGAACAAGCCUCUGAGCG | GCAACUAG | UCCCCCCA | UTR of |
| | NO: 502) | LLAKGCQLP | UGGGCAGACAGAAGAACAGCAGCGACAUCGUGCAGAUCG | CAAGCUUU | GCCCCUCC | SEQ ID |
| | | FIENPVSQV | CCCCUCAGAGUCUCGUUCUGAAGCUUCGGCCUGGCGGCG | UUGUUCUC | UCCCCUUC | NO: 50, |
| | | EILQNKPLS | AGCAGACCCUGCAGGUGCAGGUGAGACAGACCGAGGACU | GCC | CUGCACCC | ORF |
| | | VGRQKNSSD | ACCCUGUGGACCUGUACUACCUGAUGGACUUAUCCGCCA | | GUACCCCC | sequence |
| | | IVQIAPQSL | GCAUGGACGACGACCUGAACACCAUCAAGGAGCUGGGCA | | CAAACACC | of SEQ |
| | | VLKLRPGGE | GCAGACUUGCAAAGGAGAUGAGCAAGCUGACCAGCAACU | | AUUGUCAC | ID NO: |
| | | QTLQVQVRQ | UCAGACUGGGCUUCGGCAGCUUCGUGGAGAAGCCAGUUA | | ACUCCAGU | 14, and |
| | | TEDYPVDLY | GUCCUUUCAUGAAGACGACCCCAGAGGAGAUCACCAACC | | GGUCUUUG | 3′ UTR |
| | | YLMDLSASM | CUUGCAGCAGCAUCCCUUACUUCUGCCUGCCUACCUUCG | | AAUAAAGU | sequence |
| | | DDDLNTIKE | GCUUCAAGCACAUCCUGCCUCUGACCGACGACGCCGAGA | | CUGAGUGG | of SEQ |
| | | LGSRLAKEM | GAUUCAACGAGAUCGUGAGAAAGCAGAAGAUCAGCGCCA | | GCGGC | ID NO: |
| | | SKLTSNFRL | ACAUCGAUACACCUGAGGGAGGUUUCGACGCCAUCAUGC | | | 110. |
| | | GFGSFVEKP | AGGCCGCCGUGUGCAAGGAGAAGAUCGGCUGGCGAAACG | | | |
| | | VSPFMKTTP | ACAGCCUGCACCUUCUAGUGUUCGUGAGCGACGCAGACA | | | |
| | | EEITNPCSS | GCCACUUCGGCAUGGACUCUAAGCUAGCCGGCAUCGUGA | | | |
| | | IPYFCLPTF | UCCCUAACGACGGCCUGUGCCACCUGGACAAUCGUAACG | | | |
| | | GFKHILPLT | AGUACAGCAUGAGCACCGUGCUGGAGUACCCUACCAUCG | | | |
| | | DDAERFNEI | GCCAGCUGAUCGACAAGCUUGUGCAGAAUAACGUGCUGC | | | |
| | | VRKQKISAN | UGAUCUUCGCCGUGACCCAGGAGCAGGUCCACCUGUACG | | | |
| | | IDTPEGGED | AGAACUACGCCAAGCUGAUCCCUGGCGCCACCGUGGGCC | | | |
| | | AIMQAAVCK | UGCUGCAGAAGGACAGCGGCAACAUCUUACAGCUGAUUA | | | |
| | | EKIGWRNDS | UAUCAGCCUACGAGGAGCUGAGAAGCGAGGUGGAGCUGG | | | |
| | | LHLLVFVSD | AGGUGCUGGGCGACACCGAGGGCCUGAACCUGAGCUUCA | | | |
| | | ADSHFGMDS | CCGCCCUGUGCAGCAACGGCAUCCUGUUCCCUCACCAGA | | | |
| | | KLAGIVIPN | AGAAGUGUUCCCACAUGAAGGUCGGUGAUACAGCAUCAU | | | |
| | | DGLCHLDNR | UCAACGUGAGCGUGAGCAUUACCAAUUGCGAGAAGAGAA | | | |
| | | NEYSMSTVL | GCAGAAAGUUAAUAAUUAAGCCUGUCGGACUGGGCGAUA | | | |
| | | EYPTIGQLI | CUCUUGAAAUCCUAGUGAGCGCCGAGUGCGACUGUGAUU | | | |
| | | DKLVQNNVL | GCCAGAGAGAGGUGGAAGCCAAUUCUUCCAAGUGCCACC | | | |
| | | LIFAVTQEQ | ACGGCAACGGAUCCUUCCAGUGCGGCGUGUGCGCCUGCA | | | |
| | | VHLYENYAK | ACCCUGGCCACAUGGGCCCUCGAUGCGAGUGUGGAGAGG | | | |
| | | LIPGATVGL | ACAUGGUUUCUACCGACAGCUGCAAGGAAAGCCCAGGCC | | | |
| | | LQKDSGNIL | AUCCUAGCUGCAGCGGCAGAGGCGACUGCUACUGCGGCC | | | |
| | | QLIISAYEE | AGUGCGUGUGCCAUCUGAGUCCUUACGGCAGCAUCUACG | | | |
| | | LRSEVELEV | GCCCUUACUGUCAGUGCGAUAACUUCUCGUGCCUGAGAC | | | |
| | | LGDTEGLNL | ACAAGGGUUUGCUGUGCGGUGACAACGGAGAUUGUGACU | | | |
| | | SFTALCSNG | GCGGAGAGUGCGUGUGUAGAGACGGCUGGACCGGCGAGU | | | |
| | | ILFPHQKKC | ACUGCAACUGCACCACCAGCAGGGACGCCUGCGCCAGCG | | | |
| | | SHMKVGDTA | AGGACGGCGUGUUGUGUUCUGGACGUGGUGAUUGUGUGU | | | |
| | | SFNVSVSIT | GUGGCAAGUGCGUCUGCAGAAACCCGGGAGCCAGCGGCC | | | |
| | | NCEKRSRKL | CUACCUGUGAGAGGUGCCCAACUUGUGGCGAUCCUUGCA | | | |
| | | IIKPVGLGD | AUUCACGUAGGAGCUGCAUCGAGUGCUACUUGAGCGCUG | | | |
| | | TLEILVSAE | ACGGCCAGGCCCAGGAGGAGUGCGAGGAUAAGUGCAAGG | | | |
| | | CDCDCQREV | CCACCGGAGCAACUAUCUCUGAGGAAGAAUUCAGCAAGG | | | |
| | | EANSSKCHH | ACACCAGCGUGCCUUGCAGUUUGCAGGGAGAGAACGAGU | | | |
| | | GNGSFQCGV | GCCUGAUCACCUUCCUCAUCACCGCCGACAACGAGGGCA | | | |
| | | CACNPGHMG | AGACCAUCAUCCACAAUAUUAGCGAGAAGGACUGCCCUA | | | |
| | | PRCECGEDM | AGCCUCCUAACAUCCCUAUGAUCAUGCUGGGAGUGAGCU | | | |
| | | VSTDSCKES | UGGCUAUCCUGCUUAUCGGCGUGGUCUUGCUGUGUAUCU | | | |
| | | PGHPSCSGR | GGAAGCUCCUCGUCAGUUUCCACGACAGAAAGGAGGUGG | | | |
| | | GDCYCGQCV | CCAAGUUCGAGGCUGAAAGAUCUAAGGCCAAGUGGCAGA | | | |
| | | CHLSPYGSI | CCGGAACCAAUCCUCUGUACAGAGGCAGCACCAGCACCU | | | |
| | | YGPYCQCDN | UCAAGAACGUGACCUACAAGCACCGGGAGAAGCACAAGG | | | |
| | | FSCLRHKGL | UCGGCCUGAGUAGCGACGGCGGCAAGCCUAUUCCAAACC | | | |
| | | LCGDNGDCD | CACUGCUCGGUCUGGACUCAACC | | | |
| | | CGECVCRDG | | | | |
| | | WTGEYCNCT | | | | |
| | | TSRDACASE | | | | |
| | | DGVLCSGRG | | | | |
| | | DCVCGKCVC | | | | |
| | | RNPGASGPT | | | | |
| | | CERCPTCGD | | | | |
| | | PCNSRRSCI | | | | |
| | | ECYLSADGQ | | | | |
| | | AQEECEDKC | | | | |
| | | KATGATISE | | | | |
| | | EEFSKDTSV | | | | |
| | | PCSLQGENE | | | | |
| | | CLITFLITA | | | | |
| | | DNEGKTIIH | | | | |
| | | NISEKDCPK | | | | |
| | | PPNIPMIML | | | | |
| | | GVSLAILLI | | | | |
| | | GVVLLCIWK | | | | |
| | | LLVSFHDRK | | | | |
| | | EVAKFEAER | | | | |
| | | SKAKWQTGT | | | | |
| | | NPLYRGSTS | | | | |
| | | TFKNVTYKH | | | | |
| | | REKHKVGLS | | | | |
| | | SDGGKPIPN | | | | |
| | | PLLGLDST | | | | |
| | | 15 | 16 | 56 | 110 | 174 |
| Mouse | ITB6 | MGIELVCLF | AUGGGCAUCGAGCUGGUGUGCCUGUUCCUGCUACUCUUA | GGAAAUAA | UGAUAAUA | SEQ ID |
| Var- | G5 | LLLLGRNDH | GGCAGAAACGACCACGUGCAAGGCGGCUGCGCCUGGGGA | GAGAGAAA | GGCUGGAG | NO: 174 |
| iant | Cap II-b | VQGGCAWGG | GGUGCCGAGAGCUGCAGCGACUGCCUGCUGACCGGCCCU | AGAAGAGU | CCUCGGUG | consists |
| 1.1 | Poly A | AESCSDCLL | CACUGUGCGUGGUGCAGCCAGGAGAACUUCACCCACCUG | AAGAAGAA | GCCUAGCU | from 5′ |
| | Tail: | TGPHCAWCS | AGUGGCGCCGGCGAGCGAUGCGACACCCCUGCCAACCUG | AUAUAAGA | UCUUGCCC | to 3′ |
| | 100 nt | QENFTHLSG | CUGGCCAAGGGCUGCCAGCUGCCUUUCAUCGAGAACCCU | CCCCGGCG | CUUGGGCC | end: 5′ |
| | (SEQ ID | AGERCDTPA | GUGAGCAGAAUCGAGGUGCUGCAGAACAAGCCUCUGAGC | CCGCCACC | UCCCCCCA | UTR of |
| | NO: 502) | NLLAKGCQL | GUGGGCAGACAGAAGAACAGCAGCGACAUCGUGCAGAUC | | GCCCCUCC | SEQ ID |
| | | PFIENPVSR | GCCCCUCAGUCACUUGUACUGAAGUUGCGGCCUGGCAGA | | UCCCCUUC | NO: 56, |
| | | IEVLONKPL | GAGCAGACCCUGCAGGUGCAGGUGAGACAGACCGAGGAC | | CUGCACCC | ORF |
| | | SVGRQKNSS | UACCCUGUGGACCUGUACUACCUGAUGGAUCUGAGCGCC | | GUACCCCC | sequence |
| | | DIVQIAPQS | AGCAUGGACGACGACCUGAACACCAUCAAGGAACUGGGU | | CAAACACC | of SEQ |
| | | LVLKLRPGR | AGCAGACUGGCUAAGGAGAUGAGCAAGCUGACCAGCAAC | | AUUGUCAC | ID NO: |
| | | EQTLQVQVR | UUCAGACUGGGCUUCGGCAGCUUCGUGGAGAAGCCUGUU | | ACUCCAGU | 16, and |
| | | QTEDYPVDL | UCACCUUUCAUGAAGACCACCCCUGAGGAGAUCACCAAC | | GGUCUUUG | 3′ UTR |
| | | YYLMDLSAS | CCUUGCAGCAGCAUCCCUUACUUCUGCCUGCCUACCUUC | | AAUAAAGU | sequence |
| | | MDDDLNTIK | GGCUUCAAGCACAUCCUGCCUCUGACCGACGACGCGGAG | | CUGAGUGG | of SEQ |
| | | ELGSRLAKE | AGAUUCAACGAGAUCGUGAGAAAGCAGAAGAUCAGCGCC | | GCGGC | ID NO: |
| | | MSKLTSNFR | AACAUUGACACCCCGGAAGGCGGAUUCGACGCCAUCAUG | | | 110. |
| | | LGFGSFVEK | CAGGCCGCCGUGUGCAAGGAGAAGAUCGGCUGGAGAAAC | | | |
| | | PVSPFMKTT | GAUAGCCUGCAUUUGUUAGUGUUCGUGAGUGACGCAGAC | | | |
| | | PEEITNPCS | AGCCACUUCGGCAUGGACUCCAAGCUUGCCGGCAUCGUG | | | |
| | | SIPYFCLPT | AUCCCUAACGACGGCCUGUGCCACCUGGACCACAGGAAC | | | |
| | | FGFKHILPL | GAGUACAGCAUGAGCACCGUGCUGGAGUACCCUACCAUC | | | |
| | | TDDAERFNE | GGCCAGCUGAUCGACAAGCUGGUCCAGAAUAACGUGCUG | | | |
| | | IVRKQKISA | CUGAUCUUCGCCGUGACCCAGGAACAGGUUCACCUGUAC | | | |
| | | NIDTPEGGF | GAGAACUACGCCAAGCUGAUCCCUGGCGCCACCGUGGGC | | | |
| | | DAIMQAAVC | CUCCUGCAGAAGGACAGCGGCAACAUCCUCCAACUGAUC | | | |
| | | KEKIGWRND | AUAUCUGCUUACGAGGAGCUGAGAAGCGAGGUGGAGCUG | | | |
| | | SLHLLVFVS | GAGGUGCUCGGCGACACCGAGGGCCUGAACCUGAGCUUC | | | |
| | | DADSHFGMD | ACCGCCCUGUGCAACAACGGCGUGCUGUUCCCUCACCAG | | | |
| | | SKLAGIVIP | AAGAAGUGUAGUCACAUGAAGGUUGGCGAUACCGCCAGC | | | |
| | | NDGLCHLDH | UUCAACGUGACCGUGAGCGUGAGCAACUGCGAGAAGAGA | | | |
| | | RNEYSMSTV | AGCAGAAACCUUAUAAUAAAGCCAGUUGGUCUGGGCGAC | | | |
| | | LEYPTIGQL | ACACUGGAGAUCCUGGUGAGCGCCGAGUGCGACUGCGAU | | | |
| | | IDKLVQNNV | UGUCAGAGAGAGAUCGAGACAAAUUCGUCUAAGUGCCAC | | | |
| | | LLIFAVTQE | AACGGCAACGGAUCUUUCCAGUGCGGCGUGUGCACCUGC | | | |
| | | QVHLYENYA | AACCCUGGCCACAUGGGACCACACUGCGAGUGCGGAGAG | | | |
| | | KLIPGATVG | GACAUGGUGUCAACCGACAGCUGUAAGGAGAGCCCAGGC | | | |
| | | LLQKDSGNI | CAUCCUUCUUGCUCCGGCAGAGGCGACUGCUACUGCGGC | | | |
| | | LQLIISAYE | CAGUGCAUCUGCCAUCUCUCCCCUUACGGCAGCAUCUAC | | | |
| | | ELRSEVELE | GGCCCUUACUGCCAGUGCGAUAACUUCUCGUGCCUGAGA | | | |
| | | VLGDTEGLN | CACAAGGGCCUGCUCUGCGGCGACAACGGCGACUGUGAU | | | |
| | | LSFTALCNN | UGUGGUGAGUGUGUGUGCAGAGACGGCUGGACCGGCGAG | | | |
| | | GVLFPHQKK | UACUGCAACUGCACCACCAACAGAGAUAGCUGUACCAGC | | | |
| | | CSHMKVGDT | GAGGACGGUGUCCUCUGUUCCGGACGGGGUGAUUGCGUU | | | |
| | | ASFNVTVSV | UGUGGCAAGUGUGUUUGCCGUAACCCAGGAGCCAGCGGC | | | |
| | | SNCEKRSRN | CCUACCUGCGAAAGGUGCCCGACCUGUGGAGAUCCGUGU | | | |
| | | LIIKPVGLG | AACAGCAAGCGAAGUUGCAUCGAGUGCUAUCUCUCCGCA | | | |
| | | DTLEILVSA | GACGGCCAGGCCCAGGAGGAGUGCGCCGACAAGUGCAAG | | | |
| | | ECDCDCQRE | GCCAUCGGCGCUACUAUUUCGGAAGAGGACUUCAGCAAG | | | |
| | | IETNSSKCH | GACACCAGCGUUAGCUGUUCGCUGCAGGGUGAGAACGAG | | | |
| | | NGNGSFQCG | UGCCUGAUCACCUUCCUCAUCACGACCGACAACGAGGGC | | | |
| | | VCTCNPGHM | AAGACCAUCAUCCACAACAUCAACGAGAAGGACUGCCCU | | | |
| | | GPHCECGED | AAGCCUCCUAACAUCCCUAUGAUCAUGCUGGGAGUGAGC | | | |
| | | MVSTDSCKE | CUGGCCAUACUGCUUAUCGGCGUGGUUCUGCUGUGCAUU | | | |
| | | SPGHPSCSG | UGGAAGUUAUUAGUGAGUUUCCACGACAGAAAGGAGGUG | | | |
| | | RGDCYCGQC | GCCAAGUUCGAGGCCGAGAGGUCCAAGGCCAAGUGGCAG | | | |
| | | ICHLSPYGS | ACCGGCACGAACCCACUGUACAGAGGCAGCACCAGCACC | | | |
| | | IYGPYCQCD | UUCAAGAACGUCACAUACAAGCACCGGGAGAAGCACAAG | | | |
| | | NFSCLRHKG | GCCGGCCUGUCAUCUGACGGCGGCAAGCCUAUCCCGAAU | | | |
| | | LLCGDNGDC | CCACUCCUAGGACUGGAUAGUACC | | | |
| | | DCGECVCRD | | | | |
| | | GWTGEYCNC | | | | |
| | | TTNRDSCTS | | | | |
| | | EDGVLCSGR | | | | |
| | | GDCVCGKCV | | | | |
| | | CRNPGASGP | | | | |
| | | TCERCPTCG | | | | |
| | | DPCNSKRSC | | | | |
| | | IECYLSADG | | | | |
| | | QAQEECADK | | | | |
| | | CKAIGATIS | | | | |
| | | EEDFSKDTS | | | | |
| | | VSCSLQGEN | | | | |
| | | ECLITFLIT | | | | |
| | | TDNEGKTII | | | | |
| | | HNINEKDCP | | | | |
| | | KPPNIPMIM | | | | |
| | | LGVSLAILL | | | | |
| | | IGVVLLCIW | | | | |
| | | KLLVSFHDR | | | | |
| | | KEVAKFEAE | | | | |
| | | RSKAKWQTG | | | | |
| | | TNPLYRGST | | | | |
| | | STFKNVTYK | | | | |
| | | HREKHKAGL | | | | |
| | | SSDGGKPIP | | | | |
| | | NPLLGLDST | | | | |

Note:
“G5” indicates that all uracils (U) in the mRNA are replaced by N1-methylpseudouracils.
Signal sequence underlined; tag sequence italicized and underlined

### Lipid Content of LNPs

As set forth above, with respect to lipids, LNPs disclosed herein comprise an (i) ionizable lipid; (ii) sterol or other structural lipid; (iii) a non-cationic helper lipid or phospholipid; and a (iv) PEG lipid along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Nucleic acids of the present disclosure (e.g., ITB6 mRNA) are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 40-50 mol %, optionally 45-50 mol %, for example, 45-46 mol %, 46-47 mol %, 47-48 mol %, 48-49 mol %, or 49-50 mol %, for example about 45 mol %, 45.5 mol %, 46 mol %, 46.5 mol %, 47 mol %, 47.5 mol %, 48 mol %, 48.5 mol %, 49 mol %, or 49.5 mol % ionizable cationic lipid.

In some embodiments, the lipid nanoparticle comprises a molar ratio of 5-25% non-cationic lipid. For example, the lipid nanoparticle may comprise a molar ratio of 5-15 mol %, optionally 10-12 mol %, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % non-cationic lipid. In some embodiments, the lipid nanoparticle comprises a molar ratio of 25-55% sterol.

For example, the lipid nanoparticle may comprise a molar ratio of 30-45 mol %, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol % sterol.

In some embodiments, the lipid nanoparticle comprises a molar ratio of 0.5-15% PEG-modified lipid. For example, the lipid nanoparticle may comprise a molar ratio of 1-5%, optionally 1-3 mol %, for example 1.5 to 2.5 mol %, 1-2 mol %, 2-3 mol %, 3-4 mol %, or 4-5 mol % PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the lipid nanoparticle comprises a molar ratio of 40-50% ionizable cationic lipid, 5-15% non-cationic lipid, 30-45% sterol, and 1-5% PEG-modified lipid.

In some embodiments, the lipid nanoparticle comprises a molar ratio of 45-50% ionizable cationic lipid, 10-12% non-cationic lipid, 35-40% sterol, and 1-3% PEG-modified lipid. In some embodiments, the lipid nanoparticle comprises a molar ratio of 45-50% ionizable cationic lipid, 10-12% non-cationic lipid, 35-40% sterol, and 1.5-2.5% PEG-modified lipid.

### Ionizable Amino Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs. The structures of ionizable lipids set forth below include the prefix I to distinguish them from other lipids of the invention.

In some aspects, the disclosure relates to a compound of Formula (I):

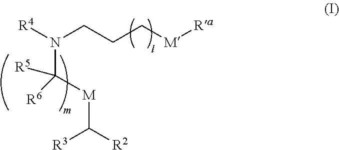

or its N-oxide, or a salt or isomer thereof,
wherein R′^{a} is R′^{branched}, wherein
   R′^{branched} is:

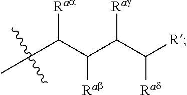

wherein

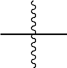

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

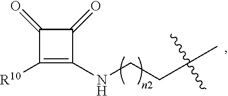

wherein

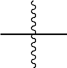

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (I), R′^{a} is R′^{branched}; R′^{branched} is

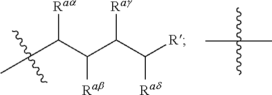

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of the compounds of Formula (I), R′^{a} is R′^{branched}; R′^{branched} is

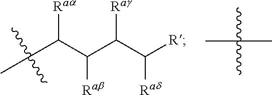

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of the compounds of Formula (I), R′^{a} is R′^{branched}; R′^{branched} is

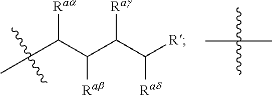

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

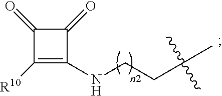

R¹⁰ is NH(C₁₋₆ alkyl); n2 is 2; R⁵ is H; each R⁶ is H; M and M′ are each —C(O)O—; R′ is a C₁₋₁₂ alkyl; l is 5; and m is 7.

In some embodiments of the compounds of Formula (I), R′^{a} is R′^{branched}; R′^{branched} is

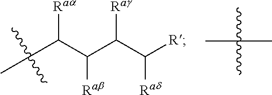

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the compound of Formula (I) is selected from:

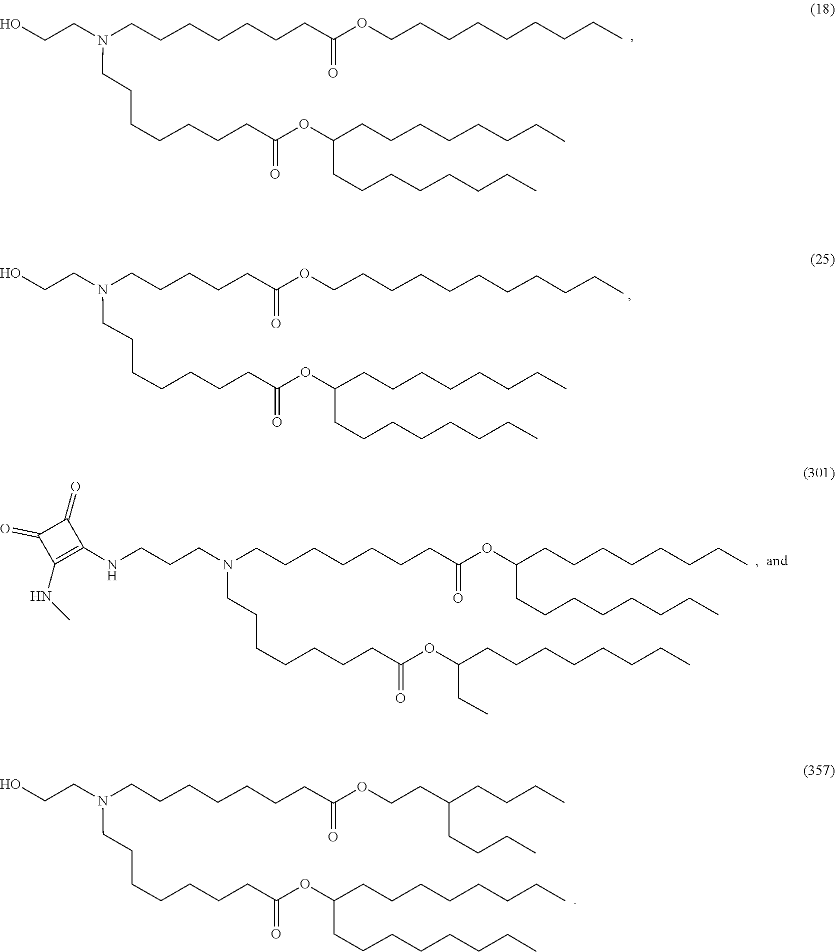 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the compound of Formula (I) is:

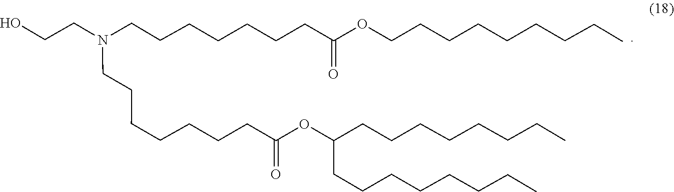 [SMILES: CCCCCCCCCOC(=O)CCCCCCCN(CCO)CCCCCCCC(=O)OC(CCCCCCCC)CCCCCCCC]

In some embodiments, the compound of Formula (I) is:

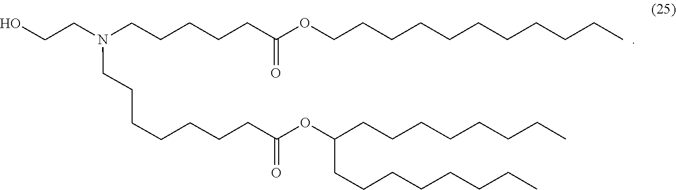 [SMILES: CCCCCCCCCCCOC(=O)CCCCCN(CCO)CCCCCCCC(=O)OC(CCCCCCCC)CCCCCCCC]

In some embodiments, the compound of Formula (I) is:

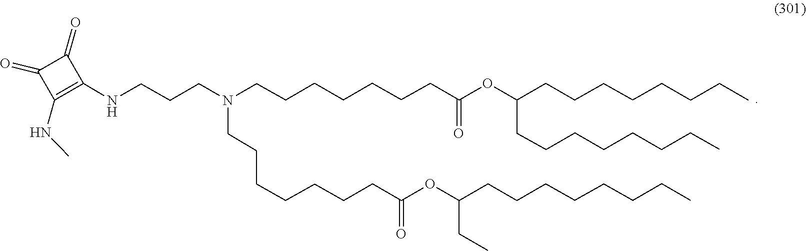 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the compound of Formula (I) is:

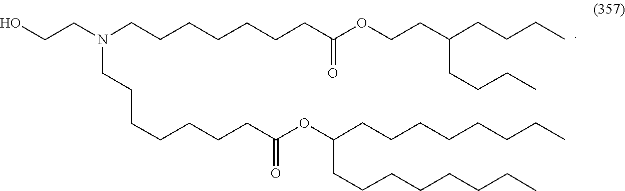 [SMILES: CCCCCCCCC(CCCCCCCC)OC(=O)CCCCCCCN(CCO)CCCCCCCC(=O)OCCC(CCCC)CCCC]

In some aspects, the disclosure relates to a compound of Formula (I-a):

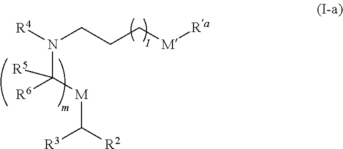

or its N-oxide, or a salt or isomer thereof,
wherein R′^{a} is R′^{branched}; wherein
   R′^{branched} is:

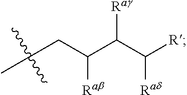

wherein

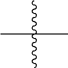

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4 and 5, and

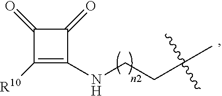

wherein

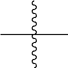

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some aspects, the disclosure relates to a compound of Formula (I-b):

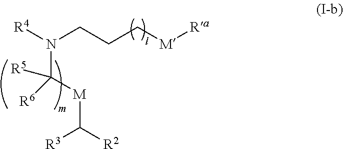

or its N-oxide, or a salt or isomer thereof,
wherein R′^{a} is R′^{branched}; wherein
   R′^{branched} is:

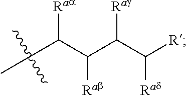

wherein

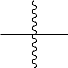

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (I) or (I-b), R′^{a} is R′^{branched}; R′^{branched} as

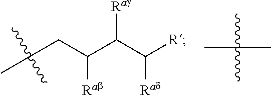

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of Formula (I) or (I-b), R′^{a} is R′^{branched}; R′^{branched} is

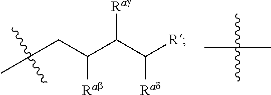

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of Formula (I) or (I-b), R′^{a} is R′^{branched}; R′^{branched} is

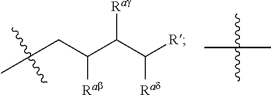

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some aspects, the disclosure relates to a compound of Formula (I-c):

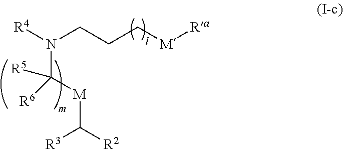

or its N-oxide, or a salt or isomer thereof,
wherein R′^{a} is R′^{branched}; wherein
   R′^{branched} is:

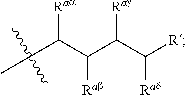

wherein

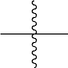

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

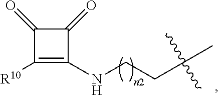

wherein

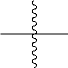

denotes a point of attachment; wherein
R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

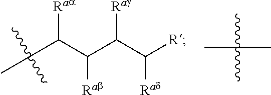

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

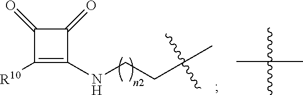

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; l is 5; and m is 7.

In some embodiments, the compound of Formula (I-c) is:

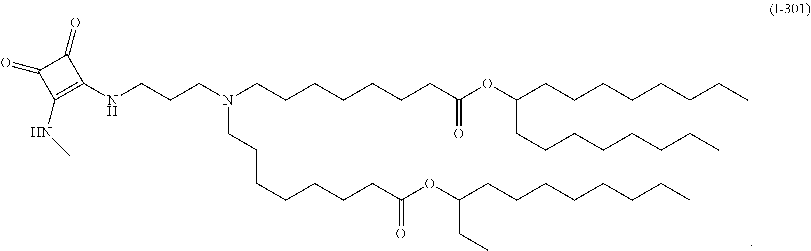 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some aspects, the disclosure relates to a compound of Formula (II):

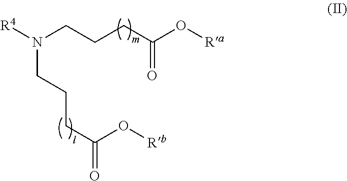

or its N-oxide, or a salt or isomer thereof,
wherein R′^{a} is R′^{branched} or R′^{cyclic}; wherein
R′^{branched} is:

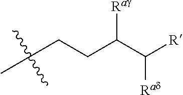

and R′^{cyclic} is:

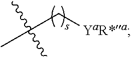

and
R′b is:

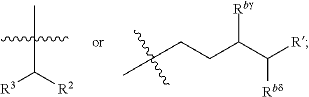

wherein

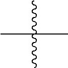

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

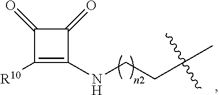

wherein

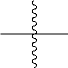

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-a):

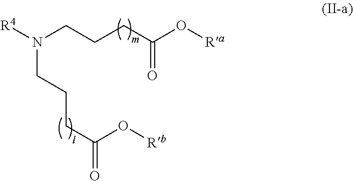

or its N-oxide, or a salt or isomer thereof,
wherein R′^{a} is R′^{branched} or R′^{cyclic}; wherein
R′^{branched} is:

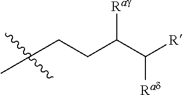

and R′^{b} is:

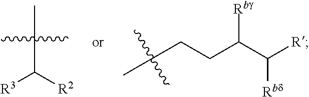

wherein

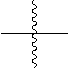

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

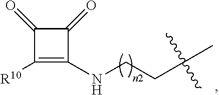

wherein

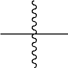

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-b):

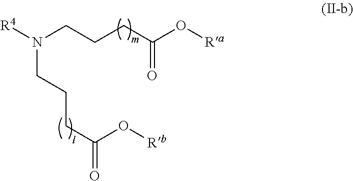

or its N-oxide, or a salt or isomer thereof,
wherein R′^{a} is R′^{branched} or R′^{cyclic}; wherein
R′^{branched} is:

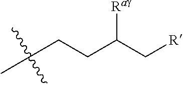

and R′^{b} is:

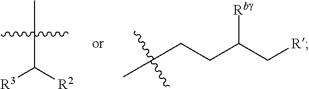

wherein

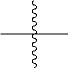

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

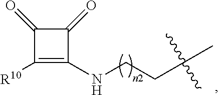

wherein

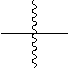

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-c):

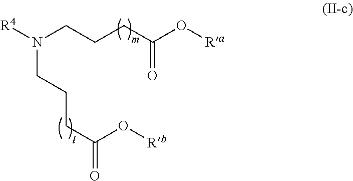

or its N-oxide, or a salt or isomer thereof,
wherein R′^{a} is R′^{branched} or R′^{cyclic}; wherein
R′^{branched} is:

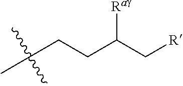

and R′^{b} is:

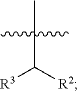

wherein

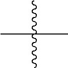

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

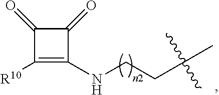

wherein

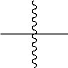

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-d):

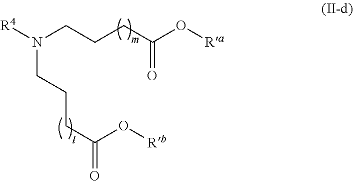

or its N-oxide, or a salt or isomer thereof,
wherein R′^{a} is R′^{branched} or R′^{cyclic}; wherein
R′^{branched} is:

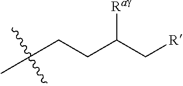

and R′^{b} is:

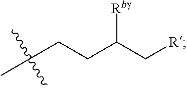

wherein

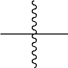

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

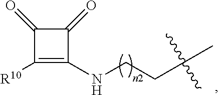

wherein

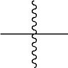

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-e):

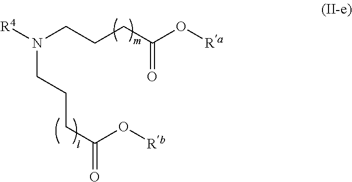

or its N-oxide, or a salt or isomer thereof,
wherein R′^{a} is R′^{branched} or R′^{cyclic}; wherein
R′^{branched} is:

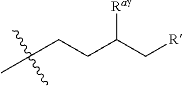

and R′^{b} is:

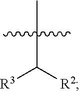

wherein

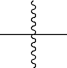

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (II), (II-a), (II-b), (II-c), (II-d), or (II-e), m and l are each independently selected from 4, 5, and 6. In some embodiments of the compound of Formula (II), (II-a), (II-b), (II-c), (II-d), or (II-e), m and l are each 5.

In some embodiments of the compound of Formula (II), (II-a), (II-b), (II-c), (II-d), or (II-e), each R′ independently is a C₁₋₁₂ alkyl. In some embodiments of the compound of Formula (II), (II-a), (II-b), (II-c), (II-d), or (II-e), each R′ independently is a C₂₋₅ alkyl.

In some embodiments of the compound of Formula (II), (II-a), (II-b), (II-c), (II-d), or (II-e), R′^{b} is:

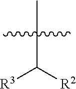

and R² and R³ are each independently a C₁₋₁₄ alkyl. In some embodiments of the compound of Formula (II), (II-a), (II-b), (II-c), (II-d), or (II-e), R′^{b} is:

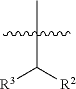

and R² and R³ are each independently a C₆₋₁₀ alkyl. In some embodiments of the compound of Formula (II), (II-a), (II-b), (II-c), (II-d), or (II-e), R′^{b} is:

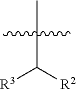

and R² and R³ are each a C₈ alkyl.

In some embodiments of the compound of Formula (II), (II-a), (II-b), (II-c), (II-d), or (II-e), R′^{branched} is:

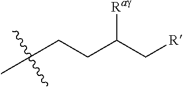

### and R′^{b} is:

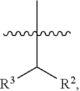

R^{aγ} is a C₁₋₁₂ alkyl and R² and R³ are each independently a C₆₋₁₀ alkyl. In some embodiments of the compound of Formula (II), (II-a), (II-b), (II-c), (II-d), or (II-e), R′^{branched} is:

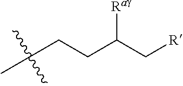

### and R′^{b} is:

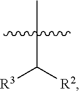

R^{aγ} is a C₂₋₆ alkyl and R² and R³ are each independently a C₆₋₁₀ alkyl. In some embodiments of the compound of Formula (II), (II-a), (II-b), (II-c), (II-d), or (II-e), R′^{branched} is:

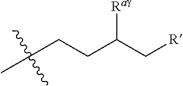

### and R′^{b} is:

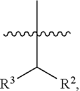

R^{aγ} is a C₂₋₆ alkyl, and R² and R³ are each a C₈ alkyl.

In some embodiments of the compound of Formula (II), (II-a), (II-b), (II-c), (II-d), or (II-e), R′^{branched} is:

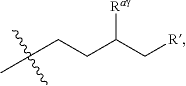

### R′^{b} is:

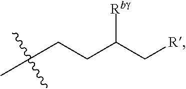

and R^{aγ} and R^{bγ} are each a C₁₋₁₂ alkyl.

In some embodiments of the compound of Formula (II), (II-a), (II-b), (II-c), (II-d), or (II-e), R′^{branched} is:

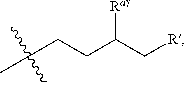

### R′^{b} is:

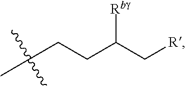

and R^{aγ} and R^{bγ} are each a C₂₋₆ alkyl.

In some embodiments of the compound of Formula (II), (II-a), (II-b), (II-c), (II-d), or (II-e), m and l are each independently selected from 4, 5, and 6 and each R′ independently is a C₁₋₁₂ alkyl. In some embodiments of the compound of Formula (II), (II-a), (II-b), (II-c), (II-d), or (II-e), m and l are each 5 and each R′ independently is a C₂₋₅ alkyl.

In some embodiments of the compound of Formula (II), (II-a), (II-b), (II-c), (II-d), or (II-e), R′^{branched} is:

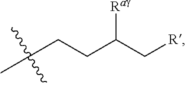

### R′^{b} is:

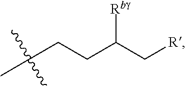

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (II), (II-a), (II-b), (II-c), (II-d), or (II-e), R′^{branched} is:

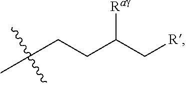

### R′^{b} is:

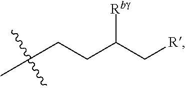

m and l are each 5, each R′ independently is a C₂₋₅ alkyl, and R^{aγ} and R^{bγ} are each a C₂₋₆ alkyl.

In some embodiments of the compound of Formula (II), (II-a), (II-b), (II-c), (II-d), or (II-e), R′^{branched} is:

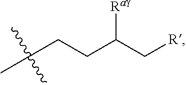

### and R′^{b} is:

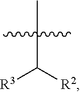

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl. In some embodiments of the compound of Formula (II), (II-a), (II-b), (II-c), (II-d), or (II-e), R′^{branched} is:

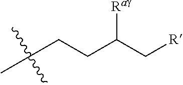

### and R′^{b} is:

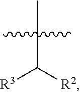

m and l are each 5, R′ is a C₂₋₅ alkyl, R^{aγ} is a C₂₋₆ alkyl, and R² and R³ are each a C₈ alkyl.

In some embodiments of the compound of Formula (II), (II-a), (II-b), (II-c), (II-d), or (II-e), R⁴ is

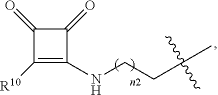

wherein R¹⁰ is NH(C₁₋₆ alkyl) and n2 is 2. In some embodiments of the compound of Formula (II), (II-a), (II-b), (II-c), (II-d), or (II-e), R⁴ is

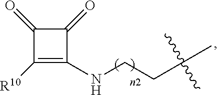

wherein R¹⁰ is NH(CH₃) and n2 is 2.

In some embodiments of the compound of Formula (II), (II-a), (II-b), (II-c), (II-d), or (II-e), R′^{branched} is:

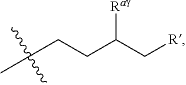

### R′^{b} is:

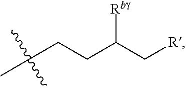

m and l are each independently selected from 4, 5, and 6, each R′ independently is a C₁₋₁₂ alkyl, R^{aγ} and R^{bγ} are each a C₁₋₁₂ alkyl, and R⁴ is

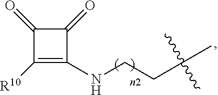

wherein R¹⁰ is NH(C₁₋₆ alkyl), and n2 is 2. In some embodiments of the compound of Formula (II), (II-a), (II-b), (II-c), (II-d), or (II-e), R′^{branched} is:

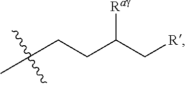

### R′^{b} is:

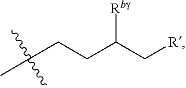

m and l are each 5, each R′ independently is a C₂₋₅ alkyl, R^{aγ} and R^{bγ} are each a C₂₋₆ alkyl, and R⁴ is

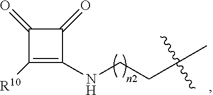

wherein R¹⁰ is NH(CH₃) and n2 is 2.

In some embodiments of the compound of Formula (II), (II-a), (II-b), (II-c), (II-d), or (II-e), R′^{branched} is:

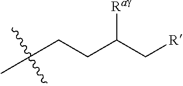

### and R′^{b} is:

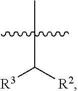

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

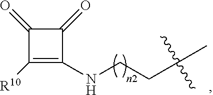

wherein R¹⁰ is NH(C₁₋₆ alkyl) and n2 is 2. In some embodiments of the compound of Formula (II), (II-a), (II-b), (II-c), (II-d), or (II-e), R′^{branched} is:

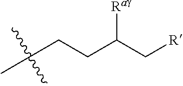

### and R′^{b} is:

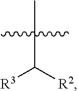

m and l are each 5, R′ is a C₂₋₅ alkyl, R^{aγ} is a C₂₋₆ alkyl, R² and R³ are each a C₈ alkyl, and R⁴ is

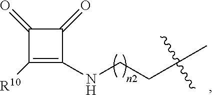

wherein R¹⁰ is NH(CH₃) and n2 is 2.

In some embodiments of the compound of Formula (II), (II-a), (II-b), (II-c), (II-d), or (II-e), R⁴ is —(CH₂)ₙOH and n is 2, 3, or 4. In some embodiments of the compound of Formula (II), (II-a), (II-b), (II-c), (II-d), or (II-e), R⁴ is —(CH₂)ₙOH and n is 2.

In some embodiments of the compound of Formula (II), (II-a), (II-b), (II-c), (II-d), or (II-e), R′^{branched} is:

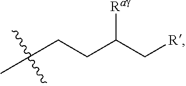

### R′^{b} is:

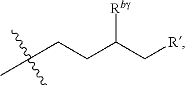

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (II), (II-a), (II-b), (II-c), (II-d), or (II-e), R′^{branched} is:

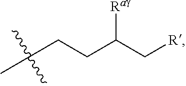

### R′^{b} is:

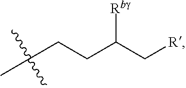

m and l are each 5, each R′ independently is a C₂₋₅ alkyl, R^{aγ} and R^{bγ} are each a C₂₋₆ alkyl, R⁴ is —(CH₂)ₙOH, and n is 2.

In some aspects, the disclosure relates to a compound of Formula (II-f):

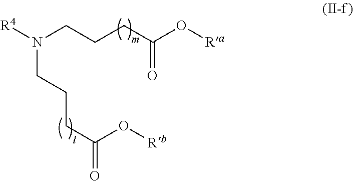

or its N-oxide, or a salt or isomer thereof,
wherein R′^{a} is R′^{branched} or R′^{cyclic}; wherein
R′^{branched} is:

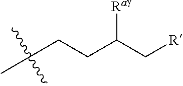

and R′^{b} is:

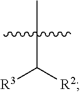

wherein

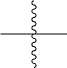

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (II-f), m and l are each 5, and n is 2, 3, or 4.

In some embodiments of the compound of Formula (II-f) R′ is a C₂₋₅ alkyl, R^{aγ} is a C₂₋₆ alkyl, and R² and R³ are each a C₆₋₁₀ alkyl.

In some embodiments of the compound of Formula (II-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some aspects, the disclosure relates to a compound of Formula (II-g):

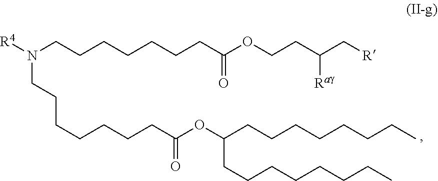

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

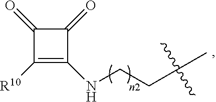

wherein

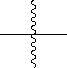

denotes a point of attachment, R¹⁰ is NH(C₁₋₆ alkyl), and n2 is selected from the group consisting of 1, 2, and 3.

In some aspects, the disclosure relates to a compound of Formula (II-h):

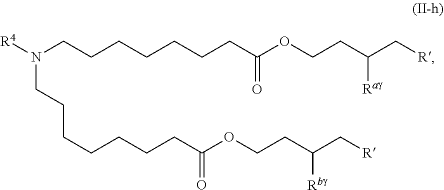

wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

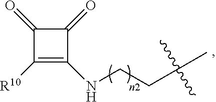

wherein

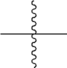

denotes a point of attachment, R¹⁰ is NH(C₁₋₆ alkyl), and n2 is selected from the group consisting of 1, 2, and 3.

In some embodiments of the compound of Formula (II-g) or (II-h), R⁴ is

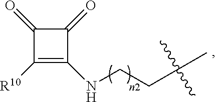

wherein
R¹⁰ is NH(CH₃) and n2 is 2.

In some embodiments of the compound of Formula (II-g) or (II-h), R⁴ is —(CH₂)₂OH.

In some aspects, the disclosure relates to a compound having the Formula (III):

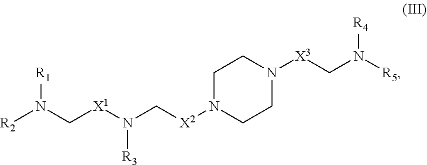

or a salt or isomer thereof, wherein
R¹, R², R³, R⁴, and R⁵ are independently selected from the group consisting of C₅₋₂₀ alkyl, C₅₋₂₀ alkenyl, —R″MR′, —R*YR″, —YR″, and —R*OR″;
each M is independently selected from the group consisting
of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl, and
wherein:
i) at least one of X¹, X², and X³ is not —CH₂—; and/or
ii) at least one of R¹, R², R³, R⁴, and R⁵ is —R″MR′.

In some embodiments, R¹, R², R³, R⁴, and R⁵ are each C₅₋₂₀ alkyl; X¹ is —CH₂—; and X² and X³ are each —C(O)—.

In some embodiments, the compound of Formula (III) is:

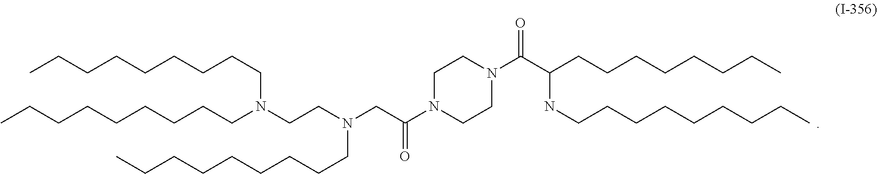 [SMILES: CCCCCCCCCNC(CCCCCCCC)C(=O)N1CCN(C(=O)CN(CCCCCCCCC)CCN(CCCCCCCCC)CCCCCCCCC)CC1]

The central amine moiety of a lipid according to any of the Formulae herein, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the amount the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) ranges from about 1 mol % to 99 mol % in the lipid composition.

In one embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) is about 45 mol % in the lipid composition.

In one specific embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) is about 40 mol % in the lipid composition.

In one specific embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) is about 50 mol % in the lipid composition.

In addition to the ionizable amino lipid disclosed herein, e.g. a compound having any of Formula (I), (I-a), (I-b), (I-c), (II), (II-a), (II-b), (II-c), (II-d), (II-e), (II-f), (II-g), (II-h), or (III), (each of these preceded by the letter I for clarity) the lipid-based composition (e.g., lipid nanoparticle) disclosed herein can comprise additional components such as cholesterol and/or cholesterol analogs, non-cationic helper lipids, structural lipids, PEG-lipids, and any combination thereof.

Additional ionizable lipids of the invention can be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)—N,N-dimethyl-3-nonydocosa-13-16-dien-1-amine (L608), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-die n-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids of the invention can also be the compounds disclosed in International Publication No. WO 2017/075531 A1, hereby incorporated by reference in its entirety.

Ionizable lipids of the invention can also be the compounds disclosed in International Publication No. WO 2015/199952 A1, hereby incorporated by reference in its entirety.

In any of the foregoing or related aspects, the ionizable lipid of the LNP of the disclosure comprises a compound included in any e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity).

In any of the foregoing or related aspects, the ionizable lipid of the LNP of the disclosure comprises a compound comprising any of Compound Nos. 18, 25, 301, and 357.

In any of the foregoing or related aspects, the ionizable lipid of the LNP of the disclosure comprises at least one compound selected from the group consisting of: Compound Nos. 18, 25, 301, and 357. In another embodiment, the ionizable lipid of the LNP of the disclosure comprises a compound selected from the group consisting of. Compound Nos. 18, 25, 301, and 357. In another embodiment, the ionizable lipid of the LNP of the disclosure comprises Compound 18. In another embodiment, the ionizable lipid of the LNP of the disclosure comprises Compound 25.

In any of the foregoing or related aspects, the synthesis of compounds of the invention, e.g. compounds comprising any of Compound Nos. 18, 25, 301, and 357, follows the synthetic descriptions in U.S. Provisional Patent Application No. 62/733,315, filed Sep. 19, 2018.

### Representative Synthetic Routes:

Compound I-182: Heptadecan-9-yl 8-((3-((2-(methylamino)-3,4-dioxocyclobut-1-en-1-yl)amino)propyl)(8-(nonyloxy)-8-oxooctyl)amino)octanoate 3-Methoxy-4-(methylamino)cyclobut-3-ene-1,2-dione

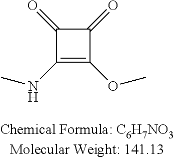

To a solution of 3,4-dimethoxy-3-cyclobutene-1,2-dione (1 g, 7 mmol) in 100 mL diethyl ether was added a 2M methylamine solution in THF (3.8 mL, 7.6 mmol) and a precipitate formed. The mixture was stirred at room temperature for 24 hours, then filtered to collect the solid. The solid was washed with diethyl ether and air-dried, then dissolved in hot EtOAc and filtered. The filtrate was allowed to cool to room temperature, then cooled to 0° C. to afford a precipitate that was isolated via filtration, washed with cold EtOAc, air-dried, then dried under vacuum to yield 3-methoxy-4-(methylamino)cyclobut-3-ene-1,2-dione (0.70 g, 5 mmol, 73%) as a solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Heptadecan-9-yl 8-((3-((2-(methylamino)-3,4-dioxocyclobut-1-en-1-yl)amino)propyl)(8-(nonyloxy)-8-oxooctyl)amino)octanoate

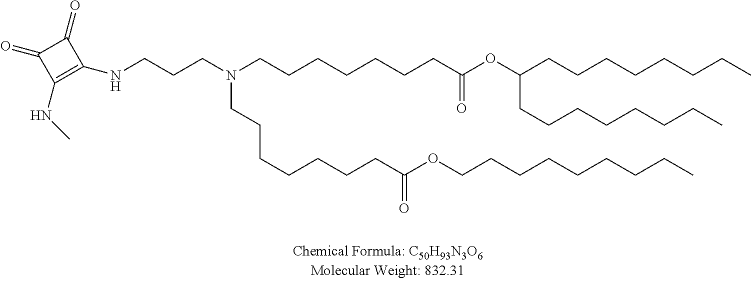 [SMILES: CCCCCCCCCOC(=O)CCCCCCCN(CCCCCCCC(=O)OC(CCCCCCCC)CCCCCCCC)CCCNc1c(NC)c(=O)c1=O]

To a solution of heptadecan-9-yl 8-((3-aminopropyl)(8-(nonyloxy)-8-oxooctyl)amino)octanoate (200 mg, 0.28 mmol) in 10 mL ethanol was added 3-methoxy-4-(methylamino)cyclobut-3-ene-1,2-dione (39 mg, 0.28 mmol). The reaction mixture stirred at room temperature for 20 hours, then concentrated in vacuo to yield a residue. The residue was purified by silica gel chromatography (0-100% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-(nonyloxy)-8-oxooctyl)amino)octanoate (138 mg, 0.17 mmol, 60%) as a solid. UPLC/ELSD: RT=3. min. MS (ES): m/z (MH⁺) 833.4 for C₅₁H₉₅N₃O₆. ¹H NMR (300 MHz, CDCl₃) δ: ppm 7.86 (br. s., 1H); 4.86 (quint., 1H, J=6 Hz); 4.05 (t, 2H, J=6 Hz); 3.92 (d, 2H, J=3 Hz); 3.20 (s, 6H); 2.63 (br. s, 2H); 2.42 (br. s, 3H); 2.28 (m, 4H); 1.74 (br. s, 2H); 1.61 (m, 8H); 1.50 (m, 5H); 1.41 (m, 3H); 1.25 (br. m, 47H); 0.88 (t, 9H, J=7.5 Hz).

### Compound I-301: Heptadecan-9-yl 8-((3-((2-(methylamino)-3,4-dioxocyclobut-1-en-1-yl)amino)propyl)(8-oxo-8-(undecan-3-yloxy)octyl)amino)octanoate

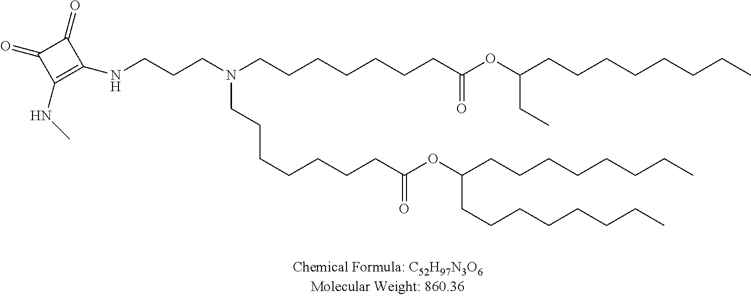 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

Compound I-301 was prepared analogously to compound 182 except that heptadecan-9-yl 8-((3-aminopropyl)(8-oxo-8-(undecan-3-yloxy)octyl)amino)octanoate (500 mg, 0.66 mmol) was used instead of heptadecan-9-yl 8-((3-aminopropyl)(8-(nonyloxy)-8-oxooctyl)amino)octanoate. Following an aqueous workup, the residue was purified by silica gel chromatography (0-50% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-oxo-8-(undecan-3-yloxy)octyl)amino)octanoate (180 mg, 32%) as a solid. HPLC/UV (254 nm): RT=6.77 min. MS (CI): m/z (MH⁺) 860.7 for C₅₂H₉₇N₃O₆. ¹H NMR (300 MHz, CDCl₃): δ ppm 4.86-4.79 (m, 2H); 3.66 (bs, 2H); 3.25 (d, 3H, J=4.9 Hz); 2.56-2.52 (m, 2H); 2.42-2.37 (m, 4H); 2.28 (dd, 4H, J=2.7 Hz, 7.4 Hz); 1.78-1.68 (m, 3H); 1.64-1.50 (m, 16H); 1.48-1.38 (m, 6H); 1.32-1.18 (m, 43H); 0.88-0.84 (m, 12H).

### Cholesterol/Structural Lipids

The LNP described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

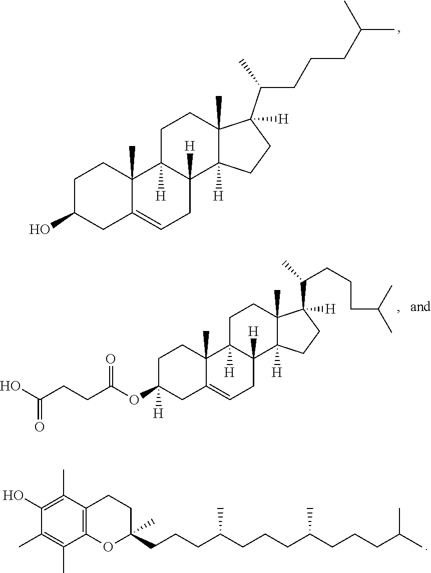

The target cell target cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. In certain embodiments, the structural lipid includes cholesterol. In another embodiment, the structural lipid can comprise cholesterol and another molecule. and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. Structural lipids can include, but are not limited to, sterols (e.g., phytosterols or zoosterols).

In certain embodiments, the structural lipid is cholesterol. In certain embodiments, the structural lipid is an analog of cholesterol.

### Non-Cationic Helper Lipids/Phospholipids

In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

### Phospholipids

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

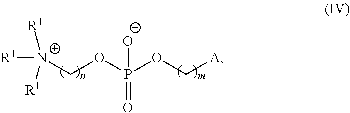

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the Formula:

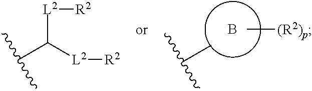

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}) C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}) S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the Formula:

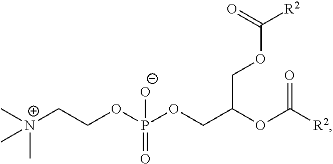

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

### Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments the compound of Formula (IV) is of one of the following Formulae:

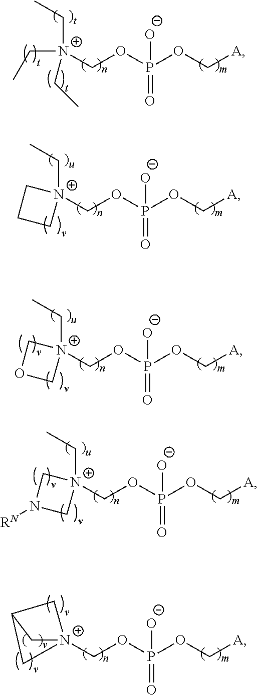

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

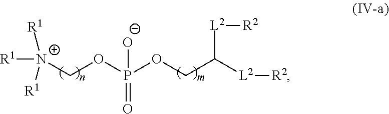

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

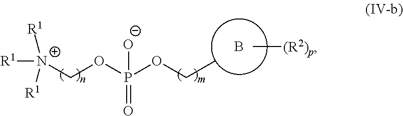

or a salt thereof.

### Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O.

In certain embodiments, the compound of Formula (IV) is of Formula (IV-c):

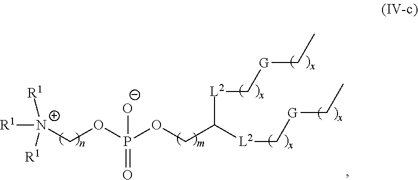

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}) C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}) S(O), OS(O), S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following Formulae:

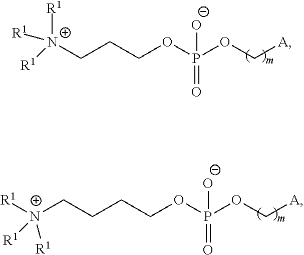

or a salt thereof.

### Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the invention is oleic acid.

In certain embodiments, the alternative lipid is one of the following.

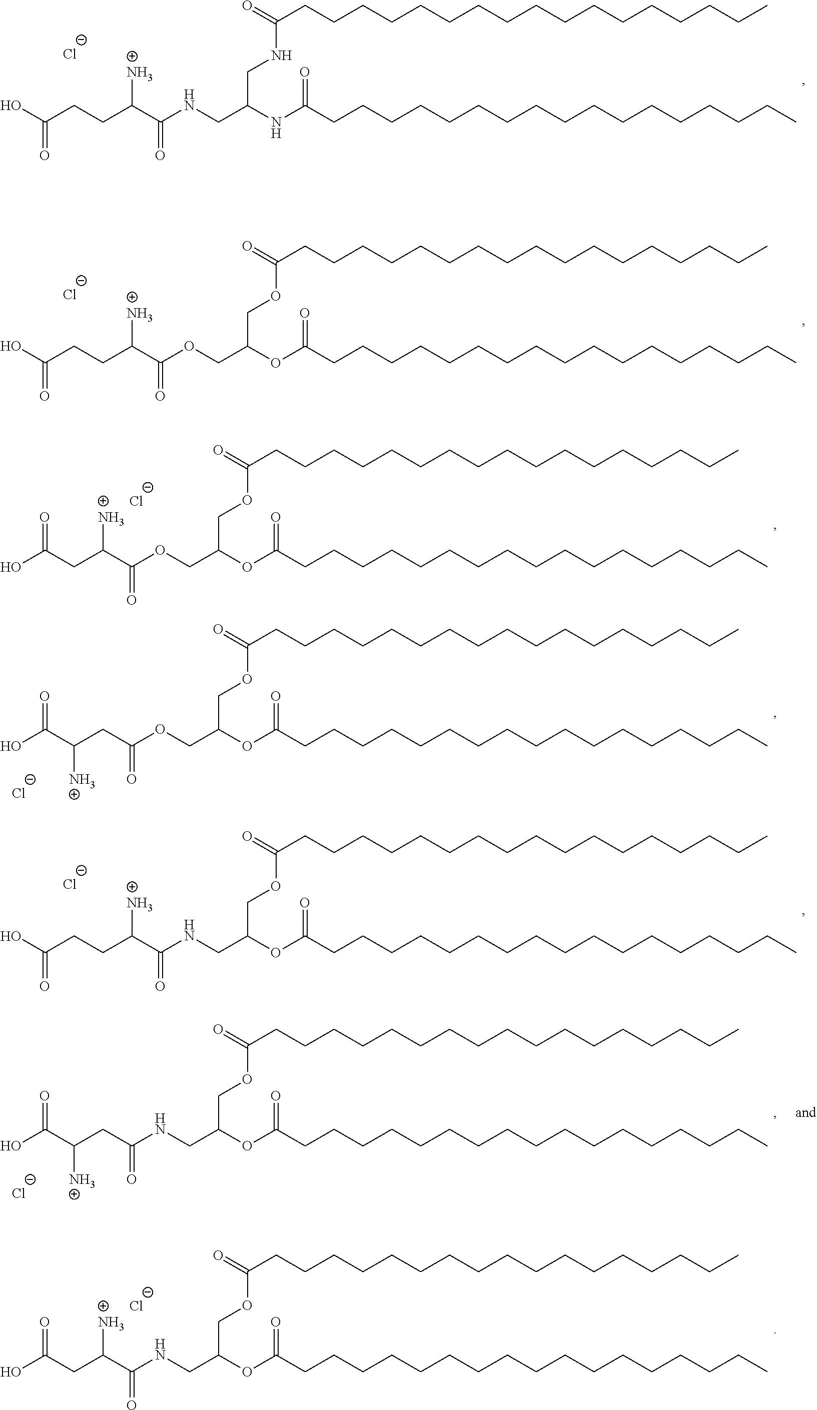

### PEG Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example a mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various Formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

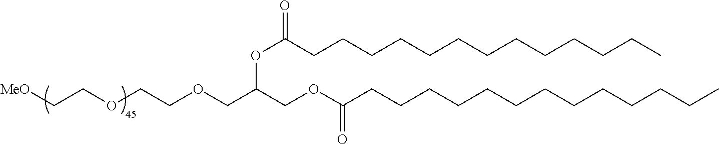

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

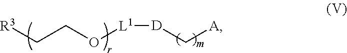

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O) C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the Formula:

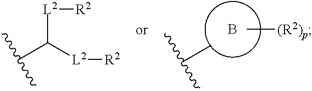

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}) C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}) S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

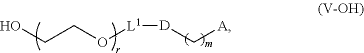

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

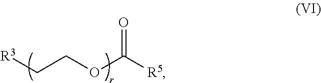

or a salt thereof, wherein:
R³ is-OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NC(O)O C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (VI) is of Formula (VI-OH).

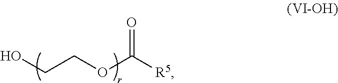

or a salt thereof. In some embodiments, r is 45. In another of the foregoing or related aspects, a PEG lipid of the invention is featured wherein r is 40-50.

In yet other embodiments the compound of Formula (VI) is:

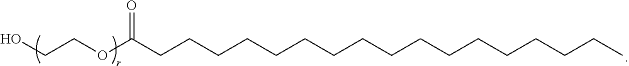

or a salt thereof.

In one embodiment, the compound of Formula (VI) is

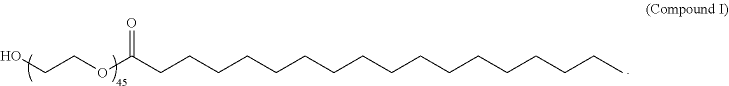

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the invention comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

The LNPs provided herein, in certain embodiments, exhibit increased PEG shedding compared to existing LNP formulations comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

### Exemplary Synthesis

### Compound: HO-PEG₂₀₀₀-Ester-C18

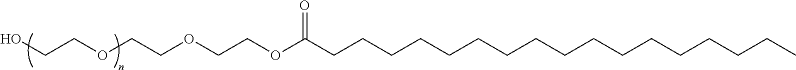

To a nitrogen filled flask containing palladium on carbon (10 wt. %, 74 mg, 0.070 mmol) was added Benzyl-PEG₂₀₀₀-ester-C18 (822 mg, 0.35 mmol) and MeOH (20 mL). The flask was evacuated and backfilled with H₂ three times, and allowed to stir at RT and 1 atm H₂ for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG200 peg lipid composition.

In some aspects, a target cell delivery lipid of the pharmaceutical compositions disclosed herein does not comprise a PEG-lipid.

In one embodiment, a target cell target cell delivery LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, a target cell target cell delivery LNP of the disclosure comprises a PEG-lipid which has a chain length longer than about 14 or than about 10, if branched.

As used herein, the term “alkyl”, “alkyl group”, or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. For example, C₁₈ alkenyl may include one or more double bonds. A C₁₈ alkenyl group including two double bonds may be a linoleyl group. Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, C₁₈ alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl”, “heteroalkenyl”, or “heteroalkynyl”, refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the Formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)₂R″″, in which each OR are alkoxy groups that can be the same or different and R″″ is an alkyl or alkenyl group), a phosphate (e.g., P(O)₄³⁻), a thiol (e.g., SH), a sulfoxide (e.g., S(O)R), a sulfinic acid (e.g., S(O)OH), a sulfonic acid (e.g., S(O)₂OH), a thial (e.g., C(S)H), a sulfate (e.g., S(O)₄²⁻), a sulfonyl (e.g., S(O)₂), an amide (e.g., C(O)NR₂, or N(R)C(O)R), an azido (e.g., N₃), a nitro (e.g., NO₂), a cyano (e.g., CN), an isocyano (e.g., NC), an acyloxy (e.g., OC(O)R), an amino (e.g., NR₂, NRH, or NH₂), a carbamoyl (e.g., OC(O)NR₂, OC(O)NRH, or OC(O)NH₂), a sulfonamide (e.g., S(O)₂NR₂, S(O)₂NRH, S(O)₂NH₂, N(R)S(O)₂R, N(H)S(O)₂R, N(R)S(O)₂H, or N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C₁₋₆ alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N→O or N+—O—). Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

### Exemplary Additional LNP Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

### LNPs Comprising Polynucleotides Encoding ITB6 Molecules

Disclosed herein are, inter alia, LNP compositions comprising polynucleotides encoding ITB6 polypeptides for use in suppressing T cells, for treating a disease associated with an aberrant T cell function, or for inhibiting an immune response in a subject. In another embodiment, the invention pertains to LNPs comprising a polynucleotide comprising an mRNA encoding an ITB6 molecule. The LNP compositions of the present disclosure can be used to reprogram dendritic cells, suppress T cells and/or induce immune tolerance in vivo or ex vivo.

In an aspect, an LNP composition comprising a polynucleotide encoding ITB6, comprises: (i) an ionizable lipid, e.g., an amino lipid; (ii) a sterol or other structural lipid; (iii) a non-cationic helper lipid or phospholipid; and (iv) a PEG-lipid.

In an aspect, an LNP composition comprising a polynucleotide encoding ITB6, comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease associated with an aberrant T cell function in a subject or a method of inhibiting an immune response in a subject, e.g., as described herein.

In an aspect, an LNP composition comprising a polynucleotide encoding an ITB6 molecule, can be administered with an additional agent, e.g., as described herein.

### Nanoparticle Compositions

In some embodiments, the pharmaceutical compositions disclosed herein are Formulated as lipid nanoparticles (LNP). Accordingly, the present disclosure also provides nanoparticle compositions comprising (i) a lipid composition comprising a delivery agent such as compound as described herein, and (ii) a polynucleotide encoding a polypeptide of the invention. In such nanoparticle composition, the lipid composition disclosed herein can encapsulate the polynucleotide encoding a polypeptide of the invention.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable amino lipid, a structural lipid, a phospholipid, and mRNA. In some embodiments, the LNP comprises an ionizable amino lipid, a PEG-modified lipid, a sterol and a structural lipid. In some embodiments, the LNP has a molar ratio of about 40-50% ionizable amino lipid; about 5-15% structural lipid; about 30-45% sterol; and about 1-5% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable amino lipid. As used herein, the term “ionizable amino lipid” has its ordinary meaning in the art and may refer to a lipid comprising one or more charged moieties. In some embodiments, an ionizable amino lipid may be positively charged or negatively charged. An ionizable amino lipid may be positively charged, in which case it can be referred to as “cationic lipid”. In certain embodiments, an ionizable amino lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

The ionizable amino lipid is sometimes referred to in the art as an “ionizable cationic lipid”. In one embodiment, the ionizable amino lipid may have a positively charged hydrophilic head and a hydrophobic tail that are connected via a linker structure.

In addition to these, an ionizable amino lipid may also be a lipid including a cyclic amine group.

In one embodiment, the ionizable amino lipid may be selected from, but not limited to, an ionizable amino lipid described in International Publication Nos. WO2013086354 and WO2013116126; the contents of each of which are herein incorporated by reference in their entirety.

In yet another embodiment, the ionizable amino lipid may be selected from, but not limited to, Formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a polypeptide are Formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electro kinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In one embodiment, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

### Therapeutic Methods

### Methods of Using the LNP Compositions

In an aspect, the disclosure provides a composition comprising a lipid nanoparticle (LNP) comprising a polynucleotide comprising an mRNA which encodes an ITB6 molecule in the treatment of a disease associated with an aberrant T cell function in a subject.

In another aspect, the disclosure provides a composition comprising a lipid nanoparticle (LNP) comprising a polynucleotide comprising an mRNA which encodes an ITB6 molecule for inhibiting an immune response in a subject.

In another aspect, the disclosure provides a composition comprising a lipid nanoparticle (LNP) comprising a polynucleotide comprising an mRNA which encodes an ITB6 molecule for inducing immune tolerance, e.g., in a subject.

In another aspect, the disclosure provides a composition comprising a lipid nanoparticle (LNP) comprising a polynucleotide comprising an mRNA which encodes an ITB6 molecule for suppressing T cells.

In another aspect, the disclosure provides a composition comprising a lipid nanoparticle (LNP) comprising a polynucleotide comprising an mRNA which encodes an ITB6 molecule for reprogramming myeloid and/or dendritic cells, e.g., to have a tolerogenic phenotype.

In another aspect, the disclosure provides a composition comprising a lipid nanoparticle (LNP) comprising a polynucleotide comprising an mRNA which encodes an ITB6 molecule for use, in the treatment of a disease associated with an aberrant T cell function in a subject.

In a related aspect, provided herein is a method of treating a disease associated with aberrant T cell function in a subject, comprising administering to the subject an effective amount of a lipid nanoparticle (LNP) comprising a lipid nanoparticle (LNP) comprising a polynucleotide comprising an mRNA which encodes an ITB6 molecule.

In embodiments of any of the methods disclosed herein, administration of the LNP results in suppression of T cell activity and/or function (e.g., T cell anergy and/or T cell apoptosis) in a population of immune cells, e.g., as compared to T cell activity and/or function in an otherwise similar population of cells which has not been contacted with the LNP composition comprising a polynucleotide comprising an mRNA which encodes an ITB6 molecule.

In some embodiments, suppression of T cell activity and/or function comprises any one, two, three, four, or all of the following: (i) reduced level and/or activity of effector cytokines IFNg, e.g., secreted IFNg, in a sample; (ii) reduction in T cell proliferation, survival and/or expansion; (iii) increased T cell apoptosis; (iv) reduction in expression and/or activity of a T cell transcription factor, e.g., T-bet; and/or (v) increase in and/or activation of T reg differentiation.

In embodiments of any of the methods disclosed herein, administration of the LNP results in a reduction in the level (e.g., expression) and/or activity of a costimulatory molecule, e.g., CD80, CD86, and/or MHCII, in a sample upon stimulation.

In embodiments of any of the methods disclosed herein, administration of the LNP results in: (i) reduced engraftment of donor cells, e.g., donor immune cells, e.g., T cells, in a subject or host, e.g., a human, rat or mouse; (ii) reduction in the level, activity and/or secretion of IFNg from engrafted donor immune cells, e.g., T cells, in a subject or host, e.g., a human, rat or mouse; and/or (iii) an absence of, prevention of, or delay in the onset of, graft vs host disease (GvHD) in a subject or a host, e.g., a human, rat or mouse.

In embodiments of any of the methods disclosed herein, administration of LNP results in reduced percentages of CD25+ and/or Tbet+ cells among intrahepatic CD8 T cells in a subject. In an embodiment, the subject has GvHD.

In embodiments of any of the methods disclosed herein, administration of the LNP results in: amelioration or reduction of acute graft vs. host disease, e.g., reduction in weight loss and/or B cell depletion, e.g., as described herein, in a subject, e.g., as measured by an assay described in Example 6.

In embodiments of any of the methods disclosed herein, administration of the LNP results in an increase in the expression level of one or more (e.g., 2, 3, 4, or 5) biomarkers comprising PMEPA1, ITGAE/CD103, SMAD7, SKIL, SKI, or any combination thereof. In some embodiments, the expression level of such a biomarker may be determined to be higher in a sample obtained from a subject sensitive or responsive to a treatment (e.g., treatment with an ITB6 mRNA) than a reference level, e.g., the median expression level of the biomarker in a sample from a group/population of subjects, e.g., subjects not having received treatment with an ITB6 mRNA; the median expression level of the biomarker in a sample from a group/population of subjects, and identified as not responding to a treatment; or the level in a sample previously obtained from the individual at a prior time (e.g., prior to treatment with an ITB6 mRNA). In embodiments of any of the methods disclosed herein, the method further comprises assessing the responsiveness of the subject to the treatment or the efficacy of the treatment. In some embodiments, treatment with the LNP is continued when the subject is responsive to the treatment. In some embodiments, treatment with the LNP is discontinued when the subject is not responsive to the treatment. In some embodiments, the subject is selected for continued treatment of the LNP when the subject is responsive to the treatment. In some embodiments, the subject is not selected for continued treatment of the LNP when the subject is not responsive to the treatment. In some embodiments, continued treatment with the LNP is selected for the subject when the subject is responsive to the treatment. In some embodiments, continued treatment with the LNP is not selected for the subject when the subject is not responsive to the treatment.

### LNP Dosing and Dosing Regimen

In some embodiments, any of the LNP disclosed herein can be administered according to a dosing interval, e.g., as described herein. In some embodiments, the dosing interval comprises an initial dose of the LNP composition and one or more subsequent doses (e.g., 1-50 doses, 5-50 doses, 10-50 doses, 15-50 doses, 20-50 doses, 25-50 doses, 30-50 doses, 35-50 doses, 40-50 doses, 45-50 doses, 1-45 doses, 1-40 doses, 1-35 doses, 1-30 doses, 1-25 doses, 1-20 doses, 1-15 doses, 1-10 doses, 1-5 doses) of the same LNP composition.

In some embodiments, the dosing interval comprises one or more doses of the LNP composition and one or more doses of an additional agent.

In some embodiments, the dosing interval is performed over at least 1 week, 2 weeks, 3 weeks, or 4 weeks.

In some embodiments, the dosing interval comprises a cycle, e.g., a seven-day cycle.

In some embodiments, the dosing interval is repeated at least 1 time, at least 2 times, at least 3 times, at least 4 times, at least 5 times, at least 6 times, at least 7 times, at least 8 times, at least 9 times, or at least 10 times. In some embodiments, the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years or 5 years.

In some embodiments, any of the LNP disclosed herein is administered daily for at least 1 week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months or 1 year. In some embodiments, the LNP composition is administered for at least 2, 3, 4, 5, or 6 consecutive days in a seven day cycle, e.g., wherein the cycle is repeated about 1-20 times.

In some embodiments of a combination therapy disclosed herein, the LNP compositions are administered according to a dosing interval, e.g., as described herein.

In some embodiments, the dosing interval comprises an initial dose of the LNP composition, or the combination comprising a first LNP composition and a second LNP composition and one or more subsequent doses (e.g., 1-50 doses, 5-50 doses, 10-50 doses, 15-50 doses, 20-50 doses, 25-50 doses, 30-50 doses, 35-50 doses, 40-50 doses, 45-50 doses, 1-45 doses, 1-40 doses, 1-35 doses, 1-30 doses, 1-25 doses, 1-20 doses, 1-15 doses, 1-10 doses, 1-5 doses) of the same LNP composition, or the same combination comprising a first LNP composition and a second LNP composition.

In some embodiments, the dosing interval comprises one or more doses of the LNP composition, or the combination comprising a first LNP composition and a second LNP composition, and one or more doses of an additional agent.

In some embodiments, the dosing interval is performed over at least 1 week, 2 weeks, 3 weeks, or 4 weeks.

In some embodiments, the dosing interval comprises a cycle, e.g., a seven-day cycle.

In some embodiments, the dosing interval is repeated at least 1 time, at least 2 times, at least 3 times, at least 4 times, at least 5 times, at least 6 times, at least 7 times, at least 8 times, at least 9 times, or at least 10 times. In some embodiments, the repeated dosing interval is performed over at least 2 weeks, 3 weeks, 4 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years or 5 years.

In some embodiments, any of the LNP disclosed herein is administered daily for at least 1 week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months or 1 year. In some embodiments, the LNP composition is administered for at least 2, 3, 4, 5, or 6 consecutive days in a seven day cycle, e.g., wherein the cycle is repeated about 1-20 times.

In some embodiments of any of the LNP compositions disclosed herein, the LNP composition is administered at a dose, e.g., total dose, of about 0.1-10 mg per kg, about 0.1-9.5 mg per kg, about 0.1-9 mg per kg, about 0.1-8.5 mg per kg, about 0.1-8 mg per kg, about 0.1-7.5 mg per kg, about 0.1-7 mg per kg, about 0.1-6.5 mg per kg, about 0.1-6 mg per kg, about 0.1-5.5 mg per kg, about 0.1-5 mg per kg, about 0.1-4.5 mg per kg, about 0.1-4 mg per kg, about 0.1-3.5 mg per kg, about 0.1-3 mg per kg, about 0.1-2.5 mg per kg, about 0.1-2 mg per kg, about 0.1-1.5 mg per kg, about 0.1-1 mg per kg, about 0.1-0.9 mg per kg, about 0.1-0.8 mg per kg, about 0.1-0.7 mg per kg, about 0.1-0.6 mg per kg, or about 0.1-0.5 mg per kg.

In some embodiments of any of the LNP compositions disclosed herein, the LNP composition is administered at a dose, e.g., total dose, of about 0.2-10 mg per kg, about, 0.3-10 mg per kg, about 0.4-10 mg per kg, about 0.5-10 mg per kg, about 0.6-10 mg per kg, about 0.7-10 mg per kg, about 0.8-10 mg per kg, about 0.9-10 mg per kg, about 1-10 mg per kg, about 1.5-10 mg per kg, about 2-10 mg per kg, about 2.5-10 mg per kg, about 3-10 mg per kg, about 3.5-10 mg per kg, about 4-10 mg per kg, about 4.5-10 mg per kg, about 5-10 mg per kg, about 5.5-10 mg per kg, about 6-10 mg per kg, about 6.5-10 mg per kg, about 7-10 mg per kg, about 7.5-10 mg per kg, about 8-10 mg per kg, about 8.5-10 mg per kg, about 9-10 mg per kg, or about 9.5-10 mg per kg.

In some embodiments of any of the LNP compositions disclosed herein, the LNP composition is administered at a dose, e.g., total dose, of about 0.1 mg per kg.

In some embodiments of any of the LNP compositions disclosed herein, the LNP composition is administered at a dose, e.g., total dose, of about 0.2 mg per kg.

In some embodiments of any of the LNP compositions disclosed herein, the LNP composition is administered at a dose, e.g., total dose, of about 0.3 mg per kg.

In some embodiments of any of the LNP compositions disclosed herein, the LNP composition is administered at a dose, e.g., total dose, of about 0.4 mg per kg.

In some embodiments of any of the LNP compositions disclosed herein, the LNP composition is administered at a dose, e.g., total dose, of about 0.5 mg per kg.

In some embodiments of any of the LNP compositions disclosed herein, the LNP composition is administered at a dose, e.g., dose of each LNP, of about 0.1-10 mg per kg, about 0.1-9.5 mg per kg, about 0.1-9 mg per kg, about 0.1-8.5 mg per kg, about 0.1-8 mg per kg, about 0.1-7.5 mg per kg, about 0.1-7 mg per kg, about 0.1-6.5 mg per kg, about 0.1-6 mg per kg, about 0.1-5.5 mg per kg, about 0.1-5 mg per kg, about 0.1-4.5 mg per kg, about 0.1-4 mg per kg, about 0.1-3.5 mg per kg, about 0.1-3 mg per kg, about 0.1-2.5 mg per kg, about 0.1-2 mg per kg, about 0.1-1.5 mg per kg, about 0.1-1 mg per kg, about 0.1-0.9 mg per kg, about 0.1-0.8 mg per kg, about 0.1-0.7 mg per kg, about 0.1-0.6 mg per kg, or about 0.1-0.5 mg per kg.

In some embodiments of any of the LNP compositions disclosed herein, the LNP composition is administered at a dose, e.g., dose of each LNP, of about 0.2-10 mg per kg, about, 0.3-10 mg per kg, about 0.4-10 mg per kg, about 0.5-10 mg per kg, about 0.6-10 mg per kg, about 0.7-10 mg per kg, about 0.8-10 mg per kg, about 0.9-10 mg per kg, about 1-10 mg per kg, about 1.5-10 mg per kg, about 2-10 mg per kg, about 2.5-10 mg per kg, about 3-10 mg per kg, about 3.5-10 mg per kg, about 4-10 mg per kg, about 4.5-10 mg per kg, about 5-10 mg per kg, about 5.5-10 mg per kg, about 6-10 mg per kg, about 6.5-10 mg per kg, about 7-10 mg per kg, about 7.5-10 mg per kg, about 8-10 mg per kg, about 8.5-10 mg per kg, about 9-10 mg per kg, or about 9.5-10 mg per kg.

In some embodiments of any of the LNP compositions disclosed herein, the LNP composition is administered at a dose, e.g., dose of each LNP, of about 0.1 mg per kg.

In some embodiments of any of the LNP compositions disclosed herein, the LNP composition is administered at a dose, e.g., dose of each LNP, of about 0.2 mg per kg.

In some embodiments of any of the LNP compositions disclosed herein, the LNP composition is administered at a dose, e.g., dose of each LNP, of about 0.3 mg per kg.

In some embodiments of any of the LNP compositions disclosed herein, the LNP composition is administered at a dose, e.g., dose of each LNP, of about 0.4 mg per kg.

In some embodiments of any of the LNP compositions disclosed herein, the LNP composition is administered at a dose, e.g., dose of each LNP, of about 0.5 mg per kg.

In some embodiments of any of the LNP compositions disclosed herein, the LNP composition is administered at a dose, e.g., dose of each polynucleotide in the LNP, of about 0.1-10 mg per kg, about 0.1-9.5 mg per kg, about 0.1-9 mg per kg, about 0.1-8.5 mg per kg, about 0.1-8 mg per kg, about 0.1-7.5 mg per kg, about 0.1-7 mg per kg, about 0.1-6.5 mg per kg, about 0.1-6 mg per kg, about 0.1-5.5 mg per kg, about 0.1-5 mg per kg, about 0.1-4.5 mg per kg, about 0.1-4 mg per kg, about 0.1-3.5 mg per kg, about 0.1-3 mg per kg, about 0.1-2.5 mg per kg, about 0.1-2 mg per kg, about 0.1-1.5 mg per kg, about 0.1-1 mg per kg, about 0.1-0.9 mg per kg, about 0.1-0.8 mg per kg, about 0.1-0.7 mg per kg, about 0.1-0.6 mg per kg, or about 0.1-0.5 mg per kg.

In some embodiments of any of the LNP compositions disclosed herein, the LNP composition is administered at a dose, e.g., dose of each polynucleotide in the LNP, of about 0.2-10 mg per kg, about, 0.3-10 mg per kg, about 0.4-10 mg per kg, about 0.5-10 mg per kg, about 0.6-10 mg per kg, about 0.7-10 mg per kg, about 0.8-10 mg per kg, about 0.9-10 mg per kg, about 1-10 mg per kg, about 1.5-10 mg per kg, about 2-10 mg per kg, about 2.5-10 mg per kg, about 3-10 mg per kg, about 3.5-10 mg per kg, about 4-10 mg per kg, about 4.5-10 mg per kg, about 5-10 mg per kg, about 5.5-10 mg per kg, about 6-10 mg per kg, about 6.5-10 mg per kg, about 7-10 mg per kg, about 7.5-10 mg per kg, about 8-10 mg per kg, about 8.5-10 mg per kg, about 9-10 mg per kg, or about 9.5-10 mg per kg.

In some embodiments of any of the LNP compositions disclosed herein, the LNP composition is administered at a dose, e.g., dose of each polynucleotide in the LNP, of about 0.1 mg per kg.

In some embodiments of any of the LNP compositions disclosed herein, the LNP composition is administered at a dose, e.g., dose of each polynucleotide in the LNP, of about 0.2 mg per kg.

In some embodiments of any of the LNP compositions disclosed herein, the LNP composition is administered at a dose, e.g., dose of each polynucleotide in the LNP, of about 0.3 mg per kg.

In some embodiments of any of the LNP compositions disclosed herein, the LNP composition is administered at a dose, e.g., dose of each polynucleotide in the LNP, of about 0.4 mg per kg.

In some embodiments of any of the LNP compositions disclosed herein, the LNP composition is administered at a dose, e.g., dose of each polynucleotide in the LNP, of about 0.5 mg per kg.

In some embodiments, any of the LNP disclosed herein is administered by a route of administration chosen from: subcutaneous, intramuscular, intravenous, intranasal, oral, intraocular, or rectal. In some embodiments, the route of administration is subcutaneous.

In some embodiments, the route of administration is intramuscular. In some embodiments, the route of administration is intravenous. In some embodiments, the route of administration is In some embodiments, the route of administration is intranasal. In some embodiments, the route of administration is oral. In some embodiments, the route of administration is intraocular. In some embodiments, the route of administration is rectal.

### Diseases and Disorders

In an embodiment of any of the methods of treatment or compositions for use disclosed herein, the subject has, or is identified as having, a disease or disorder associated with aberrant T cell function. In an embodiment, the disease is an autoimmune disease, or a disease with hyper-activated immune function. In an embodiment, an LNP disclosed herein is administered to the subject to treat or ameliorate a symptom of the disease or disorder. In an embodiment, an LNP disclosed herein is administered to a subject to inhibit an immune response in the subject.

In an embodiment, the autoimmune disease is chosen from: rheumatoid arthritis (RA); graft versus host disease (GVHD) (e.g., acute GVHD or chronic GVHD); diabetes, e.g., Type 1 diabetes; inflammatory bowel disease (IBD); lupus (e.g., systemic lupus erythematosus (SLE)); autoimmune hepatitis (e.g., Type 1 or Type 2); primary biliary cholangitis; organ transplant associated rejection; myasthenia gravis; psoriasis; or polymyositis (also known as dermatomyositis).

In an embodiment, the autoimmune disease is rheumatoid arthritis (RA). In an embodiment, the autoimmune disease is graft versus host disease (GVHD) (e.g., acute GVHD or chronic GVHD). In an embodiment, the autoimmune disease is diabetes, e.g., Type 1 diabetes. In an embodiment, the autoimmune disease is inflammatory bowel disease (IBD). In an embodiment, IBD comprises colitis, ulcerative colitis or Crohn's disease. In an embodiment, the autoimmune disease is lupus, e.g., systemic lupus erythematosus (SLE). In an embodiment, the autoimmune disease is autoimmune hepatitis, e.g., Type 1 or Type 2. In an embodiment, the autoimmune disease is primary biliary cholangitis.

In an embodiment, the autoimmune disease is organ transplant associated rejection. In an embodiment, an organ transplant associated rejection comprises allograft rejection; e.g., renal rejection, liver transplant rejection; bone marrow transplant rejection; or stem cell transplant rejection. In an embodiment, a stem cell transplant comprises a transplant of any one or all of the following types of cells: stem cells, cord blood stem cells, hematopoietic stem cells, embryonic stem cells, cells derived from or comprising mesenchymal stem cells, and/or induced stem cells (e.g., induced pluripotent stem cells). In an embodiment, the stem cell comprises a pluripotent stem cell.

In an embodiment, the autoimmune disease is myasthenia gravis. In an embodiment, the autoimmune disease is psoriasis. In an embodiment, the autoimmune disease is polymyositis.

In an embodiment the subject is a mammal, e.g., a human.

### Combination Therapies

In some embodiments, the methods of treatment or compositions for use disclosed herein, comprise administering an LNP disclosed herein in combination with an additional agent. In an embodiment, the additional agent is a standard of care for the disease or disorder, e.g., autoimmune disease. In an embodiment, the additional agent is an mRNA In some aspects, the subject for the present methods or compositions has been treated with one or more standard of care therapies. In other aspects, the subject for the present methods or compositions has not been responsive to one or more standard of care therapies.

### Biomarkers for Evaluating Therapeutic Efficacy

Provided herein are methods of assessing the responsiveness of a subject to a treatment or the efficacy of a treatment (e.g., treatment with an ITB6 mRNA). In some embodiments, the method includes acquiring (e.g., determining or measuring) the level of one or more genes in a sample from the subject that correlates with therapeutic efficacy of and comparing the level to a reference level, thereby evaluating the effectiveness of the therapy.

In some embodiments, the expression level of such a biomarker may be determined to be higher in a sample obtained from a patient sensitive or responsive to a treatment (e.g., treatment with an ITB6 mRNA) than a reference level, e.g., the median expression level of the biomarker in a sample from a group/population of patients, e.g., patients not having received treatment with an ITB6 mRNA; the median expression level of the biomarker in a sample from a group/population of patients, and identified as not responding to a treatment; or the level in a sample previously obtained from the individual at a prior time (e.g., prior to treatment with an ITB6 mRNA).

In some embodiments, the biomarker is one or more (e.g., 2, 3, 4, or 5) biomarkers comprising PMEPA1, ITGAE/CD103, SMAD7, SKIL, SKI, or any combination thereof. In some embodiments, an increase in the level of expression of one or more (e.g., 2, 3, 4, or 5) of PMEPA1, ITGAE/CD103, SMAD7, SKIL, and SKI in a sample from a subject following therapy (e.g., with an ITB6 mRNA) relative to a reference level is indicative of a response to the therapy.

In some embodiments, where the expression level of the one or more biomarkers compared to a reference expression level indicates a responsiveness to the therapy (e.g., a therapy comprising an ITB6 mRNA), the method further includes administering one or more additional doses of the therapy to the subject.

In some embodiments, an increase in expression level (e.g., an increase in the expression level of about 10%, 20%, 30%, 40%, 50%, 60%, 70%, 80%, 90%, 95%, 96%, 97%, 98%, or 99% or greater) of one or more (e.g., 2, 3, 4, or 5) of PMEPA1, ITGAE/CD103, SMAD7, SKIL, or SKI relative to a reference expression level identifies the subject as one who is responsive to therapy (e.g., a therapy comprising an ITB6 mRNA). In some embodiments, the increased expression level of one or more of PMEPA1, ITGAE/CD103, SMAD7, SKIL, or SKI is an increase of at least about 1.1×, 1.2×, 1 3×, 1.4×, 1 5×, 1.6×, 1.7×, 1 8×, 1.9×, 2×, 2.1×, 2.2×, 2.3×, 2.4×, 2.5×, 2.6×, 2.7×, 2.8×, 2.9×, 3×, 3.5×, 4×, 4.5×, 5×, 6×, 7×, 8×, 9×, 10×, 15×, 20×, 30×, 40×, 50×, 100×, 500×, or 1000× relative to a reference expression level of the one or more genes. In some embodiments, the increased expression level of one or more (e.g., 2, 3, 4, or 5) of PMEPA1, ITGAE/CD103, SMAD7, SKIL, or SKI is an increase of at least about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.5-fold, about 4-fold, about 4.5-fold, about 5-fold, about 6-fold, about 7-fold, about 8-fold, about 9-fold, about 10-fold, about 15-fold, about 20-fold, about 30-fold, about 40-fold, about 50-fold, about 100-fold, about 500-fold, or about 1,000-fold or greater relative to a reference expression level of the one or more genes.

In some instances of any of the preceding methods and assays the reference sample is obtained from the individual prior to (e.g., minutes, hours, days, weeks (e.g., 1, 2, 3, 4, 5, 6, or 7 weeks), months, or years prior to) administration of therapy. In some instances of any of the preceding methods, the sample from the individual is obtained days, weeks, months, or years following administration of the therapy. In some embodiments, the sample is obtained about 1 to about 7 days (e.g., 1, 2, 3, 4, 5, 6, or 7 days) following administration of a therapy. In some embodiments, the sample is obtained about 1 to about 10 weeks (e.g., 1, 2, 3, 4, 5, 6, 7, 8, 9, or 10 weeks) following administration of a therapy.

In some embodiments of any of the preceding methods and assays, the sample and/or the reference sample comprises a serum sample, a blood sample, a plasma sample, or a sample containing peripheral blood mononuclear cells (PBMCs).

In some embodiments, the expression level of one or more biomarkers (e.g., one or more (e.g., 2, 3, 4, or 5) of PMEPA1, ITGAE/CD103, SMAD7, SKIL, or SKI) in a sample (e.g., a blood sample, a serum sample, a plasma sample, or a sample containing PBMCs) can be determined qualitatively and/or quantitatively based on any suitable criterion known in the art, including, but not limited to, the measurement of DNA, mRNA, cDNA, proteins, protein fragments, and/or gene copy number levels in an individual.

Methodologies for measuring such biomarkers are known in the art and understood by the skilled artisan, including, but not limited to, whole genome sequencing, polymerase chain reaction (PCR) including quantitative real time PCR (qRT-PCR) and other amplification type detection methods, such as, for example, branched DNA, SISBA, TMA and the like, RNASeq, microarray analysis, gene expression profiling, whole-genome sequencing (WGS), and/or serial analysis of gene expression (“SAGE”), direct digital counting of nucleic acids (e.g., Nanostring nCounter), immunohistochemistry (“IHC”), Western blot analysis, immunoprecipitation, molecular binding assays, ELISA, ELIFA, fluorescence activated cell sorting (“FACS”), MassARRAY, proteomics, biochemical enzymatic activity assays, in situ hybridization (ISH), fluorescence in situ hybridization (FISH), Southern analysis, Northern analysis, as well as any one of the wide variety of assays that can be performed by protein, gene, and/or tissue array analysis.

In some instances of any of the preceding methods and assays, the expression level of a biomarker may be a nucleic acid expression level (e.g., a DNA expression level or an RNA expression level (e.g., an mRNA expression level)). Any suitable method of determining a nucleic acid expression level may be used. In some instances, the nucleic acid expression level is determined using RNAseq or qPCR.

In other instances of any of the preceding methods, the expression level of a biomarker may be a protein expression level. In certain instances, the method comprises contacting the sample with antibodies that specifically bind to a biomarker described herein under conditions permissive for binding of the biomarker, and detecting whether a complex is formed between the antibodies and biomarker.

Any method of measuring protein expression levels known in the art or provided herein may be used. For example, in some instances, a protein expression level of a biomarker is determined using a method selected from, but not limited to western blot, enzyme-linked immunosorbent assay (ELISA), immunoprecipitation, immunohistochemistry (IHC), flow cytometry (e.g., fluorescence-activated cell sorting (FACS™)), immunofluorescence, radioimmunoassay, dot blotting, immunodetection methods, HPLC, surface plasmon resonance, optical spectroscopy, mass spectrometry, liquid chromatography-mass spectrometry (LC-MS), nephelometry, aptamer technology, and HPLC.

### Sequence Optimization and Methods Thereof

In some embodiments, a polynucleotide of the disclosure comprises a sequence-optimized nucleotide sequence encoding a polypeptide disclosed herein, e.g., an ITB6 molecule. In some embodiments, the polynucleotide of the disclosure comprises an open reading frame (ORF) encoding an ITB6 polypeptide, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the percentage of uracil or thymine nucleobases in a sequence-optimized nucleotide sequence (e.g., encoding an ITB6 molecule, a functional fragment, or a variant thereof) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (% UTM or % TTM), relative to the wild-type (% UWT or % TWT), and relative to the total nucleotide content (% UTL or % TTL). For DNA it is recognized that thymine is present instead of uracil, and one would substitute T where U appears. Thus, all the disclosures related to, e.g., % UTM, % UWT, or % UTL, with respect to RNA are equally applicable to % TTM, % TWT, or % TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % UTM or % TTM.

In some embodiments, a uracil-modified sequence encoding an ITB6 molecule polypeptide of the disclosure has a reduced number of consecutive uracils with respect to the corresponding wild-type nucleic acid sequence. For example, two consecutive leucines can be encoded by the sequence CUUUUG, which includes a four-uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence encoding an ITB6 molecule polypeptide of the disclosure has a reduced number of uracil triplets (UUU) with respect to the wild-type nucleic acid sequence. In some embodiments, a uracil-modified sequence encoding an ITB6 molecule polypeptide has a reduced number of uracil pairs (UU) with respect to the number of uracil pairs (UU) in the wild-type nucleic acid sequence. In some embodiments, a uracil-modified sequence encoding an ITB6 molecule polypeptide of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence,” refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UUwt. In some embodiments, a uracil-modified sequence encoding an ITB6 molecule polypeptide has a % UUwt between below 100%.

In some embodiments, the polynucleotide of the disclosure comprises a uracil-modified sequence encoding an ITB6 molecule polypeptide disclosed herein. In some embodiments, the uracil-modified sequence encoding an ITB6 molecule polypeptide comprises at least one chemically modified nucleobase, e.g., 5-methoxyuracil. In some embodiments, at least 95% of a nucleobase (e.g., uracil) in a uracil-modified sequence encoding an ITB6 molecule polypeptide of the disclosure are modified nucleobases. In some embodiments, at least 95% of uracil in a uracil-modified sequence encoding an ITB6 molecule polypeptide is 5-methoxyuracil. In some embodiments, the polynucleotide comprising a uracil-modified sequence further comprises a miRNA binding site, e.g., a miRNA binding site that binds to miR-122. In some embodiments, the polynucleotide comprising a uracil-modified sequence is formulated with a delivery agent, e.g., a compound having Formula (I), e.g., any of Compounds Nos. 18, 25, 301, or 357.

In some embodiments, a polynucleotide of the disclosure (e.g., a polynucleotide) comprising a nucleotide sequence encoding ITB6 molecule polypeptide (e.g., the wild-type sequence, functional fragment, or variant thereof) is sequence optimized.

A sequence optimized nucleotide sequence (nucleotide sequence is also referred to as “nucleic acid” herein) comprises at least one codon modification with respect to a reference sequence (e.g., a wild-type sequence encoding an ITB6 molecule polypeptide). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence-optimized nucleic acids (e.g., a RNA, e.g., an mRNA) can be administered to a subject in need thereof to facilitate in vivo expression of functionally active ITB6 molecule polypeptide.

Additional and exemplary methods of sequence optimization are disclosed in International PCT application WO 2017/201325, filed on 18 May 2017, the entire contents of which are hereby incorporated by reference.

MicroRNA (miRNA) Binding Sites

Polynucleotides of the invention can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, polynucleotides including such regulatory elements are referred to as including “sensor sequences”.

In some embodiments, a polynucleotide (e.g., a ribonucleic acid (RNA), e.g., a messenger RNA (mRNA)) of the invention comprises an open reading frame (ORF) encoding a polypeptide of interest and further comprises one or more miRNA binding site(s). Inclusion or incorporation of miRNA binding site(s) provides for regulation of polynucleotides of the invention, and in turn, of the polypeptides encoded therefrom, based on tissue-specific and/or cell-type specific expression of naturally-occurring miRNAs.

The present invention also provides pharmaceutical compositions and formulations that comprise any of the polynucleotides described above. In some embodiments, the composition or formulation further comprises a delivery agent.

In some embodiments, the composition or formulation can contain a polynucleotide comprising a sequence optimized nucleic acid sequence disclosed herein which encodes a polypeptide. In some embodiments, the composition or formulation can contain a polynucleotide (e.g., a RNA, e.g., an mRNA) comprising a polynucleotide (e.g., an ORF) having significant sequence identity to a sequence optimized nucleic acid sequence disclosed herein which encodes a polypeptide. In some embodiments, the polynucleotide further comprises a miRNA binding site, e.g., a miRNA binding site that binds

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA.

microRNAs derive enzymatically from regions of RNA transcripts that fold back on themselves to form short hairpin structures often termed a pre-miRNA (precursor-miRNA). A pre-miRNA typically has a two-nucleotide overhang at its 3′ end, and has 3′ hydroxyl and 5′ phosphate groups. This precursor-mRNA is processed in the nucleus and subsequently transported to the cytoplasm where it is further processed by DICER (a RNase III enzyme), to form a mature microRNA of approximately 22 nucleotides. The mature microRNA is then incorporated into a ribonuclear particle to form the RNA-induced silencing complex, RISC, which mediates gene silencing. Art-recognized nomenclature for mature miRNAs typically designates the arm of the pre-miRNA from which the mature miRNA derives; “5p” means the microRNA is from the 5-prime arm of the pre-miRNA hairpin and “3p” means the microRNA is from the 3-prime end of the pre-miRNA hairpin. A miR referred to by number herein can refer to either of the two mature microRNAs originating from opposite arms of the same pre-miRNA (e.g., either the 3p or 5p microRNA). All miRs referred to herein are intended to include both the 3p and 5p arms/sequences, unless particularly specified by the 3p or 5p designation. As used herein, the term “microRNA (miRNA or miR) binding site” refers to a sequence within a polynucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polynucleotide of the invention comprising an ORF encoding a polypeptide of interest and further comprises one or more miRNA binding site(s). In exemplary embodiments, a 5′ UTR and/or 3′ UTR of the polynucleotide (e.g., a ribonucleic acid (RNA), e.g., a messenger RNA (mRNA)) comprises the one or more miRNA binding site(s).

A miRNA binding site having sufficient complementarity to a miRNA refers to a degree of complementarity sufficient to facilitate miRNA-mediated regulation of a polynucleotide, e.g., miRNA-mediated translational repression or degradation of the polynucleotide. In exemplary aspects of the invention, a miRNA binding site having sufficient complementarity to the miRNA refers to a degree of complementarity sufficient to facilitate miRNA-mediated degradation of the polynucleotide, e.g., miRNA-guided RNA-induced silencing complex (RISC)-mediated cleavage of mRNA. The miRNA binding site can have complementarity to, for example, a 19-25 nucleotide long miRNA sequence, to a 19-23 nucleotide long miRNA sequence, or to a 22-nucleotide long miRNA sequence. A miRNA binding site can be complementary to only a portion of a miRNA, e.g., to a portion less than 1, 2, 3, or 4 nucleotides of the full length of a naturally-occurring miRNA sequence, or to a portion less than 1, 2, 3, or 4 nucleotides shorter than a naturally-occurring miRNA sequence. Full or complete complementarity (e.g., full complementarity or complete complementarity over all or a significant portion of the length of a naturally-occurring miRNA) is preferred when the desired regulation is mRNA degradation. In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with a miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polynucleotide of the invention, the polynucleotide can be targeted for degradation or reduced translation, provided the miRNA in question is available. This can reduce off-target effects upon delivery of the polynucleotide. For example, if a polynucleotide of the invention is not intended to be delivered to a tissue or cell but ends up is said tissue or cell, then a miRNA abundant in the tissue or cell can inhibit the expression of the gene of interest if one or multiple binding sites of the miRNA are engineered into the 5′ UTR and/or 3′ UTR of the polynucleotide. Thus, in some embodiments, incorporation of one or more miRNA binding sites into an mRNA of the disclosure may reduce the hazard of off-target effects upon nucleic acid molecule delivery and/or enable tissue-specific regulation of expression of a polypeptide encoded by the mRNA. In yet other embodiments, incorporation of one or more miRNA binding sites into an mRNA of the disclosure can modulate immune responses upon nucleic acid delivery in vivo. In further embodiments, incorporation of one or more miRNA binding sites into an mRNA of the disclosure can modulate accelerated blood clearance (ABC) of lipid-comprising compounds and compositions described herein.

Conversely, miRNA binding sites can be removed from polynucleotide sequences in which they naturally occur to increase protein expression in specific tissues. For example, a binding site for a specific miRNA can be removed from a polynucleotide to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profiling in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune-response to infection, inflammation, as well as unwanted immune response after gene therapy and tissue/organ transplantation. Immune cells specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

In some embodiments, a polynucleotide of the invention comprises a miRNA binding site, wherein the miRNA binding site comprises one or more nucleotide sequences selected from Table 3C or Table 4A, including one or more copies of any one or more of the miRNA binding site sequences. In some embodiments, a polynucleotide of the invention further comprises at least one, two, three, four, five, six, seven, eight, nine, ten, or more of the same or different miRNA binding sites selected from Table 3C or Table 4A, including any combination thereof.

In some embodiments, the miRNA binding site binds to miR-142 or is complementary to miR-142. In some embodiments, the miR-142 comprises SEQ ID NO:114. In some embodiments, the miRNA binding site binds to miR-142-3p or miR-142-5p. In some embodiments, the miR-142-3p binding site comprises SEQ ID NO:202. In some embodiments, the miR-142-5p binding site comprises SEQ ID NO:204. In some embodiments, the miRNA binding site comprises a nucleotide sequence at least 80%, at least 85%, at least 90%, at least 95%, or 100% identical to SEQ ID NO:202 or SEQ ID NO:204.

In some embodiments, the miRNA binding site binds to miR-126 or is complementary to miR-126. In some embodiments, the miR-126 comprises SEQ ID NO: 205. In some embodiments, the miRNA binding site binds to miR-126-3p or miR-126-5p. In some embodiments, the miR-126-3p binding site comprises SEQ ID NO: 207. In some embodiments, the miR-126-5p binding site comprises SEQ ID NO: 209. In some embodiments, the miRNA binding site comprises a nucleotide sequence at least 80%, at least 85%, at least 90%, at least 95%, or 100% identical to SEQ ID NO: 121 or SEQ ID NO: 123.

In one embodiment, the 3′ UTR comprises two miRNA binding sites, wherein a first miRNA binding site binds to miR-142 and a second miRNA binding site binds to miR-126. In a specific embodiment, the 3′ UTR binding to miR-142 and miR-126 comprises, consists, or consists essentially of the sequence of SEQ ID NO: 249.

TABLE 3C
miR-142, miR-126, and miR-142 and miR-126 binding sites

| SEQ ID NO. | Description | Sequence |
|---|---|---|
| 114 | miR-142 | GACAGUGCAGUCACCCAUAAAGUAGAAAGCA |
| | | CUACUAACAGCACUGGAGGGUGUAGUGUUUC |
| | | CUACUUUAUGGAUGAGUGUACUGUG |
| 201 | miR-142-3p | uguaguguuuccuacuuuaugga |
| 202 | miR-142-3p binding site | uccauaaaguaggaaacacuaca |
| 203 | miR-142-5p | cauaaaguagaaagcacuacu |
| 204 | miR-142-5p binding site | aguagugcuuucuacuuuaug |
| 205 | miR-126 | CGCUGGCGACGGGACAUUAUUACUUUUGGUA |
| | | CGCGCUGUGACACUUCAAACUCGUACCGUGA |
| | | GUAAUAAUGCGCCGUCCACGGCA |
| 206 | miR-126-3p | UCGUACCGUGAGUAAUAAUGCG |
| 207 | miR-126-3p binding site | CGCAUUAUUACUCACGGUACGA |
| 208 | miR-126-5p | CAUUAUUACUUUUGGUACGCG |
| 209 | miR-126-5p binding site | CGCGUACCAAAAGUAAUAAUG |

In some embodiments, a miRNA binding site is inserted in the polynucleotide of the invention in any position of the polynucleotide (e.g., the 5′ UTR and/or 3′ UTR). In some embodiments, the 5′ UTR comprises a miRNA binding site. In some embodiments, the 3′ UTR comprises a miRNA binding site. In some embodiments, the 5′ UTR and the 3′ UTR comprise a miRNA binding site. The insertion site in the polynucleotide can be anywhere in the polynucleotide as long as the insertion of the miRNA binding site in the polynucleotide does not interfere with the translation of a functional polypeptide in the absence of the corresponding miRNA; and in the presence of the miRNA, the insertion of the miRNA binding site in the polynucleotide and the binding of the miRNA binding site to the corresponding miRNA are capable of degrading the polynucleotide or preventing the translation of the polynucleotide. In some embodiments, a miRNA binding site is inserted in at least about 30 nucleotides downstream from the stop codon of an ORF in a polynucleotide of the invention comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the invention. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a polynucleotide of the invention. In some embodiments, a miRNA binding site is inserted within the 3′ UTR immediately following the stop codon of the coding region within the polynucleotide of the invention, e.g., mRNA. In some embodiments, if there are multiple copies of a stop codon in the construct, a miRNA binding site is inserted immediately following the final stop codon. In some embodiments, a miRNA binding site is inserted further downstream of the stop codon, in which case there are 3′ UTR bases between the stop codon and the miR binding site(s). In some embodiments, three non-limiting examples of possible insertion sites for a miR in a 3′ UTR are shown in SEQ ID NOs: 248, 249, and 250, which show a 3′ UTR sequence with a miR-142-3p site inserted in one of three different possible insertion sites, respectively, within the 3′ UTR.

In some embodiments, one or more miRNA binding sites can be positioned within the 5′ UTR at one or more possible insertion sites. For example, three non-limiting examples of possible insertion sites for a miR in a 5′ UTR are shown in SEQ ID NOs: 251, 252, or 253, which show a 5′ UTR sequence with a miR-142-3p site inserted into one of three different possible insertion sites, respectively, within the 5′ UTR.

In one embodiment, a codon optimized open reading frame encoding a polypeptide of interest comprises a stop codon and the at least one microRNA binding site is located within the 3′ UTR 1-100 nucleotides after the stop codon. In one embodiment, the codon optimized open reading frame encoding the polypeptide of interest comprises a stop codon and the at least one microRNA binding site for a miR expressed in immune cells is located within the 3′ UTR 30-50 nucleotides after the stop codon. In another embodiment, the codon optimized open reading frame encoding the polypeptide of interest comprises a stop codon and the at least one microRNA binding site for a miR expressed in immune cells is located within the 3′ UTR at least 50 nucleotides after the stop codon. In other embodiments, the codon optimized open reading frame encoding the polypeptide of interest comprises a stop codon and the at least one microRNA binding site for a miR expressed in immune cells is located within the 3′ UTR immediately after the stop codon, or within the 3′ UTR 15-20 nucleotides after the stop codon or within the 3′ UTR 70-80 nucleotides after the stop codon. In other embodiments, the 3′ UTR comprises more than one miRNA binding site (e.g., 2-4 miRNA binding sites), wherein there can be a spacer region (e.g., of 10-100, 20-70 or 30-50 nucleotides in length) between each miRNA binding site. In another embodiment, the 3′ UTR comprises a spacer region between the end of the miRNA binding site(s) and the poly A tail nucleotides. For example, a spacer region of 10-100, 20-70 or 30-50 nucleotides in length can be situated between the end of the miRNA binding site(s) and the beginning of the poly A tail.

In one embodiment, a codon optimized open reading frame encoding a polypeptide of interest comprises a start codon and the at least one microRNA binding site is located within the 5′ UTR 1-100 nucleotides before (upstream of) the start codon. In one embodiment, the codon optimized open reading frame encoding the polypeptide of interest comprises a start codon and the at least one microRNA binding site for a miR expressed in immune cells is located within the 5′ UTR 10-50 nucleotides before (upstream of) the start codon. In another embodiment, the codon optimized open reading frame encoding the polypeptide of interest comprises a start codon and the at least one microRNA binding site for a miR expressed in immune cells is located within the 5′ UTR at least 25 nucleotides before (upstream of) the start codon. In other embodiments, the codon optimized open reading frame encoding the polypeptide of interest comprises a start codon and the at least one microRNA binding site for a miR expressed in immune cells is located within the 5′ UTR immediately before the start codon, or within the 5′ UTR 15-20 nucleotides before the start codon or within the 5′ UTR 70-80 nucleotides before the start codon. In other embodiments, the 5′ UTR comprises more than one miRNA binding site (e.g., 2-4 miRNA binding sites), wherein there can be a spacer region (e.g., of 10-100, 20-70 or 30-50 nucleotides in length) between each miRNA binding site.

In one embodiment, the 3′ UTR comprises more than one stop codon, wherein at least one miRNA binding site is positioned downstream of the stop codons. For example, a 3′ UTR can comprise 1, 2 or 3 stop codons. Non-limiting examples of triple stop codons that can be used include: UGAUAAUAG (SEQ TD NO:210), UGAUAGUAA (SEQ TD NO:211), UAAUGAUAG (SEQ ID NO:212), UGAUAAUAA (SEQ ID NO:213), UGAUAGUAG (SEQ ID NO: 214), UAAUGAUGA (SEQ TD NO: 215), UAAUAGUAG (SEQ ID NO: 216), UGAUGAUGA (SEQ ID NO:217), UAAUAAUAA (SEQ ID NO:218), and UAGUAGUAG (SEQ TD NO: 219). Within a 3′ UTR, for example, 1, 2, 3 or 4 miRNA binding sites, e.g., miR-142-3p binding sites, can be positioned immediately adjacent to the stop codon(s) or at any number of nucleotides downstream of the final stop codon. When the 3′ UTR comprises multiple miRNA binding sites, these binding sites can be positioned directly next to each other in the construct (i.e., one after the other) or, alternatively, spacer nucleotides can be positioned between each binding site.

In one embodiment, the 3′ UTR comprises three stop codons with a single miR-142-3p binding site located downstream of the 3rd stop codon. Non-limiting examples of sequences of 3′ UTR having three stop codons and a single miR-142-3p binding site located at different positions downstream of the final stop codon are shown in SEQ TD NOs: 237, 248, 249, and 250.

TABLE 4A
5′ UTRs, 3′UTRs, miR sequences, and miR binding sites

| SEQ ID NO: | Sequence |
|---|---|
| 220 | GCUGGAGCCUCGGUGGCCAUGCUUCUUGCCCCUUGGGCCUCCCCCCAGCCCCUCCUC |
| | CCCUUCCUGCACCCGUACCCCCUCCAUAAAGUAGGAAACACUACAGUGGUCUUUGAA |
| | UAAAGUCUGAGUGGGCGGC |
| | (3′ UTR with miR 142-3p binding site) |
| 202 | UCCAUAAAGUAGGAAACACUACA |
| | (miR 142-3p binding site) |
| 201 | UGUAGUGUUUCCUACUUUAUGGA |
| | (miR 142-3p sequence) |
| 203 | CAUAAAGUAGAAAGCACUACU |
| | (miR 142-5p sequence) |
| 221 | CCUCUGAAAUUCAGUUCUUCAG |
| | (miR 146-3p sequence) |
| 222 | UGAGAACUGAAUUCCAUGGGUU |
| | (miR 146-5p sequence) |
| 223 | CUCCUACAUAUUAGCAUUAACA |
| | (miR 155-3p sequence) |
| 224 | UUAAUGCUAAUCGUGAUAGGGGU |
| | (miR 155-5p sequence) |
| 206 | UCGUACCGUGAGUAAUAAUGCG |
| | (miR 126-3p sequence) |
| 208 | CAUUAUUACUUUUGGUACGCG |
| | (miR 126-5p sequence) |
| 225 | CCAGUAUUAACUGUGCUGCUGA |
| | (miR 16-3p sequence) |
| 226 | UAGCAGCACGUAAAUAUUGGCG |
| | (miR 16-5p sequence) |
| 227 | CAACACCAGUCGAUGGGCUGU |
| | (miR 21-3p sequence) |
| 228 | UAGCUUAUCAGACUGAUGUUGA |
| | (miR 21-5p sequence) |
| 143 | UGUCAGUUUGUCAAAUACCCCA |
| | (miR 223-3p sequence) |
| 230 | CGUGUAUUUGACAAGCUGAGUU |
| | (miR 223-5p sequence) |
| 231 | UGGCUCAGUUCAGCAGGAACAG |
| | (miR 24-3p sequence) |
| 232 | UGCCUACUGAGCUGAUAUCAGU |
| | (miR 24-5p sequence) |
| 233 | UUCACAGUGGCUAAGUUCCGC |
| | (miR 27-3p sequence) |
| 234 | AGGGCUUAGCUGCUUGUGAGCA |
| | (miR 27-5p sequence) |
| 207 | CGCAUUAUUACUCACGGUACGA |
| | (miR 126-3p binding site) |
| 235 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCC [symbol] GUGG |
| | UCUUUGAAUAAAGUCUGAGUGGGCGGC |
| | (3′ UTR with miR 126-3p binding site) |
| 236 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCCGUGGUCUUUGAAUAAAGUCUGAGUGG |
| | GCGGC |
| | (3′ UTR, no miR binding sites) |
| 237 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCCUCCAUAAAGUAGGAAACACUACAGUG |
| | GUCUUUGAAUAAAGUCUGAGUGGGCGGC |
| | (3′ UTR with miR 142-3p binding site) |
| 199 | UGAUAAUAGUCCAUAAAGUAGGAAACACUACAGCUGGAGCCUCGGUGGCCAUGCUUC |
| | UUGCCCCUUGGGCCUCCCCCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 142-3p and miR 126-3p binding sites |
| | variant 1) |
| 239 | UUAAUGCUAAUUGUGAUAGGGGU |
| | (miR 155-5p sequence) |
| 240 | ACCCCUAUCACAAUUAGCAUUAA |
| | (miR 155-5p binding site) |
| 241 | UGAUAAUAGUCCAUAAAGUAGGAAACACUACAGCUGGAGCCUCGGUGGCCAUGCUUC |
| | UUGCCCCUUGGGCCUCCAUAAAGUAGGAAACACUACAUCCCCCCAGCCCCUCCUCCC |
| | CUUCCUGCACCCGUACCCCCUCCAUAAAGUAGGAAACACUACAGUGGUCUUUGAAUA |
| | AAGUCUGAGUGGGCGGC |
| | (3′ UTR with 3 miR 142-3p binding sites) |
| 242 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCC [symbol] GUGGU |
| | CUUUGAAUAAAGUCUGAGUGGGCGGC |
| | (3′ UTR with miR 142-5p binding site) |
| 243 | UGAUAAUAG [symbol] GCUGGAGCCUCGGUGGCCAUGCUUCUU |
| | GCCCCUUGGGCC [symbol] UCCCCCCAGCCCCUCCUCCCCUUC |
| | CUGCACCCGUACCCCC [symbol] GUGGUCUUUGAAUAAAGUCU |
| | GAGUGGGCGGC |
| | (3′ UTR with 3 miR 142-5p binding sites) |
| 244 | UGAUAAUAG [symbol] GCUGGAGCCUCGGUGGCCAUGCUUCUU |
| | GCCCCUUGGGCCUCCAUAAAGUAGGAAACACUACAUCCCCCCAGCCCCUCCUCCCCU |
| | UCCUGCACCCGUACCCCC [symbol] GUGGUCUUUGAAUAAAGU |
| | CUGAGUGGGCGGC |
| | (3′ UTR with 2 miR 142-5p binding sites and 1 miR 142-3p |
| | binding site) |
| 245 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCCACCCCUAUCACAAUUAGCAUUAAGUG |
| | GUCUUUGAAUAAAGUCUGAGUGGGGGC |
| | (3′ UTR with miR 155-5p binding site) |
| 246 | UGAUAAUAGACCCCUAUCACAAUUAGCAUUAAGCUGGAGCCUCGGUGGCCAUGCUUC |
| | UUGCCCCUUGGGCCACCCCUAUCACAAUUAGCAUUAAUCCCCCCAGCCCCUCCUCCC |
| | CUUCCUGCACCCGUACCCCCACCCCUAUCACAAUUAGCAUUAAGUGGUCUUUGAAUA |
| | AAGUCUGAGUGGGCGGC |
| | (3′ UTR with 3 miR 155-5p binding sites) |
| 247 | UGAUAAUAGACCCCUAUCACAAUUAGCAUUAAGCUGGAGCCUCGGUGGCCAUGCUUC |
| | UUGCCCCUUGGGCCUCCAUAAAGUAGGAAACACUACAUCCCCCCAGCCCCUCCUCCC |
| | CUUCCUGCACCCGUACCCCCACCCCUAUCACAAUUAGCAUUAAGUGGUCUUUGAAUA |
| | AAGUCUGAGUGGGCGGC |
| | (3′ UTR with 2 miR 155-5p binding sites and 1 miR 142-3p |
| | binding site) |
| 248 | UGAUAAUAGUCCAUAAAGUAGGAAACACUACAGCUGGAGCCUCGGUGGCCAUGCUUC |
| | UUGCCCCUUGGGCCUCCCCCCAGCCCCUCCUCCCCUUCCUGCACCCGUACCCCCGUG |
| | GUCUUUGAAUAAAGUCUGAGUGGGCGGC |
| | (3′ UTR with miR 142-3p binding site, P1 insertion) |
| 249 | UGAUAAUAGGCUGGAGCCUCGGUGGCUCCAUAAAGUAGGAAACACUACACAUGCUUC |
| | UUGCCCCUUGGGCCUCCCCCCAGCCCCUCCUCCCCUUCCUGCACCCGUACCCCCGUG |
| | GUCUUUGAAUAAAGUCUGAGUGGGCGGC |
| | (3′ UTR with miR 142-3p binding site, P2 insertion) |
| 250 | UGAUAAUAGGCUGGAGCCUCGGUGGCCAUGCUUCUUGCCCCUUGGGCCUCCAUAAAG |
| | UAGGAAACACUACAUCCCCCCAGCCCCUCCUCCCCUUCCUGCACCCGUACCCCCGUG |
| | GUCUUUGAAUAAAGUCUGAGUGGGCGGC |
| | (3′ UTR with miR 142-3p binding site, P3 insertion) |
| 204 | AGUAGUGCUUUCUACUUUAUG |
| | (miR-142-5p binding site) |
| 200 | GACAGUGCAGUCACCCAUAAAGUAGAAAGCACUACUAACAGCACUGGAGGGUGUAGU |
| | GUUUCCUACUUUAUGGAUGAGUGUACUGUG |
| | (miR-142) |
| 197 | GGGAAAUAAGAGAGAAAAGAAGAGUAAGAAGAAAUAUAAGAGCCACC |
| | (5′ UTR) |
| 251 | GGGAAAUAAGAGUCCAUAAAGUAGGAAACACUACAAGAAAAGAAGAGUAAGAAGAAA |
| | UAUAAGAGCCACC |
| | (5′ UTR with miR142-3p binding site at position p1) |
| 252 | GGGAAAUAAGAGAGAAAAGAAGAGUAAUCCAUAAAGUAGGAAACACUACAGAAGAAA |
| | UAUAAGAGCCACC |
| | (5′ UTR with miR142-3p binding site at position p2) |
| 253 | GGGAAAUAAGAGAGAAAAGAAGAGUAAGAAGAAAUAUAAUCCAUAAAGUAGGAAACA |
| | CUACAGAGCCACC |
| | (5′ UTR with miR142-3p binding site at position p3) |
| 254 | ACCCCUAUCACAAUUAGCAUUAA |
| | (miR 155-5p binding site) |
| 255 | UGAUAAUAG [symbol] GCUGGAGCCUCGGUGGCCAUGCUUCUU |
| | GCCCCUUGGGCC [symbol] UCCCCCCAGCCCCUCUCCCCUUCC |
| | UGCACCCGUACCCCC [symbol] GUGGUCUUUGAAUAAAGUCUG |
| | AGUGGGCGGC |
| | (3′ UTR with 3 miR 142-5p binding sites) |
| 256 | UGAUAAUAGGCUGGAGCCUCGGUGGCCAUGCUUCUUGCCCCUUCCAUAAAGUAGGAA |
| | ACACUACAUGGGCCUCCCCCCAGCCCCUCCUCCCCUUCCUGCACCCGUACCCCCGUG |
| | GUCUUUGAAUAAAGUCUGAGUGGGGGC |
| | (3′ UTR including miR142-3p binding site) |
| 257 | UGAUAAUAGGCUGGAGCCUCGGUGGCCAUGCUUCUUGCCCCUUGGGCCUCCCCCCAG |
| | UCCAUAAAGUAGGAAACACUACACCCCUCCUCCCCUUCCUGCACCCGUACCCCCGUG |
| | GUCUUUGAAUAAAGUCUGAGUGGGCGGC |
| | (3′ UTR including miR142-3p binding site) |
| 258 | UGAUAAUAGGCUGGAGCCUCGGUGGCCAUGCUUCUUGCCCCUUGGGCCUCCCCCCAG |
| | CCCCUCCUCCCCUUCUCCAUAAAGUAGGAAACACUACACUGCACCCGUACCCCCGUG |
| | GUCUUUGAAUAAAGUCUGAGUGGGCGGC |
| | (3′UTR including miR142-3p binding site) |
| 259 | UGAUAAUAGGCUGGAGCCUCGGUGGCCAUGCUUCUUGCCCCUUGGGCCUCCCCCCAG |
| | CCCCUCCUCCCCUUCCUGCACCCGUACCCCCGUGGUCUUUGAAUAAAGUUCCAUAAA |
| | GUAGGAAACACUACACUGAGUGGGCGGC |
| | (3′UTR including miR142-3p binding site) |
| 260 | UGAUAAUAGUCCAUAAAGUAGGAAACACUACAGCUGGAGCCUCGGUGGCCUAGCUUC |
| | UUGCCCCUUGGGCCUCCCCCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 142-3p and miR 126-3p binding sites |
| | variant 2) |
| 261 | UGAUAAUAGGCUGGAGCCUCGGUGGCCUAGCUUCUUGCCCCUUGGGCCUCCCCCCAG |
| | CCCCUCCUCCCCUUCCUGCACCCGUACCCCCGUGGUCUUUGAAUAAAGUCUGAGUGG |
| | GCGGC |
| | (3′ UTR, no miR binding sites variant 2) |
| 198 | UGAUAAUAGGCUGGAGCCUCGGUGGCCUAGCUUCUUGCCCCUUGGGCCUCCCCCCAG |
| | CCCCUCCUCCCCUUCCUGCACCCGUACCCCCUCCAUAAAGUAGGAAACACUACAGUG |
| | GUCUUUGAAUAAAGUCUGAGUGGGCGGC |
| | (3′ UTR with miR 142-3p binding site variant 3) |
| 262 | UGAUAAUAGGCUGGAGCCUCGGUGGCCUAGCUUCUUGCCCCUUGGGCCUCCCCCCAG |
| | CCCCUCCUCCCCUUCCUGCACCCGUACCCCC [symbol] GUGG |
| | UCUUUGAAUAAAGUCUGAGUGGGCGGC |
| | (3′ UTR with miR 126-3p binding site variant 3) |
| 263 | UGAUAAUAGUCCAUAAAGUAGGAAACACUACAGCUGGAGCCUCGGUGGCCUAGCUUC |
| | UUGCCCCUUGGGCCUCCAUAAAGUAGGAAACACUACAUCCCCCCAGCCCCUCCUCCC |
| | CUUCCUGCACCCGUACCCCCUCCAUAAAGUAGGAAACACUACAGUGGUCUUUGAAUA |
| | AAGUCUGAGUGGGCGGC |
| | (3′ UTR with 3 miR 142-3p binding sites variant 2) |
| 264 | UGAUAAUAGUCCAUAAAGUAGGAAACACUACAGCUGGAGCCUCGGUGGCCUAGCUUC |
| | UUGCCCCUUGGGCCUCCCCCCAGCCCCUCCUCCCCUUCCUGCACCCGUACCCCCGUG |
| | GUCUUUGAAUAAAGUCUGAGUGGGCGGC |
| | (3′UTR with miR 142-3p binding site, P1 insertion variant |
| | 2) |
| 265 | UGAUAAUAGGCUGGAGCCUCGGUGGCUCCAUAAAGUAGGAAACACUACACUAGCUUC |
| | UUGCCCCUUGGGCCUCCCCCCAGCCCCUCCUCCCCUUCCUGCACCCGUACCCCCGUG |
| | GUCUUUGAAUAAAGUCUGAGUGGGCGGC |
| | (3′ UTR with miR 142-3p binding site, P2 insertion variant |
| | 2) |
| 266 | UGAUAAUAGGCUGGAGCCUCGGUGGCCUAGCUUCUUGCCCCUUGGGCCUCCAUAAAG |
| | UAGGAAACACUACAUCCCCCCAGCCCCUCCUCCCCUUCCUGCACCCGUACCCCCGUG |
| | GUCUUUGAAUAAAGUCUGAGUGGGCGGC |
| | (3′ UTR with miR 142-3p binding site, P3 insertion variant |
| | 2) |
| 267 | UGAUAAUAGGCUGGAGCCUCGGUGGCCUAGCUUCUUGCCCCUUGGGCCUCCCCCCAG |
| | CCCCUCCUCCCCUUCCUGCACCCGUACCCCCACCCCUAUCACAAUUAGCAUUAAGUG |
| | GUCUUUGAAUAAAGUCUGAGUGGGCGGC |
| | (3′UTR with miR 155-5p binding site variant 2) |
| 268 | UGAUAAUAGACCCCUAUCACAAUUAGCAUUAAGCUGGAGCCUCGGUGGCCUAGCUUC |
| | UUGCCCCUUGGGCCACCCCUAUCACAAUUAGCAUUAAUCCCCCCAGCCCCUCCUCCC |
| | CUUCCUGCACCCGUACCCCCACCCCUAUCACAAUUAGCAUUAAGUGGUCUUUGAAUA |
| | AAGUCUGAGUGGGCGGC |
| | (3′ UTR with 3 miR 155-5p binding sites variant 2) |
| 269 | UGAUAAUAGACCCCUAUCACAAUUAGCAUUAAGCUGGAGCCUCGGUGGCCUAGCUUC |
| | UUGCCCCUUGGGCCUCCAUAAAGUAGGAAACACUACAUCCCCCCAGCCCCUCCUCCC |
| | CUUCCUGCACCCGUACCCCCACCCCUAUCACAAUUAGCAUUAAGUGGUCUUUGAAUA |
| | AAGUCUGAGUGGGCGGC |
| | (3′UTR with 2 miR 155-5p binding sites and 1 miR 142-3p |
| | binding site variant 2) |
| 271 | AGGAAAUAAGAGAGAAAAGAAGAGUAAGAAGAAAUAUAAGAGCCACC |
| | 5′-UTR (v1 plus A-start) |
| 272 | GGGAAAUAAGAGAGAAAAGAAGAGUAAGAAGAAAUAUAAGACCCCGGCGCCGCCACC |
| | (5′ UTR v1.1 plus G-start) |
| 273 | AGGAAAUAAGAGAGAAAAGAAGAGUAAGAAGAAAUAUAAGACCCCGGCGCCGCCACC |
| | (5′UTR v1.1 plus A-start) |
| 274 | GGGAGAUCAGAGAGAAAAGAAGAGUAAGAAGAAAUAUAAGAGCCACC |
| | 5′ UTR 002 (upstream UTR plus G-start) |
| 80 | GGGAGACAAGCUUGGCAUUCCGGUACUGUUGGUAAAGCCACC |
| | 5′ UTR-004 (Upstream UTR plus G-start) |
| 81 | GGGAAUUAACAGAGAAAAGAAGAGUAAGAAGAAAUAUAAGAGCCACC |
| | 5′ UTR-008 (Upstream UTR plus G-start) |
| 82 | GGGAAAUUAGACAGAAAAGAAGAGUAAGAAGAAAUAUAAGAGCCACC |
| | 5′ UTR-009 (Upstream UTR plus G-start) |
| 83 | GGGAAAUAAGAGAGUAAAGAACAGUAAGAAGAAAUAUAAGAGCCACC |
| | 5′ UTR-010, (Upstream UTR plus G-start) |
| 84 | GGGAAAAAAGAGAGAAAAGAAGACUAAGAAGAAAUAUAAGAGCCACC |
| | 5′ UTR-011 (Upstream UTR plus G-start) |
| 85 | GGGAAAUAAGAGAGAAAAGAAGAGUAAGAAGAUAUAUAAGAGCCACC |
| | 5′ UTR-012 (Upstream UTR plus G-start) |
| 86 | GGGAAAUAAGAGACAAAACAAGAGUAAGAAGAAAUAUAAGAGCCACC |
| | 5′ UTR-013 (Upstream UTR plus G-start) |
| 87 | GGGAAAUUAGAGAGUAAAGAACAGUAAGUAGAAUUAAAAGAGCCACC |
| | 5′ UTR-014 (Upstream UTR plus G-start) |
| 88 | GGGAAAUAAGAGAGAAUAGAAGAGUAAGAAGAAAUAUAAGAGCCACC |
| | 5′ UTR-015 (Upstream UTR plus G-start) |
| 89 | GGGAAAUAAGAGAGAAAAGAAGAGUAAGAAGAAAAUUAAGAGCCACC |
| | 5′ UTR-016 (Upstream UTR plus G-start) |
| 90 | GGGAAAUAAGAGAGAAAAGAAGAGUAAGAAGAAAUUUAAGAGCCACC |
| | 5′ UTR-017 (Upstream UTR plus G-start) |
| 91 | UCAAGCUUUUGGACCCUCGUACAGAAGCUAAUACGACUCACUAUAGGGAAAUAAGAG |
| | AGAAAAGAAGAGUAAGAAGAAAUAUAAGAGCCACC |
| | 5′ UTR-018 (Upstream UTR) |

Stop codon = bold
miR 142-3p binding site = underline
miR 126-3p binding site = bold underline
miR 155-5p binding site = italicized
miR 142-5p binding site = italicized and bold underline

In one embodiment, the polynucleotide of the invention comprises a 5′ UTR, a codon optimized open reading frame encoding a polypeptide of interest, a 3′ UTR comprising the at least one miRNA binding site for a miR expressed in immune cells, and a 3′ tailing region of linked nucleosides. In various embodiments, the 3′ UTR comprises 1-4, at least two, one, two, three or four miRNA binding sites for miRs expressed in immune cells, preferably abundantly or preferentially expressed in immune cells.

In one embodiment, the at least one miRNA expressed in immune cells is a miR-142-3p microRNA binding site. In one embodiment, the miR-142-3p microRNA binding site comprises the sequence shown in SEQ ID NO: 202. In one embodiment, the 3′ UTR of the mRNA comprising the miR-142-3p microRNA binding site comprises the sequence shown in SEQ ID NO: 220.

In one embodiment, the at least one miRNA expressed in immune cells is a miR-126 microRNA binding site. In one embodiment, the miR-126 binding site is a miR-126-3p binding site. In one embodiment, the miR-126-3p microRNA binding site comprises the sequence shown in SEQ ID NO: 207. In one embodiment, the 3′ UTR of the mRNA of the invention comprising the miR-126-3p microRNA binding site comprises the sequence shown in SEQ ID NO: 235.

Non-limiting exemplary sequences for miRs to which a microRNA binding site(s) of the disclosure can bind include the following: miR-142-3p (SEQ ID NO: 201), miR-142-5p (SEQ ID NO: 203), miR-146-3p (SEQ ID NO: 221), miR-146-5p (SEQ ID NO: 222), miR-155-3p (SEQ ID NO: 223), miR-155-5p (SEQ ID NO: 224), miR-126-3p (SEQ ID NO: 206), miR-126-5p (SEQ ID NO: 208), miR-16-3p (SEQ ID NO: 225), miR-16-5p (SEQ ID NO: 226), miR-21-3p (SEQ ID NO: 227), miR-21-5p (SEQ ID NO: 228), miR-223-3p (SEQ ID NO: 143), miR-223-5p (SEQ ID NO: 230), miR-24-3p (SEQ ID NO: 231), miR-24-5p (SEQ ID NO: 232), miR-27-3p (SEQ ID NO: 233) and miR-27-5p (SEQ ID NO: 234). Other suitable miR sequences expressed in immune cells (e.g., abundantly or preferentially expressed in immune cells) are known and available in the art, for example at the University of Manchester's microRNA database, miRBase. Sites that bind any of the aforementioned miRs can be designed based on Watson-Crick complementarity to the miR, typically 100% complementarity to the miR, and inserted into an mRNA construct of the disclosure as described herein.

In another embodiment, a polynucleotide of the present invention (e.g., and mRNA, e.g., the 3′ UTR thereof) can comprise at least one miRNA binding site to thereby reduce or inhibit accelerated blood clearance, for example by reducing or inhibiting production of IgMs, e.g., against PEG, by B cells and/or reducing or inhibiting proliferation and/or activation of pDCs, and can comprise at least one miRNA binding site for modulating tissue expression of an encoded protein of interest.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′ UTR of the same sequence type.

In one embodiment, other regulatory elements and/or structural elements of the 5′ UTR can influence miRNA mediated gene regulation. One example of a regulatory element and/or structural element is a structured IRES (Internal Ribosome Entry Site) in the 5′ UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The polynucleotides of the invention can further include this structured 5′ UTR to enhance microRNA mediated gene regulation.

At least one miRNA binding site can be engineered into the 3′ UTR of a polynucleotide of the invention. In this context, at least two, at least three, at least four, at least five, at least six, at least seven, at least eight, at least nine, at least ten, or more miRNA binding sites can be engineered into a 3′ UTR of a polynucleotide of the invention. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a polynucleotide of the invention. In one embodiment, miRNA binding sites incorporated into a polynucleotide of the invention can be the same or can be different miRNA sites. A combination of different miRNA binding sites incorporated into a polynucleotide of the invention can include combinations in which more than one copy of any of the different miRNA sites are incorporated. In another embodiment, miRNA binding sites incorporated into a polynucleotide of the invention can target the same or different tissues in the body. As a non-limiting example, through the introduction of tissue-, cell-type-, or disease-specific miRNA binding sites in the 3′-UTR of a polynucleotide of the invention, the degree of expression in specific cell types (e.g., myeloid cells, endothelial cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′ UTR in a polynucleotide of the invention. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′ UTR. In another non-limiting example, a miRNA binding site can be engineered near the 5′ terminus of the 3′ UTR and near the 3′ terminus of the 3′ UTR.

In another embodiment, a 3′ UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In some embodiments, the expression of a polynucleotide of the invention can be controlled by incorporating at least one sensor sequence in the polynucleotide and formulating the polynucleotide for administration. As a non-limiting example, a polynucleotide of the invention can be targeted to a tissue or cell by incorporating a miRNA binding site and formulating the polynucleotide in a lipid nanoparticle comprising a ionizable lipid, including any of the lipids described herein.

A polynucleotide of the invention can be engineered for more targeted expression in specific tissues, cell types, or biological conditions based on the expression patterns of miRNAs in the different tissues, cell types, or biological conditions. Through introduction of tissue-specific miRNA binding sites, a polynucleotide of the invention can be designed for optimal protein expression in a tissue or cell, or in the context of a biological condition.

In some embodiments, a polynucleotide of the invention can be designed to incorporate miRNA binding sites that either have 100% identity to known miRNA seed sequences or have less than 100% identity to miRNA seed sequences. In some embodiments, a polynucleotide of the invention can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the polynucleotide. In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment the miRNA sequence in the 5′ UTR can be used to stabilize a polynucleotide of the invention described herein.

In another embodiment, a miRNA sequence in the 5′ UTR of a polynucleotide of the invention can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) to decrease the accessibility to the first start codon (AUG).

Matsuda showed that altering the sequence around the start codon with an LNA or EJC affected the efficiency, length and structural stability of a polynucleotide. A polynucleotide of the invention can comprise a miRNA sequence, instead of the LNA or EJC sequence described by Matsuda et al, near the site of translation initiation to decrease the accessibility to the site of translation initiation. The site of translation initiation can be prior to, after or within the miRNA sequence. As a non-limiting example, the site of translation initiation can be located within a miRNA sequence such as a seed sequence or binding site.

In some embodiments, a polynucleotide of the invention can include at least one miRNA to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a polynucleotide of the invention can be specific to the hematopoietic system. As another non-limiting example, a miRNA incorporated into a polynucleotide of the invention to dampen antigen presentation is miR-142-3p.

In some embodiments, a polynucleotide of the invention can include at least one miRNA to dampen expression of the encoded polypeptide in a tissue or cell of interest. As a non-limiting example, a polynucleotide of the invention can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a polynucleotide of the invention can comprise at least one miRNA binding site in the 3′UTR to selectively degrade mRNA therapeutics in the immune cells to subdue unwanted immunogenic reactions caused by therapeutic delivery. As a non-limiting example, the miRNA binding site can make a polynucleotide of the invention more unstable in antigen presenting cells. Non-limiting examples of these miRNAs include miR-142-5p, miR-142-3p, miR-146a-5p, and miR-146-3p.

In one embodiment, a polynucleotide of the invention comprises at least one miRNA sequence in a region of the polynucleotide that can interact with a RNA binding protein. In some embodiments, the polynucleotide of the invention (e.g., a RNA, e.g., an mRNA) comprising (i) a sequence-optimized nucleotide sequence (e.g., an ORF) encoding an immune checkpoint inhibitor polypeptide (e.g., the wild-type sequence, functional fragment, or variant thereof) and (ii) a miRNA binding site (e.g., a miRNA binding site that binds to miR-142) and/or a miRNA binding site that binds to miR-126.

### IVT Polynucleotide Architecture

In some embodiments, the polynucleotide of the present disclosure comprising an mRNA encoding an ITB6 molecule polypeptide is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded immune checkpoint inhibitor molecule polypeptide. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding an immune checkpoint inhibitor molecule polypeptide can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of an immune checkpoint inhibitor molecule polypeptide or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture are disclosed in International PCT application WO 2017/201325, filed on 18 May 2017, the entire contents of which are hereby incorporated by reference.

### 5′UTR and 3′ UTR

A UTR can be homologous or heterologous to the coding region in a polynucleotide. In some embodiments, the UTR is homologous to the ORF encoding an ITB6 molecule. In some embodiments, the UTR is heterologous to the ORF encoding an ITB6 molecule.

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO:87), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Additional exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a 3-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelan equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs. In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of the 5′ UTR or 3′ UTR sequences disclosed herein (e.g., in Table A or Table B), and any combination thereof.

The polynucleotides of the invention can comprise combinations of features. For example, the ORF can be flanked by a 5′ UTR that comprises a strong Kozak translational initiation signal and/or a 3′ UTR comprising an oligo(dT) sequence for templated addition of a poly-A tail. A 5′ UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation.

### a. 5′ UTR Sequences

5′ UTR sequences are important for ribosome recruitment to the mRNA and have been reported to play a role in translation (Hinnebusch A, et al., (2016) Science, 352:6292: 1413-6).

Disclosed herein, inter alia, is a polynucleotide, e.g., mRNA, comprising an open reading frame encoding an ITB6 polypeptide (e.g., as described herein) encoding a polypeptide, wherein the polynucleotide has a 5′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as provided in Table A or a variant or fragment thereof); (b) a coding region comprising a stop element (e.g., as described herein); and (c) a 3′-UTR (e.g., as described herein), and LNP compositions comprising the same. In an embodiment, the polynucleotide comprises a 5′-UTR comprising a sequence provided in Table A or a variant or fragment thereof (e.g., a functional variant or fragment thereof).

In an embodiment, the polynucleotide having a 5′ UTR sequence provided in Table A or a variant or fragment thereof, has an increase in the half-life of the polynucleotide, e.g., about 1.5-20-fold increase in half-life of the polynucleotide. In an embodiment, the increase in half-life is about 1.5, 2, 3, 4, 5, 6, 7, 8, 9, 10, 11, 12, 13, 14, 15, 16, 17, 18, 19, or 20-fold, or more. In an embodiment, the increase in half life is about 1.5-fold or more. In an embodiment, the increase in half life is about 2-fold or more. In an embodiment, the increase in half life is about 3-fold or more. In an embodiment, the increase in half life is about 4-fold or more. In an embodiment, the increase in half life is about 5-fold or more.

In an embodiment, the polynucleotide having a 5′ UTR sequence provided in Table A or a variant or fragment thereof, results in an increased level and/or activity, e.g., output, of the polypeptide encoded by the polynucleotide. In an embodiment, the 5′ UTR results in about 1.5-20-fold increase in level and/or activity, e.g., output, of the polypeptide encoded by the polynucleotide. In an embodiment, the increase in level and/or activity is about 1.5, 2, 3, 4, 5, 6, 7, 8, 9, 10, 11, 12, 13, 14, 15, 16, 17, 18, 19, or 20-fold, or more. In an embodiment, the increase in level and/or activity is about 1.5-fold or more. In an embodiment, the increase in level and/or activity is about 2-fold or more. In an embodiment, the increase in level and/or activity is about 3-fold or more. In an embodiment, the increase in level and/or activity is about 4-fold or more. In an embodiment, the increase in level and/or activity is about 5-fold or more.

In an embodiment, the increase is compared to an otherwise similar polynucleotide which does not have a 5′ UTR, has a different 5′ UTR, or does not have a 5′ UTR described in Table A or a variant or fragment thereof.

In an embodiment, the increase in half-life of the polynucleotide is measured according to an assay that measures the half-life of a polynucleotide, e.g., an assay described herein.

In an embodiment, the increase in level and/or activity, e.g., output, of the polypeptide encoded by the polynucleotide is measured according to an assay that measures the level and/or activity of a polypeptide, e.g., an assay described herein.

In an embodiment, the 5′ UTR comprises a sequence provided in Table A or a sequence with at least 80%, 85%, 90%, 95%, 96%, 97%, 98%, 99% or 100% identity to a 5′ UTR sequence provided in Table A, or a variant or a fragment thereof. In an embodiment, the 5′ UTR comprises a sequence with at least 80%, 85%, 90%, 95%, 96%, 97%, 98%, 99% or 100% identity to SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In an embodiment, the 5′ UTR comprises a sequence with at least 80%, 85%, 90%, 95%, 96%, 97%, 98%, 99%, or 100% identity to SEQ ID NO: 50. In an embodiment, the 5′ UTR comprises a sequence with at least 80%, 85%, 90%, 95%, 96%, 97%, 98%, 99%, or 100% identity to SEQ ID NO: 51. In an embodiment, the 5′ UTR comprises a sequence with at least 80%, 85%, 90%, 95%, 96%, 97%, 98%, 99%, or 100% identity to SEQ ID NO: 52. In an embodiment, the 5′ UTR comprises a sequence with at least 80%, 85%, 90%, 95%, 96%, 97%, 98%, 99%, or 100% identity to SEQ ID NO: 53. In an embodiment, the 5′ UTR comprises a sequence with at least 80%, 85%, 90%, 95%, 96%, 97%, 98%, 99%, or 100% identity to SEQ ID NO: 54. In an embodiment, the 5′ UTR comprises a sequence with at least 80%, 85%, 90%, 95%, 96%, 97%, 98%, 99%, or 100% identity to SEQ ID NO: 55. In an embodiment, the 5′ UTR comprises a sequence with at least 80%, 85%, 90%, 95%, 96%, 97%, 98%, 99%, or 100% identity to SEQ ID NO: 56. In an embodiment, the 5′ UTR comprises a sequence with at least 80%, 85%, 90%, 95%, 96%, 97%, 98%, 99%, or 100% identity to SEQ ID NO: 57. In an embodiment, the 5′ UTR comprises a sequence with at least 80%, 85%, 90%, 95%, 96%, 97%, 98%, 99%, or 100% identity to SEQ ID NO: 58. In an embodiment, the 5′ UTR comprises a sequence with at least 80%, 85%, 90%, 95%, 96%, 97%, 98%, 99%, or 100% identity to SEQ ID NO: 59. In an embodiment, the 5′ UTR comprises a sequence with at least 80%, 85%, 90%, 95%, 96%, 97%, 98%, 99%, or 100% identity to SEQ ID NO: 60. In an embodiment, the 5′ UTR comprises a sequence with at least 80%, 85%, 90%, 95%, 96%, 97%, 98%, 99%, or 100% identity to SEQ ID NO: 61. In an embodiment, the 5′ UTR comprises a sequence with at least 80%, 85%, 90%, 95%, 96%, 97%, 98%, 99%, or 100% identity to SEQ ID NO: 62. In an embodiment, the 5′ UTR comprises a sequence with at least 80%, 85%, 90%, 95%, 96%, 97%, 98%, 99%, or 100% identity to SEQ ID NO: 63. In an embodiment, the 5′ UTR comprises a sequence with at least 80%, 85%, 90%, 95%, 96%, 97%, 98%, 99%, or 100% identity to SEQ ID NO: 64. In an embodiment, the 5′ UTR comprises a sequence with at least 80%, 85%, 90%, 95%, 96%, 97%, 98%, 99%, or 100% identity to SEQ ID NO: 65. In an embodiment, the 5′ UTR comprises a sequence with at least 80%, 85%, 90%, 95%, 96%, 97%, 98%, 99%, or 100% identity to SEQ ID NO: 66. In an embodiment, the 5′ UTR comprises a sequence with at least 80%, 85%, 90%, 95%, 96%, 97%, 98%, 99%, or 100% identity to SEQ ID NO: 67. In an embodiment, the 5′ UTR comprises a sequence with at least 80%, 85%, 90%, 95%, 96%, 97%, 98%, 99%, or 100% identity to SEQ ID NO: 68. In an embodiment, the 5′ UTR comprises a sequence with at least 80%, 85%, 90%, 95%, 96%, 97%, 98%, 99%, or 100% identity to SEQ ID NO: 69. In an embodiment, the 5′ UTR comprises a sequence with at least 80%, 85%, 90%, 95%, 96%, 97%, 98%, 99%, or 100% identity to SEQ ID NO: 70. In an embodiment, the 5′ UTR comprises a sequence with at least 80%, 85%, 90%, 95%, 96%, 97%, 98%, 99%, or 100% identity to SEQ ID NO: 71. In an embodiment, the 5′ UTR comprises a sequence with at least 80%, 85%, 90%, 95%, 96%, 97%, 98%, 99%, or 100% identity to SEQ ID NO: 72. In an embodiment, the 5′ UTR comprises a sequence with at least 80%, 85%, 90%, 95%, 96%, 97%, 98%, 99%, or 100% identity to SEQ ID NO: 73. In an embodiment, the 5′ UTR comprises a sequence with at least 80%, 85%, 90%, 95%, 96%, 97%, 98%, 99%, or 100% identity to SEQ ID NO: 74. In an embodiment, the 5′ UTR comprises a sequence with at least 80%, 85%, 90%, 95%, 96%, 97%, 98%, 99%, or 100% identity to SEQ ID NO: 75. In an embodiment, the 5′ UTR comprises a sequence with at least 80%, 85%, 90%, 95%, 96%, 97%, 98%, 99%, or 100% identity to SEQ ID NO: 76. In an embodiment, the 5′ UTR comprises a sequence with at least 80%, 85%, 90%, 95%, 96%, 97%, 98%, 99%, or 100% identity to SEQ ID NO: 77. In an embodiment, the 5′ UTR comprises a sequence with at least 80%, 85%, 90%, 95%, 96%, 97%, 98%, 99%, or 100% identity to SEQ ID NO: 78. In an embodiment, the 5′ UTR comprises a sequence with at least 80%, 85%, 90%, 95%, 96%, 97%, 98%, 99%, or 100% identity to SEQ ID NO: 79. In an embodiment, the 5′ UTR comprises a sequence with at least 80%, 85%, 90%, 95%, 96%, 97%, 98%, 99%, or 100% identity to SEQ ID NO: 80.

In an embodiment, a 5′ UTR sequence provided in Table A has a first nucleotide which is an A. In an embodiment, a 5′ UTR sequence provided in Table A has a first nucleotide which is a G.

TABLE A
5′ UTR sequences

| SEQ ID | Sequence | |
|---|---|---|
| NO: | Name | Sequence |
| 50 | A1 | GGAAAUCGCAAAAUUUGCUCUUCGCGUUAGAUUUCUUU |
| | | UAGUUUUCUCGCAACUAGCAAGCUUUUUGUUCUCGCC |
| 51 | A5 | GGAAAUCCCCACAACCGCCUCAUAUCCAGGCUCAAGAAU |
| | | AGAGCUCAGUGUUUUGUUGUUUAAUCAUUCCGACGUGU |
| | | UUUGCGAUAUUCGCGCAAAGCAGCCAGUCGCGCGCUUGC |
| | | UUUUAAGUAGAGUUGUUUUUCCACCCGUUUGCCAGGCA |
| | | UCUUUAAUUUAACAUAUUUUUAUUUUUCAGGCUAACCU |
| | | ACGCCGCCACC |
| 52 | A6 | GGAAAUAAGAGAGAAAAGAAGAGUAAGAAGAAAUAUAA |
| | | GAUCUCCCUGAGCUUCAGGGAGCCCCGGCGCCGCCACC |
| 53 | A7 | GGAAACCCCCCACCCCCGUAAGAGAGAAAAGAAGAGUAA |
| | | GAAGAAAUAUAAGAUCUCCCUGAGCUUCAGGGAGCCCC |
| | | GGCGCCGCCACC |
| 54 | A8 | GGAGAACUUCCGCUUCCGUUGGCGCAAGCGCUUUCAUUU |
| | | UUUCUGCUACCGUGACUAAG |
| 55 | A9 | GGAAAUAAGAGAGAAAAGAAGAGUAAGAAGAAAUAUAA |
| | | GAGCCACC |
| 56 | A11 | GGAAAUAAGAGAGAAAAGAAGAGUAAGAAGAAAUAUAA |
| | (Reference) | GACCCCGGCGCCGCCACC |
| 57 | A2 | GGAAAUCGCAAAAUUUGCUCUUCGCGUUAGAUUUCUUU |
| | | UAGUUUUCUCGCAACUAGCAAGCUUUUUGUUCUCGCCGC |
| | | CGCC |
| 58 | A3 | GGAAAUCGCAAAAUUUUCUUUUCGCGUUAGAUUUCUUU |
| | | UAGUUUUCUUUCAACUAGCAAGCUUUUUGUUCUCGCCG |
| | | CCGCC |
| 59 | A4 | GGAAAUCGCAAAA(N₂)x(N₃)xCU(N₄)x(N₅)xCGCG |
| | | UUAGAUUUCUUUUAGUUUUCUN₆N₇CAACU |
| | | AGCAAGCUUUUUGUUCUCGCC(N₈CC)x |
| | | (N₂)x is a uracil and x is an integer from 0 to 5, e.g., wherein x = |
| | | 3 or 4; |
| | | (N₃)x is a guanine and x is an integer from 0 to 1; |
| | | (N₄)x is a cytosine and x is an integer from 0 to 1; |
| | | (N₅)x is a uracil and x is an integer from 0 to 5, e.g., wherein x = |
| | | 2 or 3; |
| | | N₆ is a uracil or cytosine; |
| | | N₇ is a uracil or guanine; |
| | | N₈ is adenine or guanine and x is an integer from 0 to 1. |
| 60 | A27 | GGAAAAUUUUAGCCUGGAACGUUAGAUAACUGUCCUGU |
| | | UGUCUUUAUAUACUUGGUCCCCAAGUAGUUUGUCUUCC |
| | | AAA |
| 61 | A12 | GGAAACUUUAUUUAGUGUUACUUUAUUUUCUGUUUAUU |
| | | UGUGUUUCUUCAGUGGGUUUGUUCUAAUUUCCUUGGCC |
| | | GCC |
| 62 | A13 | GGAAAAUCUGUAUUAGGUUGGCGUGUUCUUUGGUCGGU |
| | | UGUUAGUAUUGUUGUUGAUUCGUUUGUGGUCGGUUGCC |
| | | GCC |
| 63 | A14 | GGAAAAUUAUUAACAUCUUGGUAUUCUCGAUAACCAUU |
| | | CGUUGGAUUUUAUUGUAUUCGUAGUUUGGGUUCCUGCC |
| | | GCC |
| 64 | A15 | GGAAAUUAUUAUUAUUUCUAGCUACAAUUUAUCAUUGU |
| | | AUUAUUUUAGCUAUUCAUCAUUAUUUACUUGGUGAUCA |
| | | ACA |
| 65 | A16 | GGAAAUAGGUUGUUAACCAAGUUCAAGCCUAAUAAGCU |
| | | UGGAUUCUGGUGACUUGCUUCACCGUUGGCGGGCACCG |
| | | AUC |
| 66 | A17 | GGAAAUCGUAGAGAGUCGUACUUAGUACAUAUCGACUA |
| | | UCGGUGGACACCAUCAAGAUUAUAAACCAGGCCAGA |
| 67 | A18 | GGAAACCCGCCCAAGCGACCCCAACAUAUCAGCAGUUGC |
| | | CCAAUCCCAACUCCCAACACAAUCCCCAAGCAACGCCGC |
| | | C |
| 68 | A19 | GGAAAGCGAUUGAAGGCGUCUUUUCAACUACUCGAUUA |
| | | AGGUUGGGUAUCGUCGUGGGACUUGGAAAUUUGUUGUU |
| | | UCC |
| 69 | A20 | GGAAACUAAUCGAAAUAAAAGAGCCCCGUACUCUUUUA |
| | | UUUCUAUUAGGUUAGGAGCCUUAGCAUUUGUAUCUUAG |
| | | GUA |
| 70 | A21 | GGAAAUGUGAUUUCCAGCAACUUCUUUUGAAUAUAUUG |
| | | AAUUCCUAAUUCAAAGCGAACAAAUCUACAAGCCAUAU |
| | | ACC |
| 71 | A22 | GGAAAUCGUAGAGAGUCGUACUUACGUGGUCGCCAUUG |
| | | CAUAGCGCGCGAAAGCAACAGGAACAAGAACGCGCC |
| 72 | A23 | GGAAAUCGUAGAGAGUCGUACUUAGAAUAAACAGAGUC |
| | | GGGUCGACUUGUCUCUGAUACUACGACGUCACAAUC |
| 73 | A24 | GGAAAAUUUGCCUUCGGAGUUGCGUAUCCUGAACUGCC |
| | | CAGCCUCCUGAUAUACAACUGUUCCGCUUAUUCGGGCCG |
| | | CC |
| 74 | A25 | GGAAAUCUGAGCAGGAAUCCUUUGUGCAUUGAAGACUU |
| | | UAGAUUCCUCUCUGCGGUAGACGUGCACUUAUAAGUAU |
| | | UUG |
| 75 | A26 | GGAAAGCGAUUGAAGGCGUCUUUUCAACUACUCGAUUA |
| | | AGGUUGGGUAUCGUCGUGGGACUUGGAAAUUUGUUGCC |
| | | ACC |
| 76 | A28 | GGAAAUUUUUUUUUGAUAUUAUAAGAGUUUUUUUUUGA |
| | | UAUUAAGAAAAUUUUUUUUUGAUAUUAGAAGAGUAAGA |
| | | AGAAAUAUAAGACCCCGGCGCCGCCACC |
| 77 | A29 | GGAAAUAAGAGAGAAAAGAAGAGUAAGAAGAAAUAUAA |
| | | GAGCCAAAAAAAAAAAACC |
| 78 | A30 | GGAAAUCUCCCUGAGCUUCAGGGAGUAAGAGAGAAAAG |
| | | AAGAGUAAGAAGAAAUAUAAGACCCCGGCGCCGCCACC |
| 79 | A31 | GCCRCC, wherein R = A or G |
| 80 | A32 | GGAAAUCGCAAAAUUUGCUCUUCGCGUUAGAUUUCUUU |
| | | UAGUUUUCUCGCAACUAGCAAGCUUUUUGUUCUCGCC |

In an embodiment, the 5′ UTR comprises a variant of SEQ ID NO: 50. In an embodiment, the variant of SEQ ID NO: 50 comprises a nucleic acid sequence of Formula A:

(SEQ ID NO: 59)
GGAAAUCGCAAAA(N2)X(N3)X CU(N4)X(N5)XCGCGUUAGAUUU
CUUUUAGUUUUCUN6N7CAACUAGCAAGCUUUUUGUUCUCGC
C(N8CC)x,

wherein:
(N2)x is a uracil and x is an integer from 0 to 5, e.g., wherein x=3 or 4;
(N3)x is a guanine and x is an integer from 0 to 1;
(N4)x is a cytosine and x is an integer from 0 to 1;
(N5)x is a uracil and x is an integer from 0 to 5, e.g., wherein x=2 or 3;
N6 is a uracil or cytosine;
N7 is a uracil or guanine;
N8 is adenine or guanine and x is an integer from 0 to 1.

In an embodiment (N2)x is a uracil and x is 0. In an embodiment (N2)x is a uracil and x is 1. In an embodiment (N2)x is a uracil and x is 2. In an embodiment (N2)x is a uracil and x is 3. In an embodiment, (N2)x is a uracil and x is 4. In an embodiment (N2)x is a uracil and x is 5.

In an embodiment, (N3)x is a guanine and x is 0. In an embodiment, (N3)x is a guanine and x is 1.

In an embodiment, (N4)x is a cytosine and x is 0. In an embodiment, (N4)x is a cytosine and x is 1.

In an embodiment (N5)x is a uracil and x is 0. In an embodiment (N5)x is a uracil and x is 1. In an embodiment (N5)x is a uracil and x is 2. In an embodiment (N5)x is a uracil and x is 3. In an embodiment, (N5)x is a uracil and x is 4. In an embodiment (N5)x is a uracil and x is 5.

In an embodiment, N6 is a uracil. In an embodiment, N6 is a cytosine.

In an embodiment, N7 is a uracil. In an embodiment, N7 is a guanine.

In an embodiment, N8 is an adenine and x is 0. In an embodiment, N8 is an adenine and x is 1.

In an embodiment, N8 is a guanine and x is 0. In an embodiment, N8 is a guanine and x is 1.

In an embodiment, the 5′ UTR comprises a variant of SEQ ID NO: 50. In an embodiment, the variant of SEQ ID NO: 50 comprises a sequence with at least 50%, 60%, 70%, 80%, 85%, 90%, 95%, 96%, 97%, 98%, or 99% identity to SEQ ID NO: 50. In an embodiment, the variant of SEQ ID NO: 50 comprises a sequence with at least 50% identity to SEQ ID NO: 50. In an embodiment, the variant of SEQ ID NO: 50 comprises a sequence with at least 60% identity to SEQ ID NO: 50. In an embodiment, the variant of SEQ ID NO: 50 comprises a sequence with at least 70% identity to SEQ ID NO: 50. In an embodiment, the variant of SEQ ID NO: 50 comprises a sequence with at least 80% identity to SEQ ID NO: 50. In an embodiment, the variant of SEQ ID NO: 50 comprises a sequence with at least 90% identity to SEQ ID NO: 50. In an embodiment, the variant of SEQ ID NO: 50 comprises a sequence with at least 95% identity to SEQ ID NO: 50. In an embodiment, the variant of SEQ ID NO: 50 comprises a sequence with at least 96% identity to SEQ ID NO: 50. In an embodiment, the variant of SEQ ID NO: 50 comprises a sequence with at least 97% identity to SEQ ID NO: 50. In an embodiment, the variant of SEQ ID NO: 50 comprises a sequence with at least 98% identity to SEQ ID NO: 50. In an embodiment, the variant of SEQ ID NO: 50A comprises a sequence with at least 99% identity to SEQ ID NO: 50.

In an embodiment, the variant of SEQ ID NO: 50 comprises a uridine content of at least 5%, 10%, 20%, 30%, 40%, 50%, 60%, 70%, or 80%. In an embodiment, the variant of SEQ ID NO: 50 comprises a uridine content of at least 5%. In an embodiment, the variant of SEQ ID NO: 50 comprises a uridine content of at least 10%. In an embodiment, the variant of SEQ ID NO: 50 comprises a uridine content of at least 20%. In an embodiment, the variant of SEQ ID NO: 50 comprises a uridine content of at least 30%. In an embodiment, the variant of SEQ ID NO: 50 comprises a uridine content of at least 40%. In an embodiment, the variant of SEQ ID NO: 50 comprises a uridine content of at least 50%. In an embodiment, the variant of SEQ ID NO: 50 comprises a uridine content of at least 60%. In an embodiment, the variant of SEQ ID NO: 50 comprises a uridine content of at least 70%. In an embodiment, the variant of SEQ ID NO: 50 comprises a uridine content of at least 80%.

In an embodiment, the variant of SEQ ID NO: 50 comprises at least 2, 3, 4, 5, 6 or 7 consecutive uridines (e.g., a polyuridine tract). In an embodiment, the polyuridine tract in the variant of SEQ ID NO: 50 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO: 50 comprises 4 consecutive uridines. In an embodiment, the polyuridine tract in the variant of SEQ ID NO: 50 comprises 5 consecutive uridines.

In an embodiment, the variant of SEQ ID NO: 50 comprises 1, 2, 3, 4, 5, 6, 7, 8, 9, 10, 11, 12, 13, 14, or 15 polyuridine tracts. In an embodiment, the variant of SEQ ID NO: 50 comprises 3 polyuridine tracts. In an embodiment, the variant of SEQ ID NO: 50 comprises 4 polyuridine tracts. In an embodiment, the variant of SEQ ID NO: 50 comprises 5 polyuridine tracts.

In an embodiment, one or more of the polyuridine tracts are adjacent to a different polyuridine tract. In an embodiment, each of, e.g., all, the polyuridine tracts are adjacent to each other, e.g., all of the polyuridine tracts are contiguous.

In an embodiment, one or more of the polyuridine tracts are separated by 1, 2, 3, 4, 5, 6, 7, 8, 9, 10, 11, 2, 13, 14, 15, 16, 17, 18, 19, 20, 30, 40, 50, or 60 nucleotides. In an embodiment, each of, e.g., all of, the polyuridine tracts are separated by 1, 2, 3, 4, 5, 6, 7, 8, 9, 10, 11, 2, 13, 14, 15, 16, 17, 18, 19, 20, 30, 40, 50, or 60 nucleotides.

In an embodiment, a first polyuridine tract and a second polyuridine tract are adjacent to each other.

In an embodiment, a subsequent, e.g., third, fourth, fifth, sixth or seventh, eighth, ninth, or tenth, polyuridine tract is separated by 1, 2, 3, 4, 5, 6, 7, 8, 9, 10, 11, 2, 13, 14, 15, 16, 17, 18, 19, 20, 30, 40, 50, or 60 nucleotides from the first polyuridine tract, the second polyuridine tract, or any one of the subsequent polyuridine tracts.

In an embodiment, a first polyuridine tract is separated by 1, 2, 3, 4, 5, 6, 7, 8, 9, 10, 11, 2, 13, 14, 15, 16, 17, 18, 19, 20, 30, 40, 50, or 60 nucleotides from a subsequent polyuridine tract, e.g., a second, third, fourth, fifth, sixth or seventh, eighth, ninth, or tenth polyuridine tract. In an embodiment, one or more of the subsequent polyuridine tracts are adjacent to a different polyuridine tract.

In an embodiment, the 5′ UTR comprises a Kozak sequence, e.g., a GCCRCC nucleotide sequence (SEQ ID NO: 79) wherein R is an adenine or guanine. In an embodiment, the Kozak sequence is disposed at the 3′ end of the 5‘ ’UTR sequence.

In an aspect, the polynucleotide (e.g., mRNA) comprising an open reading frame encoding an ITB6 polypeptide (e.g., any one of SEQ ID NOs: 18, 2-6, 8, 10, 12, 14, 16, or 160-175) and comprising a 5′ UTR sequence disclosed herein is formulated as an LNP. In an embodiment, the LNP composition comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder, or in a method of inhibiting an immune response in a subject.

In an aspect, an LNP composition comprising a polynucleotide disclosed herein encoding a n ITB6 polypeptide, e.g., as described herein, can be administered with an additional agent, e.g., as described herein.

### b. 3′ UTR Sequences

3′UTR sequences have been shown to influence translation, half-life, and subcellular localization of mRNAs (Mayr C., Cold Spring Harb Persp Biol 2019 Oct. 1; 11(10):a034728).

Disclosed herein, inter alia, is a polynucleotide, e.g., mRNA, comprising an open reading frame encoding an ITB6 polypeptide (e.g., any one of SEQ ID NOs: 18, 2-6, 8, 10, 12, 14, 16, or 160-175) encoding a polypeptide, which polynucleotide has a 3′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as described herein); (b) a coding region comprising a stop element (e.g., as described herein); and (c) a 3′-UTR (e.g., as provided in Table B or a variant or fragment thereof), and LNP compositions comprising the same. In an embodiment, the polynucleotide comprises a 3′-UTR comprising a sequence provided in Table B or a variant or fragment thereof.

In an embodiment, the polynucleotide having a 3′ UTR sequence provided in Table B or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more. In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in Table B or a variant or fragment thereof, results in a polynucleotide with a mean half-life score of greater than 10.

In an embodiment, the polynucleotide having a 3′ UTR sequence provided in Table B or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of Table B or a variant or fragment thereof.

In an embodiment, the polynucleotide comprises a 3′ UTR sequence provided in Table B or a sequence with at least 80%, 85%, 90%, 95%, 96%, 97%, 98%, 99% or 100% identity to a 3′ UTR sequence provided in Table B, or a fragment thereof. In an embodiment, the 3′ UTR comprises a sequence with at least 80%, 85%, 90%, 95%, 96%, 97%, 98%, 99% or 100% identity to SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO:115, SEQ ID NO:136, SEQ ID NO: 137, SEQ ID NO: 138, SEQ ID NO: 139, SEQ ID NO: 140, or SEQ ID NO: 141.

In an embodiment, the 3′ UTR comprises the sequence of SEQ ID NO: 100, or a sequence with at least 80%, 85%, 90%, 95%, 96%, 97%, 98%, 99% or 100% identity to SEQ ID NO: 100. In an embodiment, the 3′ UTR comprises the sequence of SEQ ID NO: 101, or a sequence with at least 80%, 85%, 90%, 95%, 96%, 97%, 98%, 99% or 100% identity to SEQ ID NO: 101. In an embodiment, the 3′ UTR comprises the sequence of SEQ ID NO: 102, or a sequence with at least 80%, 85%, 90%, 95%, 96%, 97%, 98%, 99% or 100% identity to SEQ ID NO: 102. In an embodiment, the 3′ UTR comprises the sequence of SEQ ID NO: 103, or a sequence with at least 80%, 85%, 90%, 95%, 96%, 97%, 98%, 99% or 100% identity to SEQ ID NO: 103. In an embodiment, the 3′ UTR comprises the sequence of SEQ ID NO: 104, or a sequence with at least 80%, 85%, 90%, 95%, 96%, 97%, 98%, 99% or 100% identity to SEQ ID NO: 104. In an embodiment, the 3′ UTR comprises the sequence of SEQ ID NO: 105, or a sequence with at least 80%, 85%, 90%, 95%, 96%, 97%, 98%, 99% or 100% identity to SEQ ID NO: 105. In an embodiment, the 3′ UTR comprises the sequence of SEQ ID NO: 106, or a sequence with at least 80%, 85%, 90%, 95%, 96%, 97%, 98%, 99% or 100% identity to SEQ ID NO: 106. In an embodiment, the 3′ UTR comprises the sequence of SEQ ID NO: 107, or a sequence with at least 80%, 85%, 90%, 95%, 96%, 97%, 98%, 99% or 100% identity to SEQ ID NO: 107. In an embodiment, the 3′ UTR comprises the sequence of SEQ ID NO: 108, or a sequence with at least 80%, 85%, 90%, 95%, 96%, 97%, 98%, 99% or 100% identity to SEQ ID NO: 108. In an embodiment, the 3′ UTR comprises the sequence of SEQ ID NO: 109, or a sequence with at least 80%, 85%, 90%, 95%, 96%, 97%, 98%, 99% or 100% identity to SEQ ID NO: 109. In an embodiment, the 3′ UTR comprises the sequence of SEQ ID NO: 110, or a sequence with at least 80%, 85%, 90%, 95%, 96%, 97%, 98%, 99% or 100% identity to SEQ ID NO: 110. In an embodiment, the 3′ UTR comprises the sequence of SEQ ID NO: 111, or a sequence with at least 80%, 85%, 90%, 95%, 96%, 97%, 98%, 99% or 100% identity to SEQ ID NO: 111. In an embodiment, the 3′ UTR comprises the sequence of SEQ ID NO: 112, or a sequence with at least 80%, 85%, 90%, 95%, 96%, 97%, 98%, 99% or 100% identity to SEQ ID NO: 112. In an embodiment, the 3′ UTR comprises the sequence of SEQ ID NO: 113, or a sequence with at least 80%, 85%, 90%, 95%, 96%, 97%, 98%, 99% or 100% identity to SEQ ID NO: 113. In an embodiment, the 3′ UTR comprises the sequence of SEQ ID NO: 114, or a sequence with at least 80%, 85%, 90%, 95%, 96%, 97%, 98%, 99% or 100% identity to SEQ ID NO: 114. In an embodiment, the 3′ UTR comprises the sequence of SEQ ID NO: 115, or a sequence with at least 80%, 85%, 90%, 95%, 96%, 97%, 98%, 99% or 100% identity to SEQ ID NO: 115. In an embodiment, the 3′ UTR comprises the sequence of SEQ ID NO: 136, or a sequence with at least 80%, 85%, 90%, 95%, 96%, 97%, 98%, 99% or 100% identity to SEQ ID NO: 136. In an embodiment, the 3′ UTR comprises the sequence of SEQ ID NO: 137, or a sequence with at least 80%, 85%, 90%, 95%, 96%, 97%, 98%, 99% or 100% identity to SEQ ID NO: 137. In an embodiment, the 3′ UTR comprises the sequence of SEQ ID NO: 138, or a sequence with at least 80%, 85%, 90%, 95%, 96%, 97%, 98%, 99% or 100% identity to SEQ ID NO: 138. In an embodiment, the 3′ UTR comprises the sequence of SEQ ID NO: 139, or a sequence with at least 80%, 85%, 90%, 95%, 96%, 97%, 98%, 99% or 100% identity to SEQ ID NO: 139. In an embodiment, the 3′ UTR comprises the sequence of SEQ ID NO: 140, or a sequence with at least 80%, 85%, 90%, 95%, 96%, 97%, 98%, 99% or 100% identity to SEQ ID NO: 140. In an embodiment, the 3′ UTR comprises the sequence of SEQ ID NO: 141, or a sequence with at least 80%, 85%, 90%, 95%, 96%, 97%, 98%, 99% or 100% identity to SEQ ID NO: 141.

TABLE B
3′ UTR sequences

| SEQ ID | Sequence | |
|---|---|---|
| NO | Name | Sequence |
| 100 | B1 | UAAUAGUAAGCUGGAGCCUCCUGAGAGACCUGUGUG |
| | | AACUAUUGAGAAGAUCGGAACAGCUCCUUACUCUGA |
| | | GGAAGUUGGUACCCCCGUGGUCUUUGAAUAAAGUCU |
| | | GAGUGGGCGGC |
| 101 | B3 | UAAUAGUAAGCUGGAGCCUCACUCUCCUCUCCAUCCC |
| | | GUAUCCAGGCUGUGAAUUUUUCAAGGAAUAUAAAGA |
| | | UCGGGAUGUACCCCCGUGGUCUUUGAAUAAAGUCUG |
| | | AGUGGGCGGC |
| 102 | B4 | UGAUAGUAAGCUGGAGCCUCUAGUGACGGCAACAGG |
| | | GCUUGGUUUUUCCUUGUUGUGAAAUCGACAUCUCUG |
| | | AAGACAGGGUACCCCCGUGGUCUUUGAAUAAAGUCU |
| | | GAGUGGGCGGC |
| 103 | B5 | UGAUAGUAAGCUGGAGCCUCCUUCCAUCUAGUCACA |
| | | AAGACUCCUUCGUCCCCAGUUGCCGUCUAGGAUUGG |
| | | GCCUCCCAGUACCCCCGUGGUCUUUGAAUAAAGUCUG |
| | | AGUGGGCGGC |
| 104 | B6 | UGAUAGUAAGCUGGAGCCUCCCAUAACAUGACAUAU |
| | | CUGGAUUUUGUGCUUAGAACCUUAAAUUGGAAGCAU |
| | | UCUUAAUUGUACCCCCGUGGUCUUUGAAUAAAGUCU |
| | | GAGUGGGCGGC |
| 105 | B7 | UAAUAGUAAGCUGGAGCCUCCGGAAAACUAAAAUAG |
| | | AGAUAUUUCAAGAUUUUAUAAUUUUCAAAGACCUUU |
| | | GAAAUAUUGUACCCCCGUGGUCUUUGAAUAAAGUCU |
| | | GAGUGGGCGGC |
| 106 | B8 | UAAUAGUAAGCUGGAGCCUCUACACAUUGCUUCUAG |
| | | UUGGCAGAAAUAAUUGAUUAAAAGACCAGAAACUGU |
| | | GAUAACUGGUACCCCCGUGGUCUUUAAAUAAAGUCU |
| | | AAGUGGGCGGC |
| 107 | B9 | UGAUAAUAGGCUGGAGCCUCGGUGGCCAUGCUUCUU |
| | | GCCCCUUGGGCCUCCCCCCAGCCCCUCCUCCCCUUCC |
| | | UGCACCCGUACCCCCGUGGUCUUUGAAUAAAGUCUG |
| | | AGUGGGCGGC |
| 108 | B10 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCCCGUGGUCUUUGAAUAAAGUCUG |
| | | AGUGGGCGGC |
| 109 | B11 | UGAUAAUAGGCUGGAGCCUCGGUGGCCAUGCUUCUU |
| | | GCCCCUUGGGCCUCCCCCCAGCCCCUCCUCCCCUUCC |
| | | UGCACCCGUACCCCCCAAACACCAUUGUCACACUCCA |
| | | GUGGUCUUUGAAUAAAGUCUGAGUGGGCGGC |
| 110 | B12 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCCCCAAACACCAUUGUCACACUCCA |
| | | GUGGUCUUUGAAUAAAGUCUGAGUGGGCGGC |
| 111 | B13 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCCCUCCAUAAAGUAGGAAACACUAC |
| | | AGUGGUCUUUGAAUAAAGUCUGAGUGGGCGGC |
| 112 | B14 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCCCCGCAUUAUUACUCACGGUACGA |
| | | GUGGUCUUUGAAUAAAGUCUGAGUGGGCGGC |
| 113 | B15 | UGAUAAUAGUCCAUAAAGUAGGAAACACUACAGCUG |
| | | GAGCCUCGGUGGCCAUGCUUCUUGCCCCUUGGGCCUC |
| | | CCCCCAGCCCCUCCUCCCCUUCCUGCACCCGUACCCCC |
| | | CGCAUUAUUACUCACGGUACGAGUGGUCUUUGAAUA |
| | | AAGUCUGAGUGGGGGC |
| 114 | B16 | UGAUAAUAGUCCAUAAAGUAGGAAACACUACAGCUG |
| | | GAGCCUCGGUGGCCUAGCUUCUUGCCCCUUGGGCCUC |
| | | CAUAAAGUAGGAAACACUACAUCCCCCCAGCCCCUCC |
| | | UCCCCUUCCUGCACCCGUACCCCCUCCAUAAAGUAGG |
| | | AAACACUACAGUGGUCUUUGAAUAAAGUCUGAGUGG |
| | | GCGGC |
| 115 | B2 | CUGAGAGACCUGUGUGAACUAUUGAGAAGAUCGGAA |
| | | CAGCUCCUUACUCUGAGGAAGUUG |
| 116 | B17 | UGAUAAUAGGCUGGAGCCUCUCACACACCUCUGCCCC |
| | | UUGGGCCUCCCACUCCCAUGGCUCUGGGCGGUCCAGA |
| | | AGGAGCGUACCCCCGUGGUCUUUGAAUAAAGUCUGA |
| | | GUGGGCGGC |
| 117 | B18 | UGAUAAUAGGCUGGAGCCUCCACCGCGUUAUCCGUU |
| | | CCUCGUAGGCUGGUCCUGGGGAACGGGUCGGGGGGU |
| | | ACCCCCGUGGUCUUUGAAUAAAGUCUGAGUGGGGG |
| | | C |
| 118 | B19 | UGAUAAUAGGCUGGAGCCUCUGCCCGGCAACGGCCA |
| | | GGUCUGUGCCAAGUGUUUGCUGACGCAACCCCCACUG |
| | | GCUGGGGCUUGGUCAUGGGCCAUCAGCGCGUGCGUG |
| | | GAACCUUUUCGGCUCCUCUGCCGAUCCAUACUGCGGA |
| | | ACUCCUAGCCGCUUGUUUUGCUCGCAGCAGGUCUGG |
| | | AGCAAACAUUAUCGGGACUGAUAACUCUGUUGUCCU |
| | | GUACCCCCGUGGUCUUUGAAUAAAGUCUGAGUGGGC |
| | | GGC |
| 119 | B20 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCUUUUUUUUUUUUUUUUUUUCUUC |
| | | UUUUCUUUUUUUUCUUUUUUUUUUUUCUUUCUUUUU |
| | | UUCUUUUUUUUUCUUUUUUUUUUCUUUUUUUUUUU |
| | | UUUUUCCGUGGUCUUUGAAUAAAGUCUGAGUGGGCG |
| | | GC |
| 120 | B21 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCUUUUUUUUUUUUUUUUUUUUUUU |
| | | UUUUUUUUUUUUUUUUUUUUUUUUUUUUUUUUUUUU |
| | | UUUUUUUUUUUUUUUUUUUUUUUUUUUUUUUUUUUU |
| | | UUUUUCCGUGGUCUUUGAAUAAAGUCUGAGUGGGCG |
| | | GC |
| 133 | B22 | UAAGUCUCCAUAAAGUAGGAAACACUACAGCUGGAG |
| | | CCUCGGUGGCCUAGCUUCUUGCCCCUUGGGCCUCCAU |
| | | AAAGUAGGAAACACUACAUCCCCCCAGCCCCUCCUCC |
| | | CCUUCCUGCACCCGUACCCCCUCCAUAAAGUAGGAAA |
| | | CACUACAGUGGUCUUUGAAUAAAGUCUGAGUGGGCG |
| | | GC |
| 134 | B23 | UAAAGCGCUGGAGCCUCGGUGGCCUAGCUUCUUGCCC |
| | | CUUGGGCCUCCCCCCAGCCCCUCCUCCCCUUCCUGCA |
| | | CCCGUACCCCCUCCAUAAAGUAGGAAACACUACAGUG |
| | | GUCUUUGAAUAAAGUCUGAGUGGGCGGC |
| 135 | B24 | UAAAGCUCCCCGGGGUCCAUAAAGUAGGAAACACUA |
| | | CAGCUGGAGCCUCCUGAGAGACCUGUGUGAACUAUU |
| | | GAGAAGAUCGGAACAGCUCCUUACUCUGAGGAAGUU |
| | | GUCCAUAAAGUAGGAAACACUACAGUACCCCCUCCAU |
| | | AAAGUAGGAAACACUACAGUGGUCUUUGAAUAAAGU |
| | | CUGAGUGGGCGGC |
| 136 | B25 | UAAUAGUAAACCUCACUCACGGCCACAUUGAGUGCC |
| | | AGGCUCCGGGCUGGUUUAUAGUAGUGUAGAGCAUUG |
| | | CAGCACUUAGACUGGGGUGCUGUAGUCUUUAUUGUA |
| | | GUCUUUCCACAUACCUGAUAAUUCUUAGAUAAUUUC |
| | | UUAUUUUAAUUCCAUAAAGUAGGAAACACUACAUAA |
| | | AUCUCCAUAAAGUAGGAAACACUACAUAUUCUUCCA |
| | | UAAAGUAGGAAACACUACAUAGGCU |
| 137 | B26 | GCCUCCACCGCGUUAUCCGUUCCUCGUAGGCUGGUCC |
| | | UGGGGAACGGGUCGGCGGGUACCCCCGUGGUCUUUG |
| | | AAUAAAGUCUGAGUGGGCGGC |
| 138 | B27 | CACCGCGUUAUCCGUUCCUCGUAGGCUGGUCCUGGGG |
| | | AACGGGUCGGCGGGCCUCGGUGGCCUAGCUUCUUGCC |
| | | CCUUGGGCCCACCGCGUUAUCCGUUCCUCGUAGGCUG |
| | | GUCCUGGGGAACGGGUCGGCGGUCCCCCCAGCCCCUC |
| | | CUCCCCUUCCUGCACCCGUACCCCCCACCGCGUUAUC |
| | | CGUUCCUCGUAGGCUGGUCCUGGGGAACGGGUCGGC |
| | | GGGUGGUCUUUGAAUAAAGUCUGAGUGGGCGGC |
| 139 | B28 | UAAAGCUCCCCGGGGGCCUCGGUGGCCUAGCUUCUUG |
| | | CCCCUUGGGCCUCCCCCCAGCCCCUCCUCCCCUUCCU |
| | | GCACCCGUACCCCCGUGGUCUUUGAAUAAAGUCUGA |
| | | GUGGGCGGC |
| 140 | B29 | UAAGUCUCCGCUGGAGCCUCGGUGGCCUAGCUUCUUGCCCCUUGGGC |
| | | CUCCCCCCAGCCCCUCCUCCCCUUCCUGCACCCGUACCCCCGUGGUCUU |
| | | UGAAUAAAGUCUGAGUGGGCGGC |
| 141 | B30 | UAAGCCCCUCCGGGGGCCUCGGUGGCCUAGCUUCUUGCCCCUUGGGCC |
| | | UCCCCCCAGCCCCUCCUCCCCUUCCUGCACCCGUACCCCCGUGGUCUUU |
| | | GAAUAAAGUCUGAGUGGGCGGC |
| 142 | B31 | UAAGCCCCUCCGGGGCAAACACCAUUGUCACACUCCAGCCUCGGUGG |
| | | CCUAGCUUCUUGCCCCUUGGGCCCAAACACCAUUGUCACACUCCAUC |
| | | CCCCCAGCCCCUCCUCCCCUUCCUGCACCCGUACCCCCCAAACACCA |
| | | UUGUCACACUCCAGUGGUCUUUGAAUAAAGUCUGAGUGGGCGGC |
| 143 | B32 | UAAGCCCCUCCGGGGGCCUCGGUGGCCUAGCUUCUUGCCCCUUGGGC |
| | | CUCCCCCCAGCCCCUCCUCCCCUUCCUGCACCCGUACCCCCCAAACA |
| | | CCAUUGUCACACUCCAGUGGUCUUUGAAUAAAGUCUGAGUGGGCGGC |
| 144 | B33 | UAAGCCCCUCCGGGGGCCUCCUGAGAGACCUGUGUGAACUAUUGAGA |
| | | AGAUCGGAACAGCUCCUUACUCUGAGGAAGUUGGUACCCCCCAAACA |
| | | CCAUUGUCACACUCCAGUGGUCUUUGAAUAAAGUCUGAGUGGGCGGC |
| 145 | B34 | UAAAGCUCCCCGGGGGCCUCGGUGGCCUAGCUUCUUGCCCCUUGGGC |
| | | CUCCCCCCAGCCCCUCCUCCCCUUCCUGCACCCGUACCCCCCAAACA |
| | | CCAUUGUCACACUCCAGUGGUCUUUGAAUAAAGUCUGAGUGGGCGGC |

In an embodiment, the 3′ UTR comprises a micro RNA (miRNA) binding site, e.g., as described herein, which binds to a miR present in a human cell. In an embodiment, the 3′ UTR comprises a miRNA binding site of SEQ ID NO: 212, SEQ ID NO: 174, SEQ ID NO: 152 or a combination thereof. In an embodiment, the 3′ UTR comprises a plurality of miRNA binding sites, e.g., 2, 3, 4, 5, 6, 7 or 8 miRNA binding sites. In an embodiment, the plurality of miRNA binding sites comprises the same or different miRNA binding sites.

miR122 bs =
(SEQ ID NO: 212)
CAAACACCAUUGUCACACUCCA
miR-142-3p bs =
(SEQ ID NO: 174)
UCCAUAAAGUAGGAAACACUACA
miR-126 bs =
(SEQ ID NO: 152)
CGCAUUAUUACUCACGGUACGA

In an aspect, disclosed herein is a polynucleotide encoding a polypeptide, wherein the polynucleotide comprises: (a) a 5′-UTR, e.g., as described herein; (b) a coding region comprising a stop element (e.g., as described herein); and (c) a 3′-UTR (e.g., as described herein).

In an aspect, an LNP composition comprising a polynucleotide comprising an open reading frame encoding an ITB6 polypeptide (e.g., any one of SEQ ID NOs: 18, 2-6, 8, 10, 12, 14, 16, or 160-175) and comprising a 3′ UTR disclosed herein comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder, or in a method of inhibiting an immune response in a subject.

In an aspect, an LNP composition comprising a polynucleotide disclosed herein encoding an ITB6 polypeptide therapeutic payload or prophylactic payload, e.g., as described herein, can be administered with an additional agent, e.g., as described herein.

### Regions Having a 5′ Cap

The disclosure also includes a polynucleotide that comprises both a 5′ Cap and a polynucleotide of the present invention (e.g., a polynucleotide comprising a nucleotide sequence encoding an immune checkpoint inhibitor polypeptide).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp 5′ (5′-5′-triphosphate) linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding an ITB6 polypeptide) incorporate a cap moiety.

In some embodiments, polynucleotides of the present invention (e.g., a polynucleotide comprising a nucleotide sequence encoding an immune checkpoint inhibitor polypeptide) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-0 atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

Another exemplary cap is m⁷G-ppp-Gm-A (i.e., N7,guanosine-5′-triphosphate-2′-O-dimethyl-guanosine-adenosine).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the invention (e.g., a polynucleotide comprising a nucleotide sequence encoding an immune checkpoint inhibitor polypeptide) can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular proinflammatory cytokines, as compared, e.g., to other 5′ cap analog structures known in the art. Cap structures include, but are not limited to, 7mG(5′)ppp(5′)N,pN2p (cap 0), 7mG(5′)ppp(5′)NlmpN2p (cap 1), and 7mG(5′)-ppp(5′)NlmpN2mp (cap 2). Cap 1 is sometimes referred to as Cap C1 herein. In some embodiments, Cap C1 can optionally include an additional G at the 3′ end of the cap. In some embodiments, in Cap C1, N2 may comprise the first nucleotide of a 5′ UTR.

As a non-limiting example, capping chimeric polynucleotides post-manufacture can be more efficient as nearly 100% of the chimeric polynucleotides can be capped. This is in contrast to ˜80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Also provided herein are exemplary caps including those that can be used in co-transcriptional capping methods for ribonucleic acid (RNA) synthesis, using RNA polymerase, e.g., wild type RNA polymerase or variants thereof, e.g., such as those variants described herein. In one embodiment, caps can be added when RNA is produced in a “one-pot” reaction, without the need for a separate capping reaction. Thus, the methods, in some embodiments, comprise reacting a polynucleotide template with an RNA polymerase variant, nucleoside triphosphates, and a cap analog under in vitro transcription reaction conditions to produce RNA transcript.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap. As used here the term “cap” includes the inverted G nucleotide and can comprise one or more additional nucleotides 3′ of the inverted G nucleotide, e.g., 1, 2, or more nucleotides 3′ of the inverted G nucleotide and 5′ to the 5′ UTR, e.g., a 5′ UTR described herein.

Exemplary caps comprise a sequence of GG, GA, or GGA, wherein the underlined, italicized G is an in inverted G nucleotide followed by a 5′-5′-triphosphate group. In one embodiment, the cap comprises m⁷GpppG_{2′OMe}pA.

A trinucleotide cap, in some embodiments, comprises a compound of formula (I)

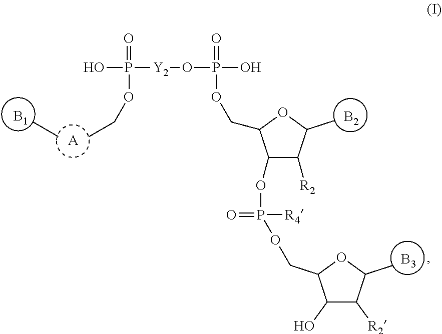

or a stereoisomer, tautomer or salt thereof, wherein

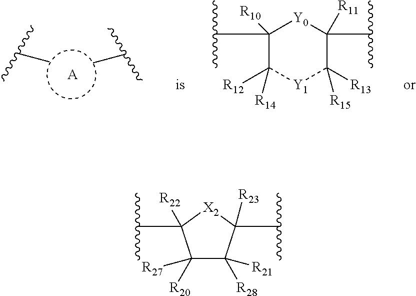

ring B1 is a modified or unmodified Guanine;
ring B2 and ring B3 each independently is a nucleobase or a modified nucleobase;
X2 is O, S(O)p, NR24 or CR25R26 in which p is 0, 1, or 2;
Y0 is O or CR6R7;
Y1 is O, S(O)n, CR6R7, or NR8, in which n is 0, 1, or 2;
each [symbol] is a single bond or absent, wherein when each [symbol] is a single bond, Y1 is O, S(O)n, CR6R7, or NR8; and when each [symbol] is absent, Y1 is void;
Y2 is (OP(O)R4)m in which m is 0, 1, or 2, or —O—(CR40R41)u-Q0-(CR42R43)v-, in which Q0 is a bond, O, S(O)r, NR44, or CR45R46, r is 0, 1, or 2, and each of u and v independently is 1, 2, 3 or 4;
each R2 and R2′ independently is halo, LNA, or OR3;
   each R3 independently is H, C1-C6 alkyl, C2-C6 alkenyl, or C2-C6 alkynyl and R3, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;
each R4 and R4′ independently is H, halo, C1-C6 alkyl, OH, SH, SeH, or BH3-;
each of R6, R7, and R8, independently, is -Q1-T1, in which Q1 is a bond or C1-C3 alkyl linker optionally substituted with one or more of halo, cyano, OH and C1-C6 alkoxy, and T1 is H, halo, OH, COOH, cyano, or Rs1, in which Rs1 is C1-C3 alkyl, C2-C6 alkenyl, C2-C6 alkynyl, C1-C6 alkoxyl, C(O)O—C1-C6 alkyl, C3-C8 cycloalkyl, C6-C10 aryl, NR31R32, (NR31R32R33)+, 4 to 12-membered heterocycloalkyl, or 5- or 6-membered heteroaryl, and Rs1 is optionally substituted with one or more substituents selected from the group consisting of halo, OH, oxo, C1-C6 alkyl, COOH, C(O)O—C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl;
each of R10, R11, R12, R13 R14, and R15, independently, is -Q2-T2, in which Q2 is a bond or C1-C3 alkyl linker optionally substituted with one or more of halo, cyano, OH and C1-C6 alkoxy, and T2 is H, halo, OH, NH2, cyano, NO2, N3, Rs2, or ORs2, in which Rs2 is C1-C6 alkyl, C2-C6 alkenyl, C2-C6 alkynyl, C3-C8 cycloalkyl, C6-C10 aryl, NHC(O)—C1-C6 alkyl, NR31R32, (NR31R32R33)+, 4 to 12-membered heterocycloalkyl, or 5- or 6-membered heteroaryl, and Rs2 is optionally substituted with one or more substituents selected from the group consisting of halo, OH, oxo, C1-C6 alkyl, COOH, C(O)O—C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl; or alternatively R12 together with R14 is oxo, or R13 together with R15 is oxo,
each of R20, R21, R22, and R23 independently is -Q3-T3, in which Q3 is a bond or C1-C3 alkyl linker optionally substituted with one or more of halo, cyano, OH and C1-C6 alkoxy, and T3 is H, halo, OH, NH2, cyano, NO2, N3, RS3, or ORS3, in which RS3 is C1-C6 alkyl, C2-C6 alkenyl, C2-C6 alkynyl, C3-C8 cycloalkyl, C6-C10 aryl, NHC(O)—C1-C6 alkyl, mono-C1-C6 alkylamino, di-C1-C6 alkylamino, 4 to 12-membered heterocycloalkyl, or 5- or 6-membered heteroaryl, and Rs3 is optionally substituted with one or more substituents selected from the group consisting of halo, OH, oxo, C1-C6 alkyl, COOH, C(O)O—C1-C6 alkyl, cyano, C1-C6 alkoxyl, amino, mono-C1-C6 alkylamino, di-C1-C6 alkylamino, C3-C8 cycloalkyl, C6-C10 aryl, 4 to 12-membered heterocycloalkyl, and 5- or 6-membered heteroaryl;
each of R24, R25, and R26 independently is H or C1-C6 alkyl;
each of R27 and R28 independently is H or OR29; or R27 and R28 together form O—R30-O; each R29 independently is H, C1-C6 alkyl, C2-C6 alkenyl, or C2-C6 alkynyl and R29, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;
R30 is C1-C6 alkylene optionally substituted with one or more of halo, OH and C1-C6 alkoxyl;
each of R31, R32, and R33, independently is H, C1-C6 alkyl, C3-C8 cycloalkyl, C6-C10 aryl, 4 to 12-membered heterocycloalkyl, or 5- or 6-membered heteroaryl;
each of R40, R41, R42, and R43 independently is H, halo, OH, cyano, N3, OP(O)R47R48, or C1-C6 alkyl optionally substituted with one or more OP(O)R47R48, or one R41 and one R43, together with the carbon atoms to which they are attached and Q0, form C4-C10 cycloalkyl, 4- to 14-membered heterocycloalkyl, C6-C10 aryl, or 5- to 14-membered heteroaryl, and each of the cycloalkyl, heterocycloalkyl, phenyl, or 5- to 6-membered heteroaryl is optionally substituted with one or more of OH, halo, cyano, N3, oxo, OP(O)R47R48, C1-C6 alkyl, C1-C6 haloalkyl, COOH, C(O)O—C1-C6 alkyl, C1-C6 alkoxyl, C1-C6 haloalkoxyl, amino, mono-C1-C6 alkylamino, and di-C1-C6 alkylamino;
   R44 is H, C1-C6 alkyl, or an amine protecting group;
   each of R45 and R46 independently is H, OP(O)R47R48, or C1-C6 alkyl optionally substituted with one or more OP(O)R47R48, and
   each of R47 and R48, independently is H, halo, C1-C6 alkyl, OH, SH, SeH, or BH3.

It should be understood that a cap analog, as provided herein, may include any of the cap analogs described in international publication WO 2017/066797, published on 20 Apr. 2017, incorporated by reference herein in its entirety.

In some embodiments, the B2 middle position can be a non-ribose molecule, such as arabinose.

In some embodiments R2 is ethyl-based.

Thus, in some embodiments, a trinucleotide cap comprises the following structure:

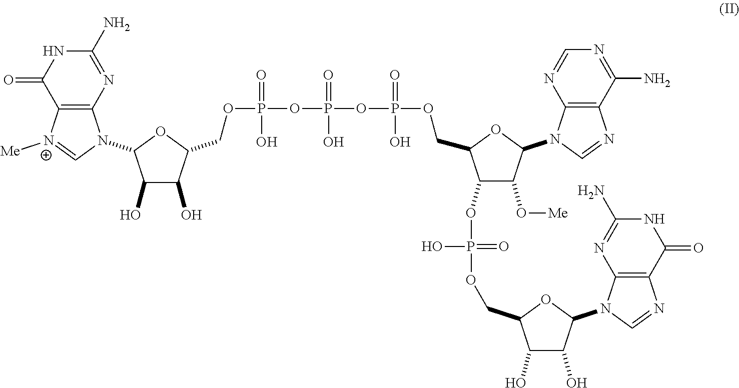

In other embodiments, a trinucleotide cap comprises the following structure:

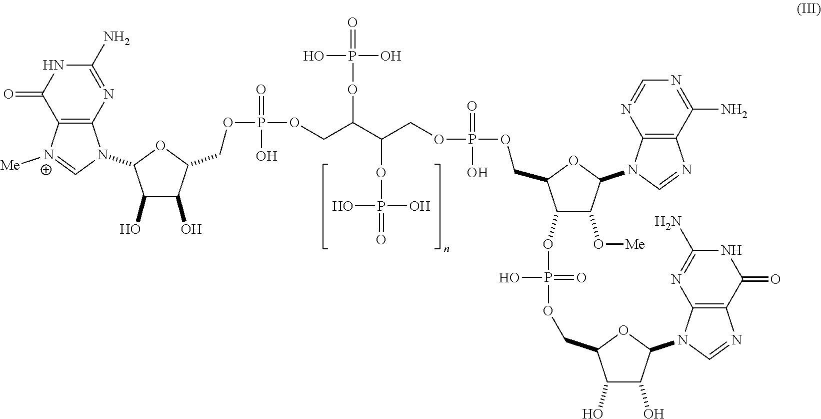

In yet other embodiments, a trinucleotide cap comprises the following structure:

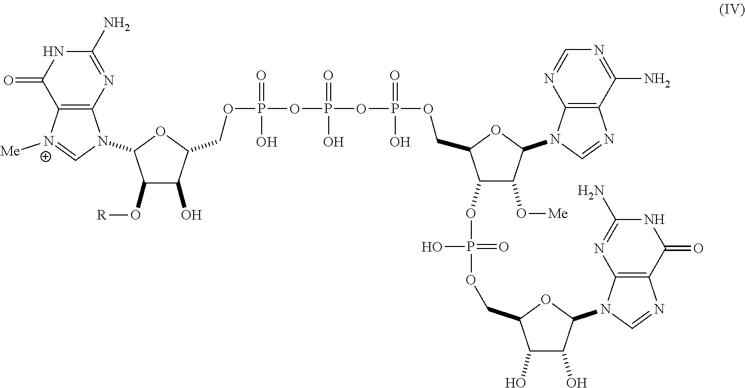

In still other embodiments, a trinucleotide cap comprises the following structure:

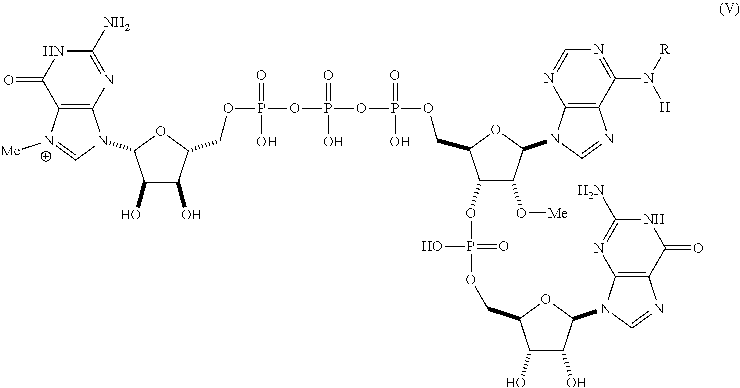

In some embodiments, R is an alkyl (e.g., C₁-C₆ alkyl). In some embodiments, R is a methyl group (e.g., C₁ alkyl). In some embodiments, R is an ethyl group (e.g., C₂ alkyl).

A dinucleotide cap, in some embodiments, comprises a compound of formula (I-b)

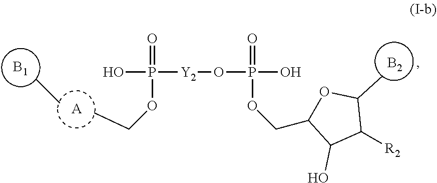

stereoisomer, tautomer or salt thereof, wherein

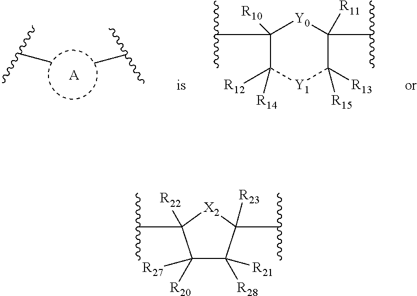

ring B1 is a modified or unmodified Guanine;
ring B2 is a nucleobase or a modified nucleobase;
X2 is O, S(O)p, NR24 or CR25R26 in which p is 0, 1, or 2;
Y0 is O or CR6R7;
Y1 is O, S(O)n, CR6R7, or NR8, in which n is 0, 1, or 2;
each --- is a single bond or absent, wherein when each --- is a single bond, Y1 is O, S(O)n, CR6R7, or NR8; and when each --- is absent, Y1 is void;
Y2 is (OP(O)R4)m in which m is 0, 1, or 2, or —O—(CR40R41)u-Q0-(CR42R43)v-, in which Q0 is a bond, O, S(O)r, NR44, or CR45R46, r is 0, 1, or 2, and each of u and v independently is 1, 2, 3 or 4;
R2 is halo, LNA, or OR3;
   each R3 independently is H, C1-C6 alkyl, C2-C6 alkenyl, or C2-C6 alkynyl and R3, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;
R4 is H, halo, C1-C6 alkyl, OH, SH, SeH, or BH3-;
each of R6, R7, and R8, independently, is -Q1-T1, in which Q1 is a bond or C1-C3 alkyl linker optionally substituted with one or more of halo, cyano, OH and C1-C6 alkoxy, and T1 is H, halo, OH, COOH, cyano, or Rs1, in which Rs1 is C1-C3 alkyl, C2-C6 alkenyl, C2-C6 alkynyl, C1-C6 alkoxyl, C(O)O—C1-C6 alkyl, C3-C8 cycloalkyl, C6-C10 aryl, NR31R32, (NR31R32R33)⁺, 4 to 12-membered heterocycloalkyl, or 5- or 6-membered heteroaryl, and Rs1 is optionally substituted with one or more substituents selected from the group consisting of halo, OH, oxo, C1-C6 alkyl, COOH, C(O)O—C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl;
each of R10, R11, R12, R13 R14, and R15, independently, is -Q2-T2, in which Q2 is a bond or C1-C3 alkyl linker optionally substituted with one or more of halo, cyano, OH and C1-C6 alkoxy, and T2 is H, halo, OH, NH2, cyano, NO2, N3, Rs2, or ORs2, in which Rs2 is C1-C6 alkyl, C2-C6 alkenyl, C2-C6 alkynyl, C3-C8 cycloalkyl, C6-C10 aryl, NHC(O)—C1-C6 alkyl, NR31R32, (NR31R32R33)⁺, 4 to 12-membered heterocycloalkyl, or 5- or 6-membered heteroaryl, and Rs2 is optionally substituted with one or more substituents selected from the group consisting of halo, OH, oxo, C1-C6 alkyl, COOH, C(O)O—C1-C6 alkyl, cyano, C1-C6 alkoxyl, NR31R32, (NR31R32R33)⁺, C3-C8 cycloalkyl, C6-C10 aryl, 4 to 12-membered heterocycloalkyl, and 5- or 6-membered heteroaryl; or alternatively R12 together with R14 is oxo, or R13 together with R15 is oxo,
each of R20, R21, R22, and R23 independently is -Q3-T3, in which Q3 is a bond or C1-C3 alkyl linker optionally substituted with one or more of halo, cyano, OH and C1-C6 alkoxy, and T3 is H, halo, OH, NH2, cyano, NO2, N3, RS3, or ORS3, in which RS3 is C1-C6 alkyl, C2-C6 alkenyl, C2-C6 alkynyl, C3-C8 cycloalkyl, C6-C10 aryl, NHC(O)—C1-C6 alkyl, mono-C1-C6 alkylamino, di-C1-C6 alkylamino, 4 to 12-membered heterocycloalkyl, or 5- or 6-membered heteroaryl, and Rs3 is optionally substituted with one or more substituents selected from the group consisting of halo, OH, oxo, C1-C6 alkyl, COOH, C(O)O—C1-C6 alkyl, cyano, C1-C6 alkoxyl, amino, mono-C1-C6 alkylamino, di-C1-C6 alkylamino, C3-C8 cycloalkyl, C6-C10 aryl, 4 to 12-membered heterocycloalkyl, and 5- or 6-membered heteroaryl;
each of R24, R25, and R26 independently is H or C1-C6 alkyl;
each of R27 and R28 independently is H or OR29; or R27 and R28 together form O—R30-O; each R29 independently is H, C1-C6 alkyl, C2-C6 alkenyl, or C2-C6 alkynyl and R29, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;
R30 is C1-C6 alkylene optionally substituted with one or more of halo, OH and C1-C6 alkoxyl;
each of R31, R32, and R33, independently is H, C1-C6 alkyl, C3-C8 cycloalkyl, C6-C10 aryl, 4 to 12-membered heterocycloalkyl, or 5- or 6-membered heteroaryl;
each of R40, R41, R42, and R43 independently is H, halo, OH, cyano, N3, OP(O)R47R48, or C1-C6 alkyl optionally substituted with one or more OP(O)R47R48, or one R41 and one R43, together with the carbon atoms to which they are attached and Q0, form C4-C10 cycloalkyl, 4- to 14-membered heterocycloalkyl, C6-C10 aryl, or 5- to 14-membered heteroaryl, and each of the cycloalkyl, heterocycloalkyl, phenyl, or 5- to 6-membered heteroaryl is optionally substituted with one or more of OH, halo, cyano, N3, oxo, OP(O)R47R48, C1-C6 alkyl, C1-C6 haloalkyl, COOH, C(O)O—C1-C6 alkyl, C1-C6 alkoxyl, C1-C6 haloalkoxyl, amino, mono-C1-C6 alkylamino, and di-C1-C6 alkylamino;
   R44 is H, C1-C6 alkyl, or an amine protecting group;
   each of R45 and R46 independently is H, OP(O)R47R48, or C1-C6 alkyl optionally substituted with one or more OP(O)R47R48, and
   each of R47 and R48, independently is H, halo, C1-C6 alkyl, OH, SH, SeH, or BH3.

Thus, in some embodiments, a dinucleotide cap comprises the following structure:

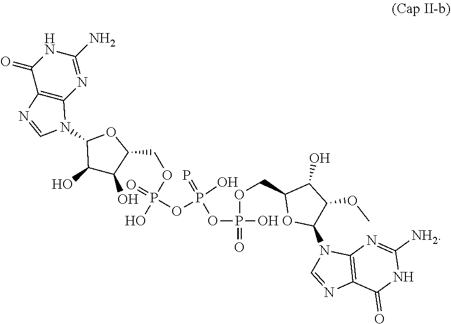

A trinucleotide cap, in some embodiments, comprises a sequence selected from the following sequences: GAA, GAC, GAG, GAU, GCA, GCC, GCG, GCU, GGA, GGC, GGG, GGU, GUA, GUC, GUG, and GUU. In some embodiments, a trinucleotide cap comprises GAA.

In some embodiments, a trinucleotide cap comprises GAC. In some embodiments, a trinucleotide cap comprises GAG. In some embodiments, a trinucleotide cap comprises GAU. In some embodiments, a trinucleotide cap comprises GCA. In some embodiments, a trinucleotide cap comprises GCC. In some embodiments, a trinucleotide cap comprises GCG. In some embodiments, a trinucleotide cap comprises GCU. In some embodiments, a trinucleotide cap comprises GGA. In some embodiments, a trinucleotide cap comprises GGC. In some embodiments, a trinucleotide cap comprises GGG. In some embodiments, a trinucleotide cap comprises GGU. In some embodiments, a trinucleotide cap comprises GUA. In some embodiments, a trinucleotide cap comprises GUC. In some embodiments, a trinucleotide cap comprises GUG. In some embodiments, a trinucleotide cap comprises GUU.

In some embodiments, a trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

In some embodiments, a trinucleotide cap comprises m⁷GpppApA. In some embodiments, a trinucleotide cap comprises m⁷GpppApC. In some embodiments, a trinucleotide cap comprises m⁷GpppApG. In some embodiments, a trinucleotide cap comprises m⁷GpppApU.

In some embodiments, a trinucleotide cap comprises m⁷GpppCpA. In some embodiments, a trinucleotide cap comprises m⁷GpppCpC. In some embodiments, a trinucleotide cap comprises m⁷GpppCpG. In some embodiments, a trinucleotide cap comprises m⁷GpppCpU. In some embodiments, a trinucleotide cap comprises m⁷GpppGpA. In some embodiments, a trinucleotide cap comprises m⁷GpppGpC. In some embodiments, a trinucleotide cap comprises m⁷GpppGpG.

In some embodiments, a trinucleotide cap comprises m⁷GpppGpU. In some embodiments, a trinucleotide cap comprises m⁷GpppUpA. In some embodiments, a trinucleotide cap comprises m⁷GpppUpC. In some embodiments, a trinucleotide cap comprises m⁷GpppUpG. In some embodiments, a trinucleotide cap comprises m⁷GpppUpU.

A trinucleotide cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

In some embodiments, a trinucleotide cap comprises m⁷G_{3′OMe}pppApA. In some embodiments, a trinucleotide cap comprises m⁷G_{3′OMe}pppApC. In some embodiments, a trinucleotide cap comprises m⁷G_{3′OMe}pppApG. In some embodiments, a trinucleotide cap comprises m⁷G_{3′OMe}pppApU. In some embodiments, a trinucleotide cap comprises m⁷G_{3′OMe}pppCpA. In some embodiments, a trinucleotide cap comprises m⁷G_{3′OMe}pppCpC. In some embodiments, a trinucleotide cap comprises m⁷G_{3′OMe}pppCpG. In some embodiments, a trinucleotide cap comprises m⁷G_{3′OMe}pppCpU. In some embodiments, a trinucleotide cap comprises m⁷G_{3′OMe}pppGpA. In some embodiments, a trinucleotide cap comprises m⁷G_{3′OMe}pppGpC. In some embodiments, a trinucleotide cap comprises m⁷G_{3′OMe}pppGpG. In some embodiments, a trinucleotide cap comprises m⁷G_{3′OMe}pppGpU. In some embodiments, a trinucleotide cap comprises m⁷G_{3′OMe}pppUpA. In some embodiments, a trinucleotide cap comprises m⁷G_{3′OMe}pppUpC. In some embodiments, a trinucleotide cap comprises m⁷G_{3′OMe}pppUpG. In some embodiments, a trinucleotide cap comprises m⁷G_{3′OMe}pppUpU. A trinucleotide cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

In some embodiments, a trinucleotide cap comprises m⁷G_{3′OMe}pppA_{2′OMe}pA. In some embodiments, a trinucleotide cap comprises m⁷G_{3′OMe}pppA_{2′OMe}pC. In some embodiments, a trinucleotide cap comprises m⁷G_{3′OMe}pppA_{2′OMe}pG. In some embodiments, a trinucleotide cap comprises m⁷G_{3′OMe}pppA_{2′OMe}pU. In some embodiments, a trinucleotide cap comprises m⁷G_{3′OMe}pppC_{2′OMe}pA. In some embodiments, a trinucleotide cap comprises m⁷G_{3′OMe}pppC_{2′OMe}pC. In some embodiments, a trinucleotide cap comprises m⁷G_{3′OMe}pppC_{2′OMe}pG. In some embodiments, a trinucleotide cap comprises m⁷G_{3′OMe}pppC_{2′OMe}pU. In some embodiments, a trinucleotide cap comprises m⁷G_{3′OMe}pppG_{2′OMe}pA. In some embodiments, a trinucleotide cap comprises m⁷G_{3′OMe}pppG_{2′OMe}pC. In some embodiments, a trinucleotide cap comprises m⁷G_{3′OMe}pppG_{2′OMe}pG. In some embodiments, a trinucleotide cap comprises m⁷G_{3′OMe}pppG_{2′OMe}pU. In some embodiments, a trinucleotide cap comprises m⁷G_{3′OMe}pppU_{2′OMe}pA. In some embodiments, a trinucleotide cap comprises m⁷G_{3′OMe}pppU_{2′OMe}pC. In some embodiments, a trinucleotide cap comprises m⁷G_{3′OMe}pppU_{2′OMe}pG. In some embodiments, a trinucleotide cap comprises m⁷G_{3′OMe}pppU_{2′OMe}pU.

A trinucleotide cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

In some embodiments, a trinucleotide cap comprises m⁷GpppA_{2′OMe}pA. In some embodiments, a trinucleotide cap comprises m⁷GpppA_{2′OMe}pC. In some embodiments, a trinucleotide cap comprises m⁷GpppA_{2′OMe}pG. In some embodiments, a trinucleotide cap comprises m⁷GpppA_{2′OMe}pU. In some embodiments, a trinucleotide cap comprises m⁷GpppC_{2′OMe}pA. In some embodiments, a trinucleotide cap comprises m⁷GpppC_{2′OMe}pC. In some embodiments, a trinucleotide cap comprises m⁷GpppC_{2′OMe}pG. In some embodiments, a trinucleotide cap comprises m⁷GpppC_{2′OMe}pU. In some embodiments, a trinucleotide cap comprises m⁷GpppG_{2′OMe}pA. In some embodiments, a trinucleotide cap comprises m⁷GpppG_{2′OMe}pC. In some embodiments, a trinucleotide cap comprises m⁷GpppG_{2′OMe}pG. In some embodiments, a trinucleotide cap comprises m⁷GpppG_{2′OMe}pU. In some embodiments, a trinucleotide cap comprises m⁷GpppU_{2′OMe}pA. In some embodiments, a trinucleotide cap comprises m⁷GpppU_{2′OMe}pC. In some embodiments, a trinucleotide cap comprises m⁷GpppU_{2′OMe}pG. In some embodiments, a trinucleotide cap comprises m⁷GpppU_{2′OMe}pU.

In some embodiments, a trinucleotide cap comprises m⁷Gpppm⁶A_{2′Ome}pG. In some embodiments, a trinucleotide cap comprises m⁷Gpppe⁶A_{2′Ome}pG.

In some embodiments, a trinucleotide cap comprises GAG. In some embodiments, a trinucleotide cap comprises GCG. In some embodiments, a trinucleotide cap comprises GUG. In some embodiments, a trinucleotide cap comprises GGG.

In some embodiments, a trinucleotide cap comprises any one of the following structures:

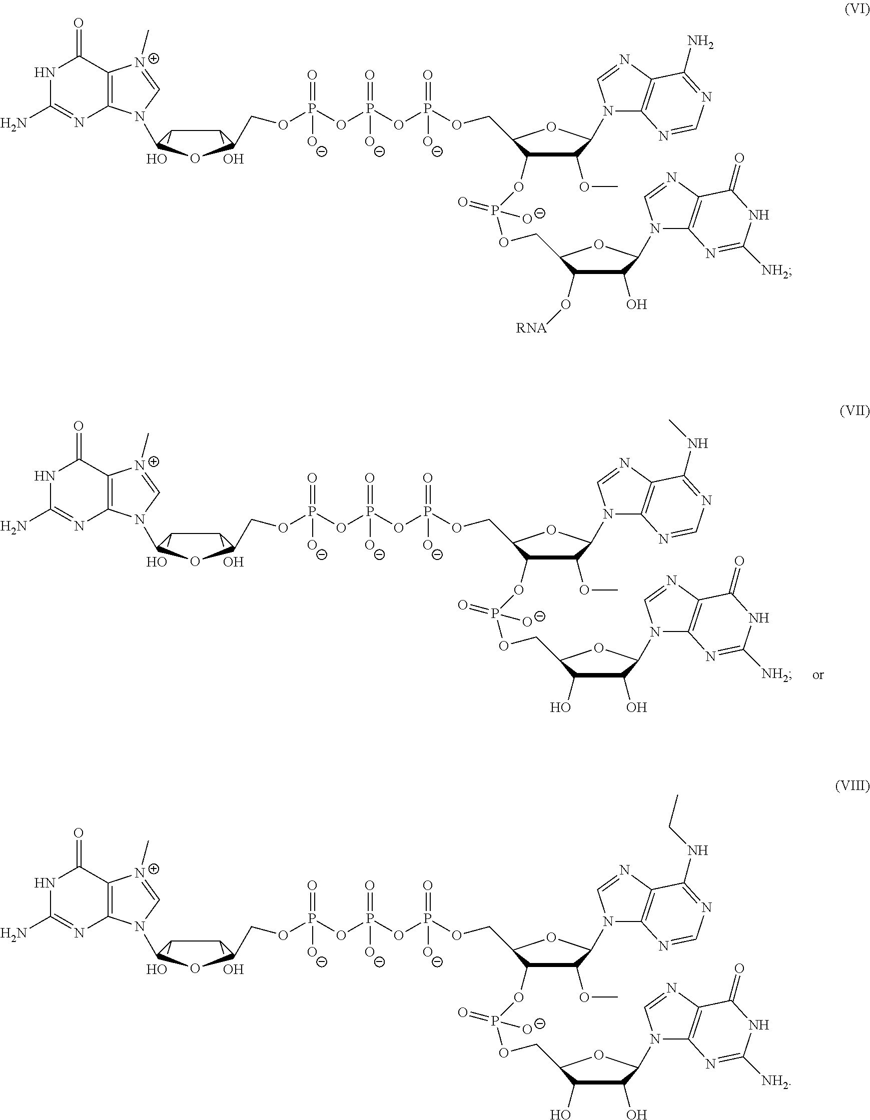

In some embodiments, the cap analog comprises a tetranucleotide cap. In some embodiments, the tetranucleotide cap comprises a trinucleotide as set forth above. In some embodiments, the tetranucleotide cap comprises ^{m7}GpppN₁N₂N₃, where N₁, N₂, and N₃ are optional (i.e., can be absent or one or more can be present) and are independently a natural, a modified, or an unnatural nucleoside base. In some embodiments, ^{m7}G is further methylated, e.g., at the 3′ position. In some embodiments, the ^{m7}G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position.

In some embodiments, the tetranucleotide cap comprises the following structure:

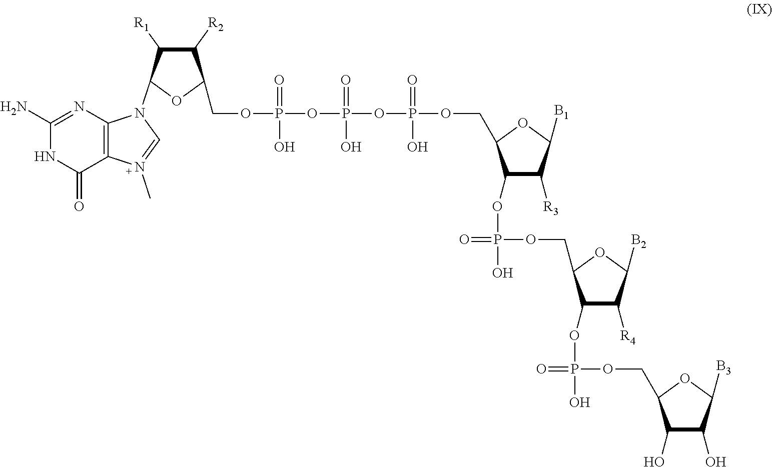

wherein B₁, B₂, and B₃ are independently a natural, a modified, or an unnatural nucleoside based; and R₁, R₂, R₃, and R₄ are independently OH or O-methyl. In some embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH. In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B₁, B₃, and B₃ are natural nucleoside bases. In some embodiments, at least one of B₁, B₂, and B₃ is a modified or unnatural base. In some embodiments, at least one of B₁, B₂, and B₃ is N₆-methyladenine. In some embodiments, B₁ is adenine, cytosine, thymine, or uracil. In some embodiments, B₁ is adenine, B₂ is uracil, and B₃ is adenine. In some embodiments, R₁ and R₂ are OH, R₃ and R₄ are O-methyl, B₁ is adenine, B₂ is uracil, and B₃ is adenine.

In some embodiments the tetranucleotide cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the tetranucleotide cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the tetranucleotide cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the tetranucleotide cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A tetranucleotide cap, in some embodiments, comprises a sequence selected from the following sequences: m⁷G_{3′OMe}pppApApN, m⁷G_{3′OMe}pppApCpN, m⁷G_{3′OMe}pppApGpN, m⁷G_{3′OMe}pppApUpN, m⁷G_{3′OMe}pppCpApN, m⁷G_{3′OMe}pppCpCpN, m⁷G_{3′OMe}pppCpGpN, m⁷G_{3′OMe}pppCpUpN, m⁷G_{3′OMe}pppGpApN, m⁷G_{3′OMe}pppGpCpN, m⁷G_{3′OMe}pppGpGpN, m⁷G_{3′OMe}pppGpUpN, m⁷G_{3′OMe}pppUpApN, m⁷G_{3′OMe}pppUpCpN, m⁷G_{3′OMe}pppUpGpN, and m⁷G_{3′OMe}pppUpUpN, where N is a natural, a modified, or an unnatural nucleoside base.

A tetranucleotide cap, in other embodiments, comprises a sequence selected from the following sequences: m⁷G_{3′OMe}pppA_{2′OMe}pApN, m⁷G_{3′OMe}pppA_{2′OMe}pCpN, m⁷G_{3′OMe}pppA_{2′OMe}pGpN, m⁷G_{3′OMe}pppA_{2′OMe}pUpN, m⁷G_{3′OMe}pppC_{2′OMe}pApN, m⁷G_{3′OMe}pppC_{2′OMe}pCpN, m⁷G_{3′OMe}pppC_{2′OMe}pGpN, m⁷G_{3′OMe}pppC_{2′OMe}pUpN, m⁷G_{3′OMe}pppG_{2′OMe}pApN, m⁷G_{3′OMe}pppG_{2′OMe}pCpN, m⁷G_{3′OMe}pppG_{2′OMe}pGpN, m⁷G_{3′OMe}pppG_{2′OMe}pUpN, m⁷G_{3′OMe}pppU_{2′OMe}pApN, m⁷G_{3′OMe}pppU_{2′OMe}pCpN, m⁷G_{3′OMe}pppU_{2′OMe}pGpN, and m⁷G_{3′OMe}pppU_{2′OMe}pUpN, where N is a natural, a modified, or an unnatural nucleoside base.

A tetranucleotide cap, in still other embodiments, comprises a sequence selected from the following sequences: m⁷GpppA_{2′OMe}pApN, m⁷GpppA_{2′OMe}pCpN, m⁷GpppA_{2′OMe}pGpN, m⁷GpppA_{2′OMe}pUpN, m⁷GpppC_{2′OMe}pApN, m⁷GpppC_{2′OMe}pCpN, m⁷GpppC_{2′OMe}pGpN, m⁷GpppC_{2′OMe}pUpN, m⁷GpppG_{2′OMe}pApN, m⁷GpppG_{2′OMe}pCpN, m⁷GpppG_{2′OMe}pGpN, m⁷GpppG_{2′OMe}pUpN, m⁷GpppU_{2′OMe}pApN, m⁷GpppU_{2′OMe}pCpN, m⁷GpppU_{2′OMe}pGpN, and m⁷GpppU_{2′OMe}pUpN, where N is a natural, a modified, or an unnatural nucleoside base.

A tetranucleotide cap, in other embodiments, comprises a sequence selected from the following sequences: m⁷G_{3′OMe}pppA_{2′OMe}pA_{2′OMe}pN, m⁷G_{3′OMe}pppA_{2′OMe}pC_{2′OMe}pN, m⁷G_{3′OMe}pppA_{2′OMe}pG_{2′OMe}pN, m⁷G_{3′OMe}pppA_{2′OMe}pU_{2′OMe}pN, m⁷G_{3′OMe}pppC_{2′OMe}pA_{2′OMe}pN, m⁷G_{3′OMe}pppC_{2′OMe}pC_{2′OMe}pN, m⁷G_{3′OMe}pppC_{2′OMe}pG_{2′OMe}pN, m⁷G_{3′OMe}pppC_{2′OMe}pU_{2′OMe}pN, m⁷G_{3′OMe}pppG_{2′OMe}pA_{2′OMe}pN, m⁷G_{3′OMe}pppG_{2′OMe}pC_{2′OMe}pN, m⁷G_{3′OMe}pppG_{2′OMe}pG_{2′OMe}pN, m⁷G_{3′OMe}pppG_{2′OMe}pU_{2′OMe}pN, m⁷G_{3′OMe}pppU_{2′OMe}pA_{2′OMe}pN, m⁷G_{3′OMe}pppU_{2′OMe}pC_{2′OMe}pN, m⁷G_{3′OMe}pppU_{2′OMe}pG_{2′OMe}pN, and m⁷G_{3′OMe}pppU_{2′OMe}pU_{2′OMe}pN, where N is a natural, a modified, or an unnatural nucleoside base.

A tetranucleotide cap, in still other embodiments, comprises a sequence selected from the following sequences: m⁷GpppA_{2′OMe}pA_{2′OMe}pN, m⁷GpppA_{2′OMe}pC_{2′OMe}pN, m⁷GpppA_{2′OMe}pG_{2′OMe}pN, m⁷GpppA_{2′OMe}pU_{2′OMe}pN, m⁷GpppC_{2′OMe}pA_{2′OMe}pN, m⁷GpppC_{2′OMe}pC_{2′OMe}pN, m⁷GpppC_{2′OMe}pG_{2′OMe}pN, m⁷GpppC_{2′OMe}pU_{2′OMe}pN, m⁷GpppG_{2′OMe}pA_{2′OMe}pN, m⁷GpppG_{2′OMe}pC_{2′OMe}pN, m⁷GpppG_{2′OMe}pG_{2′OMe}pN, m⁷GpppG_{2′OMe}pU_{2′OMe}pN, m⁷GpppU_{2′OMe}pA_{2′OMe}pN, m⁷GpppU_{2′OMe}pC_{2′OMe}pN, m⁷GpppU_{2′OMe}pG_{2′OMe}pN, and m⁷GpppU_{2′OMe}pU_{2′OMe}pN, where N is a natural, a modified, or an unnatural nucleoside base.

In some embodiments, a tetranucleotide cap comprises GGAG. In some embodiments, a tetranucleotide cap comprises the following structure:

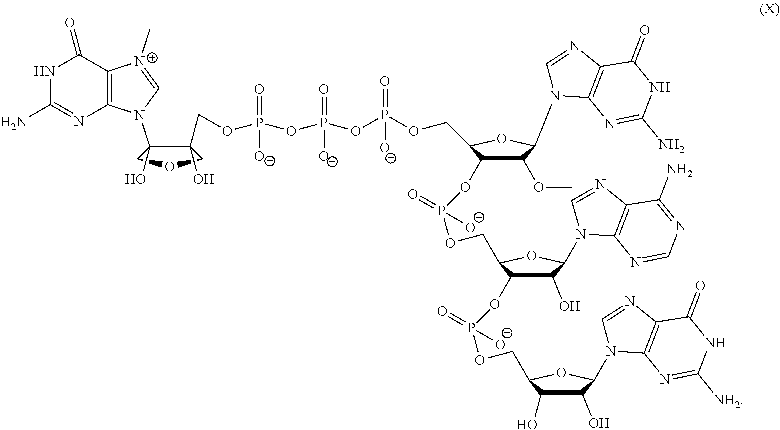

### Tails, e.g., Poly A Tails

In some embodiments, the polynucleotides of the present disclosure (e.g., a polynucleotide comprising a nucleotide sequence encoding an immune checkpoint inhibitor polypeptide) further comprise a tail, e.g., a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO:502).

(SEQ ID NO: 502)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO: 503).

(SEQ ID NO: 503)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa aaaaaaaaaa aaaaaaaaaa

In some embodiments, the polyA tail comprises an alternative nucleoside, e.g., inverted thymidine. PolyA tails comprising an alternative nucleoside, e.g., inverted thymidine, may be generated as described herein. For instance, mRNA constructs may be modified by ligation to stabilize the poly(A) tail. Ligation may be performed using 0.5-1.5 mg/mL mRNA (5′ Cap1, 3′ A100), 50 mM Tris-HCl pH 7.5, 10 mM MgCl2, 1 mM TCEP, 1000 units/mL T4 RNA Ligase 1, 1 mM ATP, 20% w/v polyethylene glycol 8000, and 5:1 molar ratio of modifying oligo to mRNA. Modifying oligo has a sequence of 5′-phosphate-AAAAAAAAAAAAAAAAAAAA-(inverted deoxythymidine (idT) (SEQ ID NO:209)) (see below). Ligation reactions are mixed and incubated at room temperature (˜22° C.) for, e.g., 4 hours. Stable tail mRNA are purified by, e.g., dT purification, reverse phase purification, hydroxyapatite purification, ultrafiltration into water, and sterile filtration. The resulting stable tail-containing mRNAs contain the following structure at the 3′end, starting with the polyA region: A100-UCUAGAAAAAAAAAAAAAAAAAAAA-inverted deoxythymidine (SEQ ID NO:211).

Modifying oligo to stabilize tail (5′-phosphate-AAAAAAAAAAAAAAAAAAAA-(inverted deoxythymidine)(SEQ ID NO:209)):

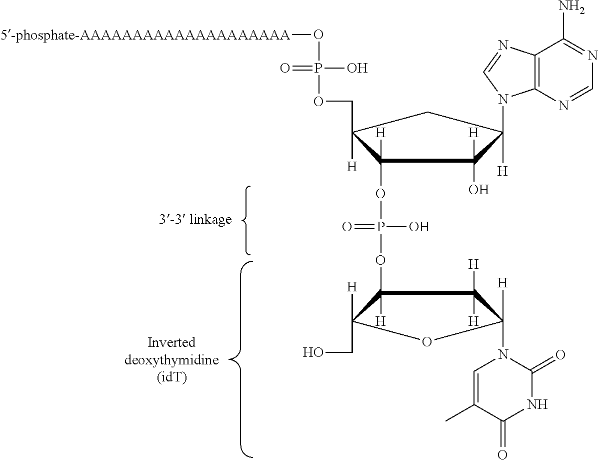

### Start Codon Region

The invention also includes a polynucleotide that comprises both a start codon region and a polynucleotide described herein (e.g., a polynucleotide comprising a nucleotide sequence encoding an immune checkpoint inhibitor polypeptide). In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide, or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The invention also includes a polynucleotide that comprises both a stop codon region and a polynucleotide described herein (e.g., a polynucleotide comprising a nucleotide sequence encoding an ITB6 polypeptide). In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

### 3′ Stabilizing Region

In some embodiments, the polynucleotides of the present disclosure (e.g., a polynucleotide comprising a nucleotide sequence encoding an ITB6 polypeptide) further comprise a 3′ stabilizing region. In an embodiment, the polynucleotide comprises: (a) a 5′-UTR (e.g., as described herein); (b) a coding region comprising a stop codon region (e.g., as described herein); (c) a 3′-UTR (e.g., as described herein), and (d) a 3′ stabilizing region. Also disclosed herein are LNP compositions comprising the same.

In an embodiment, the polynucleotide comprises a 3′ stabilizing region, e.g., a stabilized tail (e.g., as described herein). A polynucleotide containing a 3′-stabilizing region (e.g., a 3′-stabilizing region including an alternative nucleobase, sugar, and/or backbone) may be particularly effective for use in therapeutic compositions, because they may benefit from increased stability, high expression levels.

In an embodiment, the 3′ stabilizing region comprises a poly A tail, e.g., a poly A tail comprising 80-150, e.g., 120, adenines (SEQ ID NO: 370). In an embodiment, the poly A tail comprises a UCUAG sequence (SEQ ID NO: 270). In an embodiment, the poly A tail comprises about 80-120, e.g., 100, adenines upstream of SEQ ID NO: 270. In an embodiment, the poly A tail comprises about 1-40, e.g., 20, adenines downstream of SEQ ID NO: 270.

In an embodiment, the 3′ stabilizing region comprises at least one alternative nucleoside. In an embodiment, the alternative nucleoside is an inverted thymidine (idT). In an embodiment, the alternative nucleoside is disposed at the 3′ end of the 3′ stabilizing region.

In an embodiment, the 3′ stabilizing region comprises a structure of Formula VII:

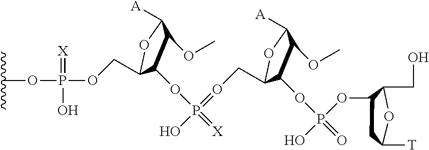

or a salt thereof, wherein each X is independently O or S, and A represents adenine and T represents Thymine.

In an aspect, disclosed herein is an LNP composition comprising a polynucleotide (e.g., an mRNA) which encodes an ITB6 molecule (e.g., an ITB6 molecule described herein), wherein the polynucleotide comprises: (a) a 5′-UTR (e.g., as described herein); (b) a coding region comprising a stop element (e.g., as described herein); (c) a 3′-UTR (e.g., as described herein) and; (d) a 3′ stabilizing region (e.g., as described herein).

In an embodiment, the LNP composition comprises: (i) an ionizable lipid (e.g., an amino lipid); (ii) a sterol or other structural lipid; (iii) a non-cationic helper lipid or phospholipid; and (iv) a PEG-lipid.

### Methods of Making Polynucleotides

### In Vitro Transcription/Enzymatic Synthesis

The present disclosure also provides methods for making a polynucleotide disclosed herein or a complement thereof. In some aspects, a polynucleotide (e.g., an mRNA) disclosed herein encoding an ITB6 molecule can be constructed using in vitro transcription.

In other aspects, a polynucleotide (e.g., an mRNA) disclosed herein encoding an ITB6 molecule can be constructed by chemical synthesis using an oligonucleotide synthesizer. In other aspects, a polynucleotide (e.g., an mRNA) disclosed herein encoding an ITB6 molecule is made by using a host cell. In certain aspects, a polynucleotide (e.g., an mRNA) disclosed herein encoding an ITB6 molecule is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding an ITB6 molecule. The resultant mRNAs can then be examined for their ability to produce protein and/or produce a therapeutic outcome.

While RNA can be made synthetically using methods well known in the art, in one embodiment an RNA transcript (e.g., mRNA transcript) is synthesized by contacting a DNA template with a RNA polymerase (e.g., a T7 RNA polymerase or a T7 RNA polymerase variant) under conditions that result in the production of RNA transcript.

In some aspects, the present disclosure provides methods of performing an IVT (in vitro transcription) reaction, comprising contacting a DNA template with the RNA polymerase (e.g., a T7 RNA polymerase, such as a T7 RNA polymerase variant) in the presence of nucleoside triphosphates and buffer under conditions that result in the production of RNA transcripts.

Other aspects of the present disclosure provide capping methods, e.g., co-transcriptional capping methods or other methods known in the art. In one embodiment, a capping method comprises reacting a polynucleotide template with a T7 RNA polymerase variant, nucleoside triphosphates, and a cap analog under in vitro transcription reaction conditions to produce RNA transcript.

IVT conditions typically require a purified linear DNA template containing a promoter, nucleoside triphosphates, a buffer system that includes dithiothreitol (DTT) and magnesium ions, and a RNA polymerase. The exact conditions used in the transcription reaction depend on the amount of RNA needed for a specific application. Typical IVT reactions are performed by incubating a DNA template with a RNA polymerase and nucleoside triphosphates, including GTP, ATP, CTP, and UTP (or nucleotide analogs) in a transcription buffer. A RNA transcript having a 5′ terminal guanosine triphosphate is produced from this reaction.

A deoxyribonucleic acid (DNA) is simply a nucleic acid template for RNA polymerase. A DNA template may include a polynucleotide encoding an ITB6 polypeptide of interest (e.g., an antigenic polypeptide). A DNA template, in some embodiments, includes a RNA polymerase promoter (e.g., a T7 RNA polymerase promoter) located 5′ from and operably linked to polynucleotide encoding an ITB6 polypeptide of interest. A DNA template may also include a nucleotide sequence encoding a polyadenylation (polyA) tail located at the 3′ end of the gene of interest.

Polypeptides of interest include, but are not limited to, biologics, antibodies, antigens (vaccines), and therapeutic proteins. The term “protein” encompasses peptides.

A RNA transcript, in some embodiments, is the product of an IVT reaction and, as will be understood by one of ordinary skill in the art, the DNA template for making an RNA molecule is known based on base complementarity. A RNA transcript, in some embodiments, is a messenger RNA (mRNA) that includes a nucleotide sequence encoding a polypeptide of interest linked to a polyA tail. In some embodiments, the mRNA is modified mRNA (mmRNA), which includes at least one modified nucleotide.

A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m5UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO4 to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, a RNA transcript (e.g., mRNA transcript) of the present disclosure may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, a RNA transcript (e.g., mRNA transcript) includes a combination of at least two (e.g., 2, 3, 4, or more) of the foregoing modified nucleobases.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The concentration of nucleoside triphosphates and cap analog present in an IVT reaction may vary. In some embodiments, NTPs and cap analog are present in the reaction at equimolar concentrations. In some embodiments, the molar ratio of cap analog (e.g., trinucleotide cap) to nucleoside triphosphates in the reaction is greater than 1:1. For example, the molar ratio of cap analog to nucleoside triphosphates in the reaction may be 2:1, 3:1, 4:1, 5:1, 6:1, 7:1, 8:1, 9:1, 10:1, 15:1, 20:1, 25:1, 50:1, or 100:1. In some embodiments, the molar ratio of cap analog (e.g., trinucleotide cap) to nucleoside triphosphates in the reaction is less than 1:1. For example, the molar ratio of cap analog (e.g., trinucleotide cap) to nucleoside triphosphates in the reaction may be 1:2, 1:3, 1:4, 1:5, 1:6, 1:7, 1:8, 1:9, 1:10, 1:15, 1:20, 1:25, 1:50, or 1:100.

The composition of NTPs in an IVT reaction may also vary. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. The same IVT reaction may include 3.75 millimolar cap analog (e.g., trinucleotide cap). In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5.

In some embodiments, a RNA transcript (e.g., mRNA transcript) includes a modified nucleobase selected from pseudouridine (ψ), 1-methylpseudouridine (m1ψ), 5-methoxyuridine (mo⁵U), 5-methylcytidine (m⁵C), α-thio-guanosine and α-thio-adenosine. In some embodiments, a RNA transcript (e.g., mRNA transcript) includes a combination of at least two (e.g., 2, 3, 4 or more) of the foregoing modified nucleobases.

In some embodiments, a RNA transcript (e.g., mRNA transcript) includes pseudouridine (ψ). In some embodiments, a RNA transcript (e.g., mRNA transcript) includes 1-methylpseudouridine (m1ψ). In some embodiments, a RNA transcript (e.g., mRNA transcript) includes 5-methoxyuridine (mo⁵U). In some embodiments, a RNA transcript (e.g., mRNA transcript) includes 5-methylcytidine (m⁵C). In some embodiments, a RNA transcript (e.g., mRNA transcript) includes α-thio-guanosine. In some embodiments, a RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment of the present invention.

In some embodiments, the buffer system contains tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:1, 1:2, 1:3, 1:4 or 1:5.

In some embodiments, the molar ratio of NTP plus cap analog (e.g., trinucleotide cap, such as GAG) to magnesium ions (Mg²⁺; e.g., MgCl₂) present in an IVT reaction is 1:1 to 1:5. For example, the molar ratio of NTP+trinucleotide cap (e.g., GAG) to magnesium ions may be 1:1, 1:2, 1:3, 1:4, or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

The addition of nucleoside triphosphates (NTPs) to the 3′ end of a growing RNA strand is catalyzed by a polymerase, such as T7 RNA polymerase, for example, any one or more of the T7 RNA polymerase variants (e.g., G47A) of the present disclosure. In some embodiments, the RNA polymerase (e.g., T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml, or 1.0 mg/ml.

In some embodiments, the polynucleotide of the present disclosure is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded ITB6 polypeptide. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding an ITB6 polypeptide therapeutic payload or prophylactic payload can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of an ITB6 polypeptide therapeutic payload or prophylactic payload, or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Exemplary methods of making a polynucleotide disclosed herein include: in vitro transcription enzymatic synthesis and chemical synthesis which are disclosed in International PCT application WO 2017/201325, filed on 18 May 2017, the entire contents of which are hereby incorporated by reference.

### Chemical Synthesis

Standard methods can be applied to synthesize an isolated polynucleotide sequence encoding an isolated polypeptide of interest, such as a polynucleotide of the invention (e.g., a polynucleotide comprising a nucleotide sequence encoding an ITB6 polypeptide). For example, a single DNA or RNA oligomer containing a codon-optimized nucleotide sequence coding for the particular isolated polypeptide can be synthesized. In other aspects, several small oligonucleotides coding for portions of the desired polypeptide can be synthesized and then ligated. In some aspects, the individual oligonucleotides typically contain 5′ or 3′ overhangs for complementary assembly.

A polynucleotide disclosed herein (e.g., a RNA, e.g., an mRNA) can be chemically synthesized using chemical synthesis methods and potential nucleobase substitutions known in the art. See, for example, International Publication Nos. WO2014093924, WO2013052523; WO2013039857, WO2012135805, WO2013151671; U.S. Publ. No. US20130115272; or U.S. Pat. No. 8,999,380 or 8,710,200, all of which are herein incorporated by reference in their entireties.

### Purification

In other aspects, a polynucleotide (e.g., an mRNA) disclosed herein encoding an ITB6 molecule can be purified. Purification of the polynucleotides (e.g., mRNA) encoding an ITB6 molecule described herein can include, but is not limited to, polynucleotide clean-up, quality assurance and quality control. Clean-up can be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide (e.g., mRNA) encoding an ITB6 molecule of the disclosure removes impurities that can reduce or remove an unwanted immune response, e.g., reducing cytokine activity.

In some embodiments, the polynucleotide (e.g., mRNA) encoding an ITB6 molecule of the disclosure is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)). In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide, which encodes an ITB6 molecule disclosed herein increases expression of the an ITB6 molecule compared to polynucleotides encoding the ITB6 molecule purified by a different purification method.

In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide encodes an ITB6 molecule. In some embodiments, the purified polynucleotide encodes a human ITB6 molecule.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC.

In another embodiment, the polynucleotides can be sequenced by methods including, but not limited to reverse-transcriptase-PCR.

### Chemical Modifications of Polynucleotides

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises N1-methyl-pseudouridine (m1ψ) substitutions at one or more or all uridine positions of the nucleic acid.

In some embodiments, a RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Quantification of Expressed Polynucleotides Encoding ITB6

In some embodiments, the polynucleotides of the present invention (e.g., a polynucleotide comprising a nucleotide sequence encoding an ITB6 polypeptide), their expression products, as well as degradation products and metabolites can be quantified according to methods known in the art.

In some embodiments, the polynucleotides of the present invention can be quantified in exosomes or when derived from one or more bodily fluid. As used herein “bodily fluids” include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes can be retrieved from an organ selected from the group consisting of lung, heart, pancreas, stomach, intestine, bladder, kidney, ovary, testis, skin, colon, breast, prostate, brain, esophagus, liver, and placenta.

In the exosome quantification method, a sample of not more than 2 mL is obtained from the subject and the exosomes isolated by size exclusion chromatography, density gradient centrifugation, differential centrifugation, nanomembrane ultrafiltration, immunoabsorbent capture, affinity purification, microfluidic separation, or combinations thereof. In the analysis, the level or concentration of a polynucleotide can be an expression level, presence, absence, truncation or alteration of the administered construct. It is advantageous to correlate the level with one or more clinical phenotypes or with an assay for a human disease biomarker.

The assay can be performed using construct specific probes, cytometry, qRT-PCR, real-time PCR, PCR, flow cytometry, electrophoresis, mass spectrometry, or combinations thereof while the exosomes can be isolated using immunohistochemical methods such as enzyme linked immunosorbent assay (ELISA) methods. Exosomes can also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of polynucleotides remaining or delivered. This is possible because the polynucleotides of the present invention differ from the endogenous forms due to the structural or chemical modifications.

In some embodiments, the polynucleotide can be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified polynucleotide can be analyzed in order to determine if the polynucleotide can be of proper size, check that no degradation of the polynucleotide has occurred. Degradation of the polynucleotide can be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Pharmaceutical Compositions

The present disclosure provides pharmaceutical formulations comprising any of the LNP compositions disclosed herein, e.g., an LNP composition comprising a polynucleotide comprising an mRNA comprising an ITB6 molecule.

In some embodiments, the composition or formulation can contain a polynucleotide comprising a sequence optimized nucleic acid sequence disclosed herein which encodes an ITB6 polypeptide. In some embodiments, the composition or formulation can contain a polynucleotide (e.g., a RNA, e.g., an mRNA) comprising a polynucleotide (e.g., an ORF) having significant sequence identity to a sequence optimized nucleic acid sequence disclosed herein which encodes an ITB6 polypeptide. In some embodiments, the polynucleotide further comprises a miRNA binding site, e.g., a miRNA binding site that binds miR-126, miR-142, miR-144, miR-146, miR-150, miR-155, miR-16, miR-21, miR-223, miR-24, miR-27 and miR-26a.

In some embodiments of the disclosure, the polynucleotides are formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intra-synovial, intrasternal, intrathecal, intrahepatic, intralesional, intracranial, intraventricular, oral, inhalation spray, topical, rectal, nasal, buccal, vaginal, or implanted reservoir intramuscular, subcutaneous, or intradermal delivery. In other embodiments, the polynucleotide is formulated for subcutaneous or intravenous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations

The polynucleotide comprising an mRNA encoding an ITB6 molecule, of the disclosure can be formulated using one or more excipients.

The present invention provides pharmaceutical formulations that comprise a polynucleotide described herein (e.g., a polynucleotide comprising a nucleotide sequence encoding an ITB6 polypeptide). The polynucleotides described herein can be formulated using one or more excipients to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In some embodiments, the pharmaceutical formulation further comprises a delivery agent comprising, e.g., a compound having the Formula (I); or a compound having the Formula (III), (IV), (V), or (VI), or any combination thereof. In some embodiments, the delivery agent comprises an ionizable amino lipid, a helper lipid (e.g., DSPC), a sterol (e.g., Cholesterol), and a PEG lipid (e.g., PEG-DMG), e.g., with a mole ratio in the range of about (i) 40-50 mol % ionizable amino lipid, optionally 45-50 mol % ionizable amino lipid, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %; (ii) 30-45 mol % sterol (e.g., cholesterol), optionally 35-42 mol % sterol, for example, 30-31 mol %, 31-32 mol %, 32-33 mol %, 33-34 mol %, 35-35 mol %, 35-36 mol %, 36-37 mol %, 37-38 mol %, 38-39 mol %, or 39-40 mol %, or 40-42 mol % sterol; (iii) 5-15 mol % helper lipid (e.g., DSPC), optionally 10-15 mol % helper lipid, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % helper lipid; and (iv) 1-5% PEG lipid, optionally 1-5 mol % PEG lipid, for example 1.5 to 2.5 mol %, 1-2 mol %, 2-3 mol %, 3-4 mol %, or 4-5 mol % PEG lipid.

A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, MD, 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ®30]), PLUORINC®F 68, POLOXAMER®188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Oxidation is a potential degradation pathway for mRNA, especially for liquid mRNA Formulations. In order to prevent oxidation, antioxidants can be added to the Formulations. Exemplary antioxidants include, but are not limited to, alpha tocopherol, ascorbic acid, ascorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

In some embodiments, the pH of polynucleotide solutions is maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition or Formulation described here can contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

The pharmaceutical composition or formulation described here can contain a bulking agent in lyophilized polynucleotide formulations to yield a “pharmaceutically elegant” cake, stabilize the lyophilized polynucleotides during long term (e.g., 36 month) storage. Exemplary bulking agents of the present invention can include, but are not limited to sucrose, trehalose, mannitol, glycine, lactose, raffinose, and combinations thereof.

In some embodiments, the pharmaceutical composition or Formulation further comprises a delivery agent. The delivery agent of the present disclosure can include, without limitation, liposomes, lipid nanoparticles, lipidoids, polymers, lipoplexes, microvesicles, exosomes, peptides, proteins, cells transfected with polynucleotides, hyaluronidase, nanoparticle mimics, nanotubes, conjugates, and combinations thereof.

Formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotides are administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

Table of contents for Examples

| | |
|---|---|
| Example 1 | Expression of ITB6 mRNA constructs in dendritic cells |
| Example 2 | Induction of Treg differentiation in vitro |
| Example 3 | Inhibition of antigen-specific CD4+ T cell proliferation in vivo |
| Example 4 | De novo Treg differentiation from naïve antigen-specific |
| | CD4+ T cells in vivo |
| Example 5 | Reduction of IFNγ production of antigen-specific CD8+ T |
| | cells upon peptide restimulation |
| Example 6 | Reduction of weight loss, host B cell depletion, and donor T cell |
| | engraftment, with concurrent Treg expansion in GvHD mice |
| Example 7 | Inhibition of donor T cell proliferation in the spleen of GvHD mice |
| Example 8 | Reduction of Tbet+ cells in CD8 T cell population in the |
| | spleen of GvHD mice |
| Example 9 | Reduction of proinflammatory cytokine levels in GvHD mice |
| Example 10 | Maintaining Treg and host lymphocyte populations in GvHD mice |
| Example 11 | Reduction of weight loss and improved survival in GvHD mice |
| Example 12 | Inhibition of antigen-specific CD4+ T cell proliferation in vivo |
| Example 13 | Evaluation of T cell-dependent antibody response |
| Example 14 | Efficacy of ITB6 LNP in acute GvHD |
| Example 15 | Biomarker Studies |
| Example 16 | Evaluation of ITB6 therapy in an EAE mouse model |

### Example 1: Expression of ITB6 mRNA Constructs in Dendritic Cells

This Example describes the expression of ITB6 mRNA constructs in dendritic cells by mRNA transfection.

5×10⁴ murine bone marrow derived dendritic cell line (JAWSII) were transfected with a human or mouse V5 tagged ITB6 mRNA construct, or mock transfected as a control. RNA solutions were prepared by diluting 1 μL of a 250 μg/mL solution with 25 μL of a reduced serum medium, OptiMEM. To prepare liposomes, a transfecting agent, lipofectamine, was incubated in OptiMEM, at room temperature for 10 minutes. The lipofectamine was then added in a 1:1 ratio to the RNA solution and the resultant mixture was incubated at room temperature for an additional five minutes to afford a solution containing liposomes. The liposome solution as added to the JAWSII cells (50 μL). After 24 hours, the JAWSII cells were fixed with BD Cytofix/Cytoperm at 4° C. for 30 minutes, then washed once with BD Perm/Wash and incubated with Anti-V5 (Alexa647) antibodies at 4° C. for 30 minutes. ITB6 expression was measured by flow cytometry.

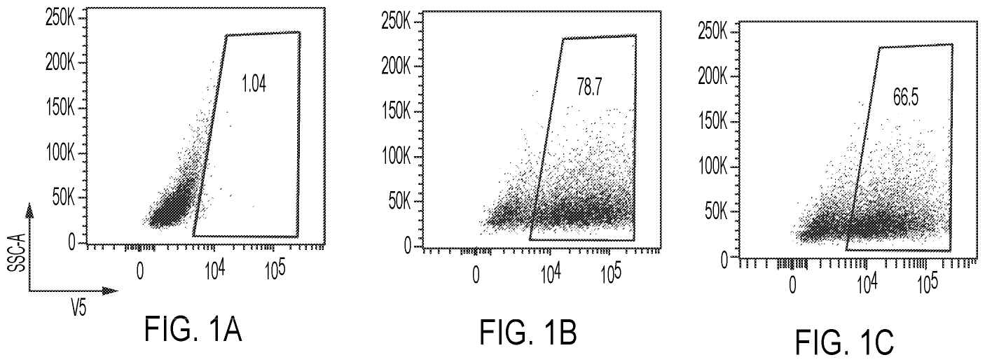

As shown in FIGS. 1A-1C, ITB6 expression was detected in JAWSII cells 24 hours after transfection of the human or mouse ITB6 mRNA construct.

### Example 2: Induction of Treg Differentiation In Vitro

This Example describes the effect of LNP formulated with ITB6 mRNA on Treg differentiation in vitro. ITB8 was also tested but was consistently less efficient than ITB6. Accordingly, subsequent studies focused on ITB6.

1×10⁵ JAWSII cells plated in X-VIVO™ 15 medium (1% HEPES and 0.1% BME) were treated with 50 ng of an LNP formulated with mouse or human ITB6 mRNA in a total culture volume of 100 μL. Control samples were treated with an LNP formulated with untranslatable mRNA (NTFIX), mRNA encoding a dead OX40L (dOX40L), and media lacking any LNP. All cultures were incubated overnight. After 20 to 24 hours, 1×10⁵ CD4+ T cells isolated from Foxp3-GFP (marker of Tregs) mice were added to the cell cultures (1:10 dendritic cell to T cell ratio), followed by 1 μg/mL anti-CD3 antibodies and 5 ng/mL IL-2, in the presence and absence of latent TGFβ(2 and 5 ng/mL) for a final cell culture volume of 200 μL. After 72 hours, cultures were measured by flow cytometry to measure the frequency of Foxp3+GFP+ cells.

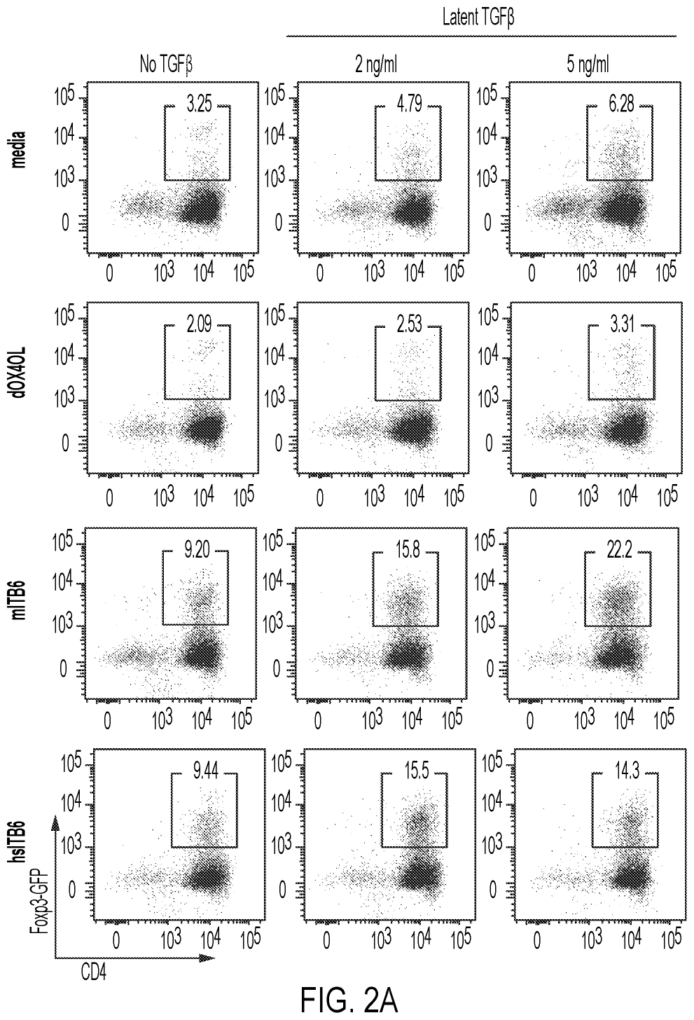

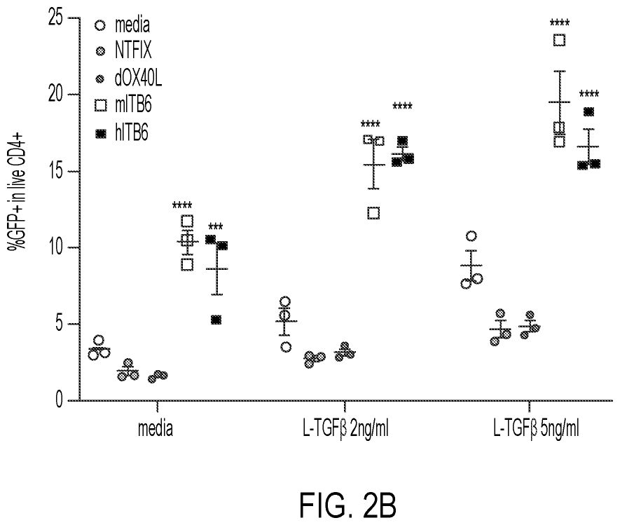

As shown in FIGS. 2A-2B, co-culturing CD4+ T cells with dendritic cells treated with an LNP formulated with human or mouse ITB6 mRNA increased the frequency of Foxp3+GFP+ cells in the CD4+ T cell population. These results indicate that ITB6-formulated LNP induced greater Treg differentiation in vitro in the presence of latent TGFβ.

### Example 3: Inhibition of Antigen-Specific CD4+ T Cell Proliferation In Vivo

This Example describes the effect of LNP formulated ITB6 mRNA on proliferation of antigen-specific CD4+ T cells in vivo.

Purified carboxyfluorescein succinimidyl ester (CFSE) labeled OTH cells (CD45.2+, 3×10⁶ cells) were adoptively transferred into CD45.1+C57BL/6 mice by intravenous injection. Recipient mice were intravenously injected with an LNP formulated with mouse or human ITB6 mRNA at the time of cell transfer, and 75 μg ovalbumin (OVA) was administered one day post transfer. One set of control mice was not subjected to an injection after adoptive transfer. Additional control mice were dosed with OVA-only or an LNP formulated with dOX40L mRNA. Four days after adoptive transfer (i.e., 3 days after OVA injection), the mice were sacrificed, and spleens were harvested for processing of immune cell populations. The immune cell populations were analyzed by flow cytometry.

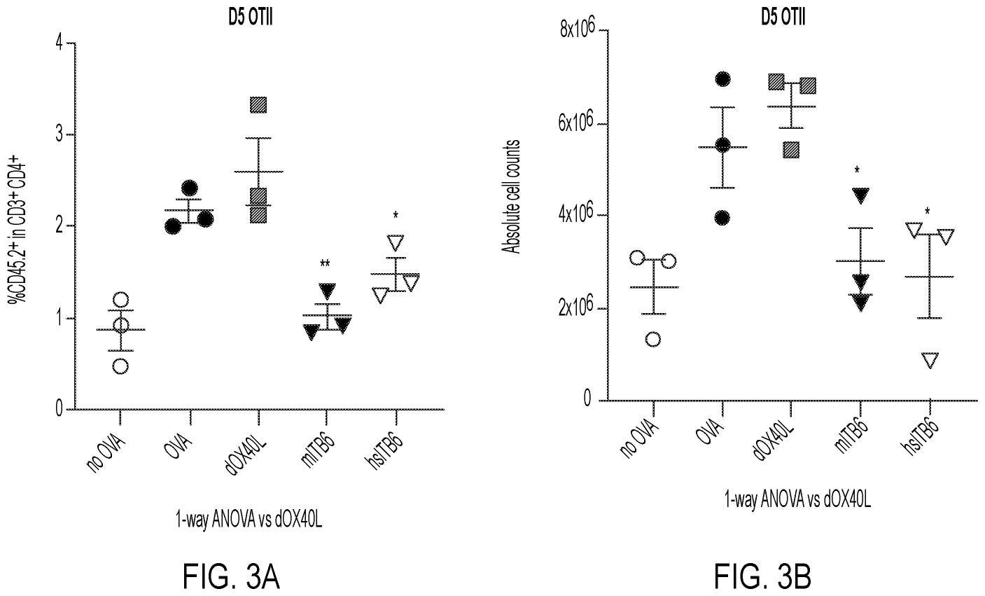

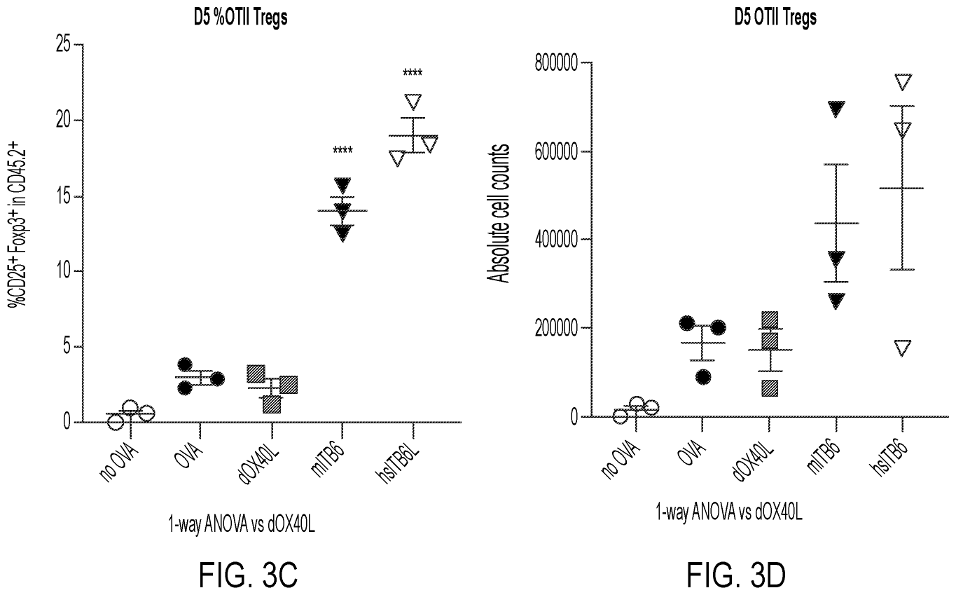

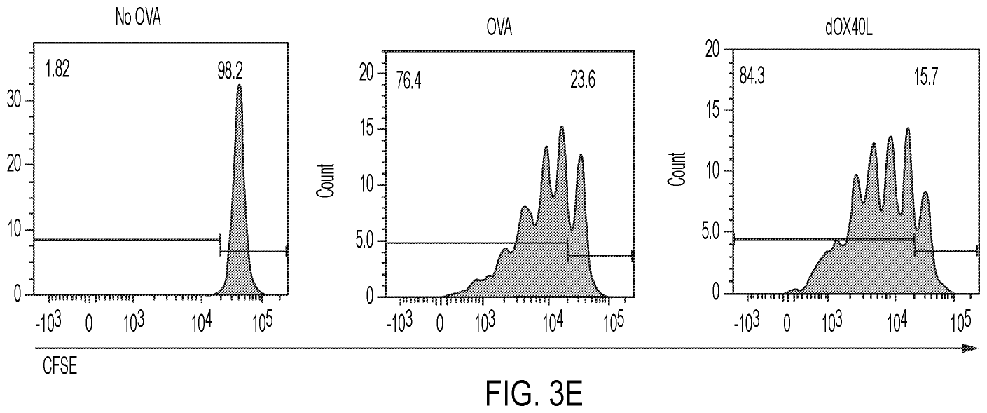

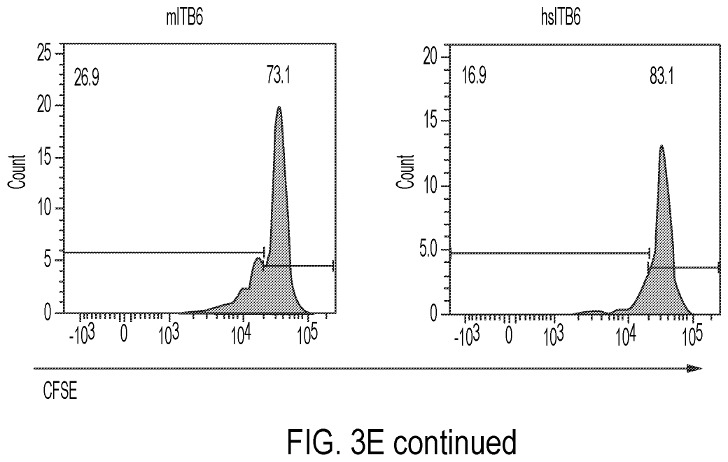

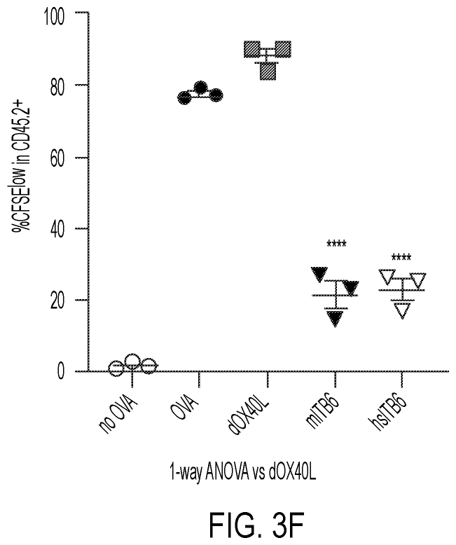

As shown in FIGS. 3A-3B, the frequency and absolute cell counts of OTII (CD45.2+CD4+) cells in the spleen decreased in mice treated with an LNP formulated with mouse or human ITB6 mRNA. As shown in FIGS. 3C-3D, the frequency and absolute cell counts of OTII regulatory T cells (Tregs, CD25+Foxp3+CD45.2+CD4+) in the spleen increased in mice treated with an LNP formulated with mouse or human ITB6 mRNA. As shown in FIGS. 3E-3F, the proliferation of OTII cells in the spleen was inhibited in mice treated with an LNP formulated with mouse or human ITB6 mRNA. These results indicate that ITB6 mRNA-formulated LNP treatment inhibited the proliferation of antigen-specific CD4+ T cells in vivo.

### Example 4: De Novo Treg Differentiation from Naïve Antigen-Specific CD4+ T Cells In Vivo

This Example describes the effect of LNP formulated ITB6 mRNA on de novo Treg differentiation from naïve antigen-specific CD4+ T cells in vivo.

Purified CFSE labeled OTII cells lacking Treg cells (RagKO-OTII cells, CD45.2+, 2×10⁶ cells) were adoptively transferred into CD45.1+C57BL/6 mice by intravenous injection. The RagKO-OTII mice lack Tregs. Recipient mice were intravenously injected with an LNP formulated mouse ITB6 mRNA at the time of cell transfer, and 75 μg ovalbumin (OVA) administered one day post transfer. One set of control mice was not subjected to an injection after adoptive transfer. Additional control mice were treated with OVA-only or an LNP formulated with dOX40L mRNA. Four days after adoptive transfer (i.e., 3 days after OVA injection), the mice were sacrificed, and spleens were harvested for processing of immune cell populations. The immune cell populations were analyzed by flow cytometry.

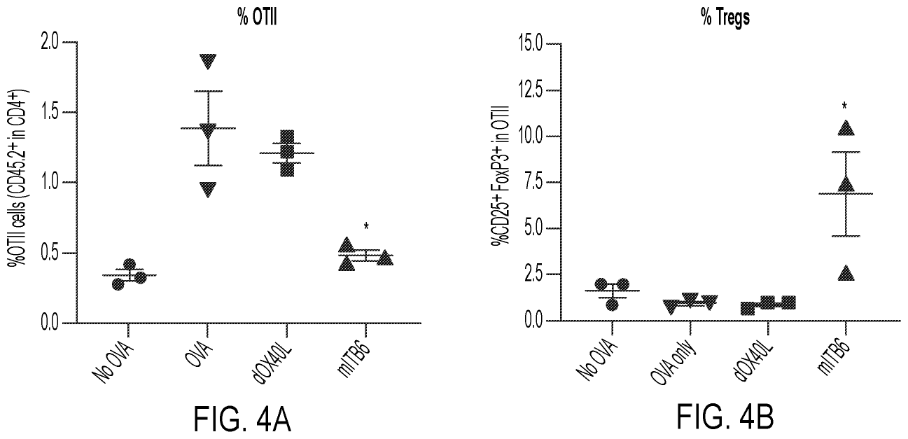

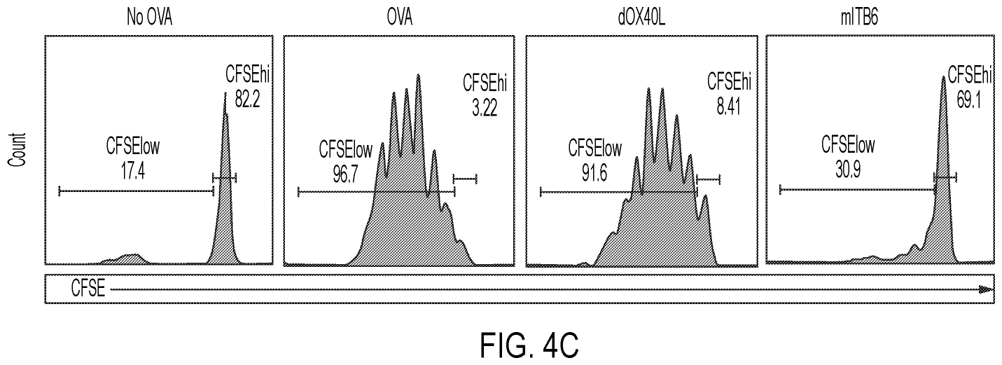

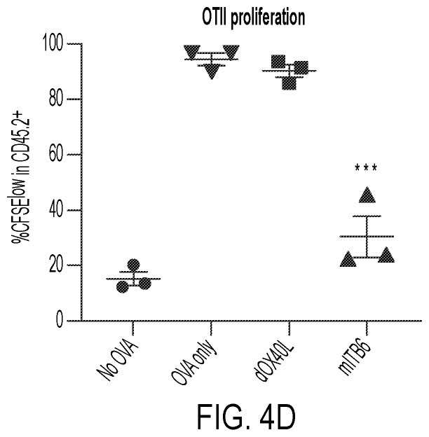

As shown in FIGS. 4A-4B, the frequency of OTH cells (CD45.2+CD4+)) in the spleen decreased in the mice treated with LNP formulated with mouse ITB6 mRNA. In contrast, the frequency of OTII Tregs increased following treatment with LNP formulated with mouse ITB6 mRNA. As shown in FIGS. 4C-4D, the proliferation of OTII cells in the spleen was inhibited in mice treated with an LNP formulated with mouse ITB6 mRNA. These results indicate that ITB6 mRNA-formulated LNP treatment supported de novo Treg differentiation from naïve antigen specific CD4+ T cells in vivo.

### Example 5: Reduction of IFNγ Production of Antigen-Specific CD8+ T Cells Upon Peptide Restimulation

This Example describes the effects of LNP formulated ITB6 mRNA on IFNγ production of antigen-specific CD8+ T cells upon peptide restimulation.

Purified CFSE labeled OTI cells (CD45.2+, 2×10⁶ cells) were adoptively transferred into CD45.1+C57BL/6 mice by intravenous injection. Recipient mice were intravenously injected with LNP formulated with mouse or human ITB6 mRNA at 0.5 mg/kg at the time of cell transfer. One day post-transfer, recipient mice were primed with 20 μg ovalbumin (OVA) by intravenous injection. One set of control mice was not injected with OVA. Additional control mice were treated with OVA-only or an LNP formulated with dOX40L mRNA. Four days after adoptive transfer (i.e., 3 days after OVA injection), the mice were sacrificed, and spleens were harvested for processing of immune cell populations. The immune cell populations were analyzed by flow cytometry. To measure the ability of antigen-specific cells to respond upon restimulation, total splenocytes were incubated in RPMI+10% FBS at 37° C. for 1 hour with 1 μg/mL of the peptide SIINFEKL (SEQ ID NO: 300) (antigen recognized by OTI). After 1 hour, Brefeldin A was added to each culture and cells were incubated for 4 hours. The cultures then underwent intracellular staining for IFNγ.

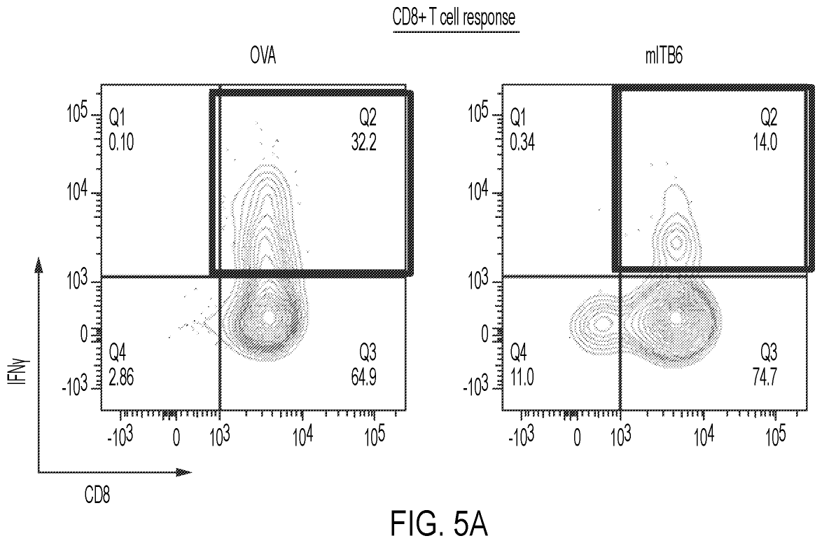

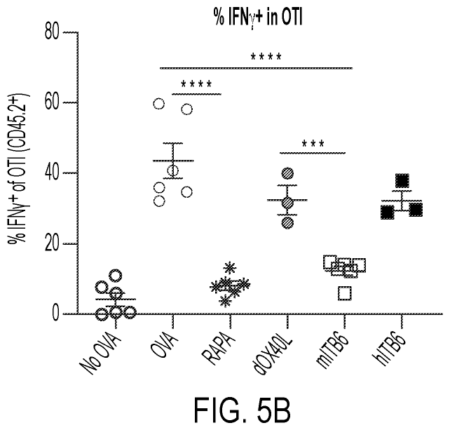

As shown in FIGS. 5A-5B, the frequency of IFNγ+ cells in OTI (CD45.2+) cells was reduced in the splenocytes of mice treated with an LNP formulated with mouse ITB6 mRNA. These results indicate that ITB6-formulated LNP treatment reduced IFNγ production in antigen-specific CD8+ T cells upon peptide restimulation.

### Example 6: Reduction of Weight Loss, Host B Cell Depletion, and Donor T Cell Engraftment, with Concurrent Treg Expansion, in GvHD Mice

This Example describes the effect of LNP formulated with ITB6 mRNA on weight loss, host B cell depletion, donor T cell engraftment, as well as Treg expansion, in an acute Graft vs Host Disease (GvHD) mouse model.

Spleen and lymph nodes were harvested from naïve donor B6-Ly5.5/Cr mice (H2-K^{b}, strain #564 Charles River Labs) and processed into single cell suspensions. The spleen and lymph node cell suspensions were then treated once with 1×ACK to lyse red blood cells. After red blood cell lysis, the pooled splenocytes and lymph node cells were resuspended at a concentration of 5×10⁷ cells/200 μL in 1×PBS. Recipient B6D2F1 mice (H2-K^{d/b}, strain #099 Charles River Labs) were administered 5×10⁷ cells via intravenous tail vein injection. At the time of cell transfer, mice were also administered either 100 μL of 1×PBS or LNP (0.5 mg/kg). Mice were treated with PBS or LNP once a week. Mice were weighed three times a week until day 22 (21 days post transfer), when mice were sacrificed for immunophenotyping. Mice were bled at various timepoints for immunophenotyping of circulating cells.

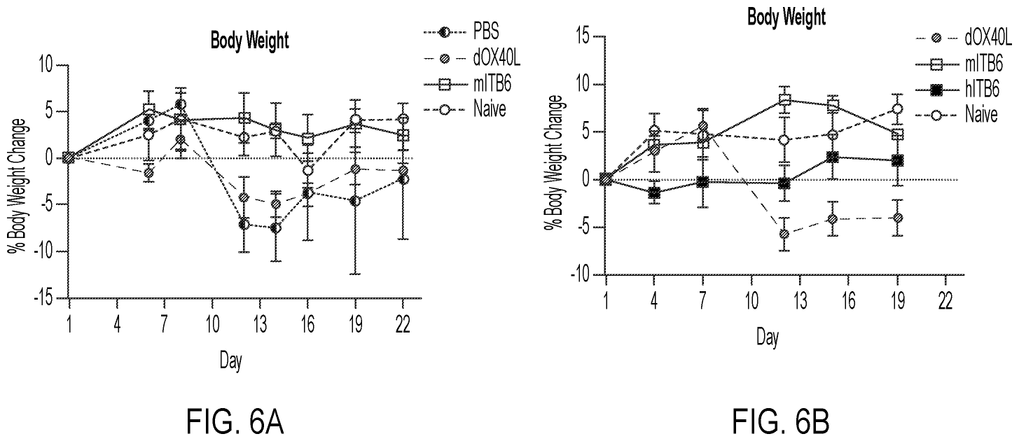

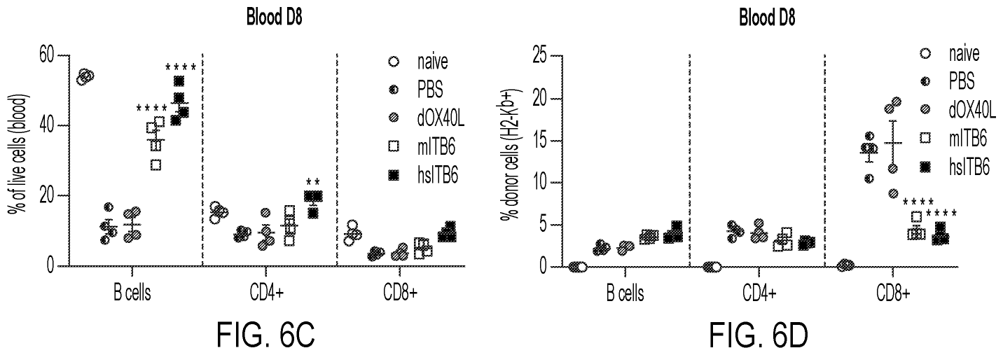

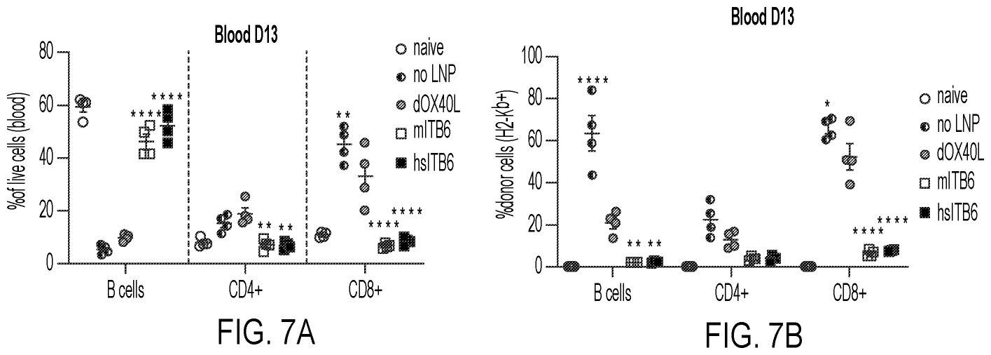

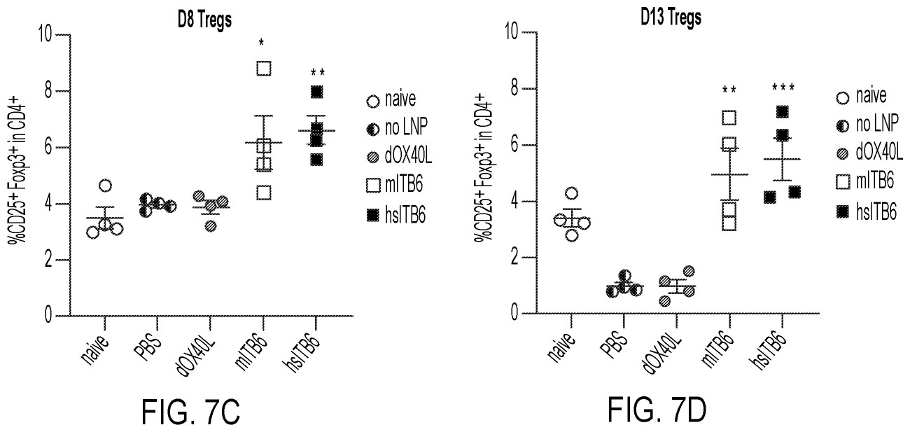

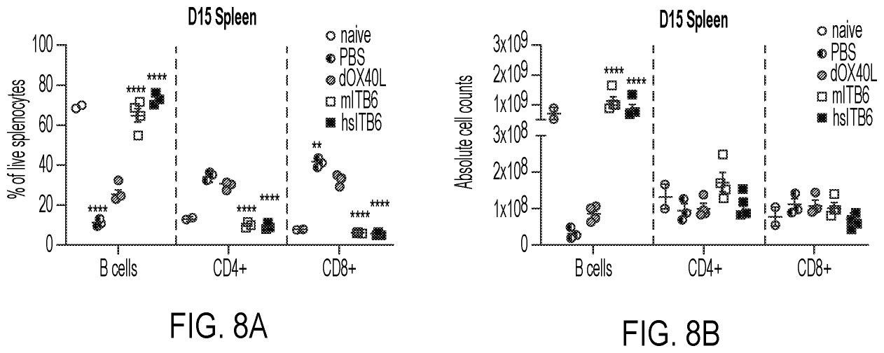

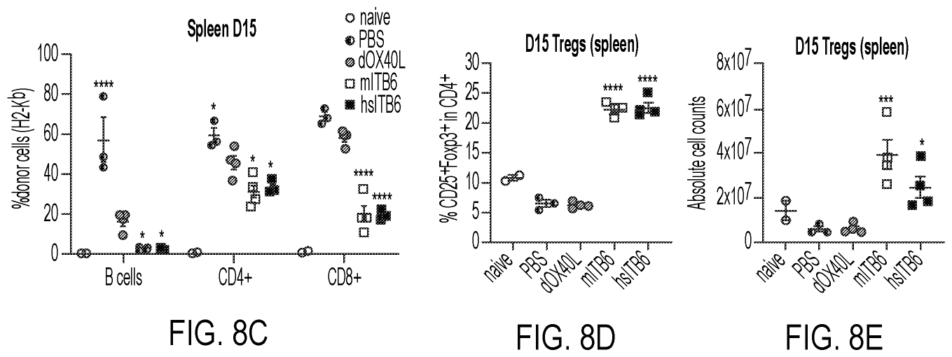

As shown in FIGS. 6A-6B, treatment of GvHD with LNP formulated ITB6 ameliorated weight loss. As shown in FIGS. 6C, 7A, and 8A-8B, treatment of GvHD with LNP formulated ITB6 rescued B cells from depletion. As shown in FIGS. 6D, 7B, and 8C, treatment of GvHD with LNP formulated ITB6 inhibited donor CD8+ T cell engraftment as early as Day 8 and through at least Day 15. As shown in FIGS. 7C-7D and 8D-8E, treatment of GvHD with LNP formulated ITB6 resulted in concurrent expansion of Tregs.

### Example 7: Inhibition of Donor T Cell Proliferation in the Spleen of GvHD Mice

This Example describes the effect of LNP formulated with ITB6 mRNA on donor T cell proliferation in an acute GvHD mouse model.

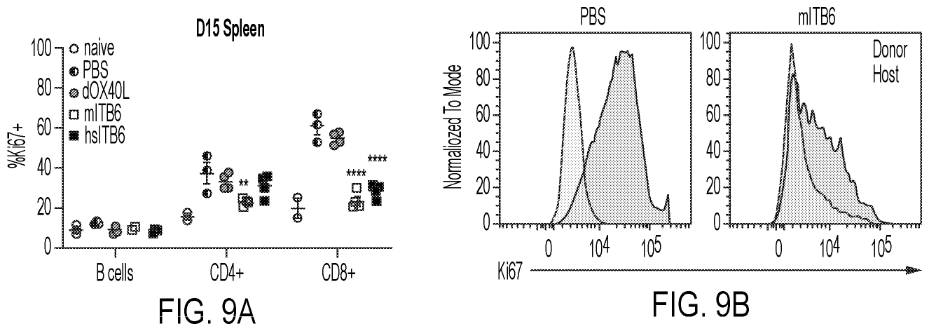

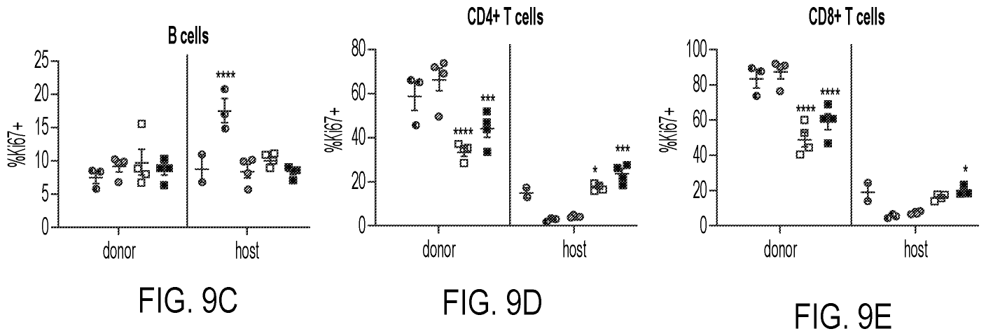

The disease model is described in Example 6. As shown in FIGS. 9A-9E, treatment of GvHD with LNP formulated with ITB6 mRNA inhibited donor T cell proliferation in the spleen.

### Example 8: Reduction of Tbet+ Cells in CD8 T Cell Population in the Spleen of GvHD Mice

This Example describes the effect of LNP formulated with ITB6 mRNA on Tbet+ cells in an acute GvHD mouse model.

The disease model is described in Example 6. Spleens were harvested at days 15 (at peak disease) and 22 from GvHD mice. Spleens harvested on day 15 were analyzed for Tbet expression in CD8+ and conventional CD4+ T cells. Tbet is a regulator of IFNγ production.

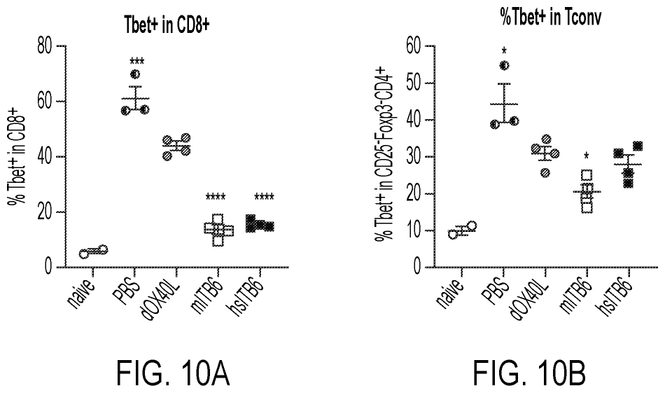

As shown in FIGS. 10A-10B, treatment with LNP formulated with ITB6 mRNA resulted in reduction of Tbet+ cells in the CD8+ T cell population in the spleen of the GvHD mice. These results indicate that treatment of GvHD with LNP formulated with ITB6 mRNA decreased inflammatory environment and decreased activation of CD8+ T cells.

### Example 9: Reduction of Proinflammatory Cytokine Levels in GvHD Mice

This Example describes the effect of LNP formulated with ITB6 mRNA on proinflammatory cytokine levels in an acute GvHD mouse model.

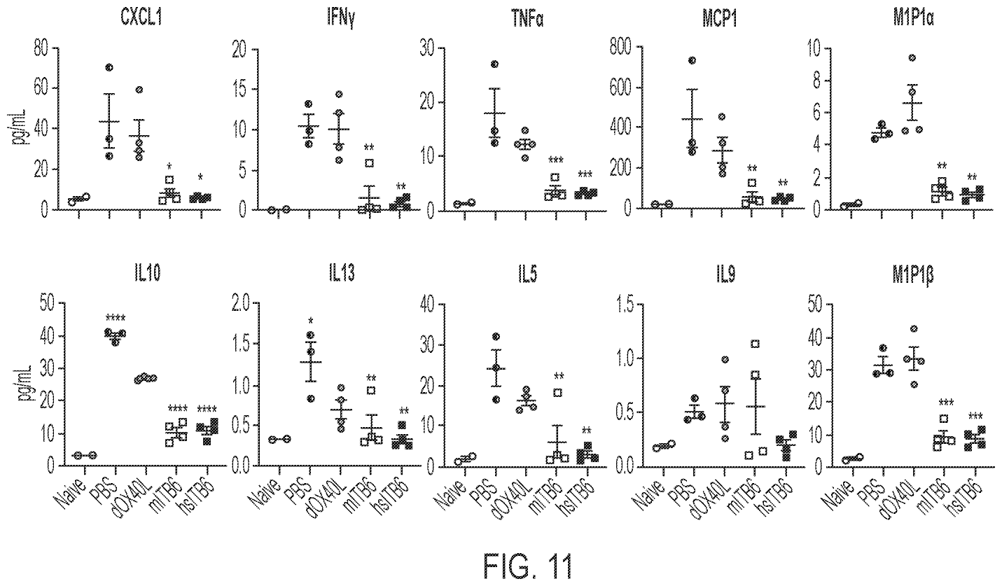

The disease model is described in Example 6. Serum collected from GvHD mice at day 15 was analyzed using a FirePlex® mouse cytokine array (Abcam) to measure cytokine levels. As shown in FIG. 11, mice treated with LNP formulated with ITB6 mRNA exhibited lower level of inflammatory cytokines, including CXCL1, IFNγ, TNFα, MCP1, M1P1α, IL10, IL13, IL5, IL9, and M1P1β.

### Example 10: Maintaining Treg and Host Lymphocyte Populations in GvHD Mice

This Example describes the effect of LNP formulated with ITB6 mRNA on maintaining Treg and host lymphocyte populations in an acute GvHD mouse model.

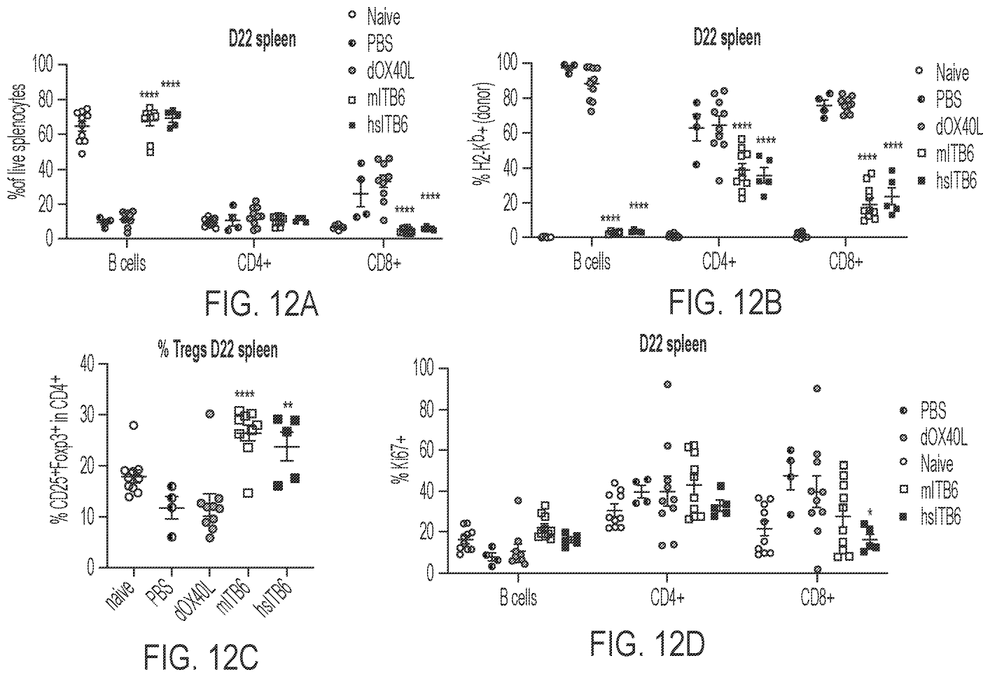

The disease model is described in Example 6. Spleens harvested on day 22, at the end of the study, were immunophenotyped for lymphocyte populations. As shown in FIGS. 12A-12D, treatment with LNP formulated with ITB6 mRNA helped maintain T reg cell and host lymphocyte populations in the spleen of the GvHD mice.

### Example 11: Reduction of Weight Loss and Improved Survival in GvHD Mice

This Example describes the effect of LNP formulated with ITB6 mRNA on weight loss and survival in a xeno-Graft vs Host Disease (GvHD) humanized mouse model.

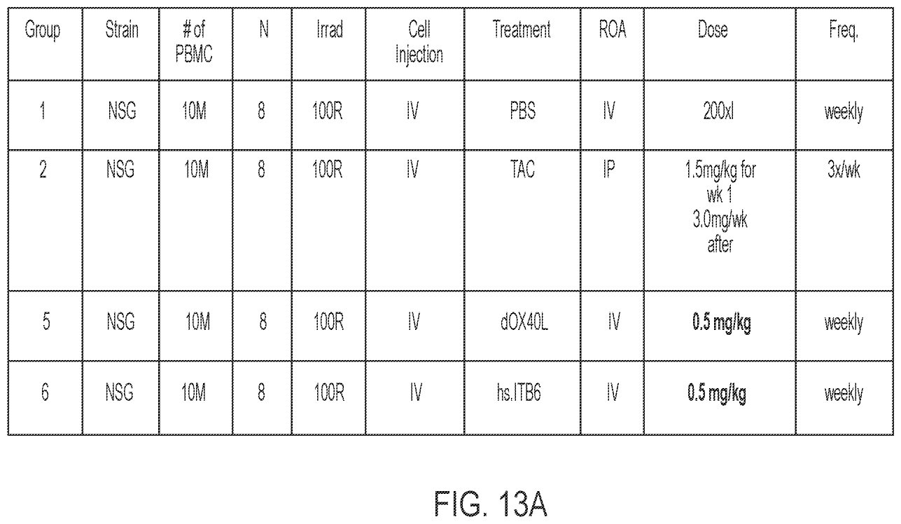

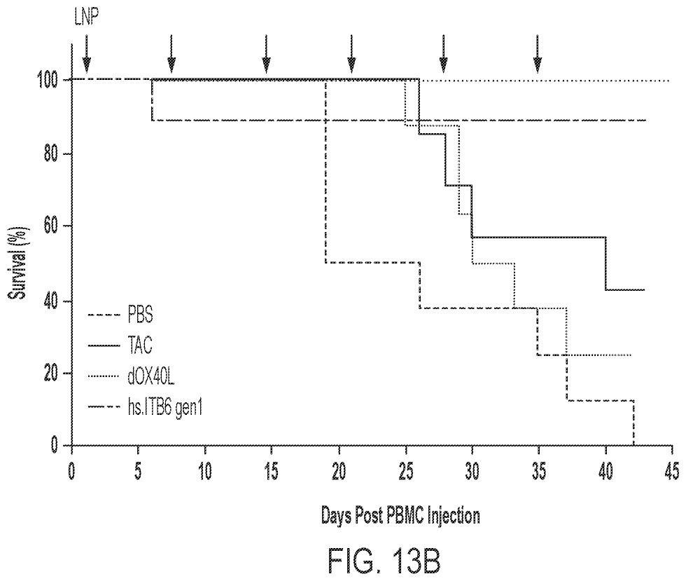

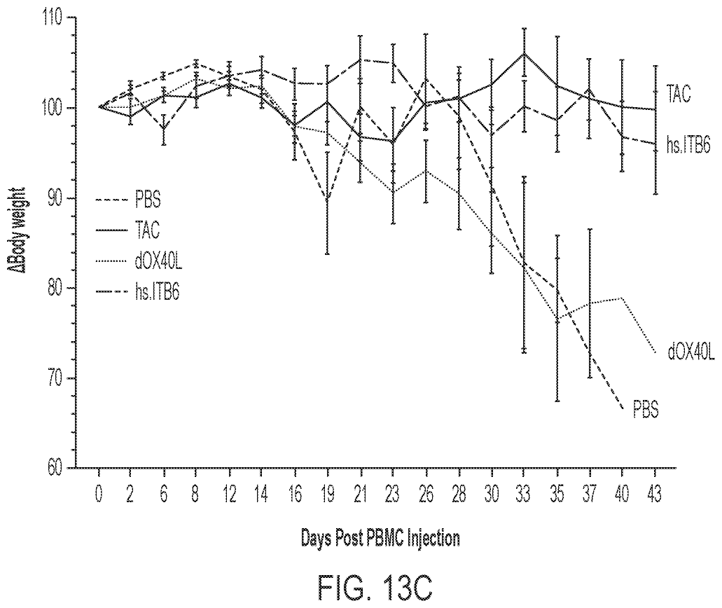

A second model of GvHD utilized introduction of human peripheral blood mononuclear cells (PBMC) into NOD-scid-gamma (NSG) mice. Briefly, 10e6 human PBMCs were injected into irradiated NSG mice to initiate disease. Mice then received weekly doses of LNP, beginning one day post transfer, throughout the 42-day study (FIG. 13A). As shown in FIG. 13B, mice receiving either PBS or negative control LNP exhibited a high degree of mortality (75-100% death). As an additional control, one arm received high dose tacrolimus to control disease. Even with the aggressive treatment of tacrolimus, ˜50% mortality was observed (FIG. 13B). In contrast, as shown in FIGS. 13B and 13C, administration of ITGB6 resulted in 90% survival with no weight loss of the animals. Despite the greatly improved survival, the human lymphocytes were still able to engraft when ITGB6 was administered. However, the CD8+ T cells showed reduced effector function as evidenced by the reduced frequency of Granzyme B positive CD8 T cells (data not shown). These results demonstrate that ITGB6 may prevent activation of the donor cells or may render them anergic.

### Example 12: Inhibition of Antigen-Specific CD4+ T Cell Proliferation In Vivo

This Example describes the effect of LNP formulated ITB6 mRNA on proliferation of antigen-specific CD4+ T cells and T regs in vivo.

Purified carboxyfluorescein succinimidyl ester (CFSE) labeled OTII cells (CD45.2+, 3×10⁶ cells) were adoptively transferred into B6.SJL-PtprcaPepcb/BoyCrl mice by intravenous injection. Recipient mice were intravenously injected with an LNP formulated with human ITB6 mRNA at a dose of 0.5 mpk, 0.33 mpk, 0.1 mpk, 0.05 mpk, or 0.033 mpk at the time of cell transfer, and 75 μg ovalbumin (OVA) was administered one day post transfer. One set of control mice was not subjected to an injection after adoptive transfer. Additional control mice were dosed with OVA-only or an LNP formulated with dOX40L mRNA. Four days after adoptive transfer (i.e., 3 days after OVA injection), the mice were sacrificed, and spleens were harvested for processing of immune cell populations. The immune cell populations were analyzed by flow cytometry.

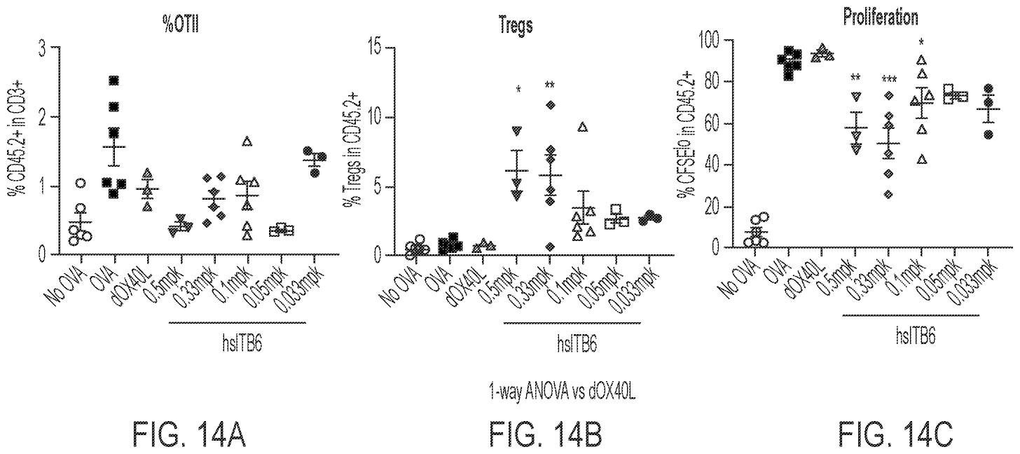

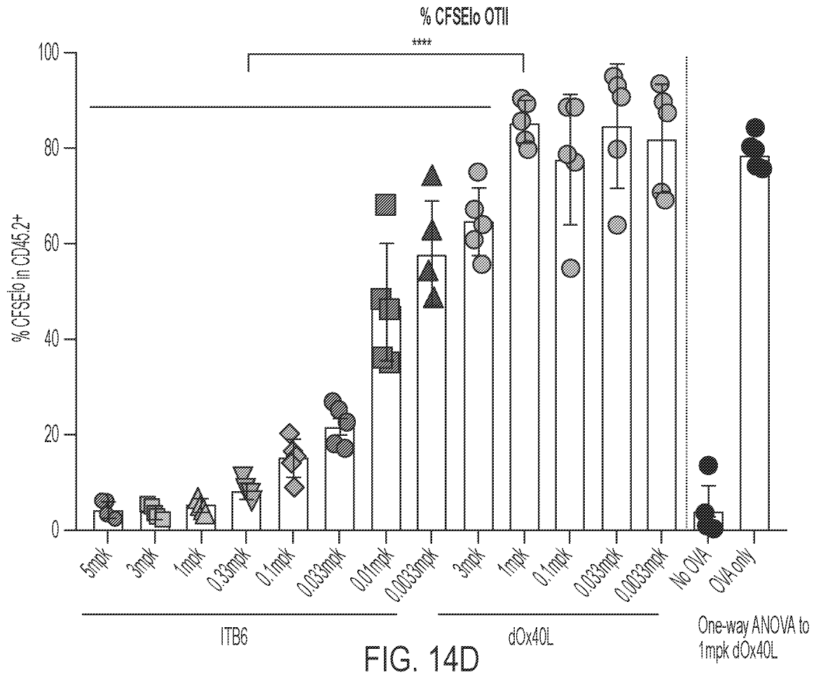

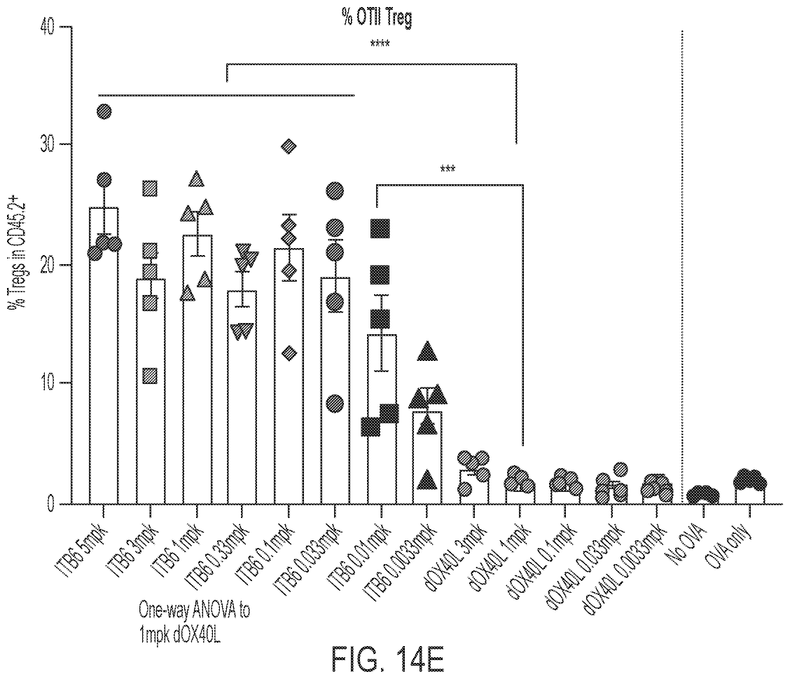

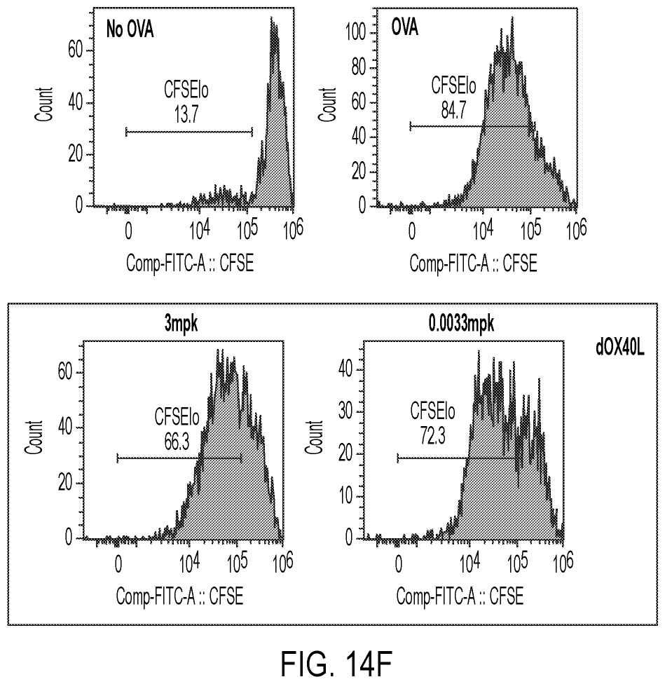

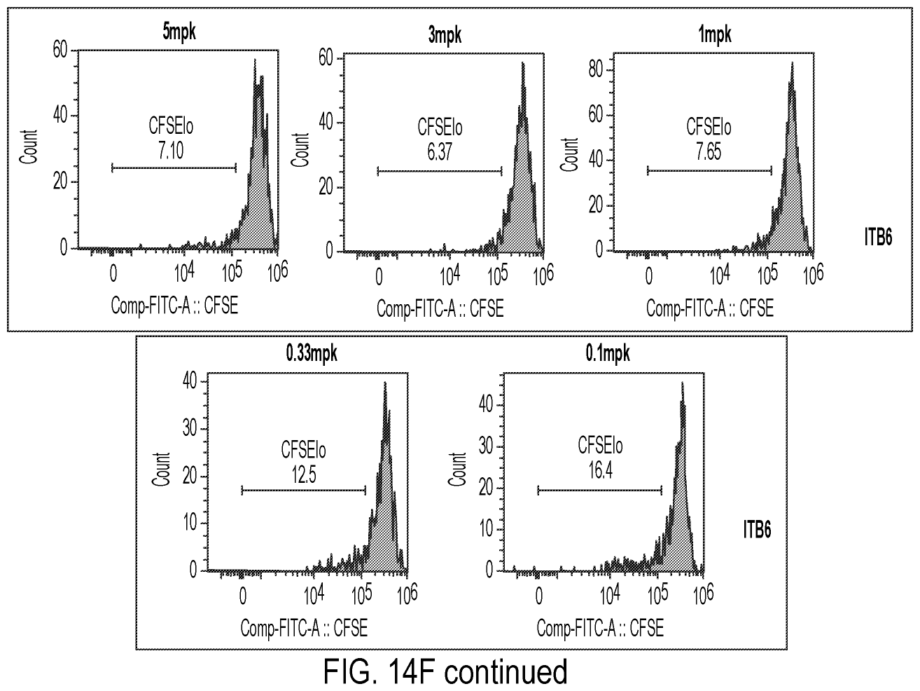

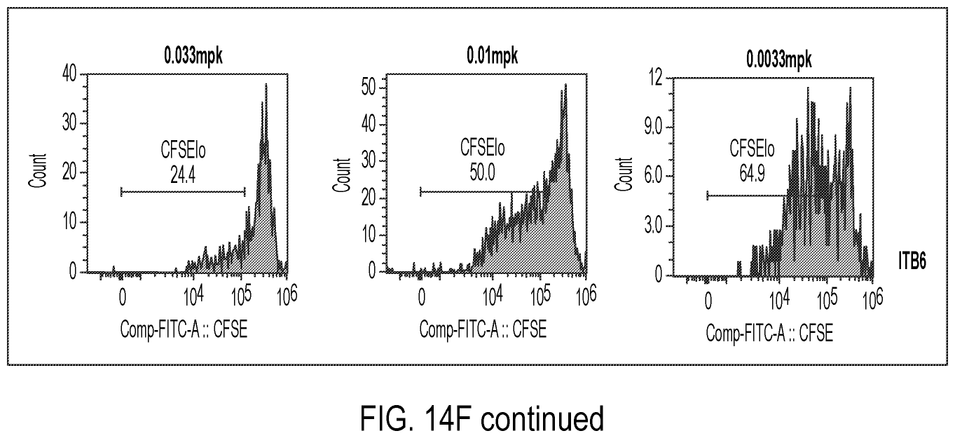

As shown in FIGS. 14A and 14D, the frequency of OTII (CD45.2+CD4+) cells in the spleen decreased in mice treated with an LNP formulated with human ITB6 mRNA in a dose-dependent manner. As shown in FIGS. 14B and 14E, the frequency of OTII regulatory T cells (Tregs, CD25+Foxp3+CD45.2+CD4+) in the spleen increased in mice treated with an LNP formulated with human ITB6 mRNA.

As shown in FIGS. 14C and 14F, the proliferation of OTII cells in the spleen was inhibited in mice treated with an LNP formulated with human ITB6 mRNA.

These results indicate that ITB6 mRNA-formulated LNP treatment inhibited the proliferation of antigen-specific CD4+ T cells in vivo.

### Example 13: Evaluation of T Cell-Dependent Antibody Response

This example describes the effect of LNP formulated with ITB6 mRNA on KLH-specific IgM and IgG responses in mice.

Briefly, mice were dosed intravenously with 250 μg keyhole limpet hemocyanin (KLH) on day 1 and boosted with 150 μg on day 15. Mice were subsequently administered LNPs formulated with mRNA encoding dOX40L (as a control) or ITB6 mRNA. In certain groups, mice were administered a single dose (day 1), two doses (day 1 & 8), or three doses (day 1, 8, & 15) of the LNP formulations. Blood was collected from mice on days 7, 14, and 21. The levels of KLH-specific IgM and IgG were measured from collected samples.

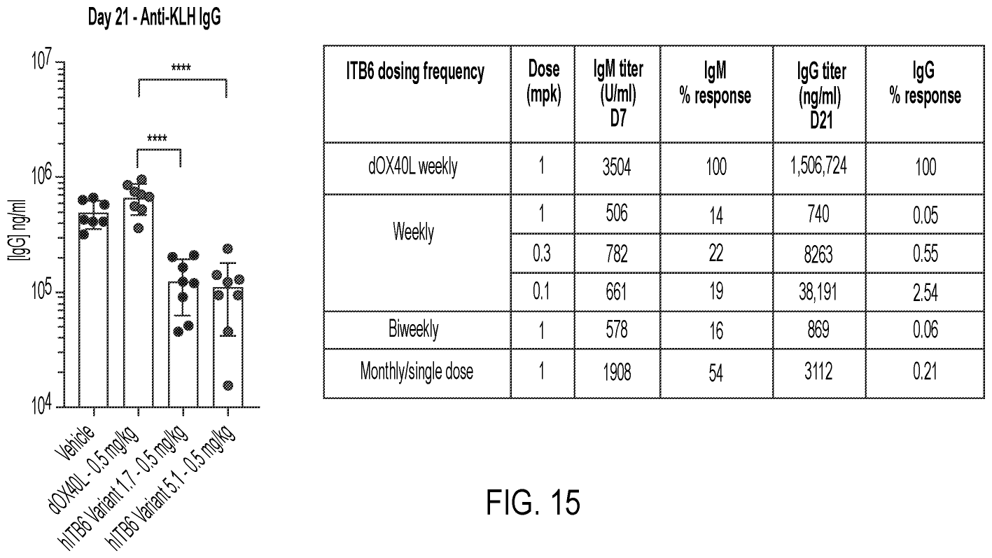

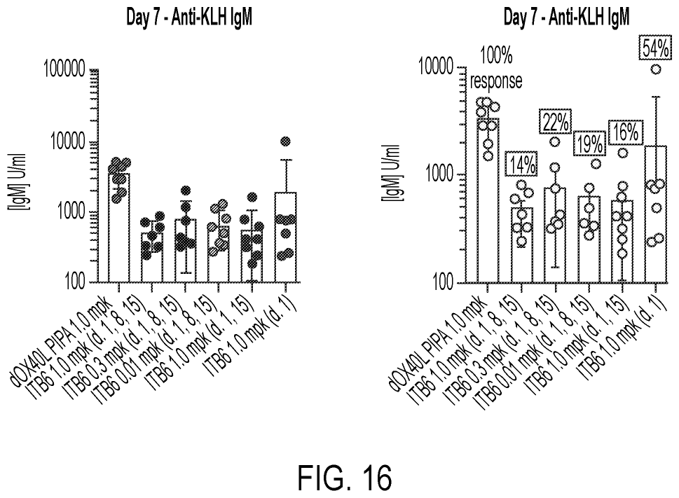

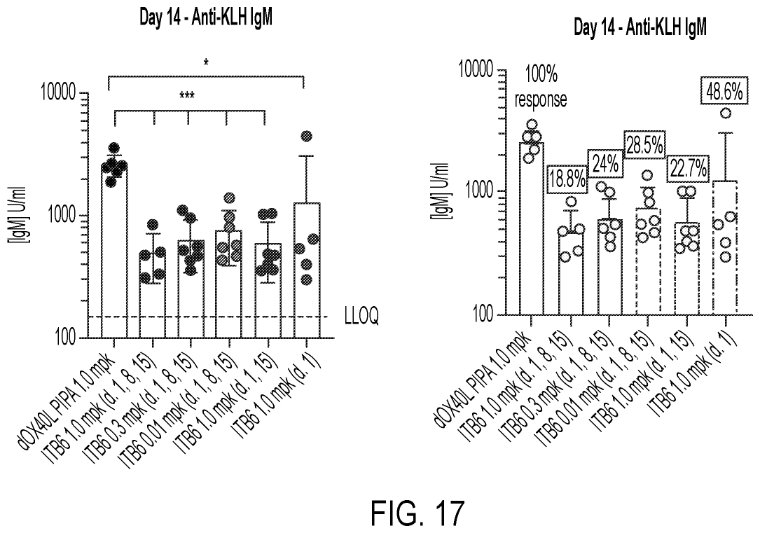

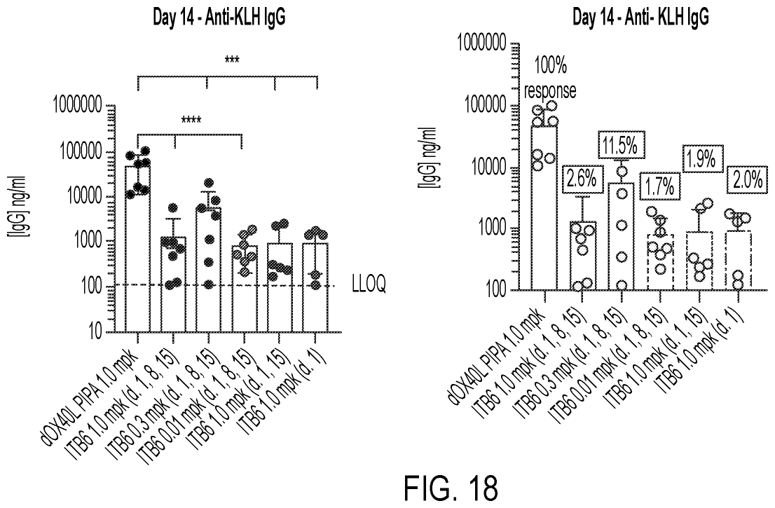

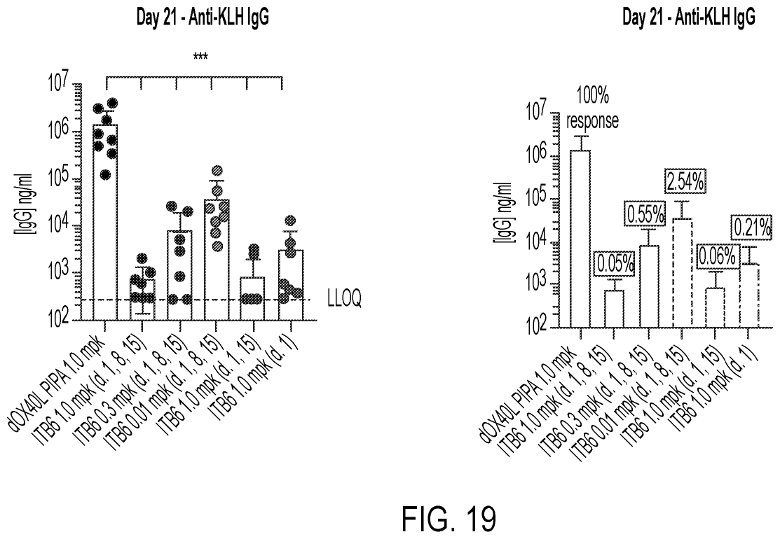

As shown in FIGS. 15-19, ITB6 reduced KLH-specific IgM and IgG levels compared to controls

### Example 14: Efficacy of ITB6 LNP in Acute GvHD

This example describes the effect of LNP formulated with ITB6 mRNA on the host lymphocyte populations in an acute GvHD mouse model.

Spleen and lymph nodes were harvested from naïve donor B6-mice (H2-K^{b/b}) and processed into single cell suspensions. The spleen and lymph node cell suspensions were then treated once with 1×ACK to lyse red blood cells. After red blood cell lysis, the pooled splenocytes and lymph node cells were resuspended in 1×PBS. Recipient B6D2F1 mice (H2-K^{d/b}) were administered 5×10⁷ cells via intravenous tail vein injection. At the time of cell transfer, mice were also administered LNP formulated with mRNA encoding dOX40L or ITB6 (0.033 mg/kg). Mice were treated with LNPs as single dose, weekly, or biweekly. Mice were sacrificed for immunophenotyping at day 22 (21 days post transfer). Mice were bled at various time points for immunophenotyping of circulating cells.

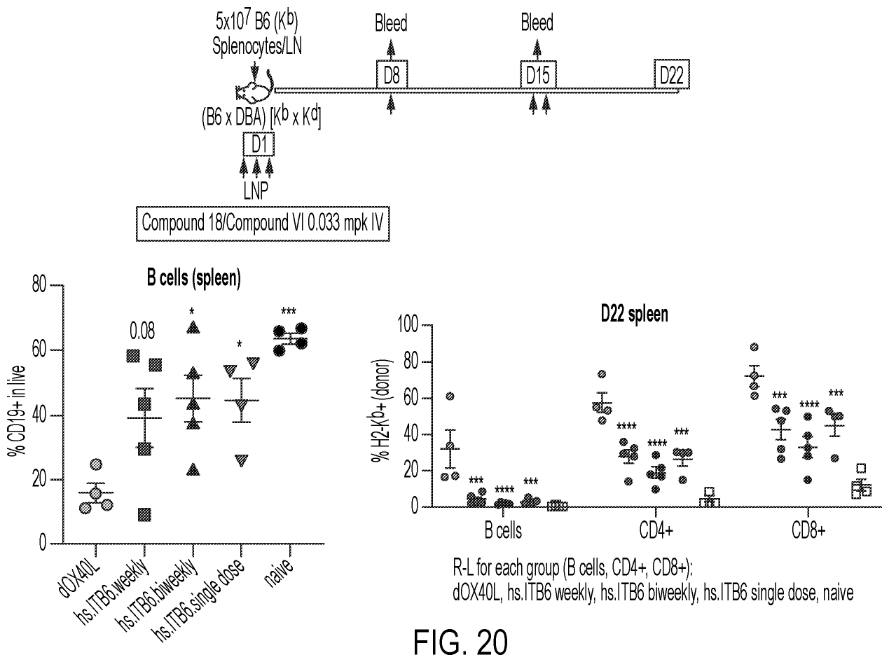

As shown in FIG. 20, treatment with LNP formulated with ITB6 mRNA helped maintain host lymphocyte populations in the spleen of the GvHD mice.

### Example 15: Biomarker Studies

This example describes a transcriptomic study that was conducted to identify and assess potential biomarkers as surrogates for measuring efficacy of ITB6 therapy that are differentially expressed following treatment with ITB6 mRNA.

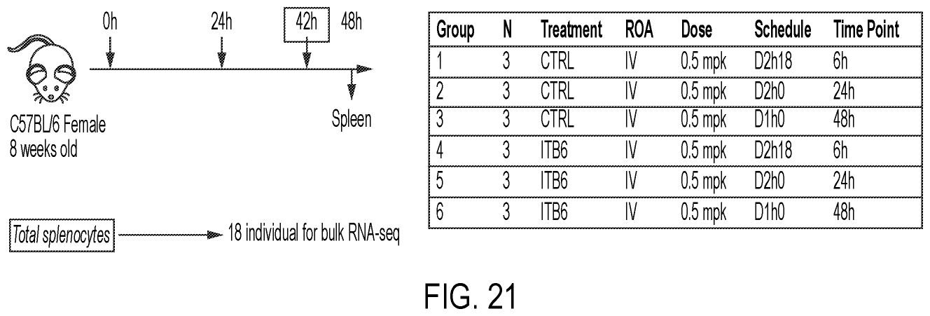

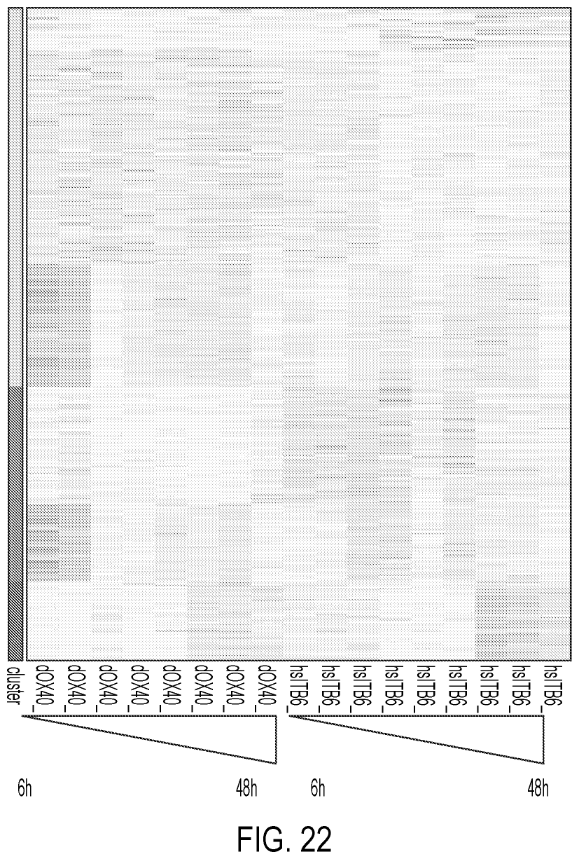

Briefly, 8-week old, female C57BL/6 mice were treated IV with either an LNP formulated with an mRNA encoding a dead OX40L (dOX40L) control or ITB6 mRNA according to the schedule indicated in FIG. 21. Animals were sacrificed at indicated time points and splenocytes were harvested, prepared, and analyzed by bulk RNA-sequencing. Raw sequencing results were aligned to mouse genome using STAR and the gene expression was quantified using HTseq count (FIG. 22).

The counts matrix from all the samples were further analyzed for identifying differentially expressed genes using custom scripts and DESeq2.

From an original list of 776 genes, 12 were identified for further investigation. To this end, further analyses were performed on PBMCs isolated from mouse, rat, and NHP dosed with ITB6 or control mRNA, and human PBMCs treated with ITB6 or control mRNA in vitro.

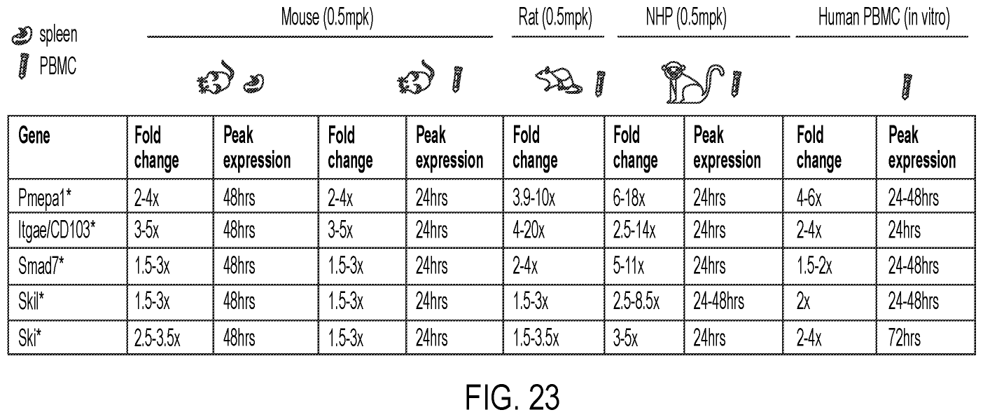

From these studies, the six genes (Pmepa1, Itgae/CD103, Smad7, Skil, and Ski) downstream of the TGFb pathway were identified as biomarkers of interest for monitoring the efficacy of ITB6 therapy (FIG. 23).

### Example 16: Evaluation of ITB6 Therapy in an EAE Mouse Model

This example describes a study conducted in a mouse model of EAE to explore the effects of treatment with LNPs formulated with mRNA encoding ITB6.

Briefly, EAE was induced in 12-week-old female C57bl/6 mice. Mice received 100 μg of rat MOG₃₅₋₅₅ in 100 μl of CFA distributed to two sites on the lower back (50 μl/site). CFA was supplemented to 2.5 mg/ml with killed M. tuberculosis (H37 RA). Mice then received 200 ng/mouse Pertussis toxin i.p. on days 0 and 2. Mice were administered LNPs formulated with dmOX40L, LNPs formulated with mRNA encoding ITB6, or vehicle controls at a dose of 0.5 mpk i.v. on days −1, 6, and 9. Mice were evaluated and scored for EAE starting on day 10.

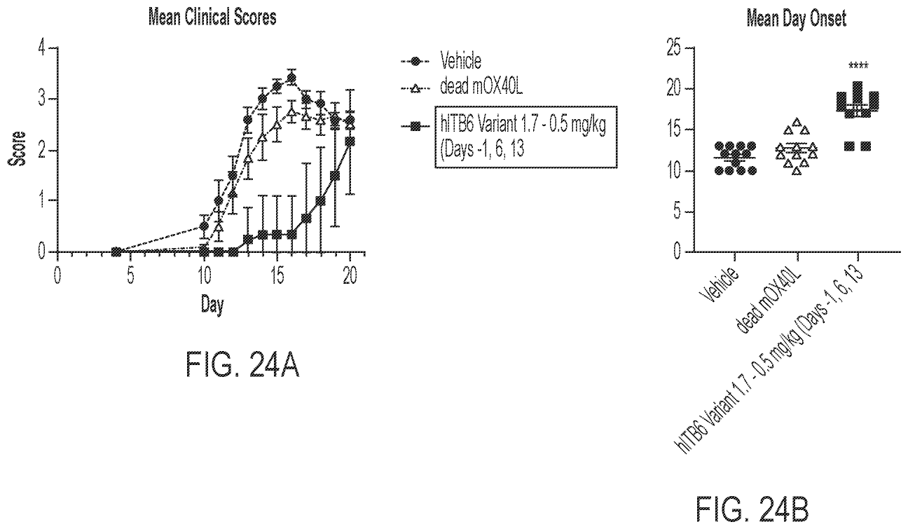

As shown in FIG. 24, treatment with LNPs formulated with mRNA encoding ITB6 at 0.5 mpk dosed IV on days −1, 6, and 13 results in decreased clinical score (FIG. 24A) and delayed disease onset (FIG. 24B).

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A lipid nanoparticle (LNP) composition comprising a polynucleotide comprising an mRNA which encodes an ITB6 molecule.

2. A lipid nanoparticle (LNP) composition for immunomodulation, e.g., for inducing immune tolerance or reprogramming dendritic cells, the composition comprising a polynucleotide comprising an mRNA which encodes an ITB6 molecule, wherein the polynucleotide comprises an mRNA.

3. The LNP composition of claim 1 or 2, wherein the ITB6 molecule comprises an amino acid sequence having at least 85%, 90%, 95%, 96%, 97%, 98%, 99% or 100% identity to an amino acid sequence of ITB6 provided in Table 1A or Table 2A, e.g., any one of SEQ ID NOs: 17, 1, 7, 9, 11, 13, or 15.

4. The LNP composition of claim 3, wherein the ITB6 molecule comprises an amino acid sequence of any one of SEQ ID NOs: 17, 1, 7, 9, 11, 13, or 15.

5. The LNP composition of claim 1 or 2, wherein the mRNA comprises a nucleic acid sequence having at least 85%, 90%, 95%, 96%, 97%, 98%, 99% or 100% identity to a nucleic acid sequence of any one of SEQ ID NOs: 17, 1, 7, 9, 11, 13, or 15.

6. The LNP composition of any one of claims 1-5, which results in suppression of T cell activity and/or function (e.g., T cell anergy, and/or T cell apoptosis) in a population of immune cells, e.g., as compared to T cell activity and/or function in an otherwise similar population of cells which has not been contacted with the LNP composition comprising a polynucleotide comprising an mRNA which encodes an ITB6 molecule.

7. The LNP composition of claim 6, wherein suppression of T cell activity and/or function comprises any one, two, three, or all of the following:
(i) increased level of Treg differentiation;
(ii) reduced T cell proliferation, survival and/or expansion (e.g., reduced CD4+ T cell proliferation, survival and/or expansion);
(iii) reduced expression, activity and/or secretion of an effector cytokine (e.g., IFNg); and/or
(iv) reduced expression and/or activity of a T cell transcription factor (e.g., T-bet).

8. The LNP composition of any one of claims 1-7, wherein the LNP composition reduces the level (e.g., expression) and/or activity of a costimulatory molecule, e.g., CD80, CD86, and/or MHCII, in a sample upon stimulation.

9. The LNP composition of any one of claims 1-8, which results in:
(i) reduced donor immune cell (e.g., T cell) proliferation;
(ii) reduced weight loss, host B cell depletion, and/or donor immune cell (e.g., T cell) engraftment, optionally with concurrent Treg expansion;
(iii) reduced Tbet+ cells in T cell population (e.g., CD8+ T cell population);
(iv) reduced expression, activity and/or secretion of a proinflammatory cytokine (e.g., IFNg); and/or
(v) maintained or increased Treg and/or host lymphocyte population.

10. The LNP composition of any one of the preceding claims, which results in amelioration or reduction of graft vs. host disease, e.g., reduction of weight loss and/or B cell depletion, e.g., as described herein, in a subject, e.g., as measured by an assay described in Example 6.

11. The LNP composition of any one of the preceding claims, which results in a reduction of blood glucose levels in a sample, e.g., a sample from a subject.

12. The LNP composition of any one of the preceding claims, wherein the polynucleotide comprising an mRNA encoding the ITB6 molecule, comprises at least one chemical modification.

13. The LNP composition of claim 12, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

14. The LNP composition of any one of the preceding claims, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

15. The LNP composition of claim 14, wherein the ionizable lipid comprises Compound 18.

16. The LNP composition of claim 14, wherein the ionizable lipid comprises Compound 25.

17. A method of modulating, e.g., suppressing, an immune response in a subject, comprising administering to the subject in need thereof an effective amount of an LNP composition comprising an mRNA which encodes an ITB6 molecule.

18. A method of treating or preventing a symptom of, a disease with aberrant T cell function, e.g., an autoimmune disease or an inflammatory disease, or a symptom thereof, comprising administering to the subject in need thereof an effective amount of an LNP composition comprising an mRNA which encodes an ITB6 molecule.

19. The method of claim 18, wherein the disease is chosen from: rheumatoid arthritis (RA); graft versus host disease (GVHD) (e.g., acute GVHD or chronic GVHD); diabetes, e.g., Type 1 diabetes; inflammatory bowel disease (IBD); lupus (e.g., systemic lupus erythematosus (SLE)), multiple sclerosis; autoimmune hepatitis (e.g., Type 1 or Type 2); primary biliary cholangitis; organ transplant associated rejection; or myasthenia gravis.

20. The method of any one of claims 17-19, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

21. The method of claim 20, wherein the ionizable lipid comprises Compound 18.

22. The method of claim 20, wherein the ionizable lipid comprises Compound 25.

23. A method of assessing the responsiveness of a subject to a therapy comprising an LNP composition comprising an mRNA which encodes an ITB6 molecule, the method comprising:
(a) measuring the expression level of one or more biomarkers (e.g., selected from the group consisting of: PMEPA1, ITGAE/CD103, SMAD7, SKIL, and SKI) in a sample from the subject collected following administration of the therapy; and
(b) comparing the expression level in the sample with a reference expression level, wherein an increase in the expression level of the one or more biomarkers is indicative of a response to the therapy.

24. The method of claim 23, wherein the one or more biomarkers are one or more of PMEPA1, ITGAE/CD103, SMAD7, SKIL, and SKI.

25. The method of claim 23 or 24, wherein the level of one or more of the one or more biomarkers in the sample from the subject following treatment is at least 2-fold greater than a reference expression level of the one or more biomarkers, wherein the reference expression level comprises:
(a) the expression level of the one or more biomarkers in a sample from the subject prior to treatment with an ITB6 mRNA; or
(b) the expression level of the one or more biomarkers in a sample from a subject who has not received the treatment with an ITB6 mRNA.
